(19) United States
(12) Patent Application Publication
(10) Pub. No.: US 2023/0337677 A1
(43) Pub. Date: Oct. 26, 2023

(54) ARYL AND PYRIDYL AMIDE PESTICIDES AND COMPOSITIONS THEREOF

(71) Applicant: University of Florida Research Foundation, Incorporated, Gainesville, FL (US)

(72) Inventors: Jeffrey R. Bloomquist, Micanopy, FL (US); Quentin R. Coquerel, Gainesville, FL (US); Gary M. Richoux, Gainesville, FL (US); Maia Tsikolia, Gainesville, FL (US); Liu Yang, Gainesville, FL (US)

(21) Appl. No.: 17/635,631

(22) PCT Filed: Aug. 28, 2020
(86) PCT No.: PCT/US20/48564

§ 371 Date: Feb. 15, 2022

Related U.S. Application Data
(60) Provisional application No. 62/893,085, filed on Aug. 28, 2019.
(60) Provisional application No. 62/959,600, filed on Jan. 10, 2020.

(51) Int. Cl. A01N 47/20; C07C 311/08; A01N 53/00; A01N 43/647; A01N 37/16; A01N 37/22; C07C 69/017; C07D 209/48; C07C 233/25; A01P 17/00; A01N 43/18; C07D 213/74; C07C 69/747; A01N 43/82; C07D 271/12; C07D 213/75; A01N 25/18; C07C 233/91; A01N 37/10; C07D 213/76; C07D 335/02; A01N 43/40; C07C 271/64; C07D 249/18; A01N 43/38; A01N 41/10; C07C 233/43; C07C 271/28; C07C 233/15; C07C 255/29; C07C 233/05; A01N 37/26; C07C 233/44; C07C 233/66

(52) U.S. Cl. CPC A01N 47/20; C07C 311/08; A01N 53/00; A01N 43/647; A01N 37/16; A01N 37/22; C07C 69/017; C07D 209/48; C07C 233/25; A01P 17/00; A01N 43/18; C07D 213/74; C07C 69/747; A01N 43/82; C07D 271/12; C07D 213/75; A01N 25/18; C07C 233/91; A01N 37/10; C07D 213/76; C07D 335/02; A01N 43/40; C07C 271/64; C07D 249/18; A01N 43/38; A01N 41/10; C07C 233/43; C07C 271/28; C07C 233/15; C07C 255/29; C07C 233/05; A01N 37/26; C07C 233/44; C07C 233/66; C07C 2601/10; C07C 2601/02

(57) ABSTRACT

The subject matter described herein is directed to compounds, synergistic compositions, and methods for repelling arthropods, such as insects. The compositions comprise aryl and pyridyl-type compounds. The compounds demonstrate strong vapor and topical repellency when applied alone and in synergistic compositions.

### CROSS REFERENCE TO RELATED APPLICATIONS

This application claims the benefit of priority to U.S. Provisional Patent Application No. 62/893,085, filed on Aug. 28, 2019, and U.S. Provisional Patent Application No. 62/959,600, filed on Jan. 10, 2020, the contents of each of which are incorporated by reference herein in their entirety for all purposes.

### GOVERNMENT INTEREST

This invention was made with government support under Cooperative Agreements 58-0208-5-001 and 59-6036-8-001 awarded by the United States Department of Agriculture, Agricultural Research Services and under U01CK000510 awarded by the Center for Disease Control. The government has certain rights in the invention.

### FIELD

The subject matter described herein is directed to insect repellent and insecticidal compounds and synergistic compositions, particularly those based on aryl and pyridyl amides.

### BACKGROUND

Biting insects are a nuisance and are also known to be carriers of disease. Compositions that repel insects, particularly insect pests such as mosquitoes, flies and fleas, are widely used to prevent animals from being bitten by such insects. The importance of preventing biting is essentially two-fold. Firstly, there are a number of insects which are capable of infecting animals with disease causing parasites, an example being the transmission of Plasmodium falciparum by mosquitoes to cause malaria. Secondly, in many cases whether disease is transmitted or not, the bite can be extremely irritating.

Insect repellents that act to repel insects from surfaces, including the skin of animals, are commercially available. They are designed to reduce or prevent the tendency of the insect to contact the surface, thereby preventing the biting. Over the years, a number of different oils, greases, ointments, sprays, and powders have been employed as insect repellents with varying degrees of success. Other delivery methods, such as fans, heated emanators, and burning coils, have also been developed with the purpose of repelling insects. Oil of citronella was reported to be an effective insect repellent as early as 1901. Another natural product, nicotine from tobacco, was used as a repellent as long ago as 1760. Since World War II, a number of synthetic insect repellents have been introduced.

One such repellent is N,N-diethyl-m-toluamide (DEET). It is a synthetic insect repellent that has demonstrated superior insect repellency in comparison to other repellents, including natural insect repellents. However, there is concern that repeated DEET exposure may have harmful consequences. For example, possible DEET side effects are irritability, confusion, insomnia, and even seizures. For these reasons, caution is generally recommended with regard to the use of DEET as an insect repellent. This is especially true when DEET is applied to children, who are more susceptible to the potentially injurious effects of exposure to DEET.

Other synthetic insecticides have been investigated. Modern insecticide compositions have to satisfy many demands, for example in relation to efficacy, persistence and spectrum of their action, and possible use. Important questions relate to toxicity, combinability with other active agents or formulating assistants, and another is that of the effort and expense of synthesizing an active ingredient. Moreover, resistance can occur. For economic and safety reasons, it would be beneficial to be able to use the least amount of active agents that provide the desired efficacy. The same is true for spatial repellents, vapor-active and deployed to keep a room, house, or tent, free of biting insects. Therefore, what is needed are highly efficacious compounds, whereby the concentrations and amounts of compounds required for efficacy is lessened. The subject matter described herein addresses the shortcomings of the art.

### BRIEF SUMMARY

In certain embodiments, the subject matter described herein is directed to a method of repelling arthropods, comprising contacting an object or locus with a compound of Formula IA, IB, IC, ID, IE, IF, or IG

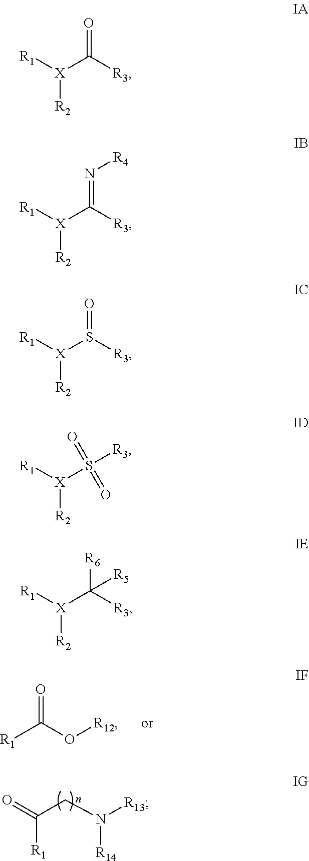

wherein,
R₁, R₂, R₃, R₄, R₅, R₆, R₁₂, R₁₃, R₁₄, X, and n are as defined herein; or, a composition comprising a compound of Formula IA, IB, IC, ID, IE, IF, or IG and a carrier;
wherein said arthropods are repelled from an adjacent area to said object or locus,
wherein said arthropods are not physically contacted with said object or locus.

In certain embodiments, the subject matter described herein is directed to a compound of Formula X:

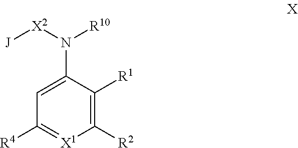

wherein,
J, X², R¹⁰, R¹, R⁴, X¹, and R² are as defined herein.

In certain embodiments, the subject matter described herein is directed to a method of repelling arthropods, comprising contacting an object or locus with a compound of Formula X, wherein said arthropods are repelled from an adjacent area to said object or locus, wherein said arthropods are not physically contacted with said locus.

In certain embodiments, the subject matter described herein is directed to a method of providing a compound of Formula IA, IB, IC, ID, IE, IF, IG, or X in an arthropod repellent medium from which a vaporized compound of Formula IA, IB, IC, ID, IE, IF, IG, or X can be dispersed.

In certain embodiments, the subject matter described herein is directed to a method of repelling an arthropod from an object or locus, comprising contacting said object or locus with a composition comprising a compound of Formula X.

In certain embodiments, the subject matter described herein is directed to a method for controlling one or more arthropods, comprising contacting said one or more arthropods with a composition comprising a compound of Formula X.

In certain embodiments, the subject matter described herein is directed to a composition comprising a compound of Formula IA, IB, IC, ID, IE, IF, IG, or X and an essential oil, wherein the composition is synergistic, in admixture with a carrier.

In certain embodiments, the subject matter described herein is directed to a method of repelling arthropods, comprising contacting an object or locus with a composition comprising an essential oil and a compound of Formula IA, IB, IC, ID, IE, IF, IG, or X, wherein said arthropods are repelled from an adjacent area to said object or locus, wherein said arthropods are not physically contacted with said object or locus, and wherein the composition is synergistic, in admixture with a carrier.

In certain embodiments, the subject matter described herein is directed to a composition comprising a compound of Formula IA, IB, IC, ID, IE, IF, IG, or X and a second compound selected from the group consisting of a pyrethroid, a compound of Formula II′, and a compound of Formula III′

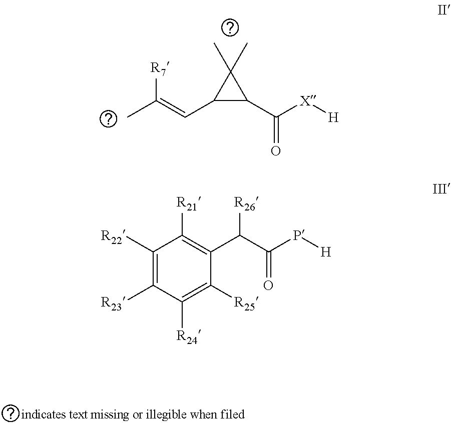

wherein,
R_{6′}, R_{7′}, R_{8′}, R_{9′}, X″, R_{22′}, R_{23′}, R_{24′}, R_{25′}, R_{21′}, R_{26′}, and P′ are as defined herein, wherein the composition is synergistic, in admixture with a carrier.

In certain embodiments, the subject matter described herein is directed to a method of repelling arthropods, comprising contacting an object or locus with a composition comprising an essential oil and a compound of Formula IA, IB, IC, ID, IE, IF, or IG; and a second compound selected from the group consisting of a pyrethroid, a compound of Formula II′, and a compound of Formula III′, wherein said arthropods are repelled from an adjacent area to said object or locus, wherein said arthropods are not physically contacted with said locus; and, wherein the composition is synergistic, in admixture with a carrier.

Other embodiments are also described.

### BRIEF DESCRIPTION OF THE DRAWINGS

FIG. 1 depicts representative DEET and control responses in a glass tube assay. Mosquitoes moved away from the DEET treated side (top right), while remaining evenly distributed on either side of the midline for the acetone negative controls (bottom). The tubes as shown were held horizontally on the bench. In certain cases, with some compounds (e.g., metofluthrin and transfluthrin), the tubes were held vertically and gave more consistent responses, presumably due to different chemical diffusion properties (Jiang et al., 2019).

FIG. 2 depicts a plot demonstrating how the fluorinated carbon chain length in several exemplary compounds affects the mortality in two species of mosquitoes.

FIG. 3 depicts repellency concentration-response curves for MTFI-5-3A alone and in the presence of TFA. When the proportion on the treated side is 0.5, it corresponds to 0% repellency and a value of 0=100% repellency, as calculated by the equation Y=(1−(X/0.5)*100. Symbols are mean+SEM. Dotted line shows the 50% repellency response level.

FIG. 4 depicts concentration-response curves for MTFI-4-3D alone and in the presence of an EC₂₀ concentration of citronella (proportion on treated side=0.4 with citronella alone), illustrating an enhanced repellency effect. When the proportion on the treated side is 0.5, it corresponds to 0% repellency and a value of 0=100% repellency, as calculated by the equation Y=(1−(X/0.5)*100. Symbols are mean+SEM. Dotted line shows the 50% repellency response level.

### DETAILED DESCRIPTION

Described herein are compounds, compositions, and methods that provide desired insect repellency. The compositions comprise aryl and pyridyl-type compounds. The compounds demonstrate excellent repellency both alone and in synergistic compositions with other compounds.

The propagation of mosquito-vectored infectious diseases such as Malaria, Zika, West-Nile, Dengue, and Yellow Fever is a growing concern. According to the World Health Organization, more than 400,000 annual deaths are related to malaria. In 2016, the CDC classified Zika as a nationally notifiable condition. What is needed are vapor active repellents and insecticides and topically active insecticides that can help prevent insect bites and reduce the risk to victims. Unfortunately, current commercial products are not potent enough or suffer from target-site mediated resistance.

As described herein, however, it has been found that certain aryl and pyridyl compounds exhibit strong repellent bioactivity and selective toxicity. The compounds are both vapor and topically active. In certain embodiments, the compounds and compositions described herein are more potent than leading commercial insecticides. The compounds additionally demonstrate excellent activity in synergistic compositions with essential oils or other compounds. Furthermore, the methods and compositions disclosed herein are effective, even at low concentrations.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±50%. In certain other embodiments, the term “about” includes the indicated amount±20%. In certain other embodiments, the term “about” includes the indicated amount±10%. In other embodiments, the term “about” includes the indicated amount±5%. In certain other embodiments, the term “about” includes the indicated amount±1%. In certain other embodiments, the term “about” includes the indicated amount±0.5% and in certain other embodiments, ±0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” or “alkene” refers to an alkyl group containing at least one carbon-carbon double bond and having from 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂-C₂₀ alkynyl), 2 to 8 carbon atoms (i.e., C₂-C₈ alkynyl), 2 to 6 carbon atoms (i.e., C₂-C₆ alkynyl) or 2 to 4 carbon atoms (i.e., C₂-C₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylalkyl” or “Aralkyl” refers to the group “aryl-alkyl-”, such as benzyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”.

“Hydrazino” refers to —NHNH₂.

“Imino” refers to a group —C(NR^{y})R^{z}, wherein R^{y} and R^{Z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen.

“Hydroxyalkyl” refers to an alkyl group as defined above, wherein one or more (e.g., 1 to 6, or 1 to 3) hydrogen atoms are replaced by a hydroxy group.

“Hydroxy” refers to an —“OH” group.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₅ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocycle ring contains 4- or 6-ring atoms, it is also referred to herein as a 4- or 6-membered heterocycle. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Alkyl-haloalkoxy” refers to “-alkyl-haloalkoxy.” In certain embodiments, the alkyl chain of the alkyl-haloalkoxy may be substituted.

“Haloalkyl-haloalkoxy” refers to “-haloalkyl-haloalkoxy.”

“Cyano-alkyl” refers to “-alkyl-cyano.”

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, 1 to 4, or 1 to 3) hydrogen atom is replaced by a bond to a non-hydrogen atom such as, but not limited to alkyl, alkenyl, alkynyl, alkoxy, alkylthio, amino, amidino, aryl, aralkyl, carboxyl, carboxyl ester, cyano, cycloalkyl, cycloalkylalkyl, halo, haloalkyl, haloalkoxy, hydroxyalkyl, heteroalkyl, heteroaryl, heteroarylalkyl, heterocyclyl, heterocyclylalkyl, —NHNH₂, ═NNH₂, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{h}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, 1 to 4, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and R¹ are taken together with the atoms to which they are attached to form a heterocyclyl ring optionally substituted with oxo, halo or alkyl optionally substituted with oxo, halo, amino, hydroxyl, or alkoxy.

As used herein, wherein “alkyl” is said to be optionally substituted with sulfur or oxygen, said sulfur or oxygen can replace one or more carbons in the “alkyl.”

The compounds of the invention, or their agrochemically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic, optically pure, and enantiomerically/diastereomerically enriched forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

As used herein, “OR” refers to the Orlando insecticide-susceptible strain of Ae. aegypti.

As used herein, “PR” refers to the pyrethroid-resistant Puerto Rico (PR) strain of Ae. aegypti.

As used herein, the term “non-toxic” is intended to convey a composition that, while highly effective in killing and/or repelling targeted insect pests, is safe to use around humans, particularly small children, and pets.

Additional definitions may also be provided below as appropriate.

### II. Compounds

In certain embodiments, the subject matter described herein is directed to compounds of Formula IA, IB, IC, ID, IE, IF, or IG

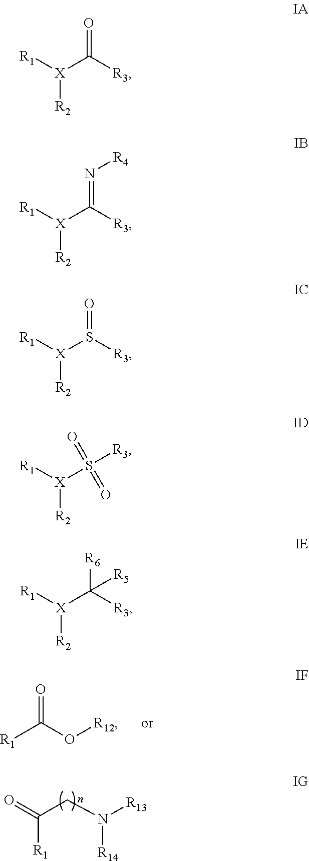

wherein,
X is selected from O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl wherein said heteroaryl, phenyl, cycloalkyl, or heterocycyl is optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, (5- to 10-membered heterocyclyl)-C₁-C₃ alkyl, wherein R₁₆ and R₁₇ are selected from hydrogen, linear or branched C₁-C₁₂ alkyl, or alkene; and said C₆-C₁₀ aryl or (C₆-C₁₀ aryl)-C₁-C₃ alkyl is optionally substituted with one or two substituents, each of which is independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

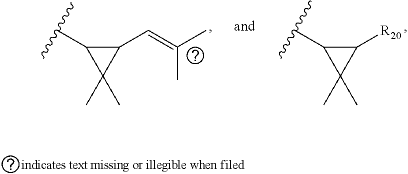

where R₁₈, R₁₉, R₂₀, R₂₁, and R₂₂ are each independently selected from the group consisting of cyano, C₁-C₃ alkyl, halogen, haloalkyl, hydrogen, and phenyl, wherein said phenyl is optionally substituted one, two, or three times with halogen;
wherein said cyano-alkyl is optionally substituted with halogen and said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from hydrogen, halogen, haloalkyl, or C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, or C₁-C₃ alkyl, wherein said heterocyclyl or phenyl is optionally substituted with one or two substituents independently selected from halogen, haloalkyl, or linear or branched C₁-C₃ alkyl;
or,
wherein R₁₃ and R₁₄, together with the nitrogen to which each is attached, form a 5-10 membered heterocyclyl or C₆-C₁₀ heteroaryl; and
n is 0 or 1.

In certain embodiments, R₃ is selected from the group consisting of C₁-C₁₂ alkyl, haloalkyl, alkoxy, haloalkoxy alkene, cyano, alkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

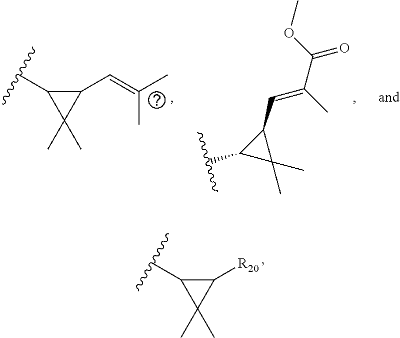

where R₁₈, R₁₉, R₂₀, R₂₁, and R₂₂ are each independently selected from the group consisting of cyano, C₁-C₃ alkyl, halogen, haloalkyl, hydrogen, and phenyl, wherein said phenyl is optionally substituted one, two, or three times with halogen; wherein said cyano-alkyl is optionally substituted with halogen and said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen.

In certain embodiments, X is N.

In certain embodiments, R₁ is selected from the group consisting of 6-membered heteroaryl, 6-membered heteroaryl-C₁-C₃ alkyl, phenyl, benzyl, and (5- to 10-membered heterocyclyl)-C₁-C₃ alkyl wherein said heteroaryl, phenyl, or heterocycyl is optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl.

In certain embodiments, R₁ is phenyl or pyridinyl optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl.

In certain embodiments, halogen is fluorine, chlorine, or bromine.

In certain embodiments, R₁ is selected from the group consisting of

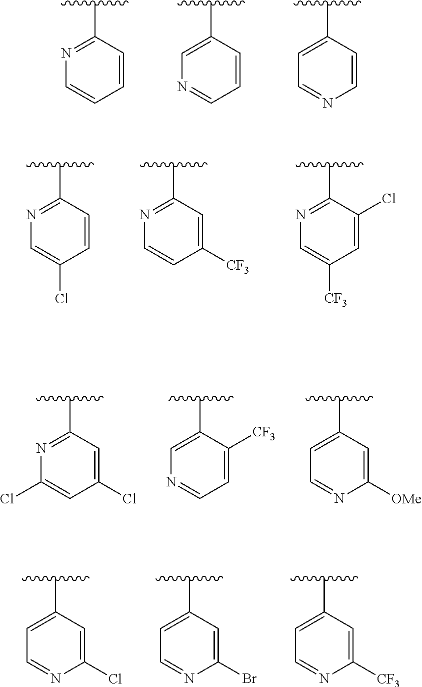

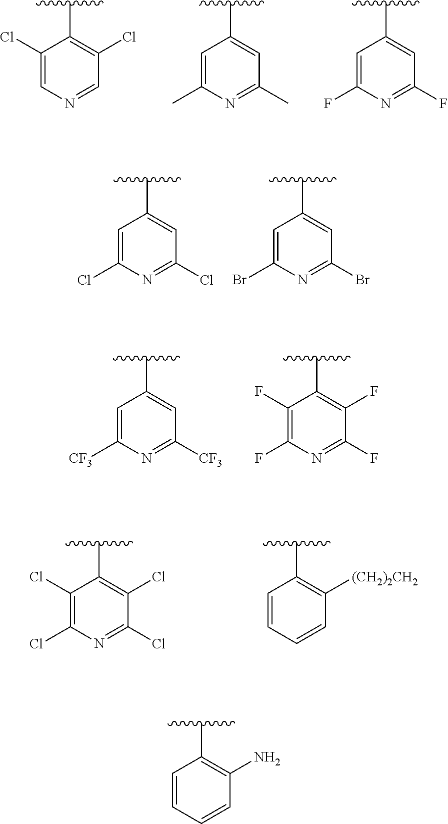

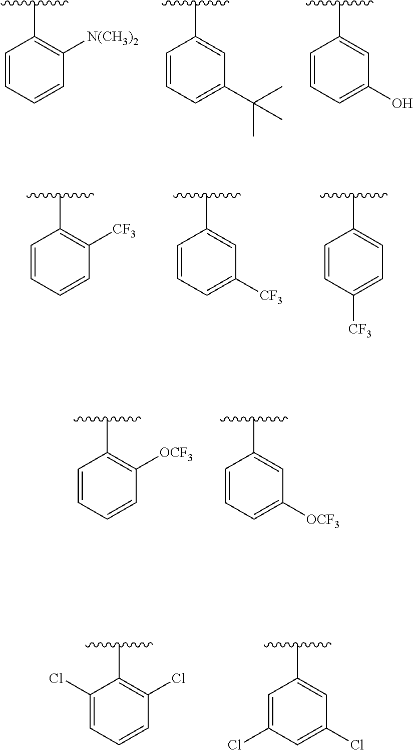

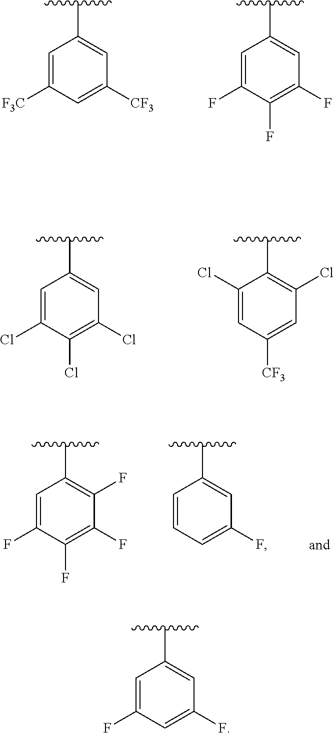

In certain embodiments, R₁ is selected from

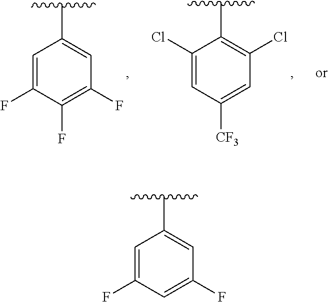 [SMILES: *c1cc(F)c(F)c(F)c1 ; *c1c(Cl)cc(C(F)(F)F)cc1Cl ; *c1cc(F)cc(F)c1]

In certain embodiments, R₁ is

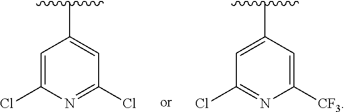 [SMILES: *c1cc(Cl)nc(Cl)c1 ; "or" ; *c1cc(Cl)nc(C(F)(F)F)c1]

In certain embodiments, R₂ is selected from the group consisting of

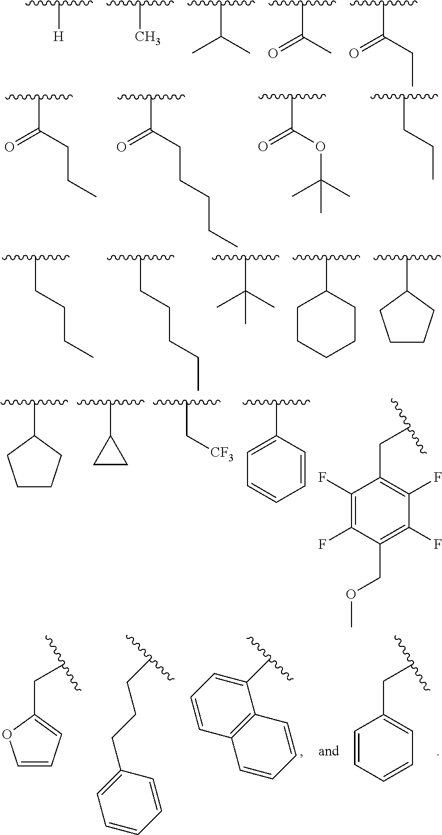

In certain embodiments, R₂ is hydrogen.

In certain embodiments, R₃ is selected from the group consisting of

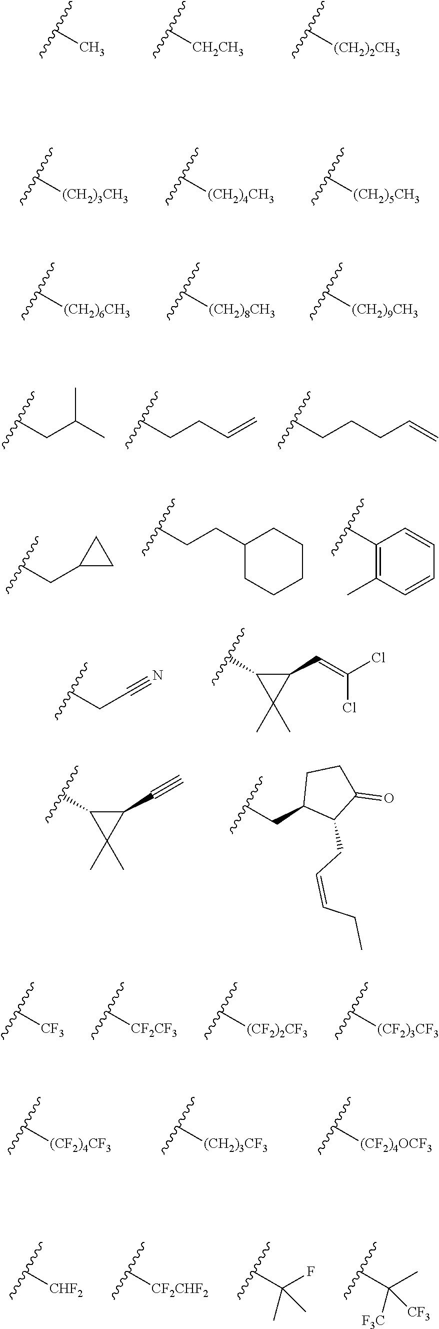

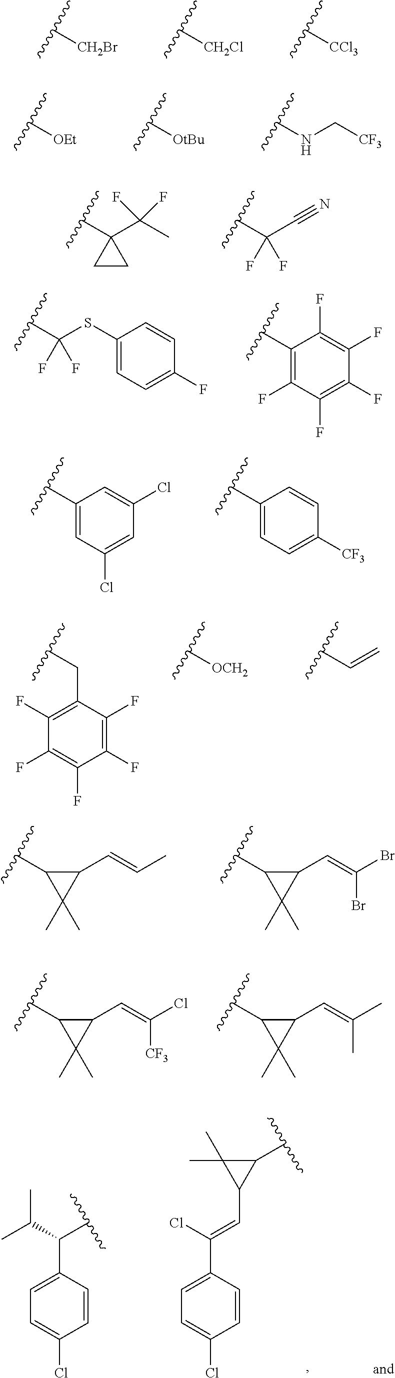

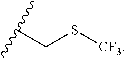 [SMILES: *CSC(F)(F)F]

In certain embodiments, R₃ is selected from

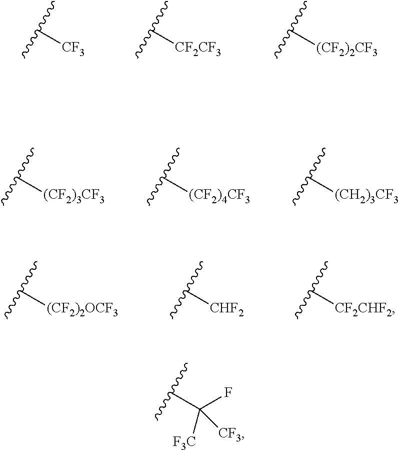

or C₁-C₆ alkyl.

In certain embodiments, R₃ is selected from methyl, ethyl, propyl, butyl, or pentyl.

In certain embodiments, R₃ is selected from

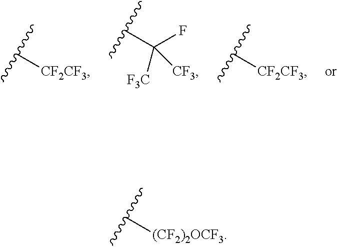

In certain embodiments, the compound is of Formula IA.

In certain embodiments, the compound of Formula IA is selected from the group consisting of

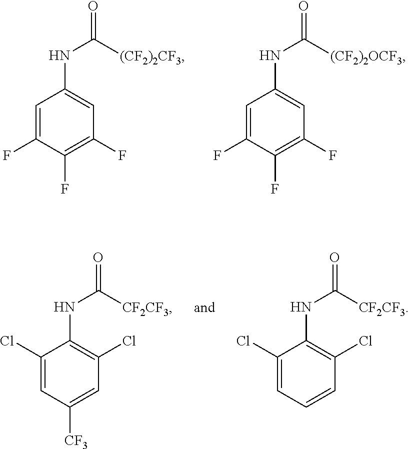

In certain embodiments, the compound of Formula IA is selected from the group consisting of

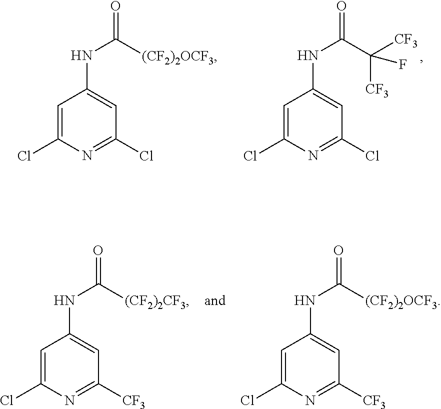

In certain embodiments, the compound of Formula IA, IB, IC, ID, IE, IF, or IG is selected from the group consisting of

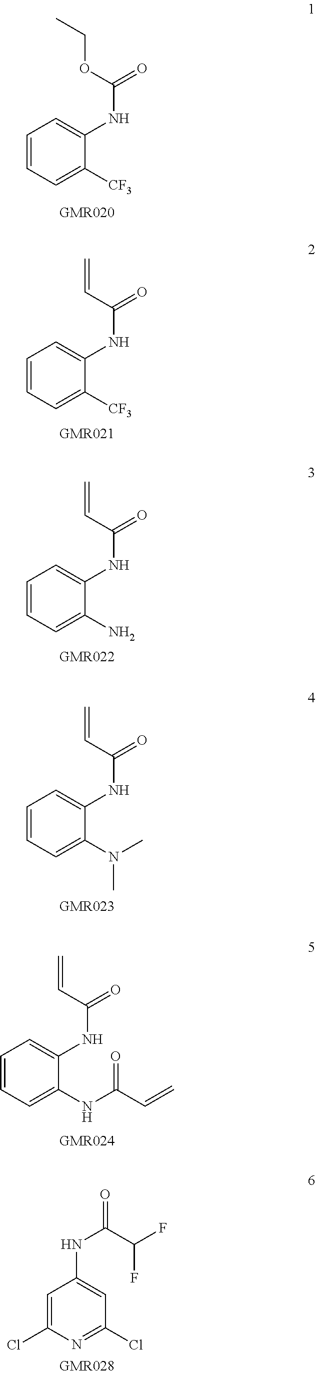 [SMILES: CCOC(=O)Nc1ccccc1C(F)(F)F ; C=CC(=O)Nc1ccccc1C(F)(F)F ; C=CC(=O)Nc1ccccc1N ; C=CC(=O)Nc1ccccc1N(C)C ; C=CC(=O)Nc1ccccc1NC(=O)C=C ; O=C(Nc1cc(Cl)nc(Cl)c1)C(F)F]

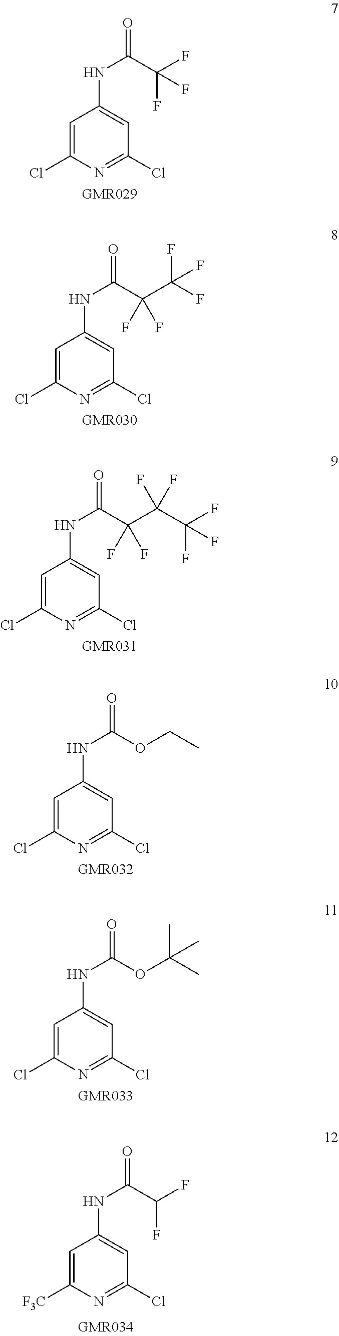 [SMILES: O=C(Nc1cc(Cl)nc(Cl)c1)C(F)(F)F ; O=C(Nc1cc(Cl)nc(Cl)c1)C(F)(F)C(F)(F)F ; O=C(Nc1cc(Cl)nc(Cl)c1)C(F)(F)C(F)(F)C(F)(F)F ; CCOC(=O)Nc1cc(Cl)nc(Cl)c1 ; CC(C)(C)OC(=O)Nc1cc(Cl)nc(Cl)c1 ; O=C(Nc1cc(Cl)nc(C(F)(F)F)c1)C(F)F]

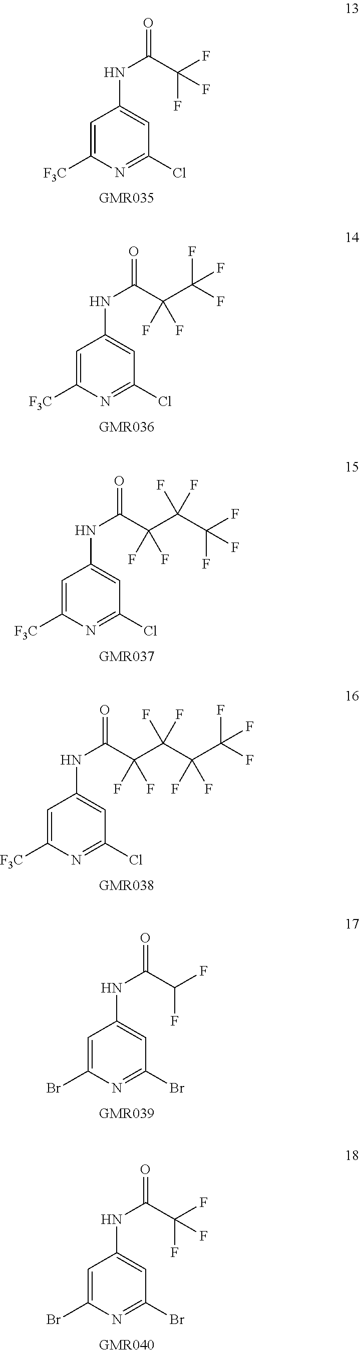 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(C(F)(F)F)c1)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

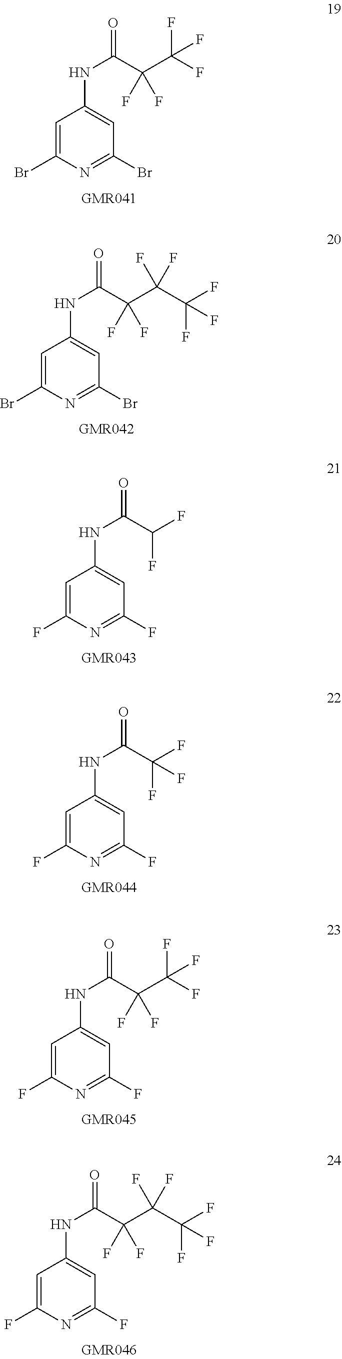 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; O=C(Nc1cc(F)nc(F)c1)C(F)F ; O=C(Nc1cc(F)nc(F)c1)C(F)(F)F ; O=C(Nc1cc(F)nc(F)c1)C(F)(F)C(F)(F)F ; O=C(Nc1cc(F)nc(F)c1)C(F)(F)C(F)(F)C(F)(F)F]

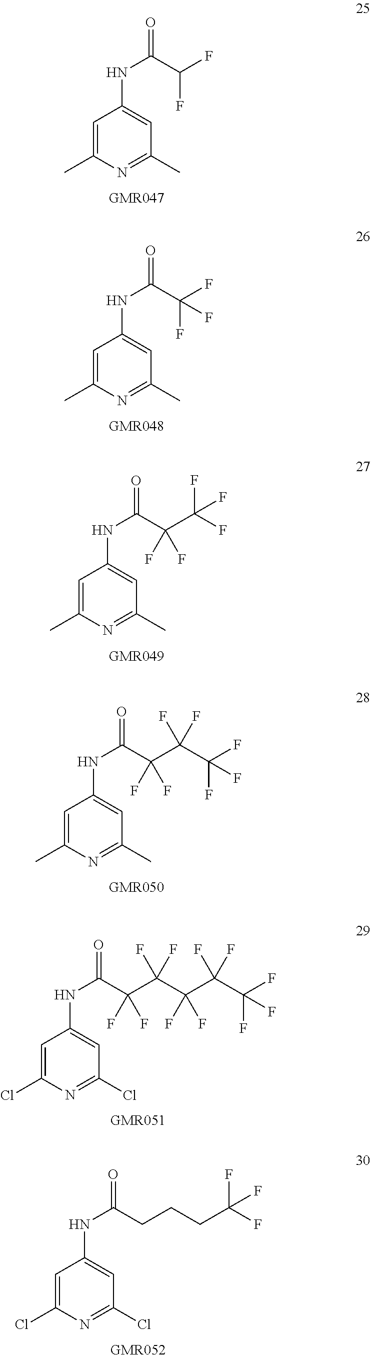 [SMILES: Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1 ; O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1]

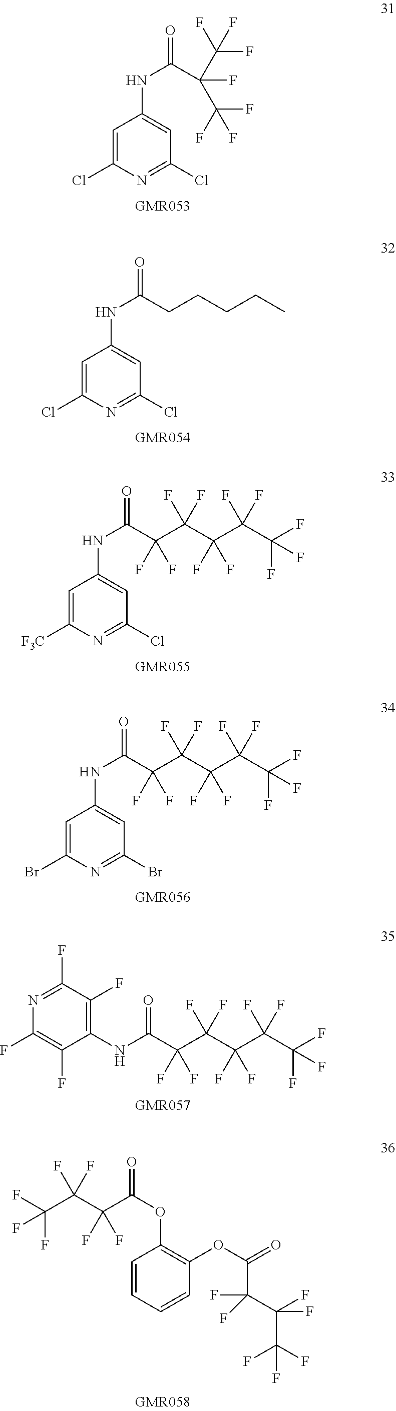 [SMILES: O=C(Nc1cc(Cl)nc(Cl)c1)C(F)(C(F)(F)F)C(F)(F)F ; CCCCCC(=O)Nc1cc(Cl)nc(Cl)c1 ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F ; O=C(Nc1c(F)c(F)nc(F)c1F)C(F)(F)C(F)(F)C(F)(F)C(F)(F)C(F)(F)F ; O=C(Oc1ccccc1OC(=O)C(F)(F)C(F)(F)C(F)(F)F)C(F)(F)C(F)(F)C(F)(F)F]

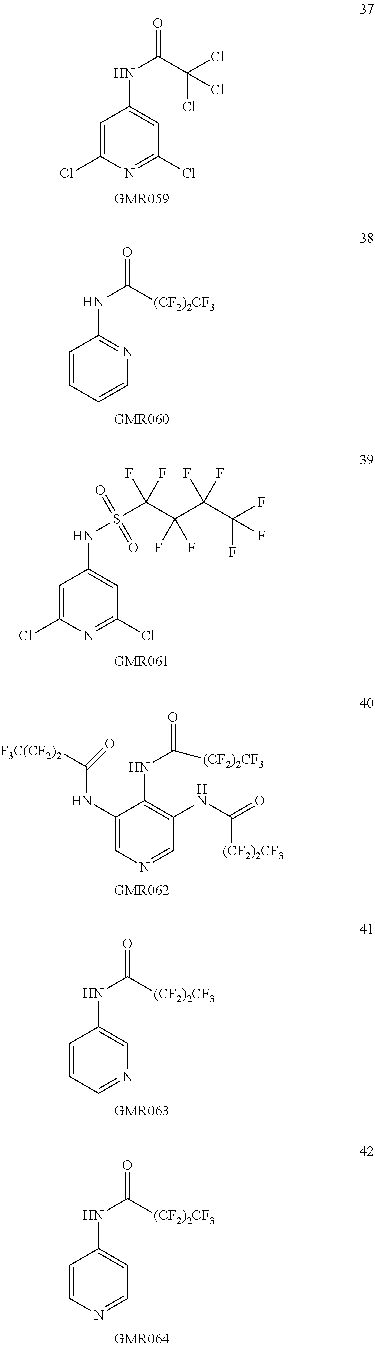

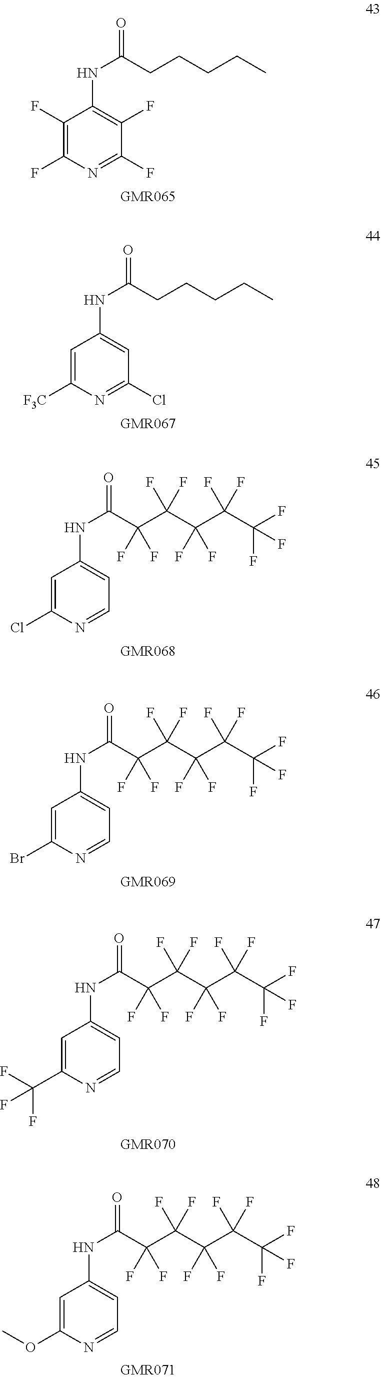

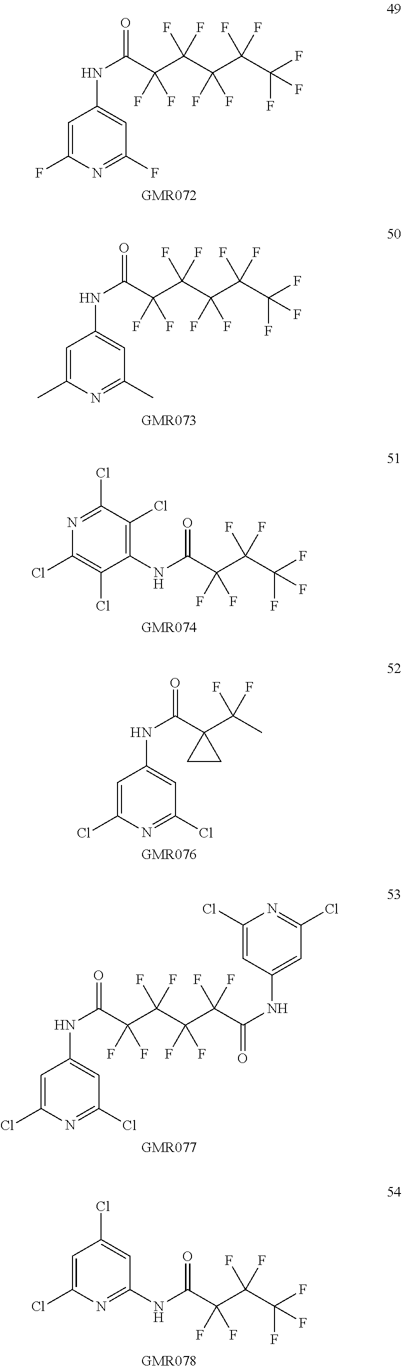 [SMILES: O=C(Nc1cc(F)nc(F)c1)C(F)(F)C(F)(F)C(F)(F)C(F)(F)C(F)(F)F ; Cc1cc(NC(=O)C(F)(F)C(F)(F)C(F)(F)C(F)(F)C(F)(F)F)cc(C)n1 ; O=C(Nc1c(Cl)c(Cl)nc(Cl)c1Cl)C(F)(F)C(F)(F)C(F)(F)F ; CC(F)(F)C1(C(=O)Nc2cc(Cl)nc(Cl)c2)CC1 ; O=C(Nc1cc(Cl)nc(Cl)c1)C(F)(F)C(F)(F)C(F)(F)C(F)(F)C(=O)Nc1cc(Cl)nc(Cl)c1 ; O=C(Nc1cc(Cl)cc(Cl)n1)C(F)(F)C(F)(F)C(F)(F)F]

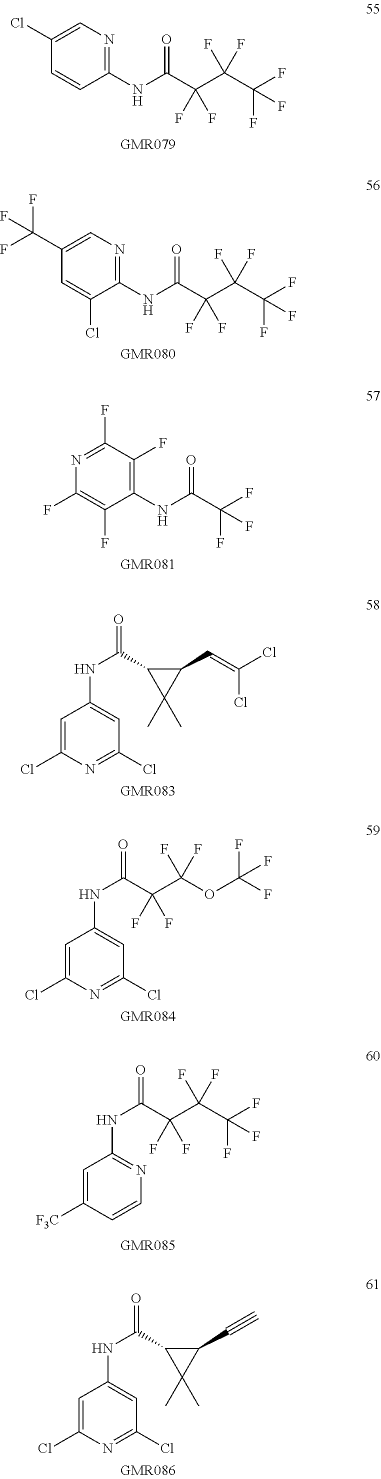

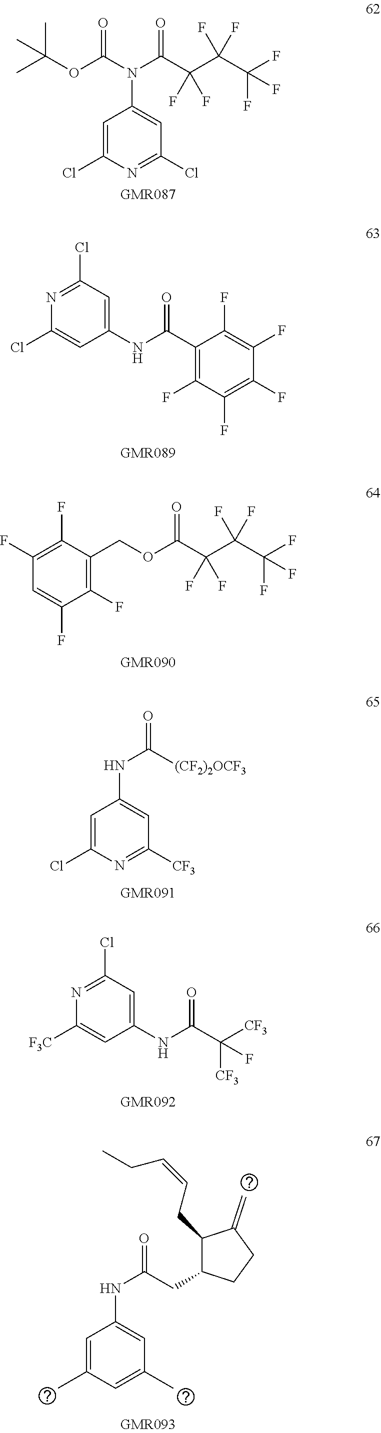

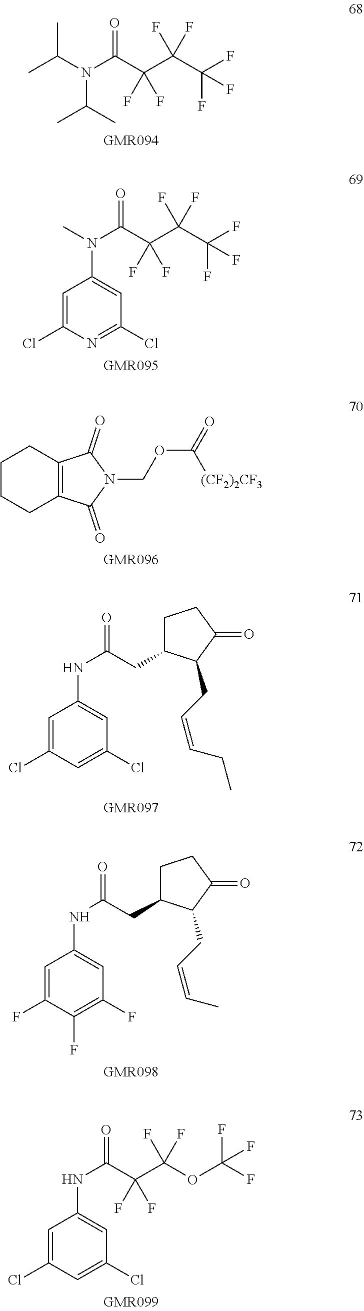

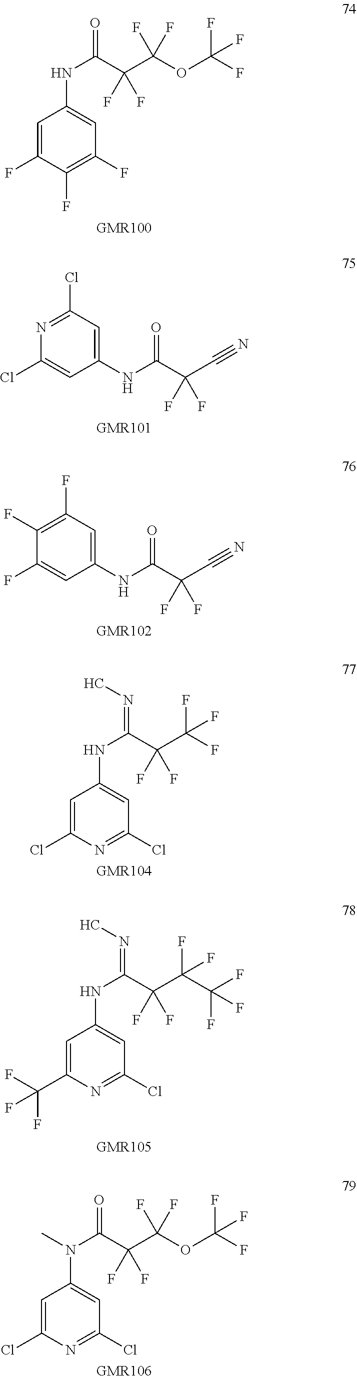 [SMILES: O=C(Nc1cc(F)c(F)c(F)c1)C(F)(F)C(F)(F)OC(F)(F)F ; N#CC(F)(F)C(=O)Nc1cc(Cl)nc(Cl)c1 ; N#CC(F)(F)C(=O)Nc1cc(F)c(F)c(F)c1 ; C/N=C(\Nc1cc(Cl)nc(Cl)c1)C(F)(F)C(F)(F)F ; C/N=C(\Nc1cc(Cl)nc(C(F)(F)F)c1)C(F)(F)C(F)(F)C(F)(F)F ; CN(C(=O)C(F)(F)C(F)(F)OC(F)(F)F)c1cc(Cl)nc(Cl)c1]

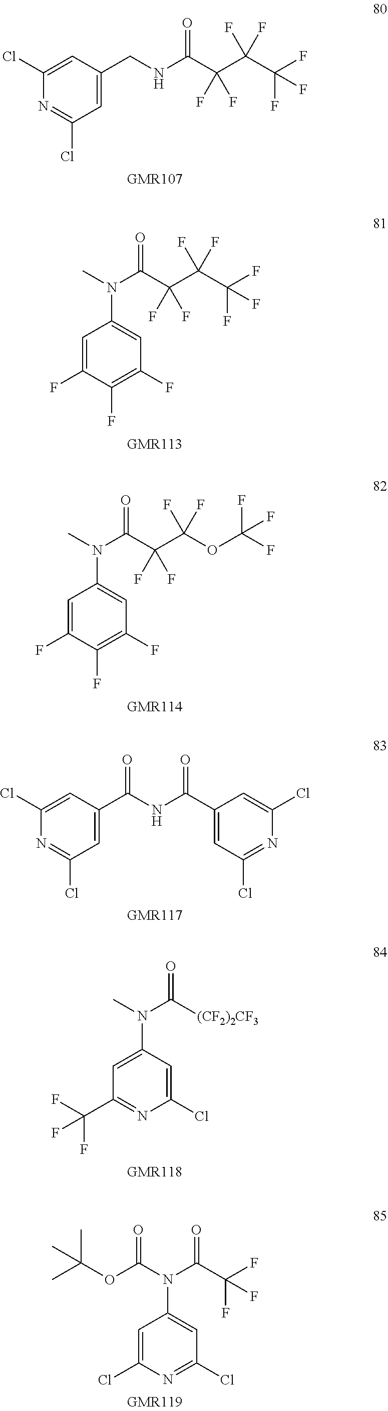

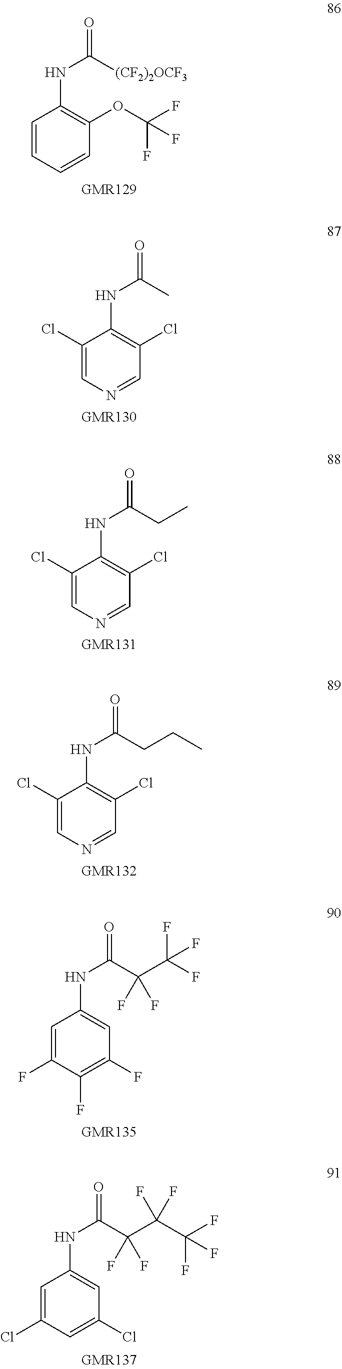

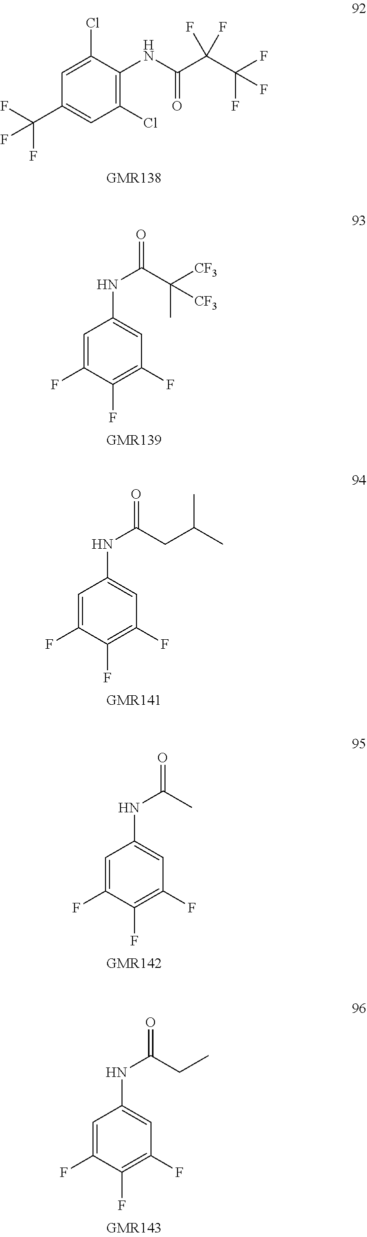 [SMILES: O=C(Nc1c(Cl)cc(C(F)(F)F)cc1Cl)C(F)(F)C(F)(F)F ; CC(C(=O)Nc1cc(F)c(F)c(F)c1)(C(F)(F)F)C(F)(F)F ; CC(C)CC(=O)Nc1cc(F)c(F)c(F)c1 ; CC(=O)Nc1cc(F)c(F)c(F)c1 ; CCC(=O)Nc1cc(F)c(F)c(F)c1]

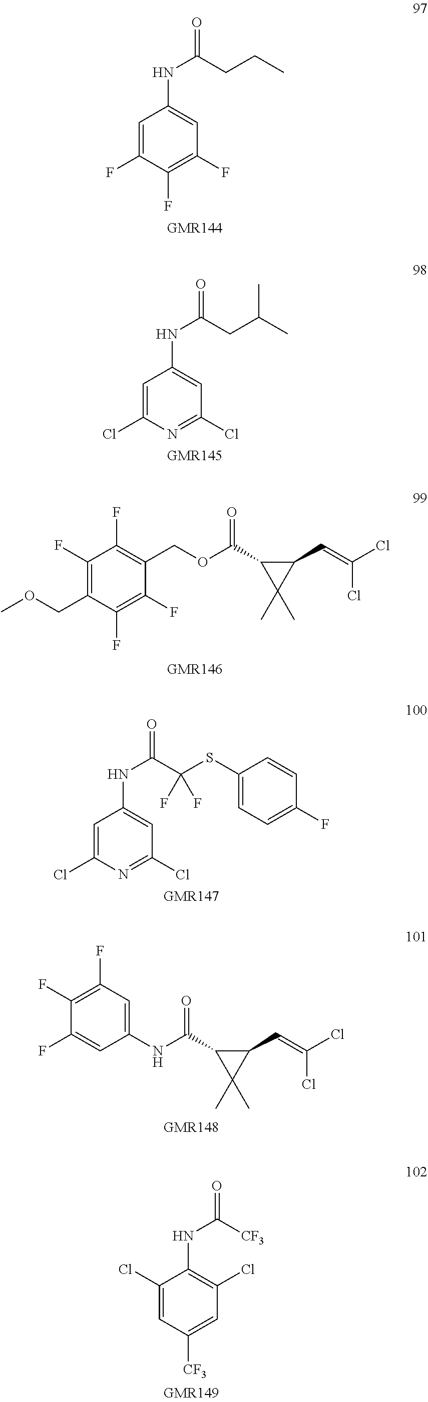 [SMILES: CCCC(=O)Nc1cc(F)c(F)c(F)c1 ; CC(C)CC(=O)Nc1cc(Cl)nc(Cl)c1 ; COCc1c(F)c(F)c(COC(=O)[C@@H]2[C@@H](C=C(Cl)Cl)C2(C)C)c(F)c1F ; O=C(Nc1cc(Cl)nc(Cl)c1)C(F)(F)Sc1ccc(F)cc1 ; CC1(C)[C@H](C=C(Cl)Cl)[C@H]1C(=O)Nc1cc(F)c(F)c(F)c1 ; O=C(Nc1c(Cl)cc(C(F)(F)F)cc1Cl)C(F)(F)F]

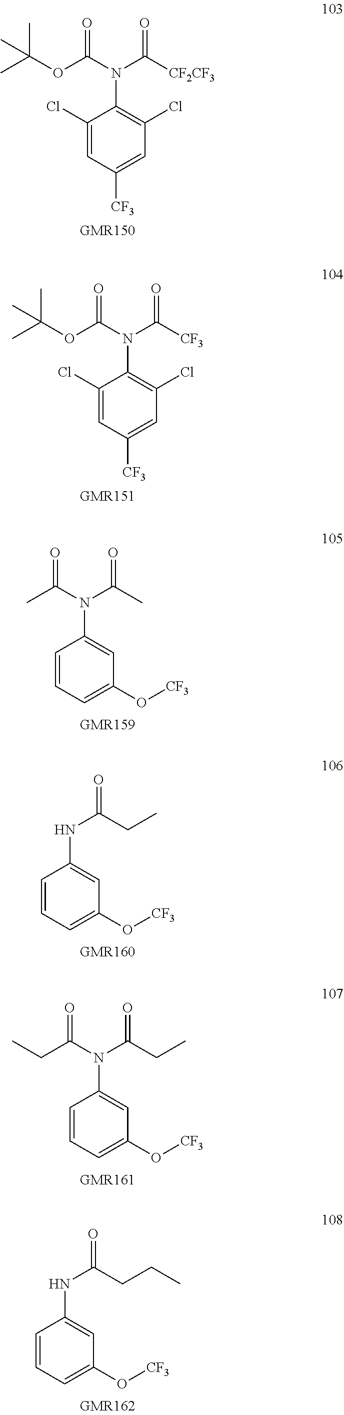

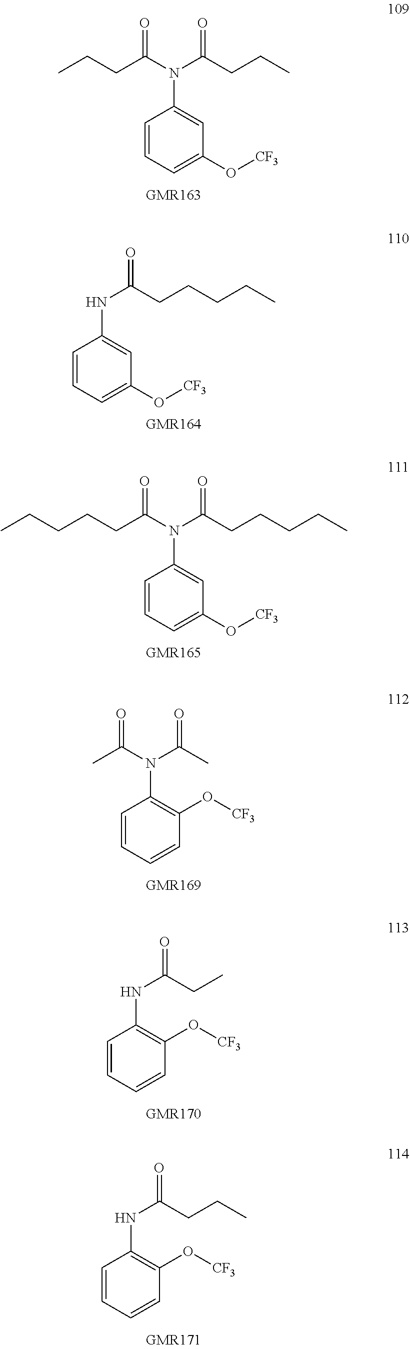

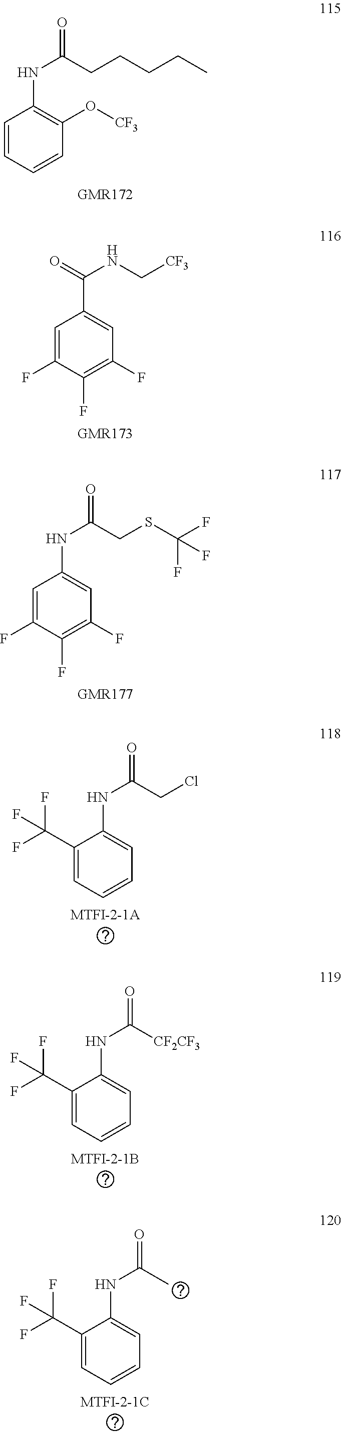

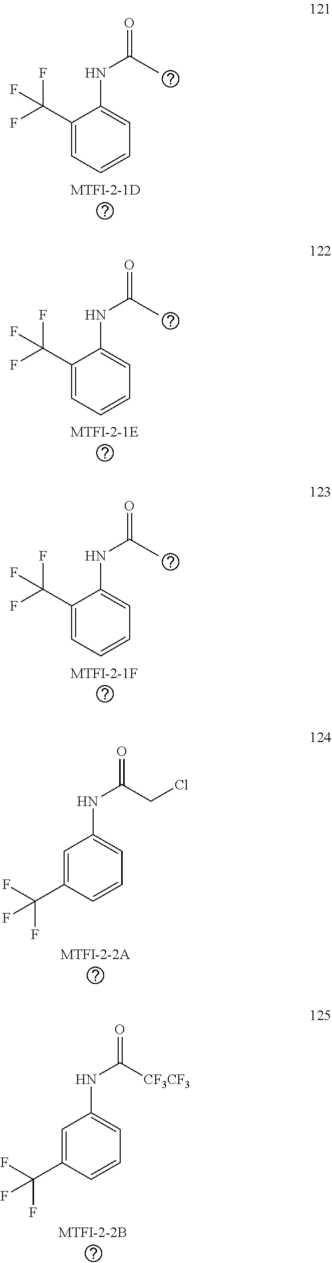

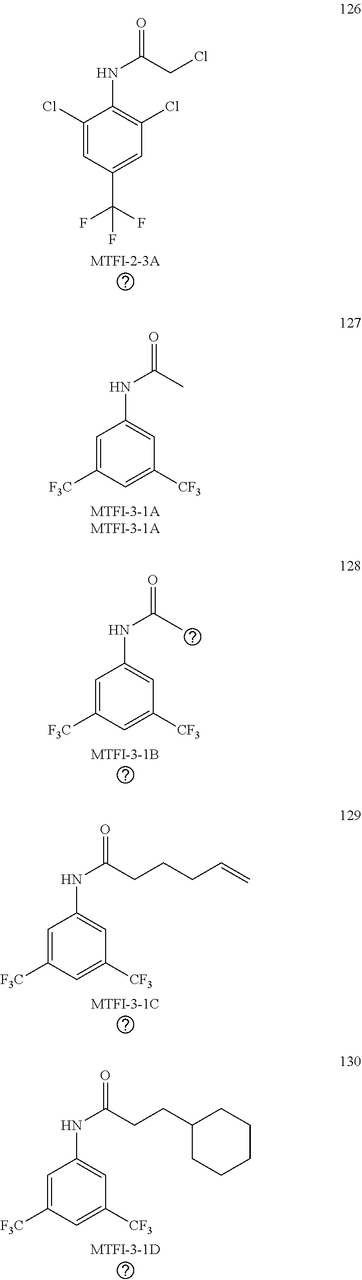 [SMILES: O=C(CCl)Nc1c(Cl)cc(C(F)(F)F)cc1Cl ; CC(=O)Nc1cc(C(F)(F)F)cc(C(F)(F)F)c1 ; CC(=O)Nc1cc(C(F)(F)F)cc(C(F)(F)F)c1 ; C=CCCCC(=O)Nc1cc(C(F)(F)F)cc(C(F)(F)F)c1 ; O=C(CCC1CCCCC1)Nc1cc(C(F)(F)F)cc(C(F)(F)F)c1]

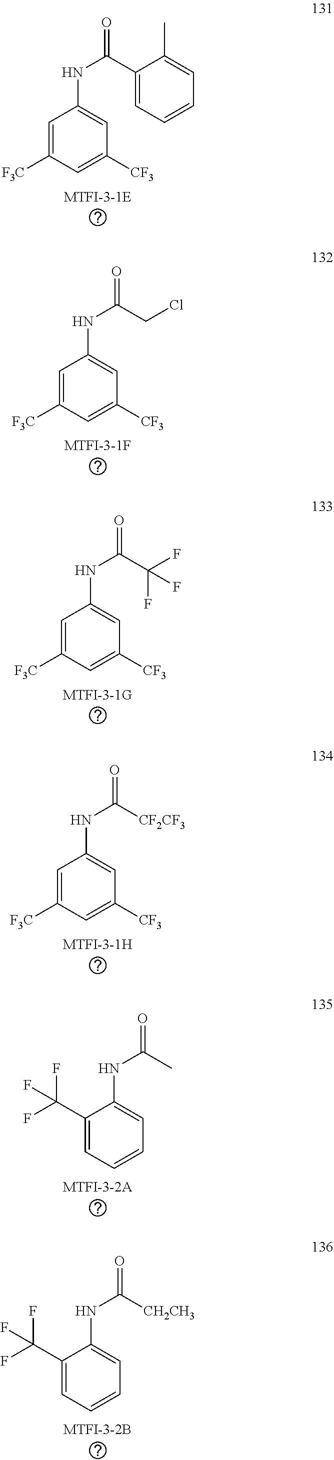

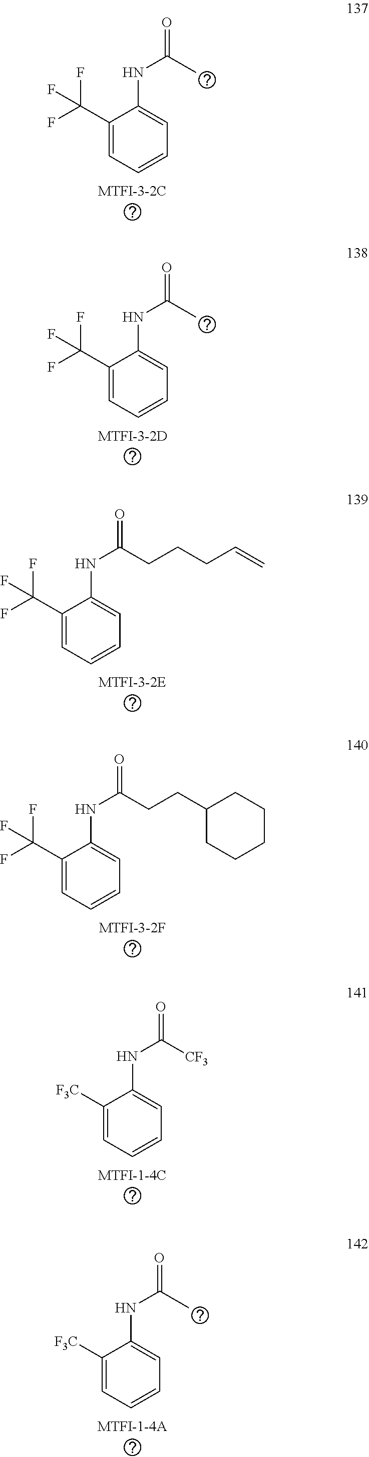 [SMILES: CC(=O)Nc1ccccc1C(F)(F)F ; CC(=O)Nc1ccccc1C(F)(F)F ; C=CCCCC(=O)Nc1ccccc1C(F)(F)F ; O=C(CCC1CCCCC1)Nc1ccccc1C(F)(F)F ; O=C(Nc1ccccc1C(F)(F)F)C(F)(F)F ; CC(=O)Nc1ccccc1C(F)(F)F]

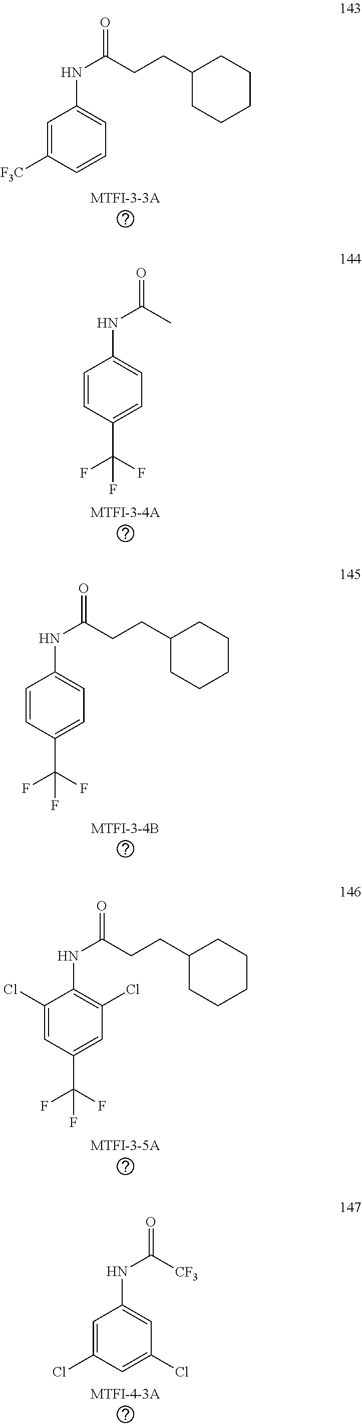 [SMILES: O=C(CCC1CCCCC1)Nc1cccc(C(F)(F)F)c1 ; CC(=O)Nc1ccc(C(F)(F)F)cc1 ; O=C(CCC1CCCCC1)Nc1ccc(C(F)(F)F)cc1 ; O=C(CCC1CCCCC1)Nc1c(Cl)cc(C(F)(F)F)cc1Cl ; O=C(Nc1cc(Cl)cc(Cl)c1)C(F)(F)F]

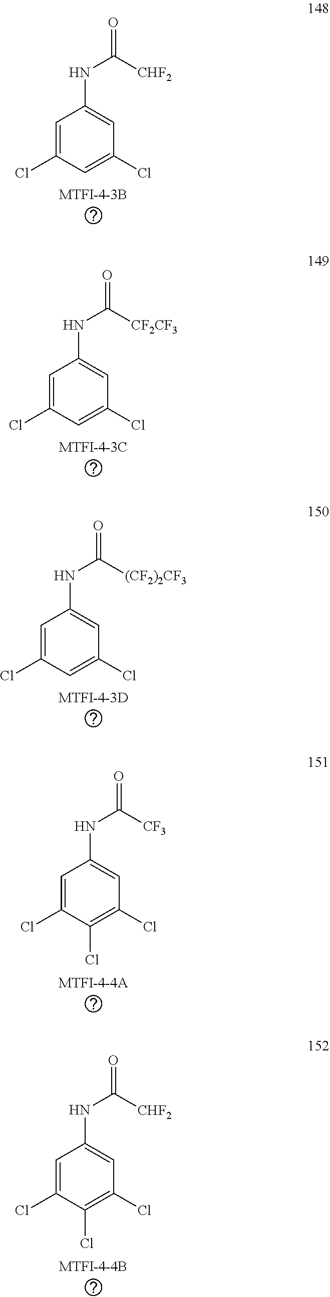

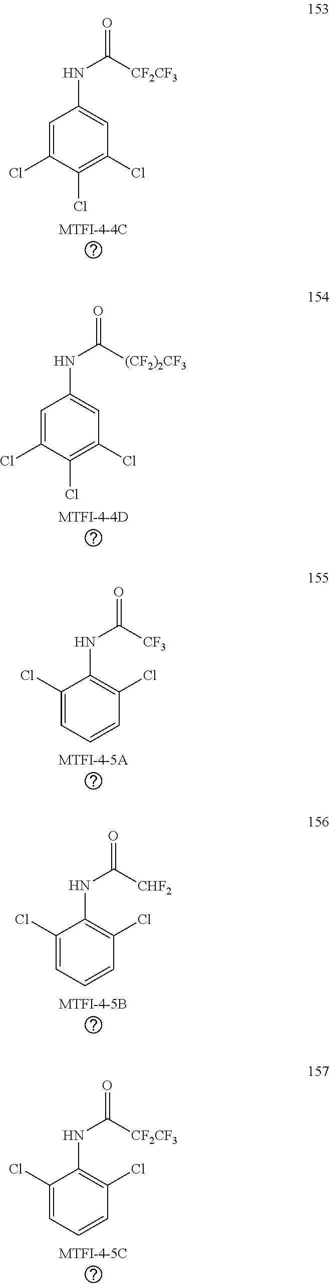

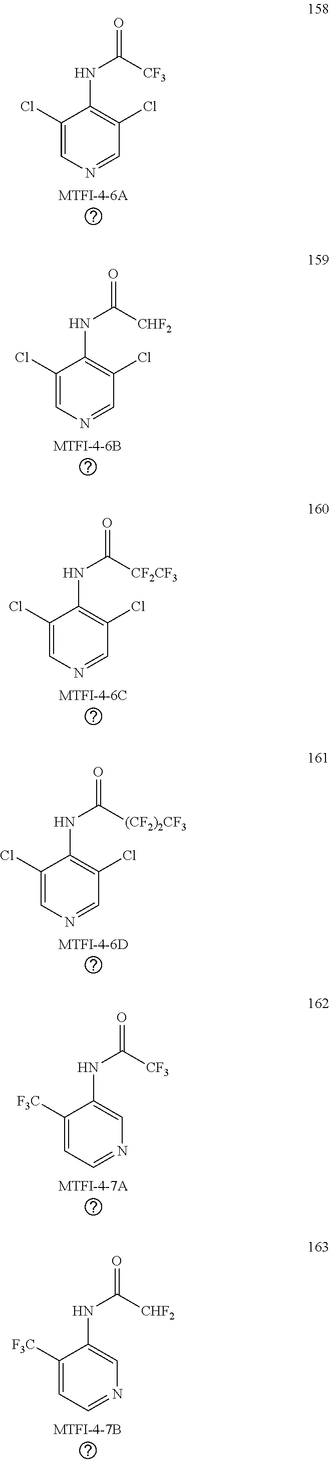

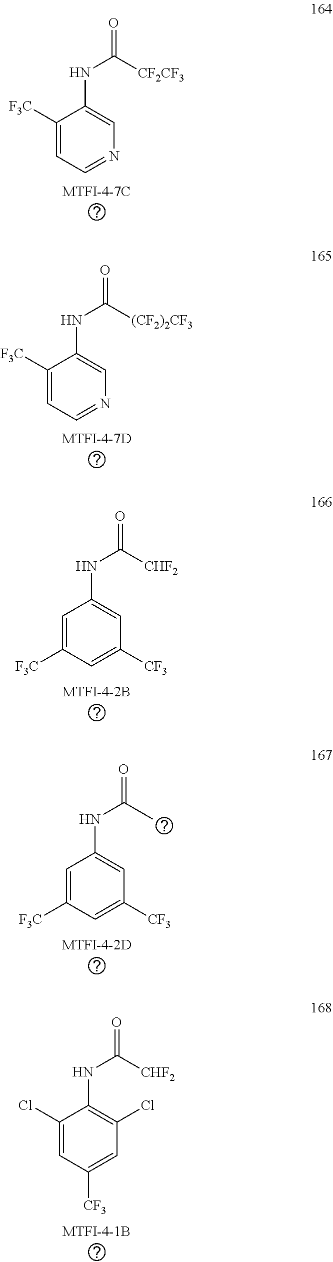

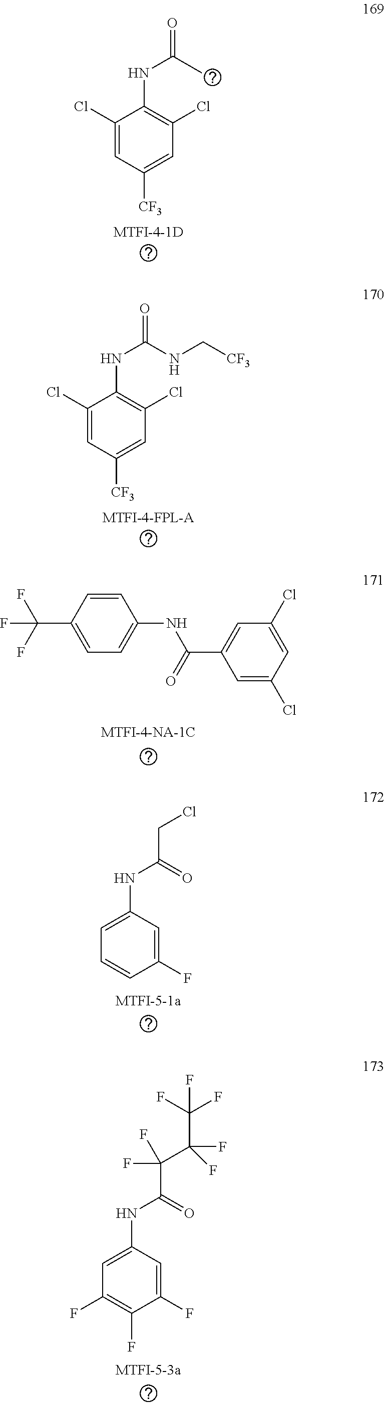 [SMILES: CC(=O)Nc1c(Cl)cc(C(F)(F)F)cc1Cl ; O=C(NCC(F)(F)F)Nc1c(Cl)cc(C(F)(F)F)cc1Cl ; O=C(Nc1ccc(C(F)(F)F)cc1)c1cc(Cl)cc(Cl)c1 ; O=C(CCl)Nc1cccc(F)c1 ; O=C(Nc1cc(F)c(F)c(F)c1)C(F)(F)C(F)(F)C(F)(F)F]

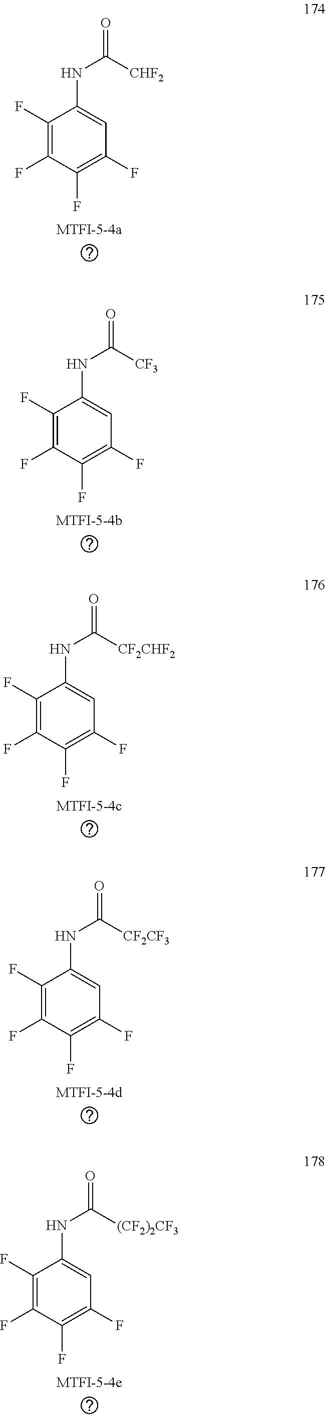

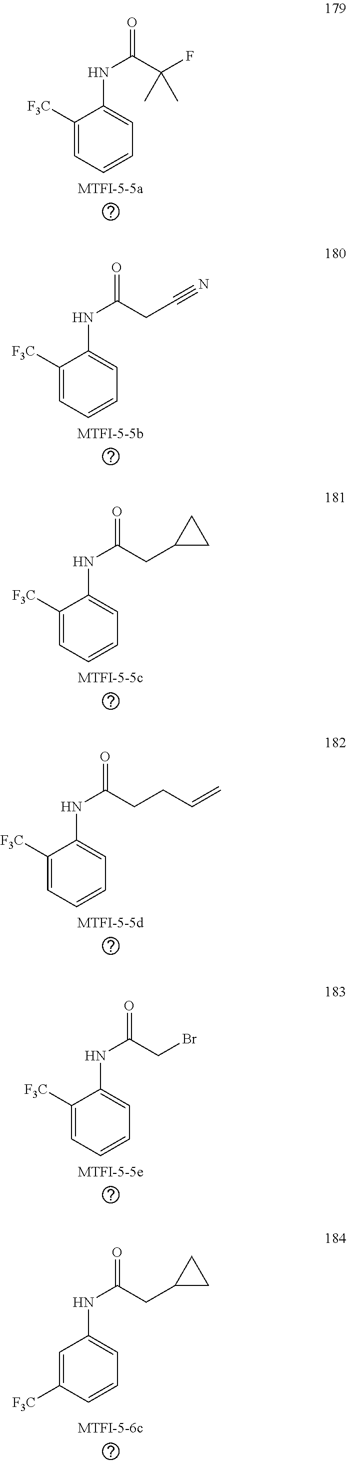 [SMILES: CC(C)(F)C(=O)Nc1ccccc1C(F)(F)F ; N#CCC(=O)Nc1ccccc1C(F)(F)F ; O=C(CC1CC1)Nc1ccccc1C(F)(F)F ; C=CCCC(=O)Nc1ccccc1C(F)(F)F ; O=C(CBr)Nc1ccccc1C(F)(F)F ; O=C(CC1CC1)Nc1cccc(C(F)(F)F)c1]

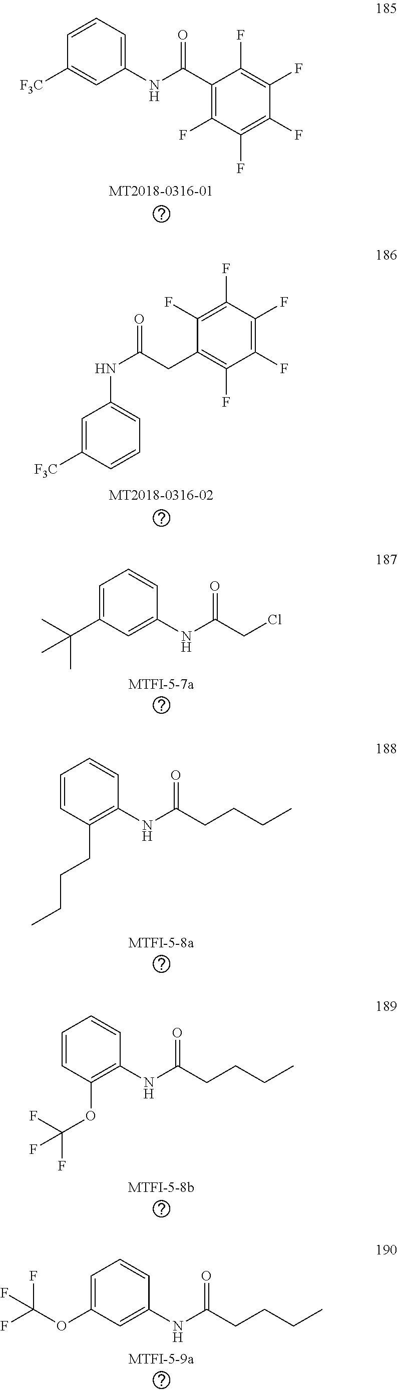 [SMILES: O=C(Nc1cccc(C(F)(F)F)c1)c1c(F)c(F)c(F)c(F)c1F ; O=C(Cc1c(F)c(F)c(F)c(F)c1F)Nc1cccc(C(F)(F)F)c1 ; CC(C)(C)c1cccc(NC(=O)CCl)c1 ; CCCCC(=O)Nc1ccccc1CCCC ; CCCCC(=O)Nc1ccccc1OC(F)(F)F ; CCCCC(=O)Nc1cccc(OC(F)(F)F)c1]

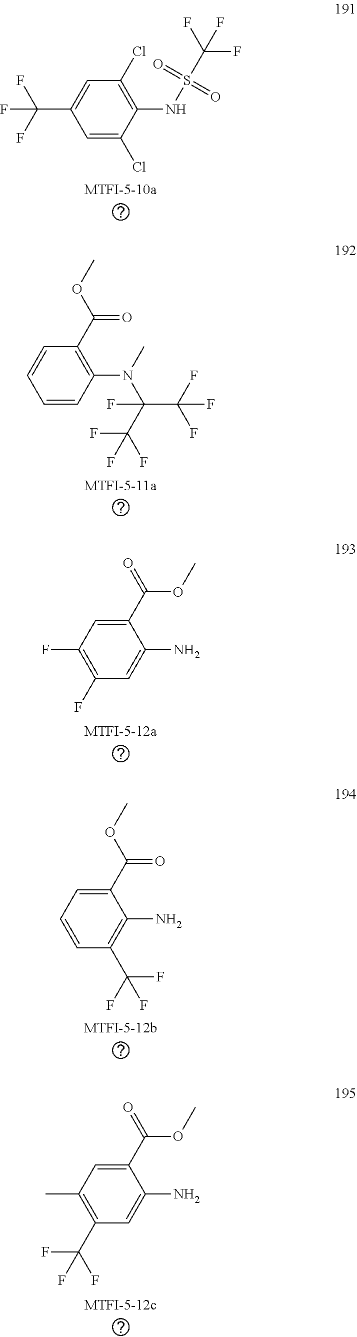

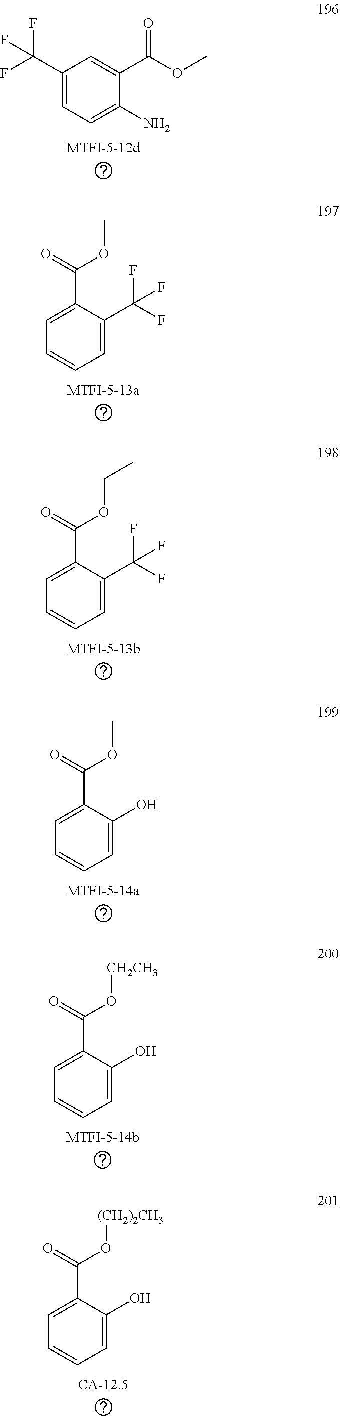

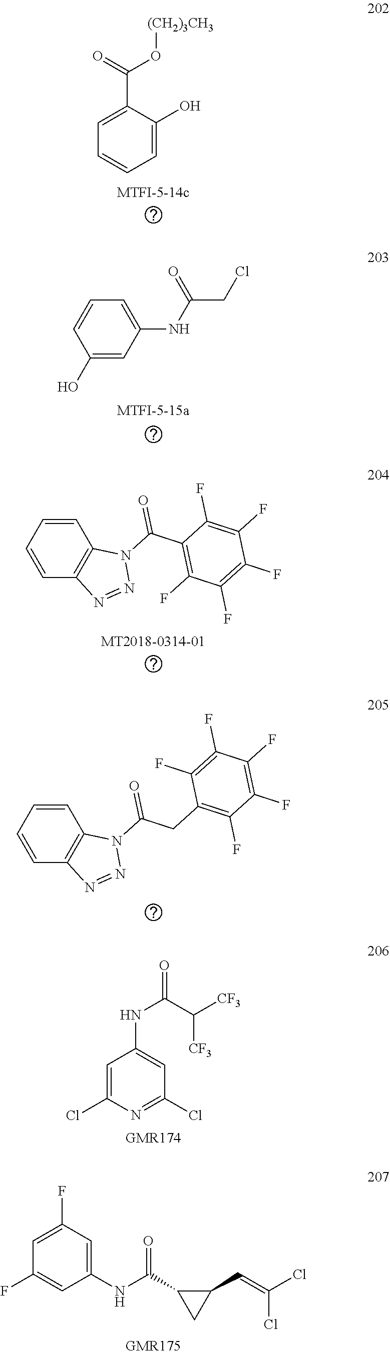

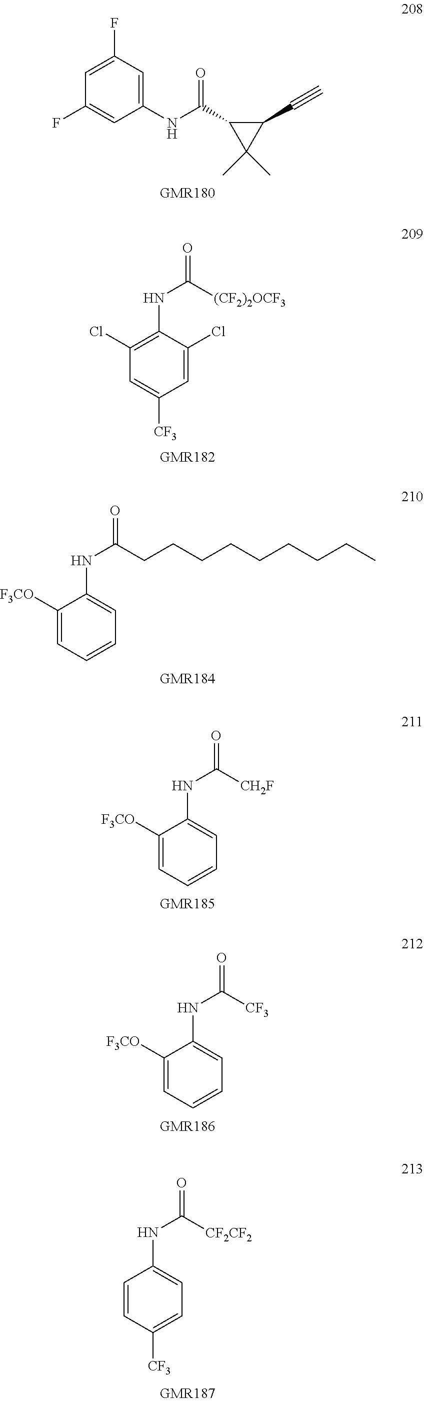

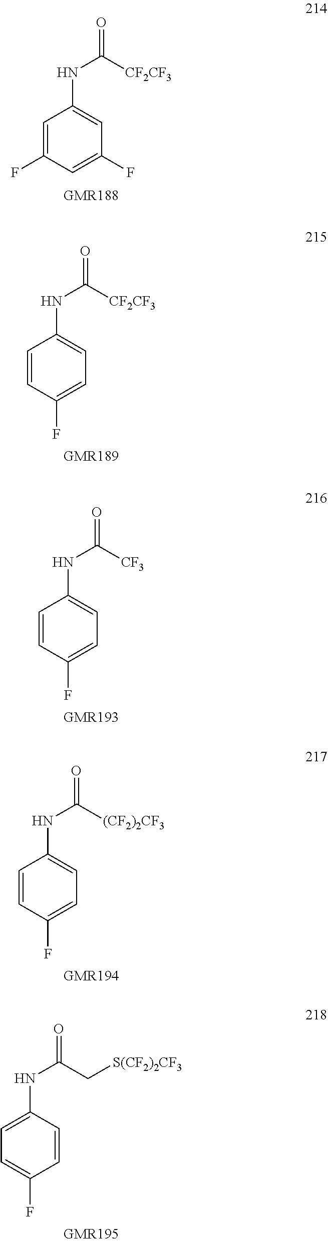

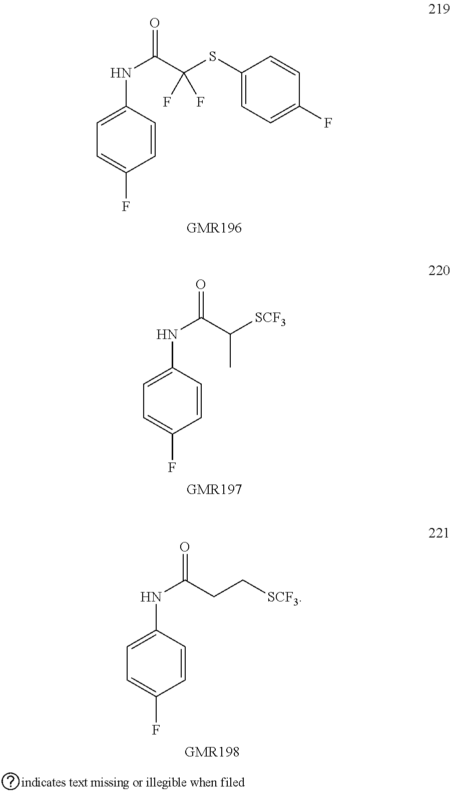

In certain embodiments, the subject matter described herein is directed to a compound of Formula X

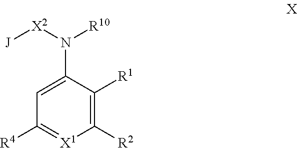

wherein,
when R¹ is hydrogen, X is CR³ wherein R³ is fluorine, and R² and R⁴ are each fluorine;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₃CF₃, —CF₂CF₂OCF₃, —CF₂CN, —(CF₂)₂CF₃, —CH₂SCF₃,

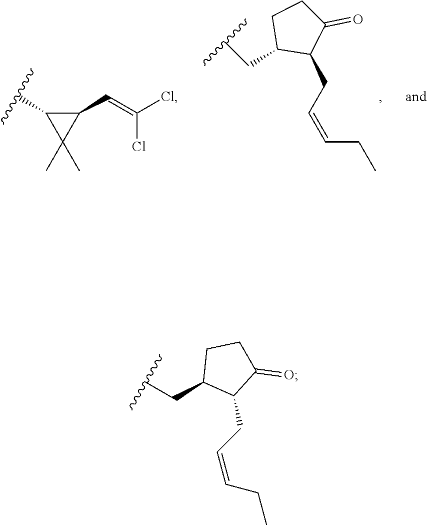

or,
when X¹ is CR³ wherein R³ is fluorine and R¹, R², and R⁴ are each fluorine;
X² is selected from the group consisting of C(O), S(O), S(O)₂, —CH(OH), and —CHCF₃; R¹⁰ is hydrogen or C₁-C₆ alkyl; and
J is selected from the group consisting of —CHF₂, —CF₃, —CF₂CHF₂, —CF₂CF₃, —(CF₂)₂CF₃;
or,
when R¹ is hydrogen, X¹ is N, and R⁴ and R² are each chlorine;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃, —(CF₂)₂CF₃, —(CF₂)₃CF₃, —C(CF₃)₂H,

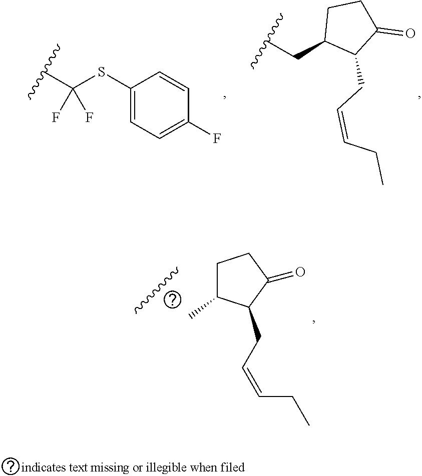

—CF₂CN, —(CH₂)₃CF₃, —C(CF₃)₂F, —CCl₃,

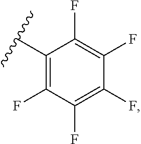 [SMILES: *c1c(F)c(F)c(F)c(F)c1F]

—(CH₂)₄CF₃,

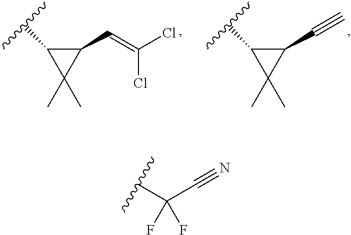

—CHF₂, —CF₃, and —CF₂CF₃;
or,
when R¹ is hydrogen, X¹ is N, and R⁴ and R² are each fluorine;
X² is selected from the group consisting of C(O), S(O), S(O)₂, —CH(OH), and —CHCF₃;
R¹⁰ is hydrogen or C₁-C₆ alkyl; and
J is selected from the group consisting of —CHF₂, —CF₃, —CF₂CF₃, and —(CF₂)₄CF₃;
or
when R¹ is hydrogen, X¹ is CR³ wherein R³ is hydrogen, R⁴ is chlorine, and R² is chlorine,
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃,

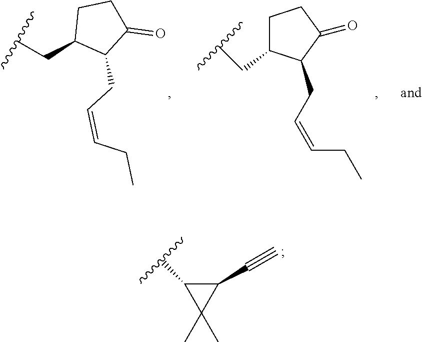

or,
when R¹ is hydrogen, X¹ is N, and R⁴ and R² are each bromine;
X² is selected from the group consisting of C(O), S(O), S(O)₂, —CH(OH), and —CHCF₃;
R¹⁰ is hydrogen or C₁-C₆ alkyl; and
J is selected from the group consisting of —CF₂H, —CF₃, —CF₂CF₃, —(CF₂)₂CF₃, and —(CF₂)₄CF₃;
or,
when R¹ is hydrogen, X¹ is CR³ wherein R³ is hydrogen, one of R⁴ and R² is chlorine and the other is —CF₃;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₄CF₃, —CF(CF₃)₂, —(CF₂)₂OCF₃, —(CF₂)₂CF₃, —CHF₂, —CF₃, and —(CF₂)CF₃;
or,
when R¹ is hydrogen, X¹ is N, and R⁴ and R² are C₁-C₃ alkyl;
X² is selected from the group consisting of C(O), S(O), S(O)₂, —CH(OH), and —CHCF₃;
R¹⁰ is hydrogen or C₁-C₆ alkyl; and
J is selected from the group consisting of —CF₃, —CF₂CF₃, —CHF₂, and —(CF₂)₄CF₃; or,
when R¹ is hydrogen, X¹ is CR³ wherein R³ is hydrogen, R⁴ is —CF₃, and R² is —CF₃;
X² is selected from the group consisting of C(O), S(O), S(O)₂, —CH(OH), and —CHCF₃;
R¹⁰ is hydrogen or C₁-C₆ alkyl; and
J is —(CF₂)₂CF₃.

In certain embodiments, X² is C(O).

In certain embodiments, R¹⁰ is hydrogen.

In certain embodiments, R¹⁰ is C₁-C₃ alkyl.

In certain embodiments, R¹⁰ is methyl.

In certain embodiments, R¹ is hydrogen, X¹ is CR³ wherein R³ is fluorine, and R² and R⁴ are each fluorine, J is selected from the group consisting of —(CF₂)₂CF₃,

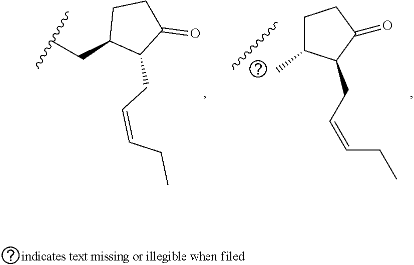

—CF₂CF₂OCF₃, —CH₂SCF₃, —CF₂CN, and

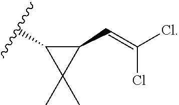 [SMILES: *[C@@H]1[C@@H](C=C(Cl)Cl)C1(C)C]

In certain embodiments, R¹ is hydrogen, X is CR³ wherein R³ is fluorine, and R² and R⁴ are each fluorine, J is —(CF₂)₂CF₃.

In certain embodiments, the compound of Formula X has the structure

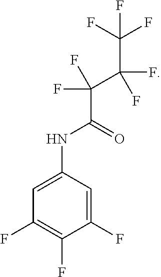 [SMILES: O=C(Nc1cc(F)c(F)c(F)c1)C(F)(F)C(F)(F)C(F)(F)F]

In certain embodiments, R¹ is hydrogen, X¹ is CR³ wherein R³ is fluorine, and R² and R⁴ are each fluorine, J is selected from the group consisting of —(CF₂)₃CF₃, —(CF₂)₂CF₃, and —CF₂CF₂OCF₃.

In certain embodiments, X¹ is CR³, R³ is fluorine and R¹, R², and R⁴ are each fluorine, and J is selected from the group consisting of —CHF₂, —CF₃, —CF₂CHF₂, —CF₂CF₃, —(CF₂)₂CF₃.

In certain embodiments, R¹ is hydrogen, X¹ is N, and R⁴ and R² are each chlorine, and J is selected from the group consisting of —CHF₂, —CF₃, —CF₂CF₃, —(CF₂)₂CF₃,

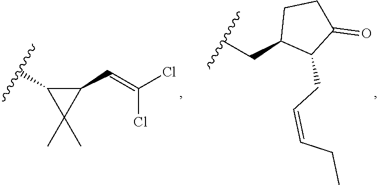

—(CF₂)₄CF₃, —(CF₂)₃CF₃, —(CH₂)₃CF₃, —C(CF₃)₂F, —(CH₂)₄CH₃, —(CF₂)₂OCF₃,

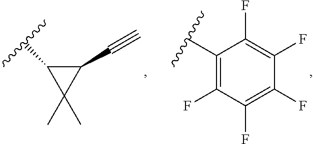

### —CCl₃,

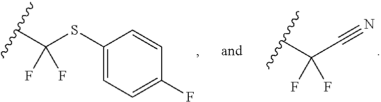 [SMILES: *C(F)(F)Sc1ccc(F)cc1 ; *C(F)(F)C#N]

In certain embodiments, wherein R¹ is hydrogen, X¹ is N, and R⁴ and R² are each chlorine, and J is —(CF₂)₂CF₃ or —(CF₂)₂OCF₃.

In certain embodiments, wherein R¹ is hydrogen, X¹ is N, and R⁴ and R² are each fluorine, and J is selected from the group consisting of —CHF₂, —CF₃, —CF₂CF₃, and —(CF₂)₄CF₃.

In certain embodiments, wherein R¹ is hydrogen, X is CR³ wherein R³ is hydrogen, R⁴ is chlorine, and R² is chlorine, and J is

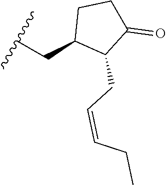 [SMILES: *C[C@H]1CCC(=O)[C@@H]1C/C=C\CC]

or —(CF₂)₂OCF₃.

In certain embodiments, wherein R¹ is hydrogen, X¹ is N, and R⁴ and R² are each bromine, and J is selected from the group consisting of —CF₂H, —CF₃, —CF₂CF₃, —(CF₂)₂CF₃, and —(CF₂)₄CF₃.

In certain embodiments, wherein R¹ is hydrogen, X¹ is CR³ wherein R³ is hydrogen, one of R⁴ and R² is chlorine and the other is —CF₃, and J is selected from the group consisting of —(CF₂)₂OCF₃, —CHF₂, —CF₃, —(CF₂)CF₃, —(CF₂)₂CF₃, —(CF₂)₄CF₃, —(CH₂)₄CH₃, and —CF(CF₃)₂.

In certain embodiments, wherein R¹ is hydrogen, X¹ is CR³ wherein R³ is hydrogen, one of R⁴ and R² is chlorine and the other is —CF₃, and J is —(CF₂)₂CF₃.

In certain embodiments, wherein R¹ is hydrogen, X¹ is N, and R⁴ and R² are each methyl, and J is selected from the group consisting of —CF₃, —CF₂CF₃, —CHF₂, and —(CF₂)₄CF₃.

In certain embodiments, wherein R¹ is hydrogen, X¹ is CR³ wherein R³ is hydrogen, R⁴ is —CF₃, and R² is —CF₃, and J is —(CF₂)₂CF₃.

In certain embodiments, J is

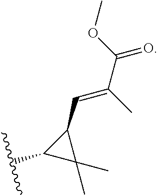 [SMILES: *[C@@H]1[C@@H](/C=C(\C)C(=O)OC)C1(C)C]

In certain embodiments, the compound of Formula X is selected from the group consisting of

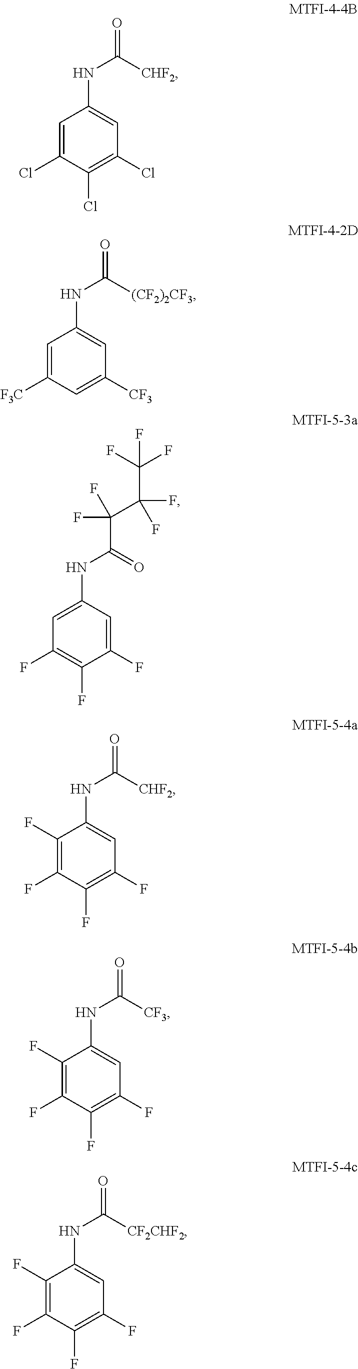

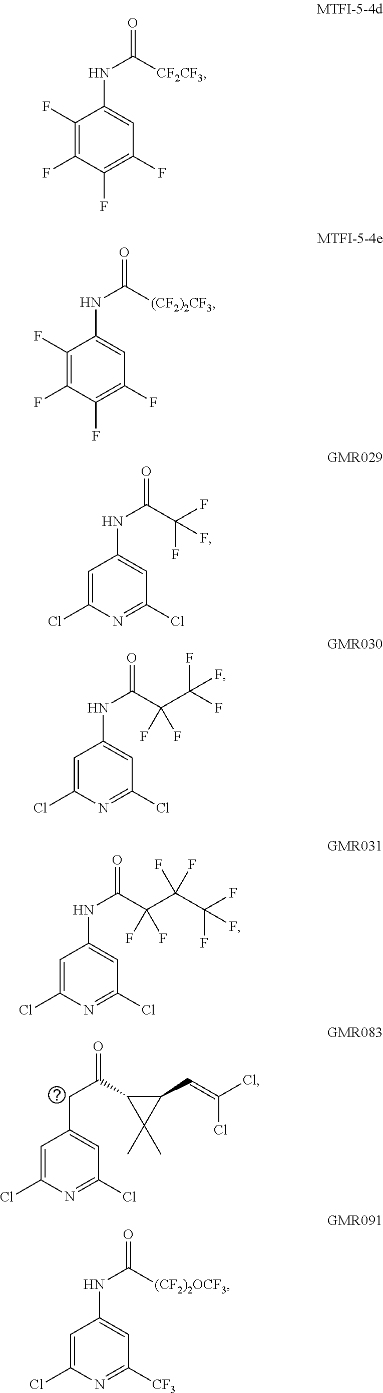

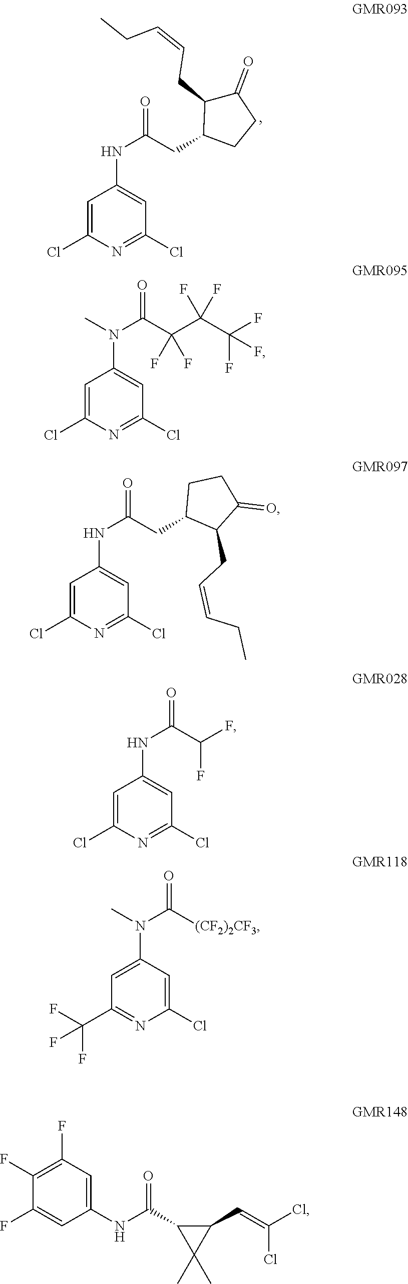

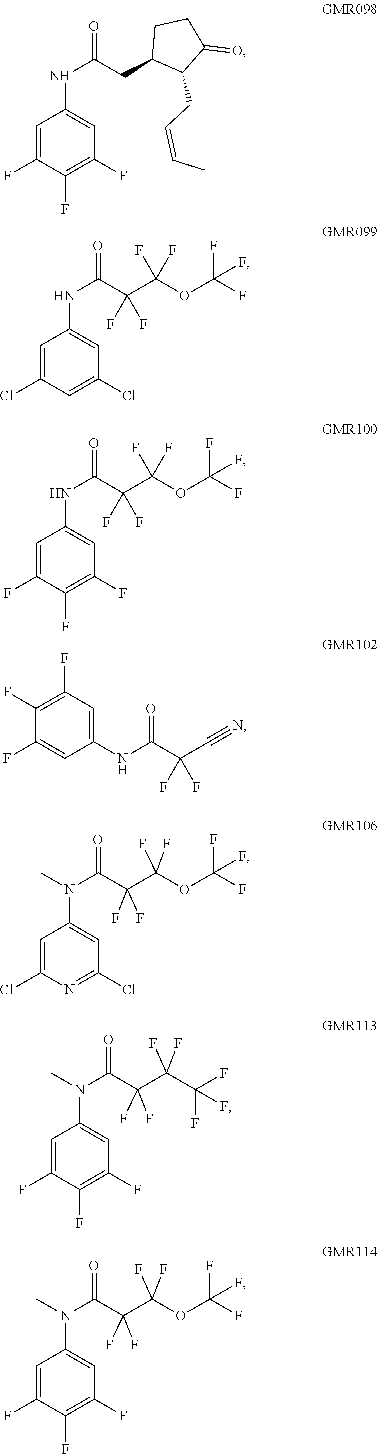

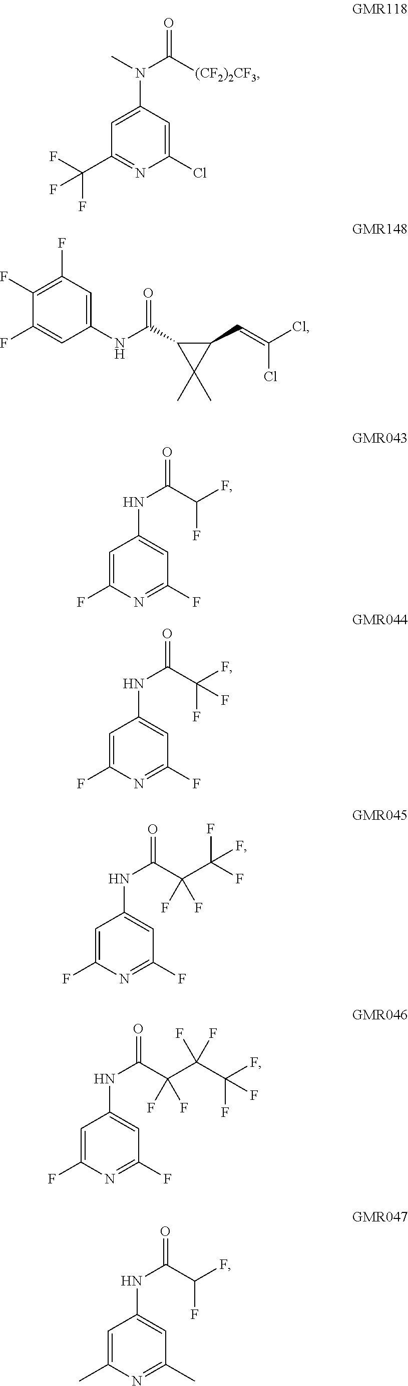

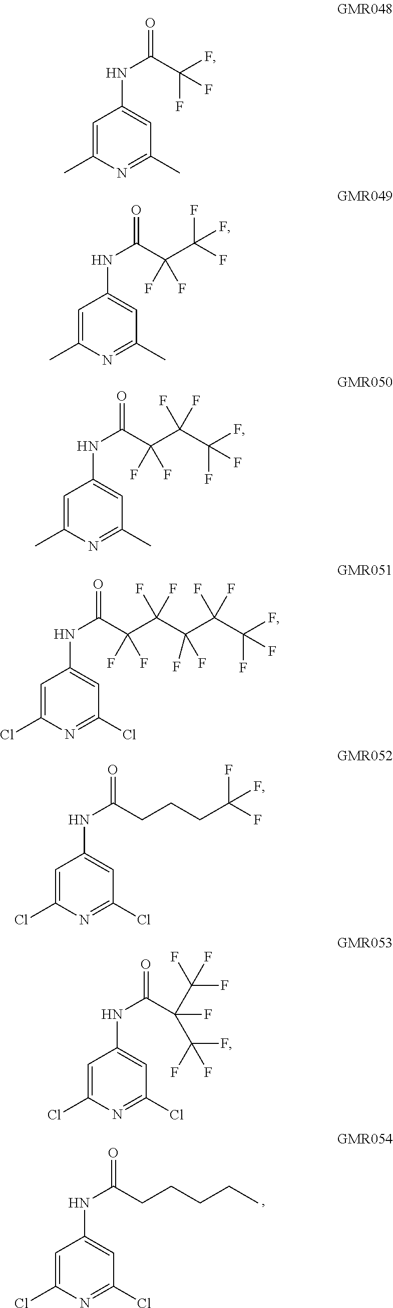

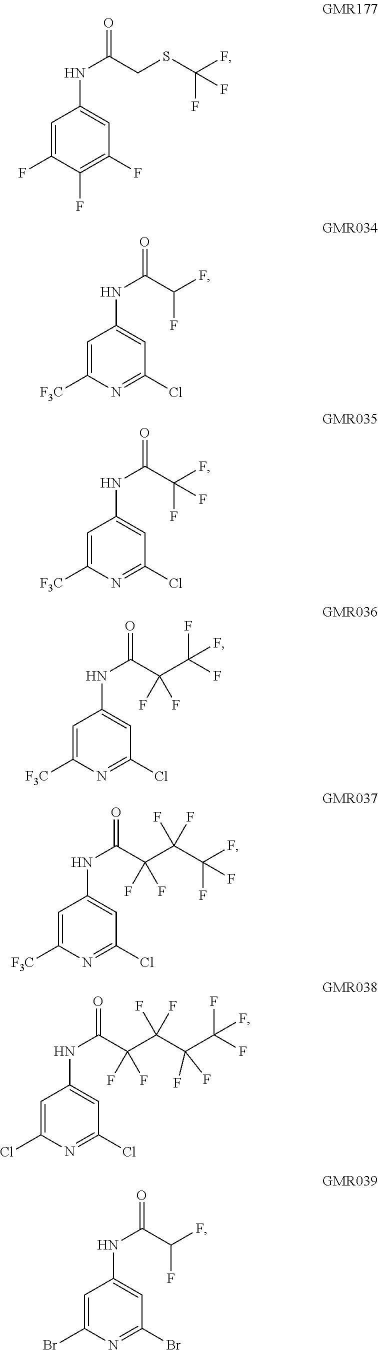

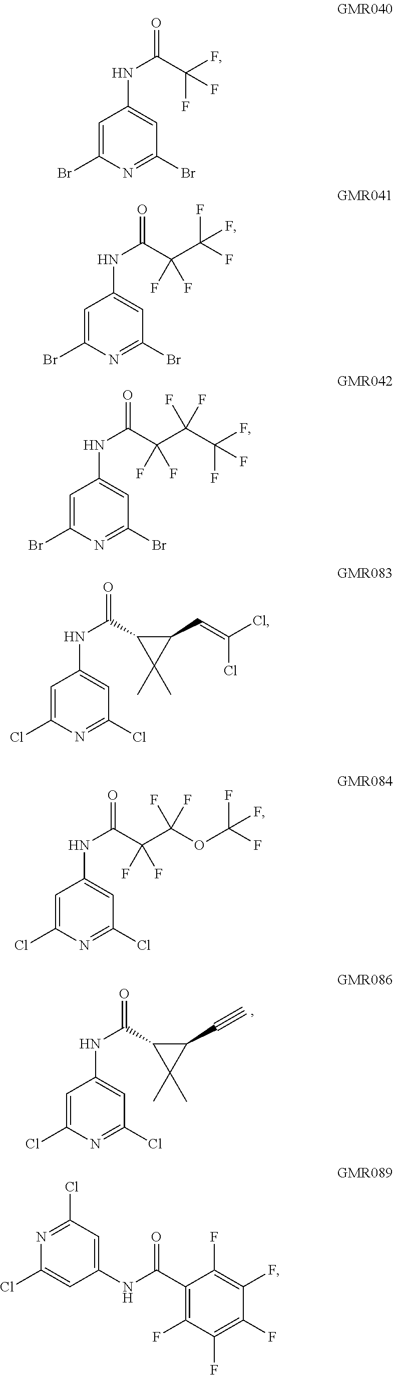

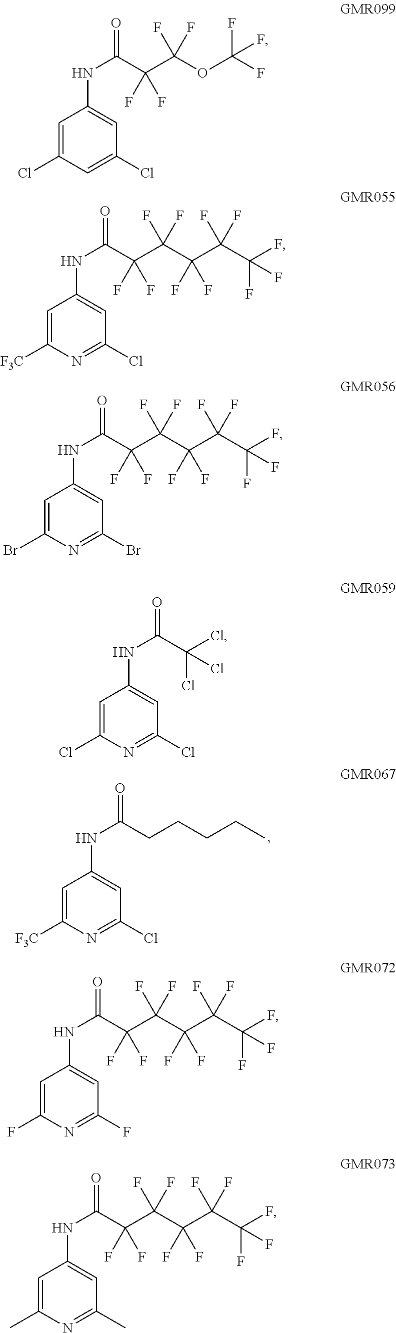

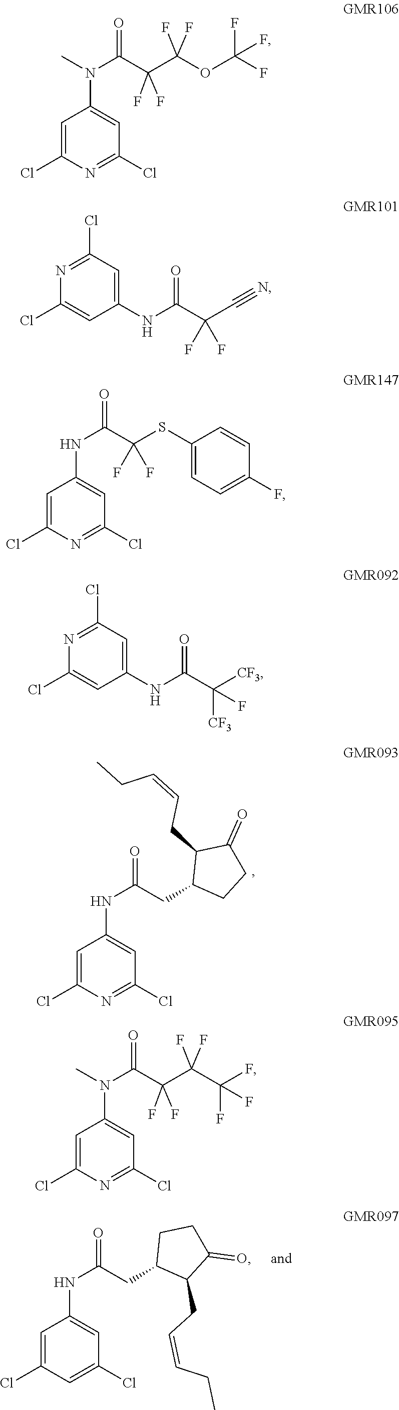

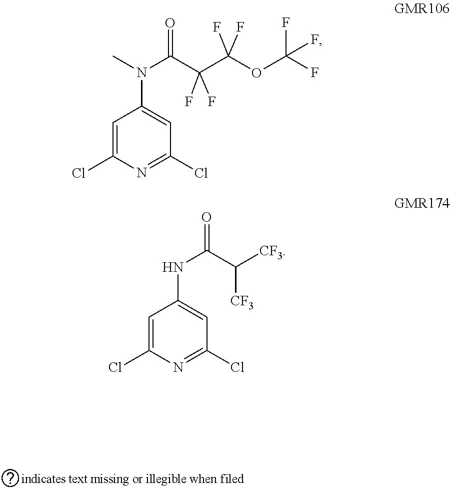

In certain embodiments, the subject matter disclosed herein is directed to a compound selected from the group consisting of

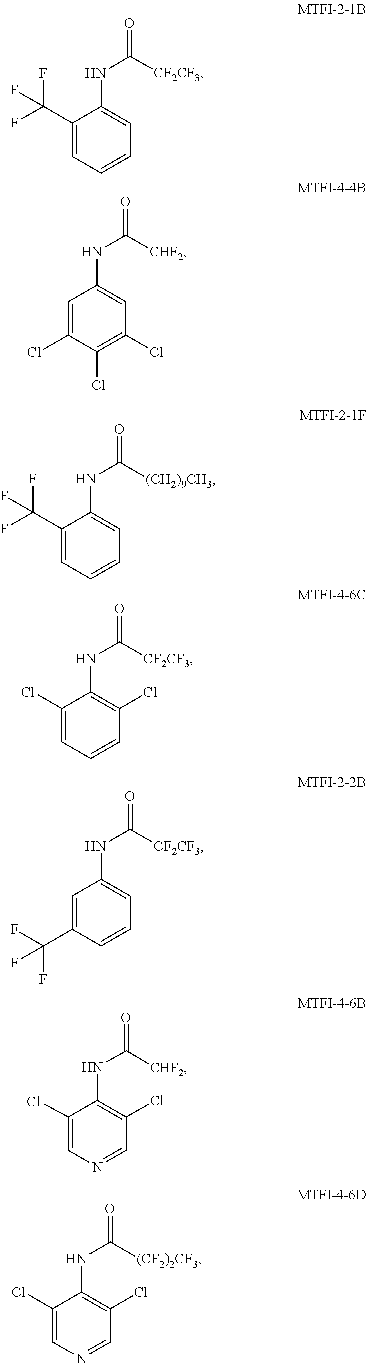

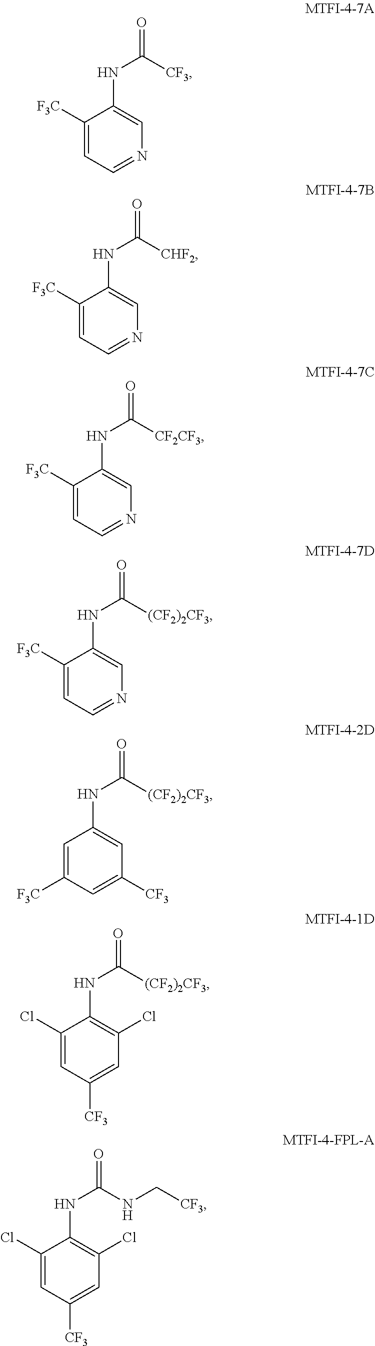

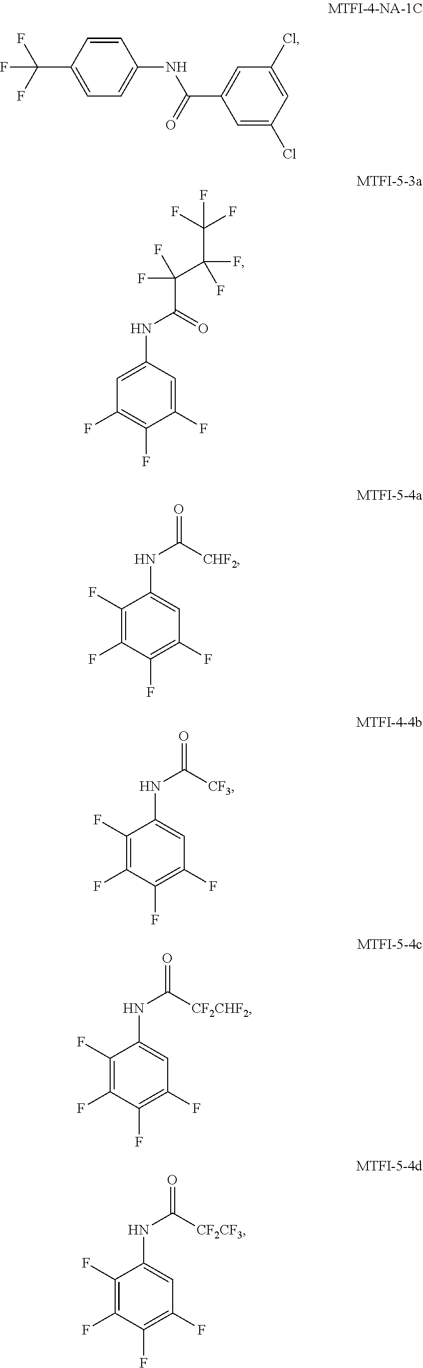

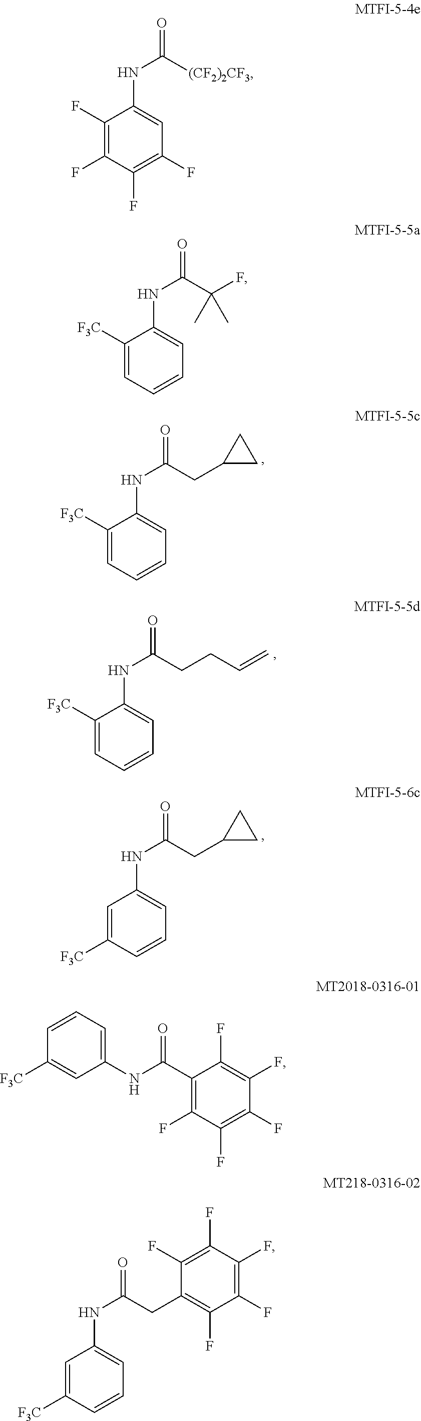

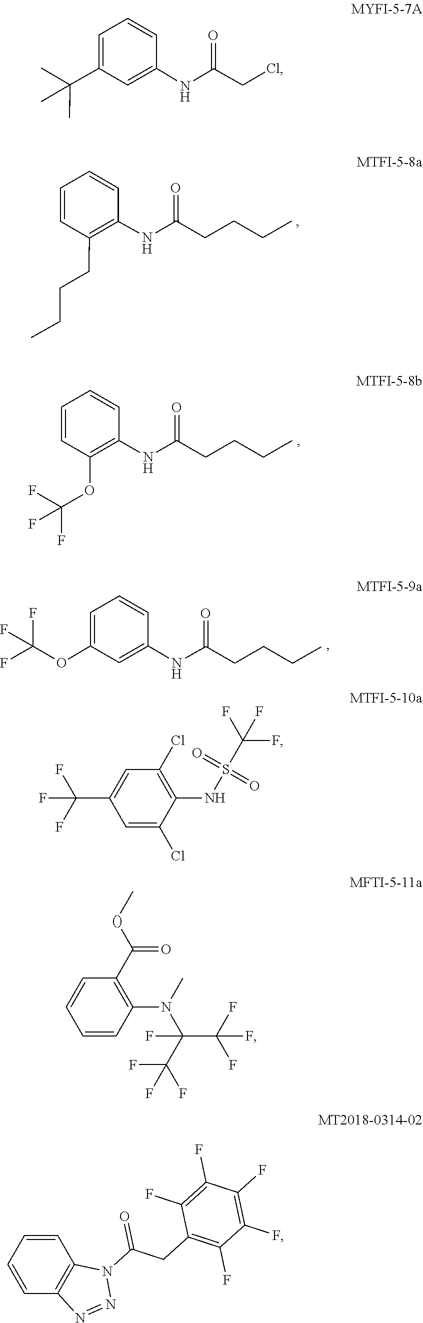

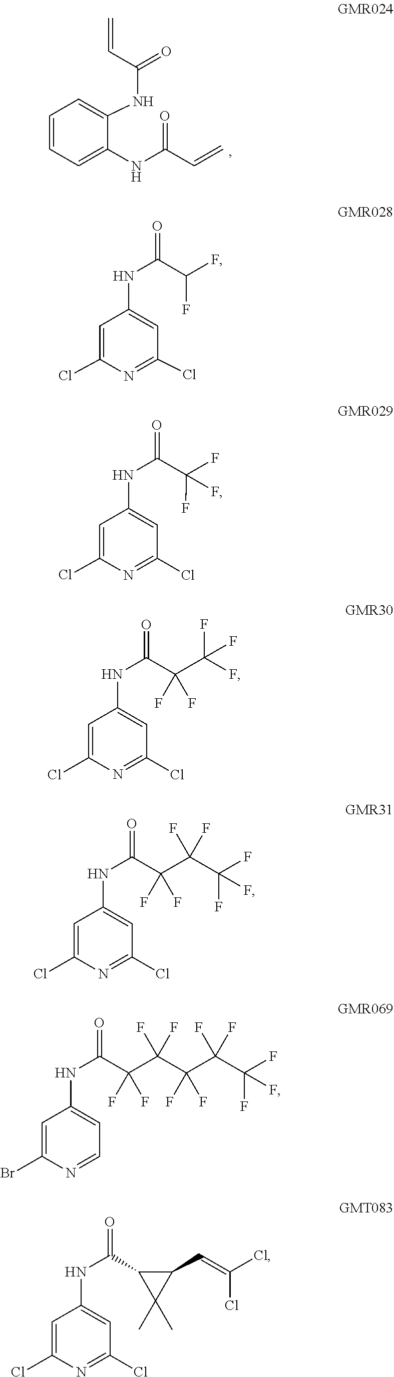

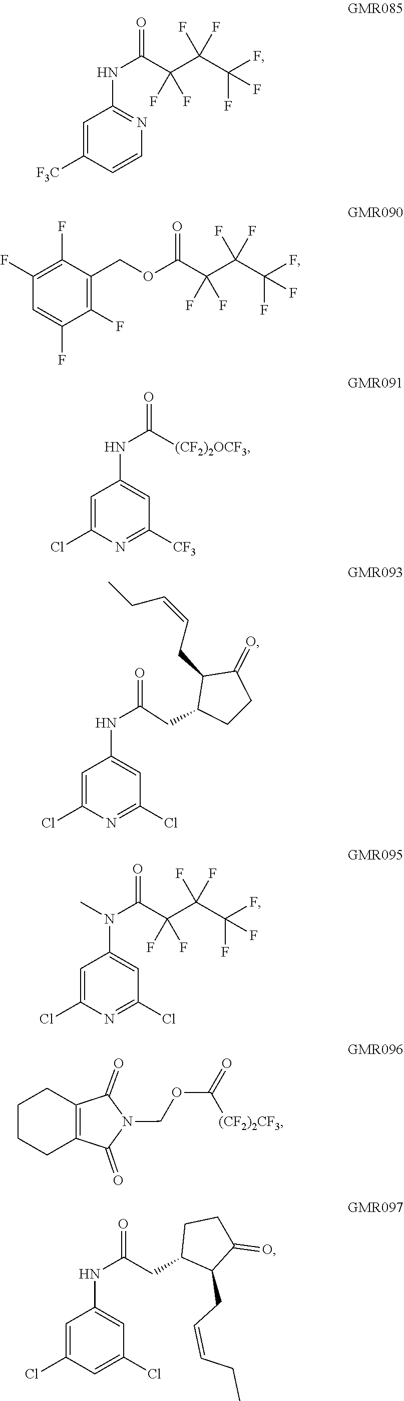

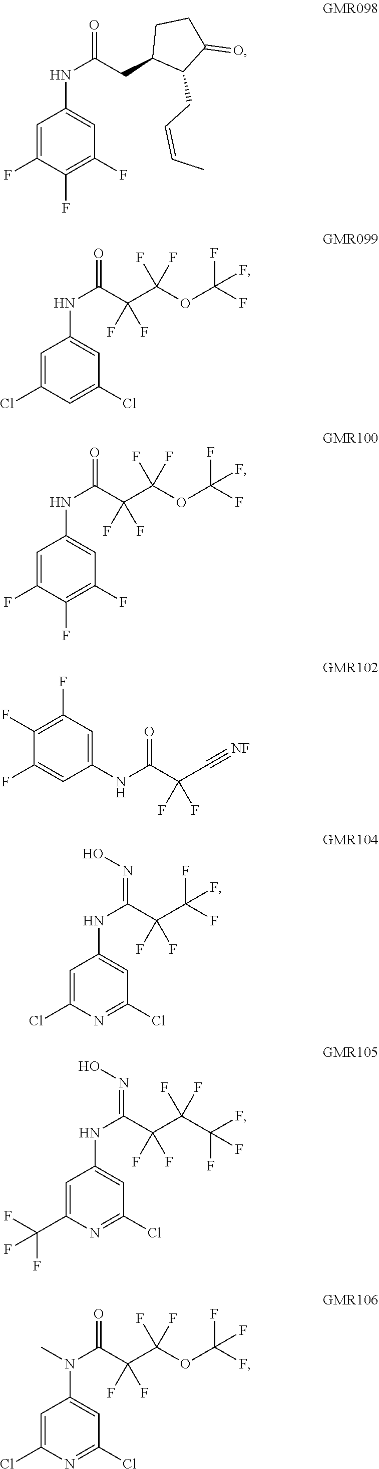

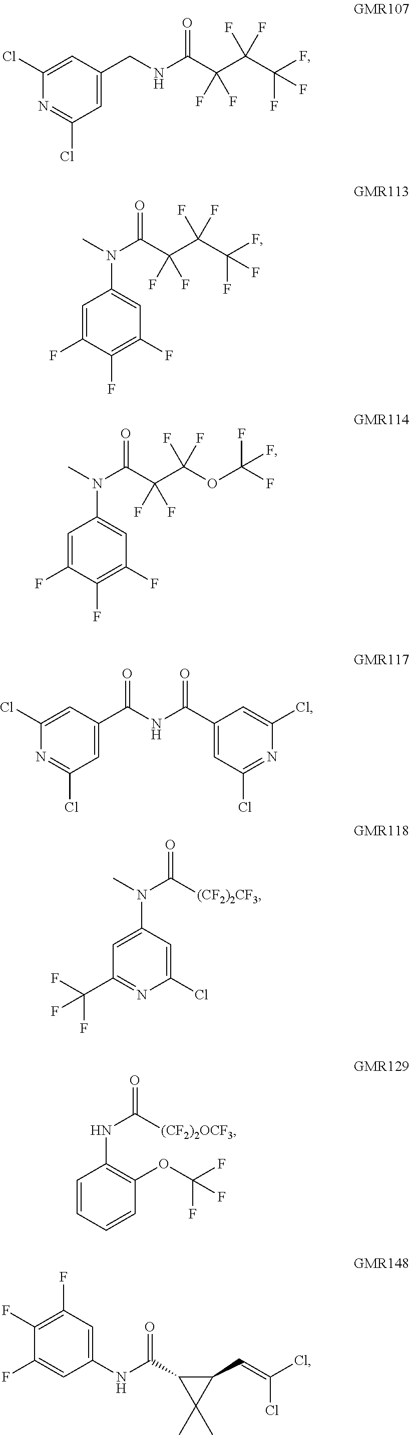

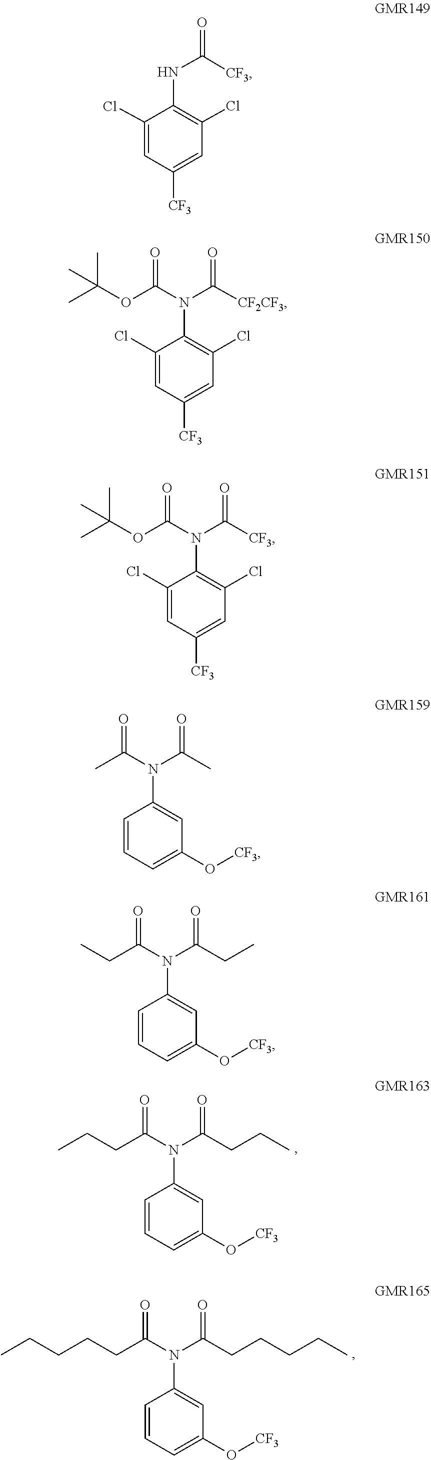

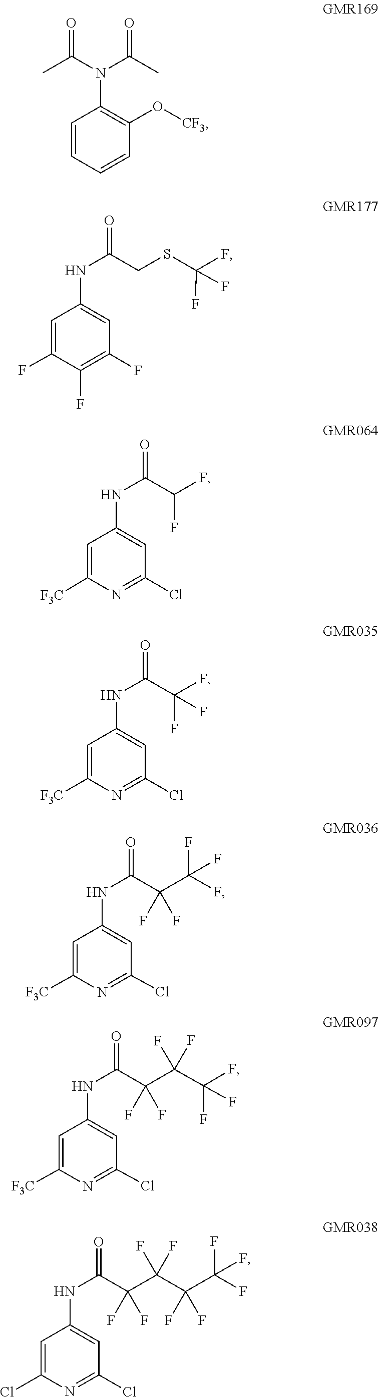

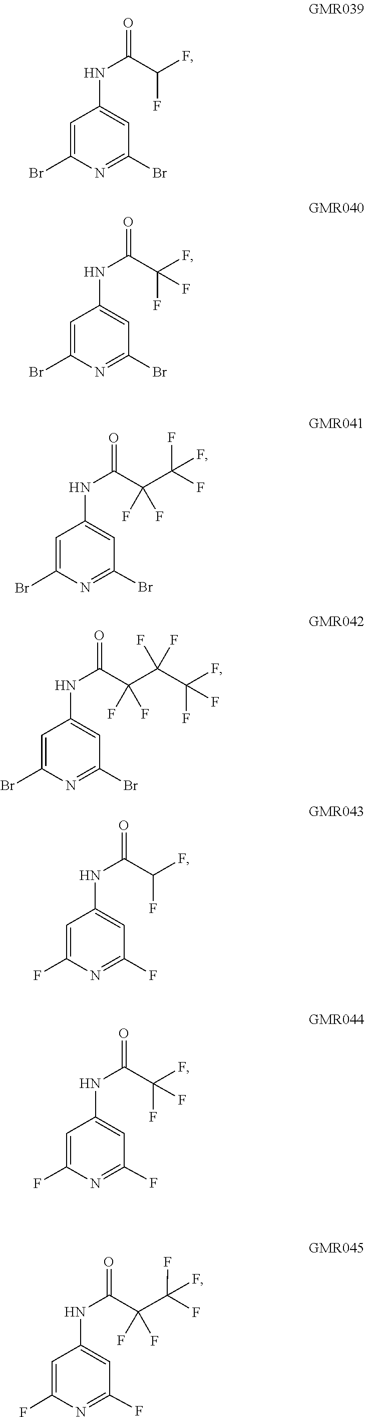

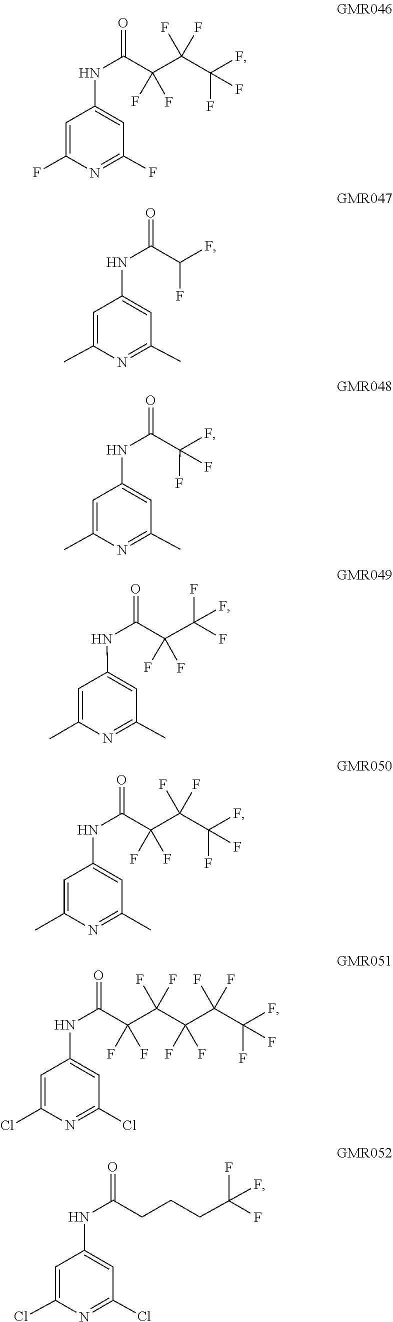

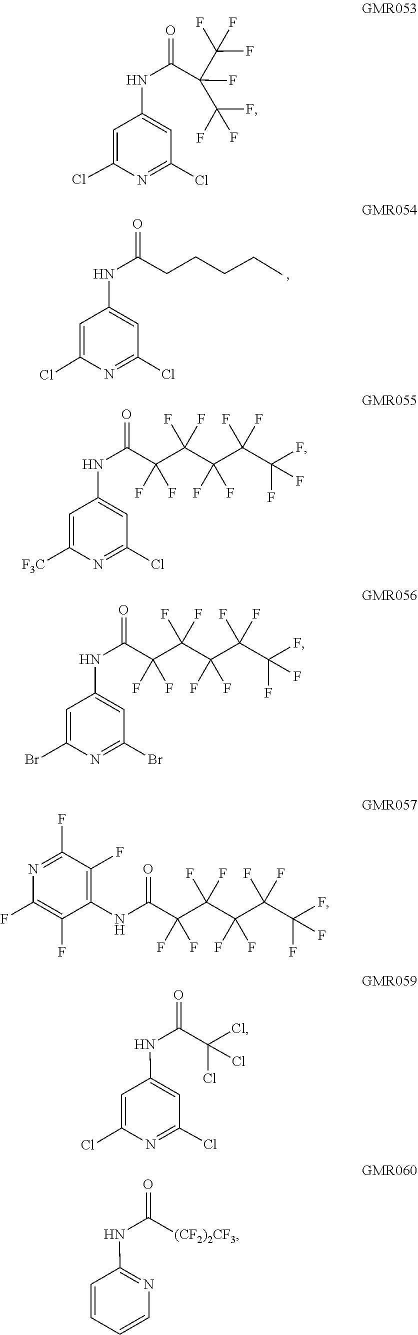

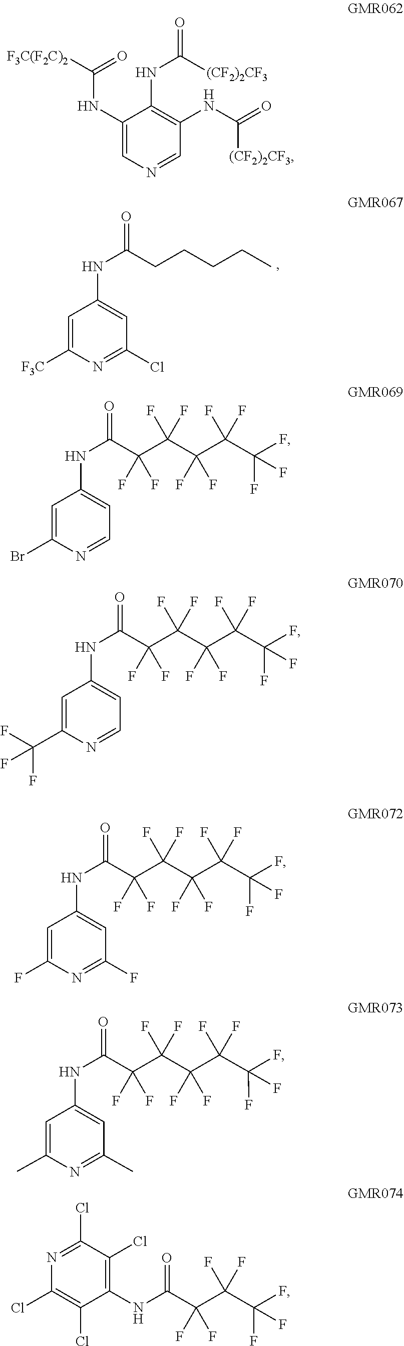

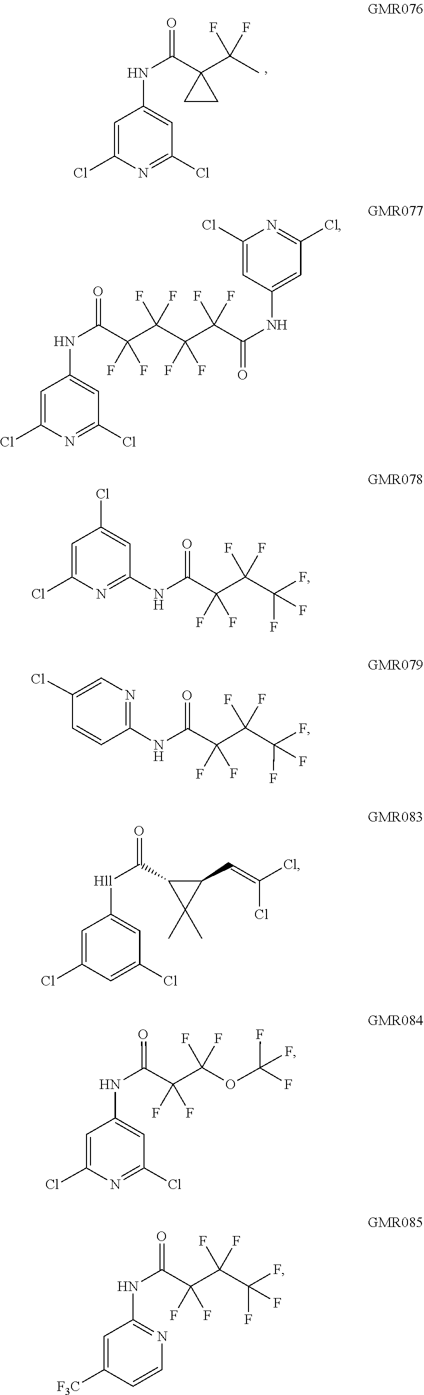

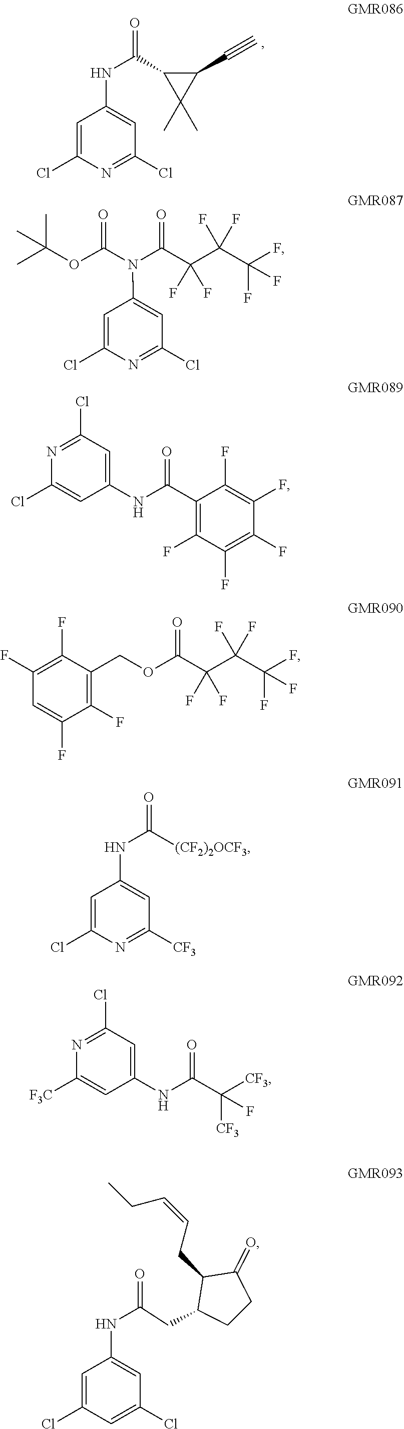

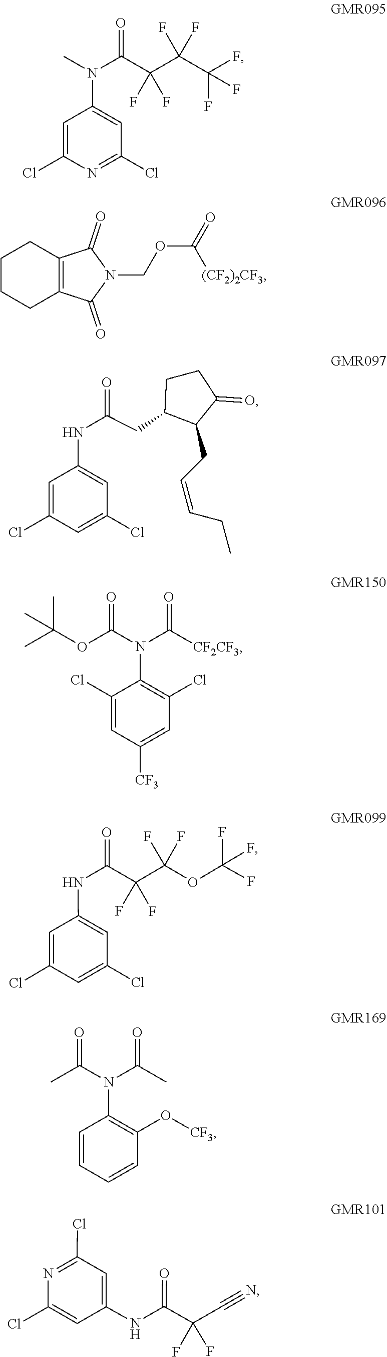

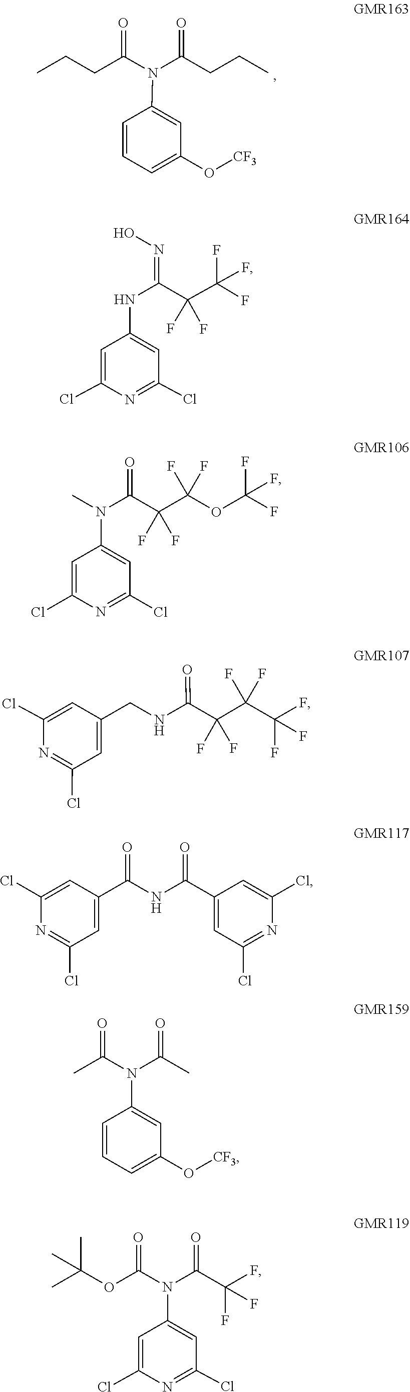

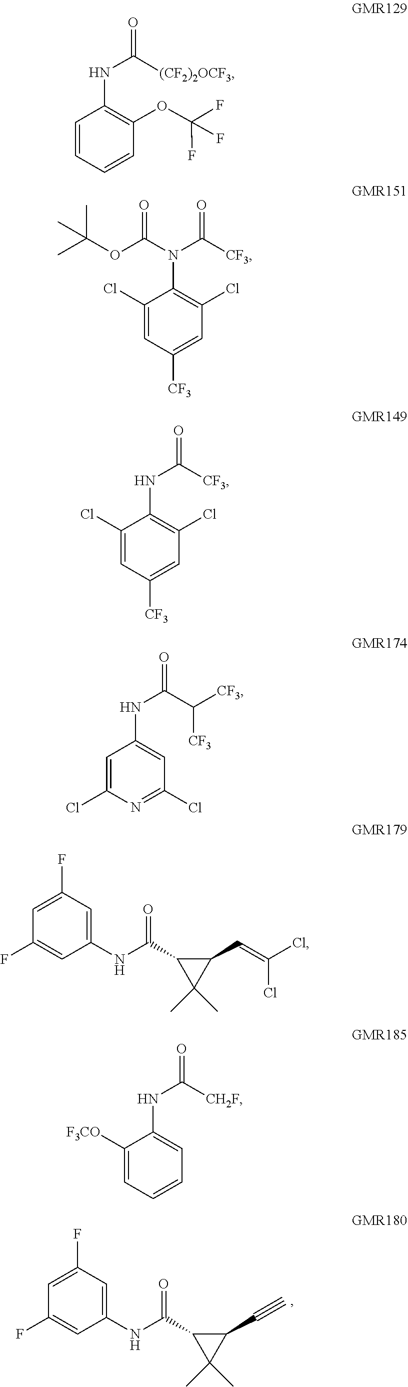

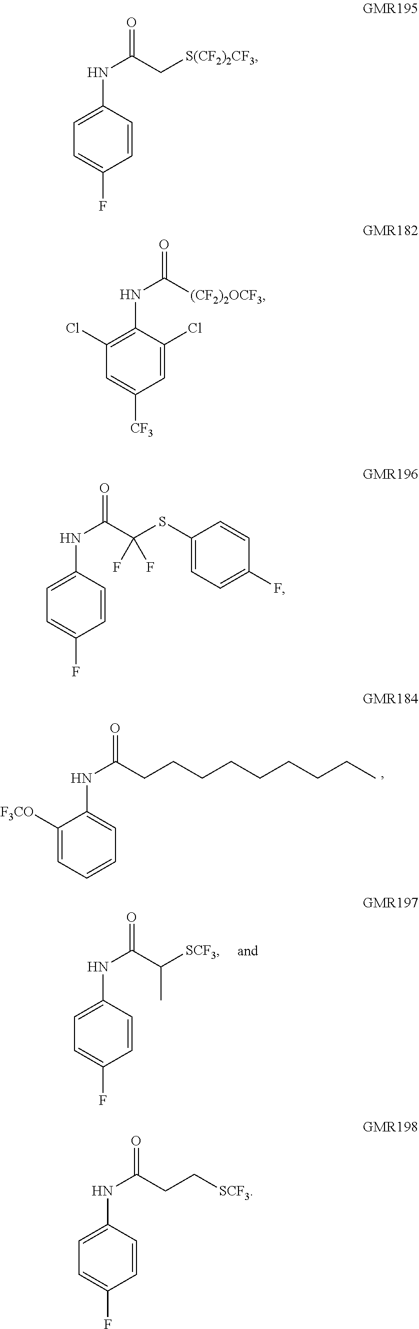

In certain embodiments, the subject matter disclosed herein is directed to a compound of Formula II′:

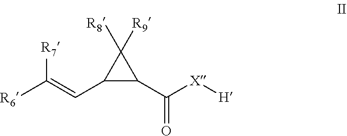

wherein R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, haloaryl, or C₃-C₁₂ heterocycloalkyl; R_{8′} and R_{9′} are each independently selected from hydrogen haloalkyl, alkenyl, alkynyl, and C₁-C₆ alkyl; and
X″ is selected from the group consisting of O, NH, and S.

In certain embodiments, R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl.

In certain embodiments, X″ is O.

In certain embodiments, R_{6′} and R_{7′} are each independently selected from halo or C₁-C₆ alkyl.

In certain embodiments, R_{6′} and R_{7′} are each halo. In certain embodiments, R_{6′} and R_{7′} are each chloro.

In certain embodiments, R_{8′} and R_{9′} are each methyl.

In certain embodiments, the compound of Formula II is

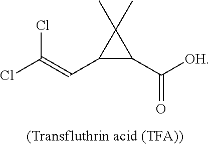

In certain embodiments, the subject matter disclosed herein is directed to a compound of Formula III′:

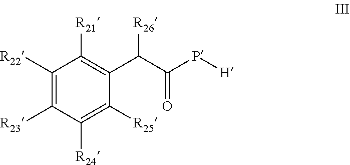

R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is selected from the group consisting of O, NH, and S; and
R_{26′} is selected from the group consisting of hydrogen, haloalkyl, alkenyl, alkynyl, and linear or branched C₁-C₆ alkyl.

In certain embodiments, P′ is O.

In certain embodiments, R_{23′} is halo. In certain embodiments, R_{23′} is chloro.

In certain embodiments, R_{21′}, R_{22′}, R_{24′}, and R_{25′} are each hydrogen.

In certain embodiments, R_{26′} is branched C₁-C₆ alkyl. In certain embodiments, R_{26′} is isopropyl.

In certain embodiments, R_{27′} is hydrogen.

### III. Compositions

The repellent compositions described herein may take various forms, including solutions, dusts, granular formulations, and emulsions. They may be prepared from concentrates, such as emulsifiable concentrates and wettable powders. In addition to the active agents, the compositions can also comprise various inert ingredients, depending upon the form taken by the composition.

If prepared from an emulsifiable concentrate, the composition may contain a surfactant such as, for example, a mixture of a polyethylene oxide with a blend of oil soluble non-ionic and anionic sulfonates. The surfactant normally comprises between about 1 and 15% by weight of the emulsifiable concentrate.

Wettable powders can also be used to make the repellent compositions. Compositions so made may be applied to the area to be protected as emulsions in water or other liquid diluents. Typical among the carriers employed in wettable powders are walnut flour, cane sugar, fuller's earth, attapulgite clays, kaolin clays, silicas and other highly absorbent, readily wetted carriers. The wettable powders themselves generally are prepared to contain about 5 to 80% by weight of the active component, depending on the absorbency of the carrier. A wettable powder usually also contains a small amount of a surfactant.

Granular repellent compositions, wherein the active component is carried on relatively coarse particles as the carrier, are also useful in repelling crawling insect pests. Dry dusts, in which the active component is admixed with finely divided solids such as talc, attapulgite clay, kieselguhr, and other organic and inorganic solids, which act as carriers for the active component, also find utility. These finely divided solids usually have an average particle size of less than about 50 microns.

Pressurized sprays such as aerosols, in which the active component is present in solution or in a finely divided form, may also be used.

The concentration of the active agent(s) in any of the repellent compositions may vary in the range from about 0.00001% to about 10% by weight, depending on the formulation. A very broad latitude in the type of repellent composition and the concentration of the active agent(s) within the aforesaid range is possible.

In certain embodiments, the subject matter disclosed herein is directed to a compound of Formula IA, IB, IC, ID, IE, IF, or IG in admixture with a carrier.

In certain embodiments, the subject matter disclosed herein is directed to a compound of Formula IA, IB, IC, ID, IE, IF, or IG in admixture with a carrier, wherein said compound is selected from the group consisting of

| | |
|---|---|
| MTFI-2-1B | MTFI-3-1B |
| MTFI-2-1F | MTFI-3-1C |
| MTFI-2-2B | MTFI-3-1D |
| MTFI-3-2E | MTFI-3-2F |
| MTFI-3-3A | MTFI-3-4B |
| MTFI-3-5A | MTFI-4-4B |
| MTFI-4-6B | MTFI-4-6C |
| MTFI-4-6D | MTFI-4-7A |
| MTFI-4-7B | MTFI-4-7C |
| MTFI-4-7D | MTFI-4-2D |
| MTFI-4-1D | MTFI-4-FPL-A |
| MTFI-4-NA-1C | MTFI-5-3a |
| MTFI-5-4a | MTFI-5-4b |
| MTFI-5-4c | MTFI-5-4d |
| MTFI-5-4e | MTFI-5-5a |
| MTFI-5-5c | MTFI-5-5d |
| MTFI-5-6c | MT2018-0316-01 |
| MT2018-0316-02 | MTFI-5-7a |
| MTFI-5-8a | MTFI-5-8b |
| MTFI-5-9a | MTFI-5-10a |
| MTFI-5-11a | MT2018-0314-02 |
| GMR024 | GMR028 |
| GMR029 | GMR030 |
| GMR031 | GMR069 |
| GMR083 | GMR085 |
| GMR090 | GMR091 |
| GMR093 | GMR095 |
| GMR096 | GMR097 |
| GMR098 | GMR099 |
| GMR100 | GMR102 |
| GMR104 | GMR105 |
| GMR106 | GMR107 |
| GMR113 | GMR114 |
| GMR117 | GMR118 |
| GMR129 | GMR148 |
| GMR149 | GMR150 |
| GMR151 | GMR159 |
| GMR161 | GMR163 |
| GMR165 | GMR169 |
| GMR177 | GMR034 |
| GMR035 | GMR036 |
| GMR037 | GMR038 |
| GMR039 | GMR040 |
| GMR041 | GMR042 |
| GMR043 | GMR044 |
| GMR045 | GMR046 |
| GMR047 | GMR048 |
| GMR049 | GMR050 |
| GMR051 | GMR052 |
| GMR053 | GMR054 |
| GMR055 | GMR056 |
| GMR057 | GMR059 |
| GMR060 | GMR062 |
| GMR067 | GMR069 |
| GMR070 | GMR072 |
| GMR073 | GMR074 |
| GMR076 | GMR077 |
| GMR078 | GMR079 |
| GMR083 | GMR084 |
| GMR085 | GMR086 |
| GMR087 | GMR089 |
| GMR090 | GMR091 |
| GMR092 | GMR093 |
| GMR095 | GMR096 |
| GMR097 | GMR098 |
| GMR099 | GMR100 |
| GMR101 | GMR102 |
| GMR104 | GMR106 |
| GMR107 | GMR117 |
| GMR118 | GMR119 |
| GMR129 | GMR147 |
| GMR149 | GMR150 |
| GMR151 | GMR159 |
| GMR163, and | GMR169. |

In certain embodiments, the compositions disclosed herein comprise a compound of Formula IA, IB, IC, ID, IE, IF, or IG and an essential oil, wherein the composition is synergistic, in admixture with a carrier, in any molar or weight ratio.

In certain embodiments, the compositions disclosed herein comprise about 0.01-99.99%, 1-99%, 5-95%, 10-90%, 20-80%, 30-70%, 40-60%, or 50% of a compound of Formula IA, IB, IC, ID, IE, IF, or IG and 99.99-0.01%, 99-1%, 95-5%, 90-10%, 80-20%, 70-30%, 60-40%, or 50% of an essential oil. In certain embodiments, the compound of Formula IA, IB, IC, ID, IE, IF, or IG comprises about 1%, 2%, 3%, 4%, 5%, 10%, 15%, 20%, 2%5, 30%, 40%, 50%, 60%, 65%, 70%, 75%, 80%, 90%, 95%, 96%, 97%, or 98% of the composition in combination with an essential oil, wherein the composition is synergistic, in admixture with a carrier.

In certain embodiments, the compositions disclosed herein comprise a compound of Formula IA, IB, IC, ID, IE, IF, or IG, and citronella oil, wherein the composition is synergistic, in admixture with a carrier, in any molar or weight ratio.

In certain embodiments, the compositions disclosed herein comprise about 0.01-99.99%, 1-99%, 5-95%, 10-90%, 20-80%, 30-70%, 40-60%, or 50% of a compound of Formula IA, IB, IC, ID, IE, IF, or IG and 99.99-0.01%, 99-1%, 95-5%, 90-10%, 80-20%, 70-30%, 60-40%, or 50% of citronella oil, wherein the composition is synergistic, in admixture with a carrier.

In certain embodiments, the compositions disclosed herein comprise a Formula IA, IB, IC, ID, IE, IF, or IG and a second compound selected from a pyrethroid, a compound of Formula II′, or a compound of Formula III′, wherein the composition is synergistic, in admixture with a carrier, in any weight or molar ratio.

In certain embodiments, the compositions disclosed herein comprise about 0.01-99.99%, 1-99%, 5-95%, 10-90%, 20-80%, 30-70%, 40-60%, or 50% of a compound of Formula IA, IB, IC, ID, IE, IF, or IG and 99.99-0.01%, 99-1%, 95-5%, 90-10%, 80-20%, 70-30%, 60-40%, or 50% of a second compound selected from a pyrethroid, a compound of Formula II′, or a compound of Formula III′, wherein the composition is synergistic, in admixture with a carrier. In certain embodiments, the compound of Formula IA, IB, IC, ID, IE, IF, or IG comprises about 1%, 2%, 3%, 4%, 5%, 10%, 15%, 20%, 2%5, 30%, 40%, 50%, 60%, 65%, 70%, 75%, 80%, 90%, 95%, 96%, 97%, or 98% of the composition in combination with a second compound selected from a pyrethroid, a compound of Formula II′, or a compound of Formula III′, wherein the composition is synergistic, in admixture with a carrier.

In certain embodiments, the compositions disclosed herein comprise about 0.01-99.99%, 1-99%, 5-95%, 10-90%, 20-80%, 30-70%, 40-60%, or 50% of a compound of Formula IA, IB, IC, ID, IE, IF, or IG and about 99.99-0.01%, 99-1%, 95-5%, 90-10%, 80-20%, 70-30%, 60-40%, or 50% of TFA, wherein the composition is synergistic, in admixture with a carrier. In certain embodiments, the compound of Formula IA, IB, IC, ID, IE, IF, or IG comprises about 1%, 2%, 3%, 4%, 5%, 10%, 15%, 20%, 2%5, 30%, 40%, 50%, 60%, 65%, 70%, 75%, 80%, 90%, 95%, 96%, or 98% of the composition in combination with TFA.

“Synergistic” as used herein refers to a combination which is more effective than the additive effects of the two or more single agents. A determination of a synergistic interaction between two active compounds may, in some embodiments, be based on the results obtained from the experimental information described herein. The results of these experiments can be analyzed using, for example, the Bliss activity method.

### IV. Methods of Repelling Insects

In repelling arthropods, such as insect pests according to the methods, the locus from which such insect pests are to be repelled will be contacted with a repellent amount of the compound of Formula IA, IB, IC, ID, IE, IF, IG, or X, or a synergistic composition. A repellent amount of the compound of Formula IA, IB, IC, ID, IE, IF, IG, or X, or a synergistic composition can vary somewhat, depending on the arthropod or species of insect to be repelled, the nature of the locus, including the type of surface, from which the insects are to be repelled, and so forth, but generally between about 1 mg/m² and 200 mg/m² is a repellent amount. As is well known in the art, the degree of effectiveness of the repellent may vary with the formulation and the method of application.

In carrying out the methods, the compound of Formula IA, IB, IC, ID, IE, IF, IG, or X, or a synergistic composition may be applied in any suitable fashion to the area in which the repellency of arthropods or insect pests, including acarids, is desired. Means of effective applications are well known in the art.

In certain embodiments, the subject matter described herein is directed to a method of repelling arthropods, comprising contacting an object or locus with a compound of Formula IA, IB, IC, ID, IE, IF, IG, or X, wherein the arthropods are repelled from an adjacent area to said object or locus, wherein the arthropods are not physically contacted with the locus. In certain embodiments, the arthropods are insects.

In certain embodiments, the subject matter described herein is directed to a method of repelling arthropods, comprising contacting an object or locus with a compound of Formula IA, IB, IC, ID, IE, IF, IG, or X, or a composition comprising a compound of Formula IA, IB, IC, ID, IE, IF, IG, or X and a carrier, wherein the arthropods are repelled from an adjacent area to said object or locus, wherein the arthropods are not physically contacted with the locus. In certain embodiments, the arthropods are insects.

In certain embodiments, the subject matter described herein is directed to a method of providing a compound of Formula IA, IB, IC, ID, IE, IF, IG, or X in an arthropod repellent medium from which a vaporized compound of Formula IA, IB, IC, ID, IE, IF, IG, or X can be dispersed. Non-limiting examples of suitable media include alcohols such as ethanol, glycerin and polyethylene glycol; ketones such as acetone; ethers such as tetrahydrofuran and dioxane; aliphatic hydrocarbons or petroleum distillates such as gasoline, naphtha, mineral spirits, tar, hexane, kerosene, toluene, xylene, limonene, turpentine, paraffin and petroleum benzene; esters such as ethyl acetate; and essential oils such as pine oil or citronella oil. In certain embodiments, the arthropod repellent medium is an insect repellent medium.

In certain embodiments, vaporization of the repellent may be assisted by thermal volatilization. Thermal volatilization may proceed by flame, ionizing radiation, oven, sunlight, electrical pulse, laser, gas heating element, or electric-powered heating element, such as induction heating, chemical reaction, microwave irradiation, ultrasound or a mixture thereof.

In certain embodiments, the subject matter is directed to a method for controlling one or more arthropods, comprising exposing said one or more arthropods to a composition comprising a compound of Formula IA, IB, IC, ID, IE, IF, or IG and a carrier; wherein said composition is a vapor. When the composition is a vapor, the composition can be used as a spatial repellent, wherein the composition can be dispensed into an area to repel arthropods, wherein the arthropods do not come into contact with the locus.

The spatial arthropod repellent compounds disclosed herein may be formulated into any suitable composition to dispense a suitable amount of the repellent compound into an environmental area in which it is desired to repel arthropods, wherein the arthropods do not come into contact with the locus. This repellency effective amount would typically range from about 1 mg/cm² down to about 1 pg/cm² or from about 100 g/cm² down to about 30 ng/cm² of the base area of the environmental area in which repellency is to be sought. In certain embodiments, the spatial arthropod repellent compounds disclosed herein may be formulated into any suitable composition to dispense a suitable amount of the repellent compound onto a locus, wherein the insects are repelled at least 1 cm, 2 cm, 1 in, 2 in, 3 in, 4 in, 5 in, 6 in, 7 in, 8 in, 9 in, 10 in, 11 in, 1 foot, 2 feet, 3 feet, 4 feet, 5 feet, 6 feet, 7 feet, 8 feet, 9 feet, 10 feet, 11 feet, 12 feet, 13 feet, 14 feet, 15 feet, 16 feet, 17 feet, 18 feet, 19 feet, 20 feet, 21 feet, 22 feet, 23 feet, 24 feet, or 25 feet from the locus. In certain embodiments, arthropod repellent compounds disclosed herein are effective both in the vapor phase as spatial repellents and on contact. In certain embodiments, the arthropod repellent compounds are insect repellent compounds.

In certain embodiments, the subject matter disclosed herein is directed to a method of repelling an arthropod from a locus, comprising contacting said locus with a non-toxic effective arthropod repellent composition comprising a compound of Formula IA, IB, IC, ID, IE, IF, or IG, and a carrier. In certain embodiments, the arthropod is an insect.

In certain embodiments, the subject matter disclosed herein is directed to a method of repelling an arthropod from a locus, comprising contacting said locus with a non-toxic effective arthropod repellent composition comprising a compound of Formula IA, IB, IC, ID, IE, IF, or IG and a carrier, wherein said compound is selected from the group consisting of

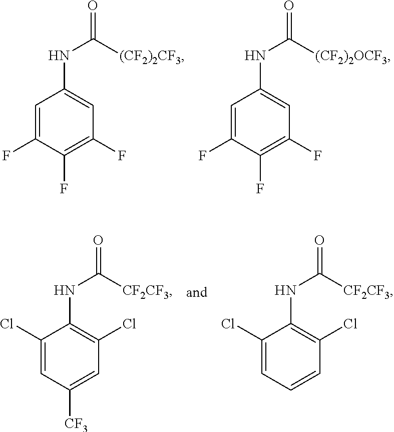

wherein said arthropods are repelled from an adjacent area to said object or locus, wherein said arthropods are not physically contacted with said object or locus.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula X and a carrier.

In certain embodiments, the subject matter disclosed herein is directed to a method of repelling an arthropod from a locus to which said arthropod is normally attracted, comprising contacting said locus with a non-toxic effective arthropod repellent a composition comprising a compound of Formula X and a carrier.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula IA, IB, IC, ID, IE, IF, or IG selected from the group consisting of

| | |
|---|---|
| MTFI-2-1B | MTFI-2-1F |
| MTFI-2-2B | MTFI-4-4B |
| MTFI-4-6B | MTFI-4-6C |
| MTFI-4-6D | MTFI-4-7A |
| MTFI-4-7B | MTFI-4-7C |
| MTFI-4-7D | MTFI-4-2D |
| MTFI-4-1D | MTFI-4-FPL-A |
| MTFI-4-NA-1C | MTFI-5-3a |
| MTFI-5-4a | MTFI-5-4b |
| MTFI-5-4c | MTFI-5-4d |
| MTFI-5-4e | MTFI-5-5a |
| MTFI-5-5c | MTFI-5-5d |
| MTFI-5-6c | MT2018-0316-01 |
| MT2018-0316-02 | MTFI-5-7a |
| MTFI-5-8a | MTFI-5-8b |
| MTFI-5-9a | MTFI-5-10a |
| MTFI-5-11a | MT2018-0314-02 |
| GMR024 | GMR028 |
| GMR029 | GMR030 |
| GMR031 | GMR069 |
| GMR083 | GMR085 |
| GMR090 | GMR147 |
| GMR093 | GMR095 |
| GMR096 | GMR097 |
| GMR098 | GMR099 |
| GMR100 | GMR102 |
| GMR104 | GMR105 |
| GMR106 | GMR107 |
| GMR113 | GMR114 |
| GMR117 | GMR118 |
| GMR129 | GMR148 |
| GMR149 | GMR150 |
| GMR151 | GMR159 |
| GMR161 | GMR163 |
| GMR165 | GMR169 |
| GMR177 | GMR034 |
| GMR035 | GMR036 |
| GMR037 | GMR038 |
| GMR039 | GMR040 |
| GMR041 | GMR042 |
| GMR043 | GMR044 |
| GMR045 | GMR046 |
| GMR047 | GMR048 |
| GMR049 | GMR050 |
| GMR051 | GMR052 |
| GMR053 | GMR054 |
| GMR055 | GMR056 |
| GMR057 | GMR059 |
| GMR060 | GMR062 |
| GMR067 | GMR069 |
| GMR070 | GMR072 |
| GMR073 | GMR074 |
| GMR076 | GMR077 |
| GMR078 | GMR079 |
| GMR083 | GMR084 |
| GMR085 | GMR086 |
| GMR087 | GMR089 |
| GMR090 | GMR091 |
| GMR092 | GMR093 |
| GMR095 | GMR096 |
| GMR097 | GMR106 |
| GMR099 | GMR117 |
| GMR101 | GMR119 |
| GMR104 | GMR150 |
| GMR107 | GMR159 |
| GMR129 | GMR149 |
| GMR151 | GMR163, and |
| GMR169, | |

and a carrier.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula IA, IB, IC, ID, IE, IF, or IG and an essential oil, wherein the composition is synergistic, in intimate admixture with a carrier.

In certain embodiments, the subject matter disclosed herein is directed to a method of repelling arthropods, comprising contacting an object or locus with a composition comprising a compound of Formula IA, IB, IC, ID, IE, IF, or IG and an essential oil, wherein said arthropods are repelled from an adjacent area to said object or locus, wherein said arthropods are not physically contacted with said object or locus; and, wherein the composition is synergistic, in admixture with a carrier.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula X and an essential oil, wherein the composition is synergistic, in intimate admixture with a carrier.

In certain embodiments, the subject matter disclosed herein is directed to a method of repelling arthropods, comprising contacting an object or locus with a composition comprising a compound of Formula IA, IB, IC, ID, IE, IF, or IG and, a second compound selected from the group consisting of a pyrethroid, a compound of Formula II′, and a compound of Formula III‘

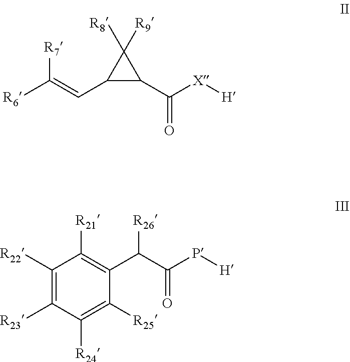

wherein,
X″ is O, NH, or S; R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl; and,
wherein said arthropods are repelled from an adjacent area to said object or locus, wherein said arthropods are not physically contacted with said locus; and, wherein the composition is synergistic, in admixture with a carrier.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula IA, IB, IC, ID, IE, IF, or IG in an amount of about 0.01-99.99%, 1-99%, 5-95%, 10-90%, 20-80%, 30-70%, 40-60%, or 50% and an essential oil in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of a compound of Formula IA, IB, IC, ID, IE, IF, or IG.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula IA, IB, IC, ID, IE, IF, or IG and a second compound selected from a pyrethroid, a compound of Formula II′, or compound of Formula III′.

In certain embodiments, the subject matter disclosed herein is directed to a method of repelling a species of insect from a locus to which said species of insect is normally attracted, comprising contacting on said locus a non-toxic effective insect repellent of a composition comprising a compound of Formula X and a second compound selected from a pyrethroid, a compound of Formula II′, or compound of Formula III′.

In certain embodiments, the subject matter disclosed herein is directed to a method of repelling a species of insect from a locus to which said species of insect is normally attracted, comprising contacting on said locus a non-toxic effective insect repellent a compound of Formula IA, IB, IC, ID, IE, IF, or IG in an amount of about 0.01-99.99%, 1-99%, 5-95%, 10-90%, 20-80%, 30-70%, 40-60%, or 50%; and a second compound selected from a pyrethroid, a compound of Formula II′, or compound of Formula III′ in an amount of about 99.99-0.01%, 99-1%, 95-5%, 90-10%, 80-20%, 70-30%, 60-40%, or 50%, wherein the composition is synergistic, in intimate admixture with a carrier.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula IA, IB, IC, ID, IE, IF, or IG.

In certain embodiments, in any of the methods described herein, the compound of Formula IA, IB, IC, ID, IE, IF, or IG can be effective at repelling or controlling arthropods at a concentration of about 10 μg/cm², 20 μg/cm², 30 μg/cm², 40 μg/cm², 50 μg/cm², 60 μg/cm², 70 μg/cm², 80 μg/cm², 90 μg/cm², 100 μg/cm², 110 μg/cm², 120 μg/cm², 130 μg/cm², 140 μg/cm², 150 μg/cm², 160 μg/cm², 170 μg/cm², 180 μg/cm², 190 μg/cm², or 200 μg/cm².

In certain embodiments, the arthropods repelled by the compounds, compositions, or methods disclosed herein are Hemiptera Insects, selected from Delphacidae (planthoppers), such as Laodelphax striatellus (small brown planthopper), Nilaparvata lugens (brown planthopper), Sogatella furcifera (white-backed rice planthopper); Deltocephalidae (leafhoppers), such as Nephotettix cincticeps (green rice leafhopper), Recilia dorsalis (zig-zag rice leaf hopper), Nephotettix virescens (green rice leafhopper); Aphididae (aphids), stink bugs, Aleyrodidae (whiteflies), scales, Tingidae (lace bugs), or Psyllidae (suckers).

In certain embodiments, the arthropods repelled by the compounds, compositions, or methods disclosed herein are Lepidoptera Insects, selected from Pyralidae, such as Chi/o suppressalis (rice stem borer), Cnaphalocrocis medina/is (rice leafroller), Plodia interpunctella (Indian meal moth); Noctuidae, such as Spodoptera litura (tobacco cutworm), Pseudaletia separata (rice armyworm), Mamestra brasicae (cabbage armyworm); Pieridae, such as Pieris rapae crucivora (common cabbageworm); Tortricidae, such as Adoxophyes spp.; Carposinidae; Lyonetiidae; Lymantriidae; Plusiinae; Agrotis spp. such as Agrotissegetum (turnip cutworm), or Agrotis ipsilon (black cutworm); Helicoverpa spp.; Heliothis spp.; Plutella xylostella; Parnara guttala (rice skipper); Tinea pelionella (casemaking clothes moth); or Tineola bisselliella (webbing clothes moth).

In certain embodiments, the arthropods repelled by the compounds, compositions, or methods disclosed herein are Diptera Insects, Culex spp., such as Culex pipiens pallens (common mosquito), Culex tritaeniorhynchus, Aedes spp., such as Aedes aegypti, Aedes albopictus; Anopheles spp., such as Anopheles sinensis; Chironomidae (midges); Muscidae, such as Musca domestica (housefly), Muscina sabulans (false stablefly), Musca autumnahs (face fly), Fannia canicularis (little housefly); Calliphoridae; Sarcophagidae; Anthomyiidae, such as Delia platura (seedcorn maggot), Delia antiqua (onion maggot); Tephritidae (fluit flies); Drosophilidae; Psychodidae (moth flies); Tabanidae; Simuliidae (black flies); Stomoxyidae (stable flies); Phoridae; or Ceratopogonidae (biting midges).

In certain embodiments, the arthropods repelled by the compounds, compositions, or methods disclosed herein are Coleoptera Insects (Beetles), several nonlimiting examples of which include Corn rootworms, such as Diabrotica virgifera (western corn rootworm), Diabrotica undecimpwictata howardi (southern corn rootworm); Scarabaeidae (scarabs), such as Anomala cuprea (cupreous chafer), Anomala rufocuprea (soybean beetle); Curculionidae (weevils), such as Sitophilus zeamais (maize weevil), Lissorhoptrus oryzophilus (ricewater weevil), ball weevil, Callosobruchus chinensis (adzuki bean weevil); Dermestidae, such as Authrenus verbasci (varied carpet beetle), Altagenus unicolor japonicus (black carpet beetle); Tenebrionidae (darkling beetles), such as Tenebrio molitor (yellow mealworm), or Tribolium castaneum (red flour beetle); Chrysomelidae (leaf beetles) such as Oulema oryzae (rice leaf beetle), Phyllotreta striolata (striped flea beetle), Aulacophorafemoralis (cucurbit leaf beetle); Anobiidae; Epilachna spp. such as Epilachna vigintioctonmnctata (twenty-eight-spotted ladybird); Lyctidae (powderpost beetles), Bostrychidae (false powderpost beetles), or Cerambycidae, Paederusffuscipes (robe beetle).

In certain embodiments, the arthropods repelled by the compounds, compositions, or methods disclosed herein are Dictyoptera Insects, such as the following: Blattella germanica (German cockroach); Periplanetafuliginosa (smokybrown cockroach); Periplaneta americana (American cockroach); Periplaneta brunnea (brown cockroach); or Blatta orienialis (oriental cockroach).

In certain embodiments, the arthropods repelled by the compounds, compositions, or methods disclosed herein are Thysanoptera Insects (Thrips), such as Thrips palmi, Flankliniella occidentalis (western flower thrips), or Thrips hawaiiensis (flower thrips).

In certain embodiments, the arthropods repelled by the compounds, compositions, or methods disclosed herein are Hymenoptera Insects, such as Formicidae (ants); Vespidae (hornets); Polistes spp. (long-legged wasps); Bethylidae; or Tenthredinidae (sawflies), such as Athalis rosae ruficornis (cabbage sawfly).

In certain embodiments, the arthropods repelled by the compounds, compositions, or methods disclosed herein are Orthoptera Insects, such as Gryllotalpidae (mole crickets); or Acrididae (grasshoppers).

In certain embodiments, the arthropods repelled by the compounds, compositions, or methods disclosed herein are Siphonaptera Insects (Fleas), such as Ctenocephalides canis (dog flea); Ctenocephalidesfelis (cat flea); or Pulex irritans.

In certain embodiments, the arthropods repelled by the compounds, compositions, or methods disclosed herein are Anoplura Insects (Lice), such as Pediculus corporis (body louse); Pediculus humainms (head louse); or Pthiruspubis (crab louse).

In certain embodiments, the arthropods repelled by the compounds, compositions, or methods disclosed herein are Isoptera Insects, such as Reticulitermes speratus; Coptotermes formosanus (Formosan subterranean termite).

In certain embodiments, the arthropods repelled by the compounds, compositions, or methods disclosed herein are Harmful Acarina, such as Ixodidae (Ticks): Boophilus microplus; Haemaphysalis longiconis Tetranychidae (spider mites): Tetranychus cinnabarinus (carmine spider mite); Tetranychus urticae (two-spotted spider mite); Tetranychus kanzawai (Kanzawa spider mite); Panonychus citri (citrus red mite); Panonychus ulmi (European red mite); House-dust Mites: Acaridae such as Tyrophagus putrescentiae (copra mite), Aleuroglyphus ovatus (brown legged grain mite); Dermanyssidae such as Dermatophagoides farinae (American house dust mite), Dermatophagoides pteronyssinus; mites parasitizing honeybees, such as Varroa jacobsoni; Euvarroa sinhai, Acarapis woodi; Tropilaelaps clareae; Glycyphagidae, such as Glycyphagus privatus, Glycyphagus domesticus, Glycyphagus destructor; Cheyletidae, such as Chelacaropsis malaccensis, Cheyletus fortis; Tarsonemidae; Chortoglyphus spp.; Haplochthonius spp. Chilognatha (millipedes), such as Oxydus spp.; Chilopoda (centipedes), such as red centipede; wood lice, such as Porcellio spp., Porcellionides spp.; and pill bugs, such as Armadillidium spp.

In certain embodiments, the compounds as disclosed herein may be used to repel insects. In certain embodiments, the compounds as disclosed herein may also be used to repel arthropods. Non-limiting examples of arthropods include spiders, crabs, centipedes, millipedes, and scorpions. In certain embodiments, the arthropod repelled by the compounds, compositions, or methods disclosed herein is an arthropod selected from the group consisting of a fly, spider, butterfly, crab, mosquito, centipede, tick, millipede, and scorpion. In certain embodiments, the arthropod repelled by the compounds, compositions, or methods disclosed herein is selected from the group consisting of a fly, spider, butterfly, crab, mosquito, centipede, tick, millipede, scorpion, roach, ant, termite, silverfish, and wasp.

In certain embodiments, the insect is a mosquito. In certain embodiments, the mosquito genera is selected from the group consisting of Culex, Anopheles, and Aedes. In certain embodiments, the mosquito is of the species, Aedes aegypti.

In certain embodiments, the subject matter described herein is directed to a method of controlling one or more insects, comprising contacting the insects with a composition comprising a compound of Formula IA, IB, IC, ID, IE, IF, or IG and a carrier.

In certain embodiments, the subject matter described herein is directed to a method of controlling one or more insects, comprising contacting the insects with a composition comprising a compound of Formula X and a carrier.

In certain embodiments, the subject matter described herein is directed to a method of controlling one or more insects, comprising contacting the insects with a composition comprising a compound of Formula IA, IB, IC, ID, IE, IF, or IG and a carrier, wherein said compound is selected from the group consisting of

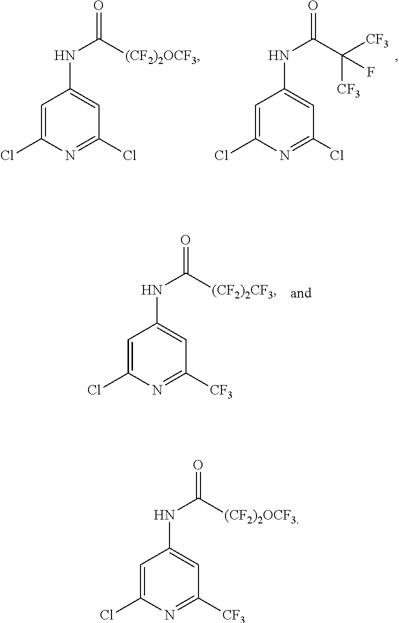

In certain embodiments, the subject matter described herein is directed to a method of controlling one or more insects, comprising contacting the insects with a synergistic composition comprising a compound of Formula IA, IB, IC, ID, IE, IF, or IG in an amount of about 0.01-99.99%, 1-99%, 5-95%, 10-90%, 20-80%, 30-70%, 40-60%, or 50% and a second compound selected from a pyrethroid, a compound of Formula II′, or a compound of Formula III′ in an amount of about 99.99-0.01%, 99-1%, 95-5%, 90-10%, 80-20%, 70-30%, 60-40%, or 50%, wherein the synergistic composition produces, when the insects are brought into contact with the synergistic composition, a combined toxicant effect greater than the sum of the separate toxicant effects from the compound of Formula IA, IB, IC, ID, IE, IF, or IG and the second compound, at comparable concentrations.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula IA, IB, IC, ID, IE, IF, or IG.

In certain embodiments, the compositions described herein comprise two or more compounds or active agents wherein the combination of the two or more compounds or active agents is synergistic. In certain embodiments, the two or more compounds or active agents each independently have a concentration of about 0.1, 1.0, 2, 3, 4, 5, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 800, or 1000 μg/cm².

In certain embodiments, the subject matter described herein is directed to a method of controlling one or more insects, comprising contacting the insects with a synergistic composition comprising a compound of Formula IA, IB, IC, ID, IE, IF, or IG in an amount of about 0.01-99.99%, 1-99%, 5-95%, 10-90%, 20-80%, 30-70%, 40-60%, or 50% and an essential oil in an amount of about 99.99-0.01%, 99-1%, 95-5%, 90-10%, 80-20%, 70-30%, 60-40%, or 50%, wherein the synergistic composition produces, when the insects are brought into contact with the synergistic composition, a combined toxicant effect greater than the sum of the separate toxicant effects from the compound of Formula IA, IB, IC, ID, IE, IF, or IG and the essential oil, at comparable concentrations.

In certain embodiments, in the above method, the composition contains about 0.0001 to 999,999, 0.0001 to 100,000, 0.001 to 1, 0.05 to 100, 10 to 300, 60 to 700, 50 to 75, 25 to 100, 2 to 10, 100 to 500, or 300 to 1000, 800 to 10,000 parts per million of a compound of Formula IA, IB, IC, ID, IE, IF, or IG.

As used herein, “controlling one or more insects” refers to mitigating, reducing, or eliminating a population of one or more insects. In certain embodiments, the synergistic methods disclosed herein achieve a combined toxicant effect greater than 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% than the sum of the separate toxicant effects from the compound of Formula IA, IB, IC, ID, IE, IF, or IG and the second compound or the essential oil, at comparable concentrations.

In certain embodiments, the toxicant effect refers to the vapor toxicity of the composition.

In certain embodiments, the toxicant effect refers to the topical toxicity of the composition.

### V. Methods of Preparing Compounds and Agrochemically Acceptable Salts Thereof

The present compounds of Formula IA, IB, IC, ID, IE, IF, IG, or X can be prepared using analogous synthetic routes disclosed in M. Tsikolia et al. (2013) Pestic. Biochem. Physiol. 107, 138-147 and M. Tsikolia et al. (2018) Pestic. Biochem. Physiol. 151, 40-46.

General Procedures for the Preparation of the Compounds

Method A. Triethylamine (2 equiv) was added to a solution of amine (1 equiv) in dichloromethane (5 mL/mmol) at 0° C. followed by the addition of anhydride (1.1 equiv) and 4-dimethylaminopyridine (0.2 equiv). The solution was warmed to room temperature and stirred for 16 hours. The reaction was quenched with NaHCO_{3 (aq)}, and the mixture was extracted with dichloromethane. The organic layers were collected, washed with brine, dried with magnesium sulfate, filtered, concentrated in vacuo, and purified on a silica gel column.

Method B. Under N₂, nBuLi (2.5M, 0.99 equiv) was added to a mixture of amine (2 equiv) in tetrahydrofuran (5 ml/mmol) at −78° C. The mixture was warmed to 0° C., stirred for one hour, and then cooled to −78° C. Ester (1 equiv) was then added dropwise, the solution was warmed to room temperature, and stirred for 16 hours. The reaction was quenched with NHCl_{(aq)}, the solvent was removed through rotoryevaporation, and the mixture was extracted with dichloromethane. The organic layers were collected, washed with brine, dried with magnesium sulfate, filtered, concentrated in vacuo, and purified on a silica gel column.

Compounds can be synthesized by synthetic routes that include processes analogous to those well-known in the chemical arts, particularly in light of the description contained herein, and those for other heterocycles described in: Comprehensive Heterocyclic Chemistry IL, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database). DTT refers to dithiothreitol. DHAA refers to dehydroascorbic acid.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula IA, IB, IC, ID, IE, IF, IG, or X may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or agrochemically acceptable salts thereof.

Compositions comprising two or more compounds can be prepared simply by contacting one with the other.

The General Procedures and Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

### VI. Pyrethroids

As used herein, non-limiting examples of pyrethroids include permethrins, tetramethrin, metoflurthrin, bifenthrin, kappa-bifenthrin, kadethrin, allethrin, bioallethrin, cyfluthrin, beta-cyfluthrin, deltamethrin, cypermethrin, alpha-cypermethrin, beta-cypermethrin, theta-cypermethrin, zeta-cypermethrin, cyphenothrin, esfenvalerate, fenvalerate, flumethrin, tefluthrin, kappa-tefluthrin, phenothrin, etofenprox, fluvalinate, acrinathrin, halfenprox, flubrocythrinate, bioethanomethrin, brofenvalerate, brofluthrinate, bromethrin, butethrin, chlorempenthrin, cylethrin, cycloprothrin, cyhalothrin, gamma-cyhalothrin, lambda-cyhalothrin, dimethfluthrin, dimethrin, empenthrin, chloroprallethrin, fenfluthrin, fenpirithrin, fenpropathrin, flucythrinate, fluvalinate, tau-fluvalinate, furamethrin, furethrin, heptafluthrin, imiprothrin, japothrins, methothrin, metofluthrin, epsilon-metofluthrin, momfluorothrin, epsilon-momfluorothrin, pentmethrin, biopermethrin, transpermethrin, profluthrin, proparthrin, pyresmethrin, renofluthrin, meperfluthrin, resmethrin, bioresmethrin, cismethrin, terallethrin, tetramethylfluthrin, tralocythrin, tralomethrin, valerate, flufenprox, halfenprox, protrifenbute, silafluofen, sulfoxime, thiofluoximate, and transfluthrin.

### VII. Essential Oils

As used herein, non-limiting examples of essential oils include Almond Oil Bitter, Almond Oil Sweet, Amyris Oil W.I., Angelica Root Oil, Angelica Seed Oil, Anise Oil Chinese, Anise Oil Spanish, Anise Oil Vietnam, Aniseed Oil, Aniseed Oil Terpenes, Apricot Kernel Oil, Armoise Oil, Artemesia Afra, Asafoetida Oil, Avocado Oil, Balsam Copaiba, Balsam Copaiba Oil, Balsam Fir Canada, Balsam Peru, Balsam Peru Oil, Balsam Tolu PG, Basil Oil Comores Type, Basil Oil Indian, Basil Oil Linalool Type, Bay Oil Terpeneless Type, Bay Oil W.I., Bay Oil W.I. Bleached & Filtered, Bay Terpenes PG, Beeswax Absolute, Benzoin Siam Resin, Bergamot Oil, Bergamot Oil B.F., Bergamot Oil Residues, Bergamot Oil Terpenes, Birch Tar Crude, Birch Tar Rectified, Black Currant Bud Absolute, Blood Orange Oil, Bois de Rose Oil, Boronia Absolute Tasmanian, Brominated Vegetable Oil, Buchu Leaf Oil Betulina, Buchu Leaf Oil Crenulata, Buchu Oil, Sulfur Fractions 40%, Cabreuva Oil, Cade Oil, Cade Oil Rectified, Cajeput Oil, Calamus Oil, Camphor Oil 1.070, Camphor Oil Chinese, Camphor Oil White Taiwan, Cananga Oil, Caraway Oil, Cardamom Oil, Carrot Seed Oil, Carvene, Cascarilla Bark Oil, Cassia Oil, Catnip Oil, Cedarleaf Oil, Cedarwood Oil Atlas, Cedarwood Oil Chinese, Cedarwood Oil Texas, Cedarwood Oil Texas Redistilled, Cedarwood Oil Virginiana, Celery Seed Oil, Chamomile Oil Blue, Chamomile Oil Roman, Chamomile Oil Wild Maroc, Cinnamon Bark Oil, Cinnamon Leaf Oil Ceylon, Cistus Absolute, Cistus Oil, Citronella Oil Java 85/35, Citronella Oil Chinese 85/35, Citronella Oil Ceylon, Citronella Terpenes, Clove Bud Indonesian, Clove Bud Oil Prime, Clove Leaf Oil Indonesian 70/80, Clove Leaf Oil Madagascar 80/82, Clove Leaf Oil Redistilled, Clove Stem Oil, Clove Terpenes, Coffee Oil, Cognac Oil Green, Cognac Oil White, Coriander Herb Oil (Cilantro), Coriander Seed Oil, Cornmint Oil 50% Dementh. India, Cornmint Oil 50% Dementh. China, Cornmint Oil Rectified, Cubeb Oil, Cumin Seed Oil, Cypress Oil, Davana Oil, Dillseed Oil, Dillweed Oil, Eucalyptus Citriodora Brazil, Eucalyptus Citriodora Chinese, Eucalyptus Oil 70/75, Eucalyptus Oil 80/85, Eucalyptus Oil Terpenes, Eucalyptus Smithii Oil S.A., Evening Primrose, Fennel Oil, Fennel Oil Bitter, Fennel Oil Sweet, Fir Balsam Absolute, Fir Balsam Concrete, Fir Balsam Canadian, Fir Needle Oil Canadian, Fir Needle Oil Siberian, Fir Needle Oil Chinese, Galangal Root Oil, Galbanum Oil, Galbanum Resinoid, Garlic Oil Chinese, Garlic Oil Mexican, Genet Absolute, Geranium Oil Chinese, Geranium Oil Egyptian, Ginger Oil Chinese, Ginger Oil Fresh, Ginger Oil Indian, Grapefruit Oil 5×, Grapefruit Oil Pink C.P., Grapefruit Oil Washed, Grapefruit Oil White C.P., Grapefruit Terpenes, Grapeseed Oil, Gum Benzoin Siam Resin, Gurjon Balsam Oil, Guaiacwood Oil, Helichrysum Oil, Hemlock Oil, Ho Leaf Oil, Hop Oil, Horseradish Oil, Howood Oil 95%, Hyssop Oil, Jasmine Absolute, Jojoba Oil Purified White, Juniperberry Oil, Labdanum Gum Refined, Lanyana Oil, Laurel Leaf Oil, Lavandin Abrialis, Lavandin Grosso, Lavender Absolute Bulgarian, Lavender Oil 16%, Lavender Oil 40/42 French, Lavender Oil Bulgarian, Lavender Oil Spike, Lemon Essence Oil, Lemon Oil Argentina, Lemon Oil Brazil, Lemon Oil California 5×, Lemon Oil California Type, Lemon Oil Distilled, Lemon Oil Israeli, Lemon Oil Washed, Lemon Terpenes, Lemongrass Oil Guatemalan, Lemongrass Oil East Indian, Lemongrass Terpenes, Lemongrass Oil Terpeneless, Lime Essence Oil Phase, Lime Oil Mexican C.P., Types A & B, Lime Oil Distilled Mexican, Lime Oil Distilled Terpenes, Lime Oil Expressed Terpenes, Lime Oil Peru Distilled, Lime Oil Washed, Lime Sesqui Fractions, Lime Terpenes, Litsea Cubeba Oil, Litsea Terpenes, Lovage Leaf Oil, Lovage Root Oil, Mandarin Essence Oil Phase, Mandarin Oil Brazil, Mandarin Oil Green, Mandarin Oil Italian, Mandarin Oil Red Argentina, Mandarin Terpenes, Mandarin Petitgrain Terpeneless, Marjoram Oil Spanish, Marjoram Oil Sweet Egyptian, Massoia Bark Oil, Melissa Oil, Mentha Arvensis Oil, Mentha Citrata Oil, Mentha Piperita Oil, Milfoil Oil, Mimosa Absolute, Mousse de Arbre, Mousse de Pin Absolute, Mustard Oil Natural, Mustard Oil Synthetic, Myrrh Oil, Myrtle Oil, Neroli Oil, Nutmeg Oil, Nutmeg Oil Ceylon, Nutmeg Oil E.I., Nutmeg Terpenes, Oakmoss Absolute Green, Ocotea Cymbarum, Olibanum Oil, Olibanum Resin, Opoponax Oil, Onion Oil Egyptian, Onion Oil Mexican, Orange Juice Oil BJ N&A, Orange Essence Oil Phase, Orange Oil 5×, Orange Oil 10×, Orange Oil Bitter Ivory Coast Type, Orange Oil Bitter West Indian, Orange Oil Bitter Brazil, Orange Oil S.A., Orange Oil Midseason, Orange Oil Valencia, Orange Oil Terpeneless, Orange Terpenes, Organic Artemesia Oil, Organic Balsam Copaiba Oil, Organic Bois de Rose Oil, Organic Dillweed Oil, Organic Juniper Berry Oil, Organic Lavender Oil, Bulgarian, Organic Peppermint Oil, Organic Tea Tree Oil, Organic Wormwood Oil European, Organic Zdravetz Oil, Origanum Oil, Orris Root Concrete 8% Irone, Palmarosa Oil, Parsley Leaf Oil, Parsley Seed Oil, Patchouli Oil Indonesian, Patchouli Oil Light, Patchouli Oil Molecular Distilled, Pennyroyal Oil, Pepper Oil Black, Peppermint Oil Hotchkiss Type, Peppermint Oil Mitcham Type, Peppermint Oil Piperita Chinese, Peppermint Oil Piperita Indian, Peppermint Oil Piperita Redistilled, Peppermint Oil Piperita Williamette, Peppermint Oil Piperita Yakima, Peppermint Oil Terpenes, Perilla Oil, Petitgrain Oil S.A., Petitgrain Terpenes, Pimento Berry Oil, Pimento Leaf Oil, Pink Pepper Oil (Schinus Molle), Pinus Oil Pumilio, Pinus Oil Sylvestris, Rose Absolute Bulgarian, Rose Absolute Maroc, Rose Concrete Bulgarian, Rose Oil Bulgarian, Rose Oil Maroc, Rose Oil Turkish, Rose Water Concentrate, Rosemary Oil Maroc, Rosemary Oil Spanish, Rosemary Oil Tunisian, Rue Oil, Sage Oil 30%, Sage Oil 50%, Sage Oil Clary, Sage Oil Spanish, Sandalwood Oil Australian, Sandalwood Oil East Indian, Sandalwood Oil Indonesian, Sandalwood Oil Sri Lanka, Sassafras Oil, Savory Oil, Siamwood Oil, Spearmint Oil 65% Indian, Spearmint Oil Chinese 60%, Spearmint Oil Chinese 80%, Spearmint Oil Native, Spearmint Oil Terpeneless, Spearmint Terpenes, Spike Lavender Oil, Spikenard Oil, Spruce Absolute, Spruce Oil Canadian, Styrax Gum Honduras, Styrax Oil Honduras, Tagetes Oil Argentina, Tagetes Oil South African, Tagetes Oil Zimbabwe, Tangerine Oil 5×, Tangerine Oil 10×, Tangerine Oil CP China, Tangerine Oil CP Cravo, Tangerine Oil CP Florida, Tangerine Terpenes, Tarragon Oil, Tea Tree Oil, Thuja Oil, Thyme Oil Red, Thyme Oil White, Tolu Balsam, Tonka Bean Absolute, Perfume Grade, Treemoss Absolute, Tuberose Absolute, Valerian Root Oil, Vetiver Oil Indonesian, Vetiver Terpenes, Vetiver Oil Brazil, Violet Leaf Absolute, Wintergreen Oil Redistilled, Wormwood Oil American, Wormwood Oil European, Yarrow Oil (Milfoil), Ylang Oil #1, Ylang Oil #2, Ylang Oil #3, Ylang Oil Extra, and Zdravetz Oil.

The subject matter described herein includes the following embodiments:

1. A compound selected from the group consisting of Formula IA, IB. IC, ID, LE, IF, or IG

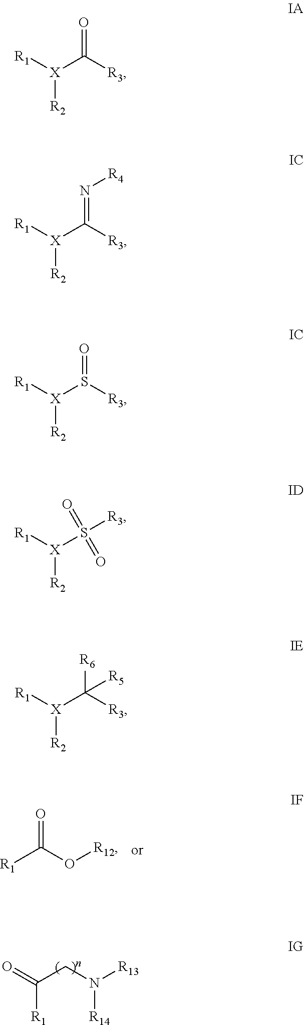

wherein,
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl wherein said heteroaryl, phenyl, cycloalkyl, or heterocycyl is optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, (5- to 10-membered heterocyclyl)-C₁-C₃ alkyl, wherein R₁₆ and R₁₇ are selected from hydrogen, linear or branched C₁-C₁₂ alkyl, or alkene; and said C₆-C₁₀ aryl or (C₆-C₁₀ aryl)-C₁-C₃ alkyl is optionally substituted with one or two substituents, each of which is independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl, and

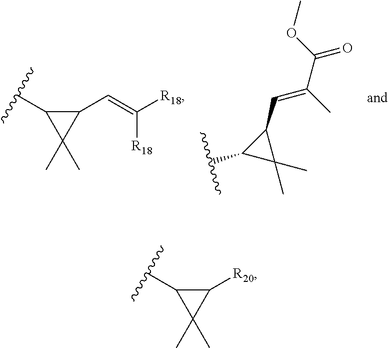

where R₁₈, R₁₉, R₂₀, R₂₁, and R₂₂ are each independently selected from the group consisting of cyano, C₁-C₃ alkyl, halogen, haloalkyl, hydrogen, and phenyl, wherein said phenyl is optionally substituted one, two, or three times with halogen;
wherein said cyano-alkyl is optionally substituted with halogen and said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from hydrogen, halogen, haloalkyl, or C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, or C₁-C₃ alkyl, wherein said heterocyclyl or phenyl is optionally substituted with one or two substituents independently selected from halogen, haloalkyl, or linear or branched C₁-C₃ alkyl;
or,
wherein R₁₃ and R₁₄, together with the nitrogen to which each is attached, form a 5-10 membered heterocyclyl or C₆-C₁₀ heteroaryl; and
n is 0 or 1.

2. The compound of embodiment 1, wherein X is N.

3. The compound of embodiment 1 or 2, wherein R₁ is selected from the group consisting of 6-membered heteroaryl, 6-membered heteroaryl-C₁-C₃ alkyl, phenyl, benzyl, and (5- to 10-membered heterocyclyl)-C₁-C₃ alkyl wherein said heteroaryl, phenyl, or heterocycyl is optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl.

4. The compound of any one of embodiments 1-3, wherein R₁ is phenyl or pyridinyl optionally substituted with one, two, three, four, or five substituents, each of which is independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from hydrogen, C₁-C₆ alkyl, alkoxy, alkene, or haloalkyl.

5. The compound of any one of embodiments 1-4, wherein said halogen is fluorine, chlorine, or bromine.

6. The compound of any one of embodiments 1-5, wherein R₁ is selected from the group consisting of

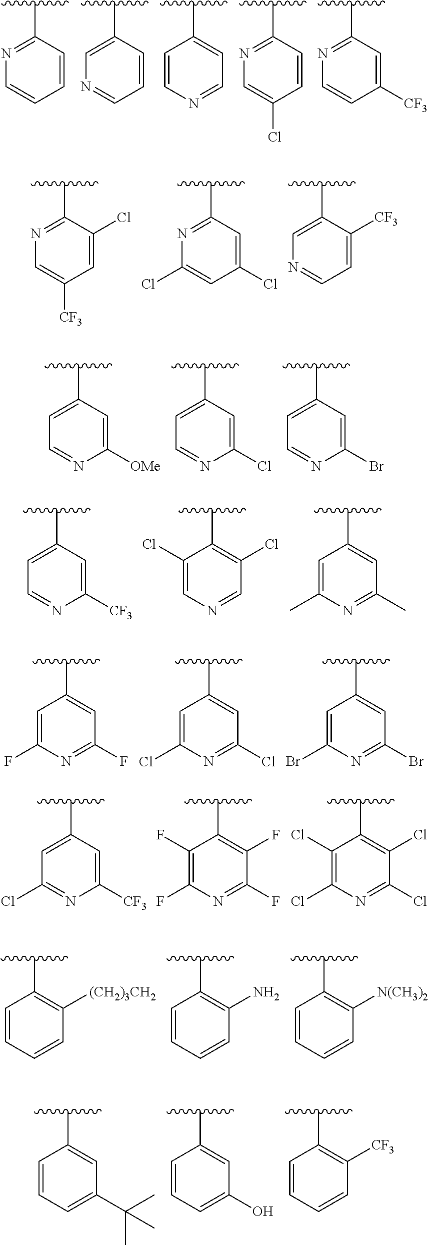

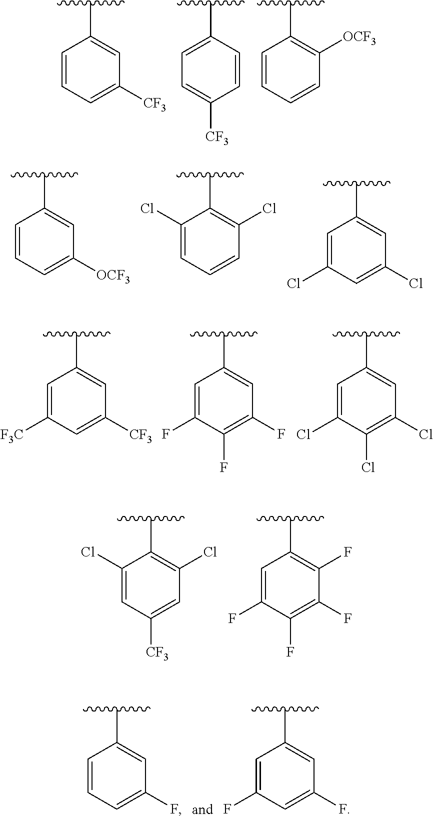

7. The compound of any one of embodiments 1-6, wherein R₁ is selected from

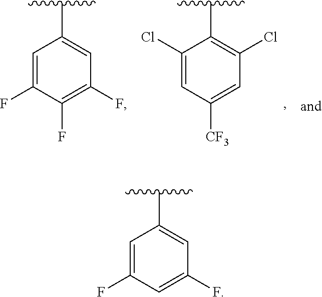 [SMILES: *c1cc(F)c(F)c(F)c1 ; *c1c(Cl)cc(C(F)(F)F)cc1Cl ; *c1cc(F)cc(F)c1]

8. The compound of any one of embodiments 1-7, wherein R₁ is

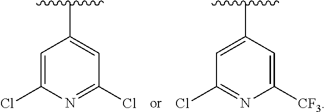 [SMILES: *c1cc(Cl)nc(Cl)c1 ; *c1cc(Cl)nc(C(F)(F)F)c1]

9. The compound of any one of embodiments 1-8, wherein R₂ is selected from the group consisting of

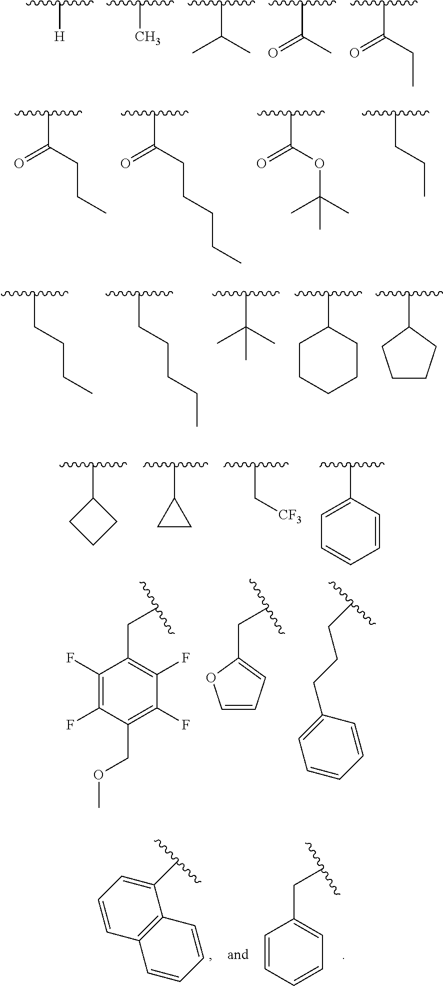

10. The compound of any one of embodiments 1-9, wherein R₂ is hydrogen.

11. The compound of any one of embodiments 1-10, wherein R₃ is selected from the group consisting of

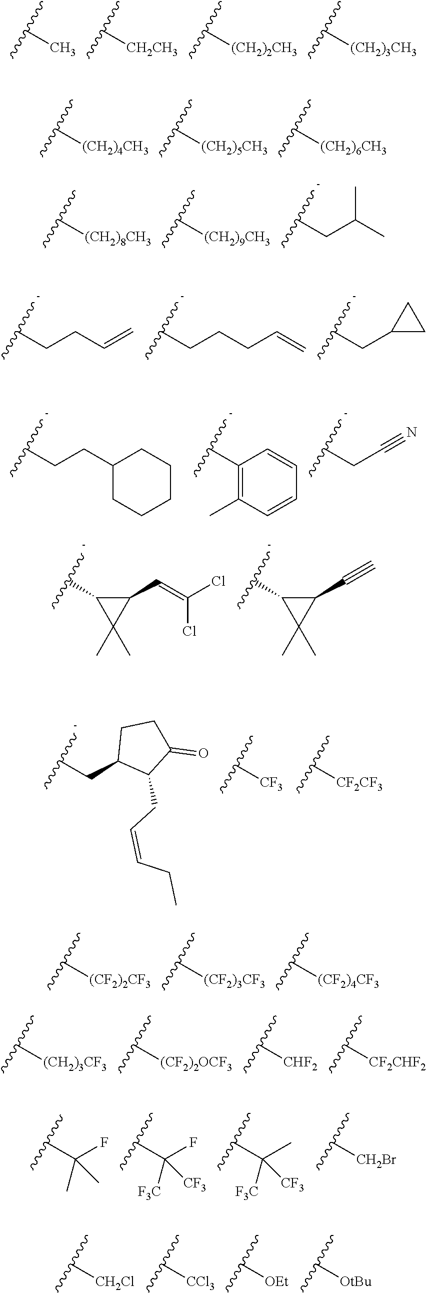

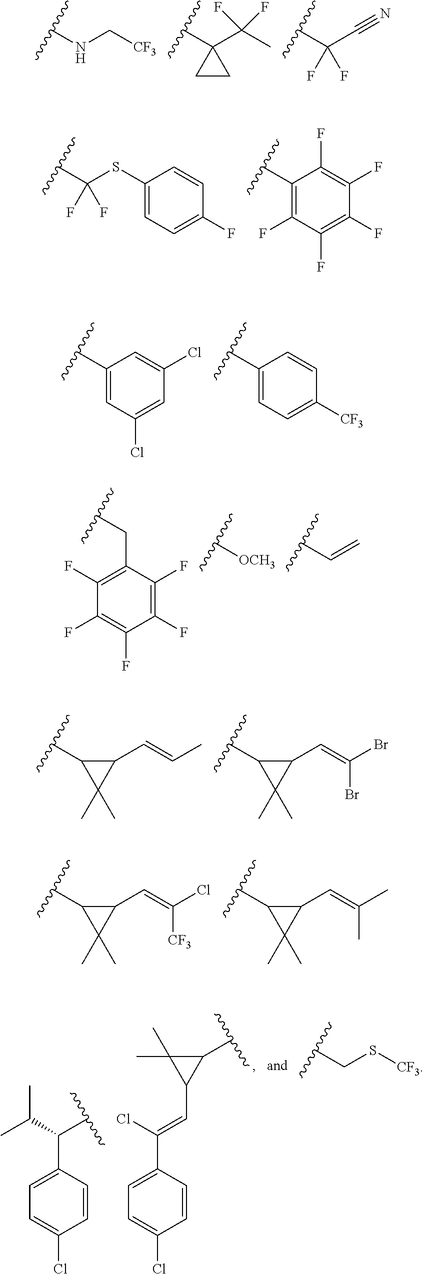

12. The compound of any one of embodiments 1-11, wherein R₃ is selected from the group consisting of

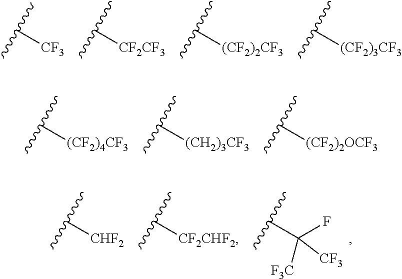

and C₁-C₆ allyl.

13. The compound of any one of embodiments 1-12, wherein R₃ is selected from the group consisting of

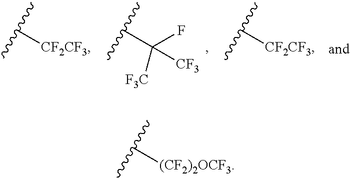

14. The compound of any one of embodiments 1-13, wherein said compound is of Formula IA and is selected from the group consisting of

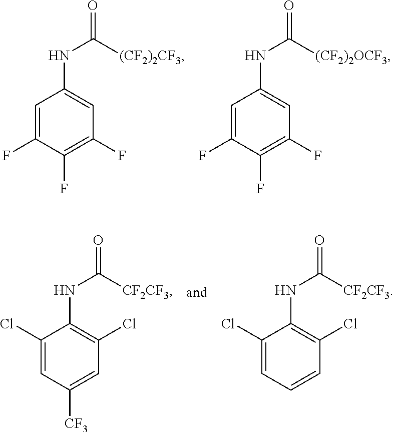

15. The compound of any one of embodiments 1-13, wherein said compound is of Formula IA and is selected from the group consisting of

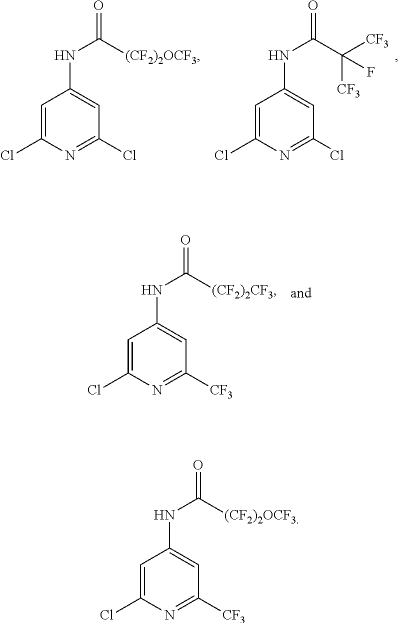

16. The compound of any one of embodiments 1-13, wherein said compound is selected from the group consisting of

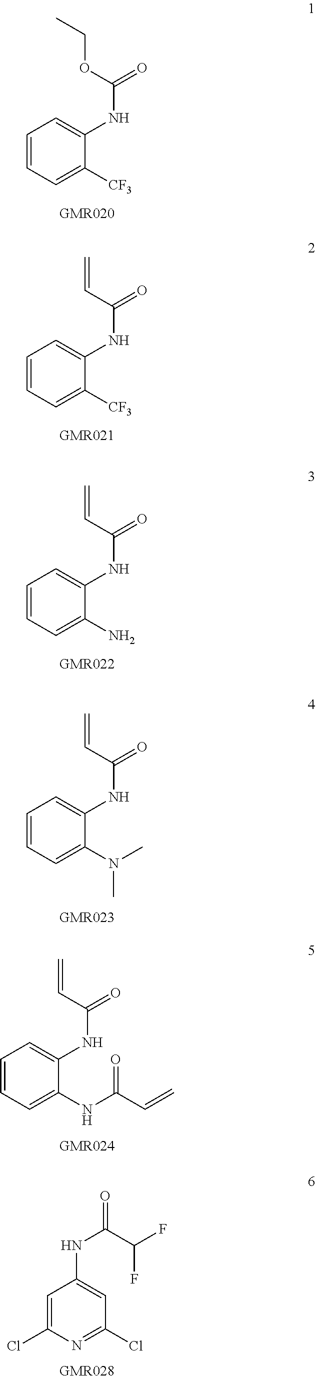 [SMILES: CCOC(=O)Nc1ccccc1C(F)(F)F ; C=CC(=O)Nc1ccccc1C(F)(F)F ; C=CC(=O)Nc1ccccc1N ; C=CC(=O)Nc1ccccc1N(C)C ; C=CC(=O)Nc1ccccc1NC(=O)C=C ; O=C(Nc1cc(Cl)nc(Cl)c1)C(F)F]

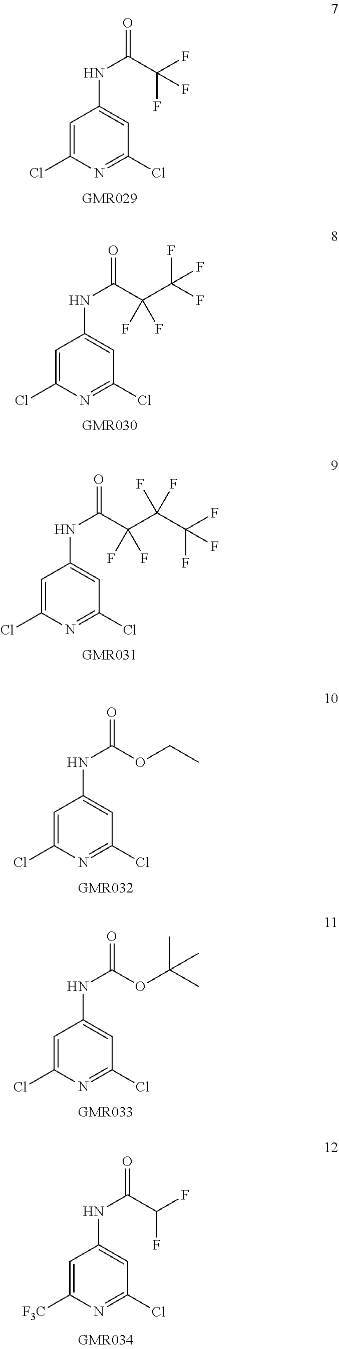 [SMILES: O=C(Nc1cc(Cl)nc(Cl)c1)C(F)(F)F ; O=C(Nc1cc(Cl)nc(Cl)c1)C(F)(F)C(F)(F)F ; O=C(Nc1cc(Cl)nc(Cl)c1)C(F)(F)C(F)(F)C(F)(F)F ; CCOC(=O)Nc1cc(Cl)nc(Cl)c1 ; CC(C)(C)OC(=O)Nc1cc(Cl)nc(Cl)c1 ; O=C(Nc1cc(Cl)nc(C(F)(F)F)c1)C(F)F]

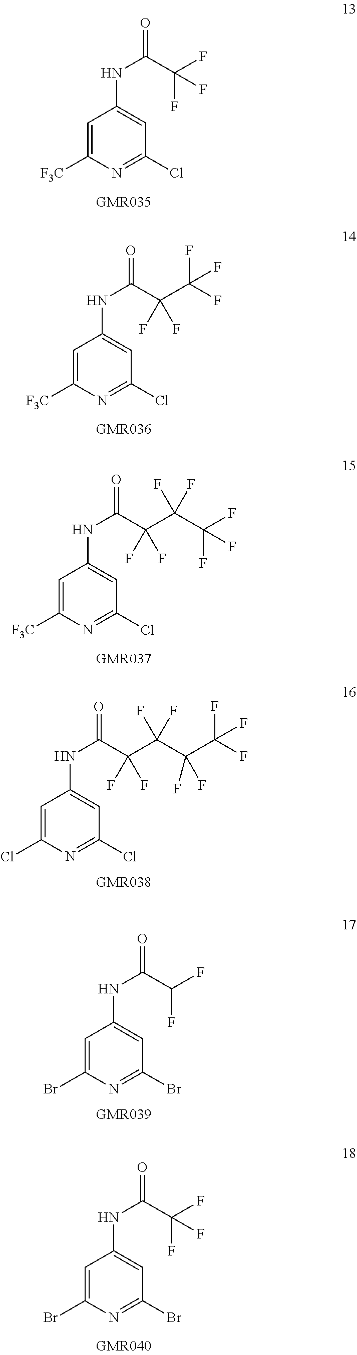 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(Cl)c1)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

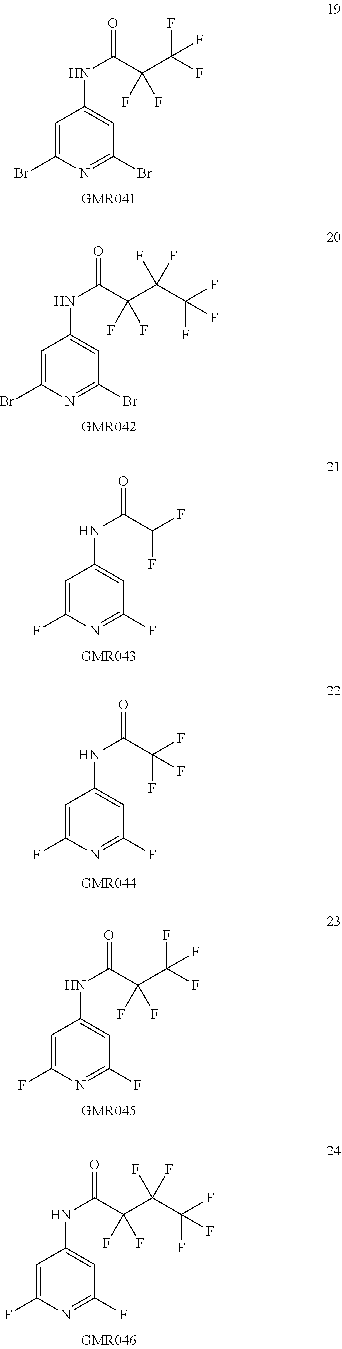 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; O=C(Nc1cc(F)nc(F)c1)C(F)F ; O=C(Nc1cc(F)nc(F)c1)C(F)(F)F ; O=C(Nc1cc(F)nc(F)c1)C(F)(F)C(F)(F)F ; O=C(Nc1cc(F)nc(F)c1)C(F)(F)C(F)(F)C(F)(F)F]

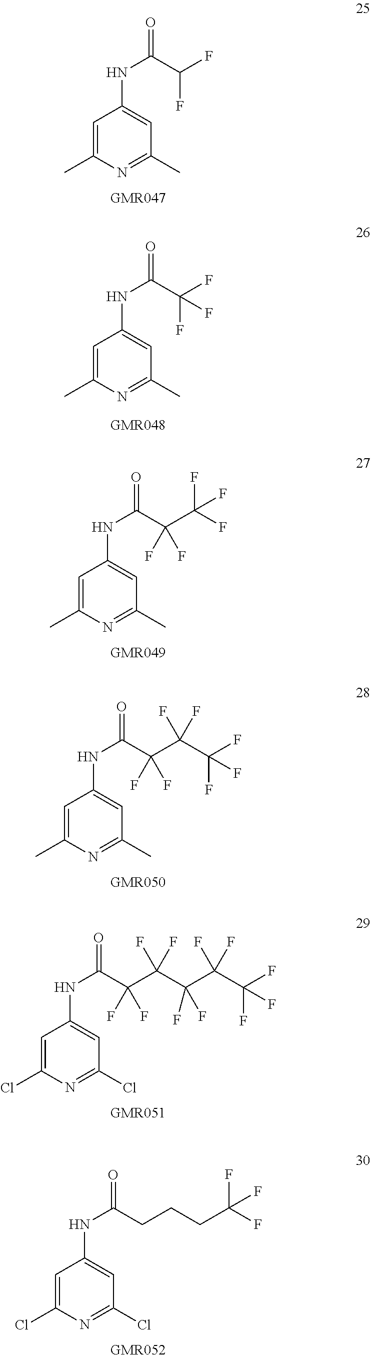 [SMILES: Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1 ; O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1]

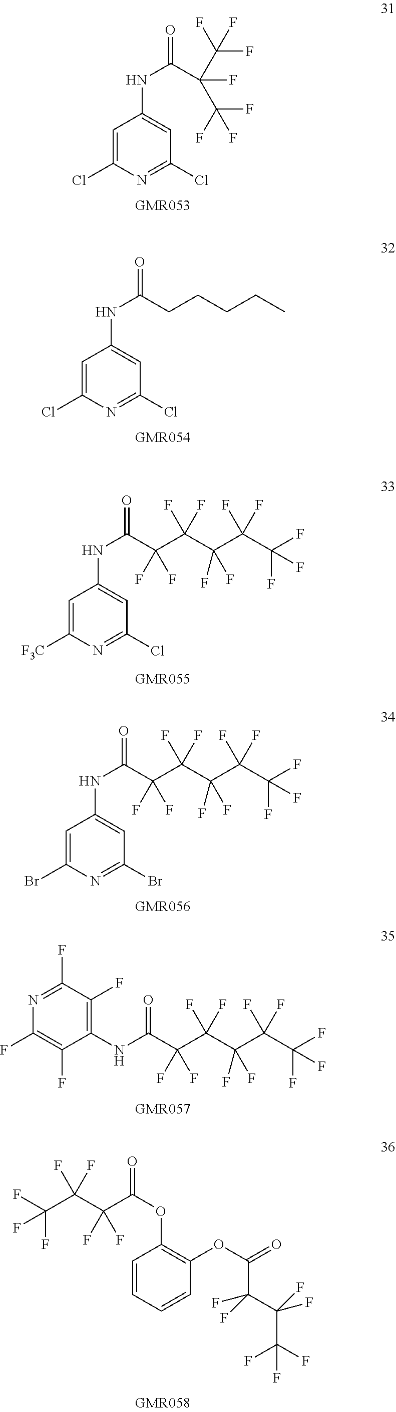 [SMILES: O=C(Nc1cc(Cl)nc(Cl)c1)C(F)(C(F)(F)F)C(F)(F)F ; CCCCCC(=O)Nc1cc(Cl)nc(Cl)c1 ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F ; O=C(Nc1c(F)c(F)nc(F)c1F)C(F)(F)C(F)(F)C(F)(F)C(F)(F)C(F)(F)F ; O=C(Oc1ccccc1OC(=O)C(F)(F)C(F)(F)C(F)(F)F)C(F)(F)C(F)(F)C(F)(F)F]

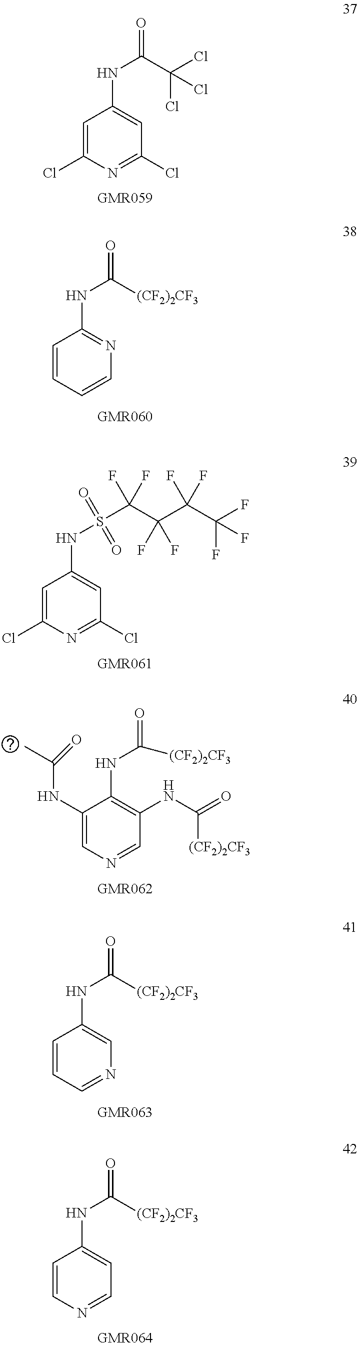

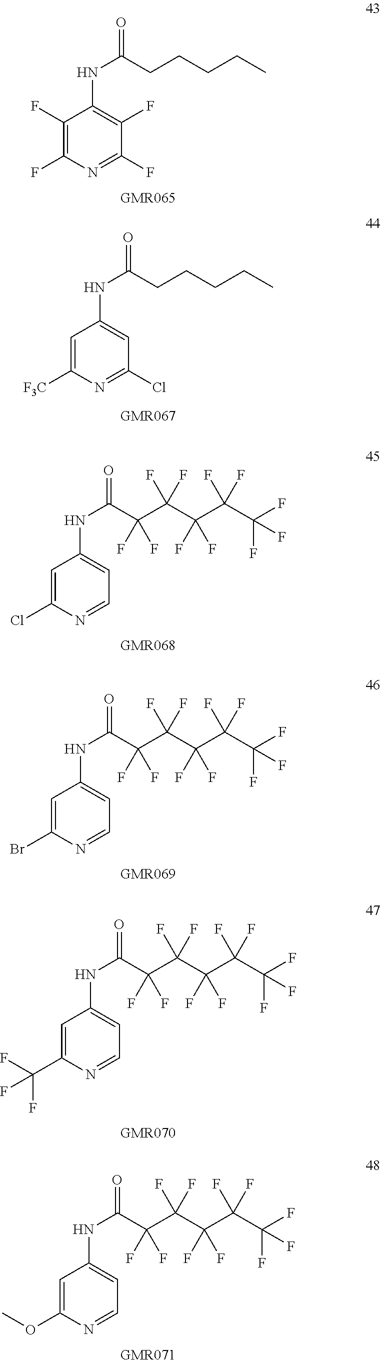

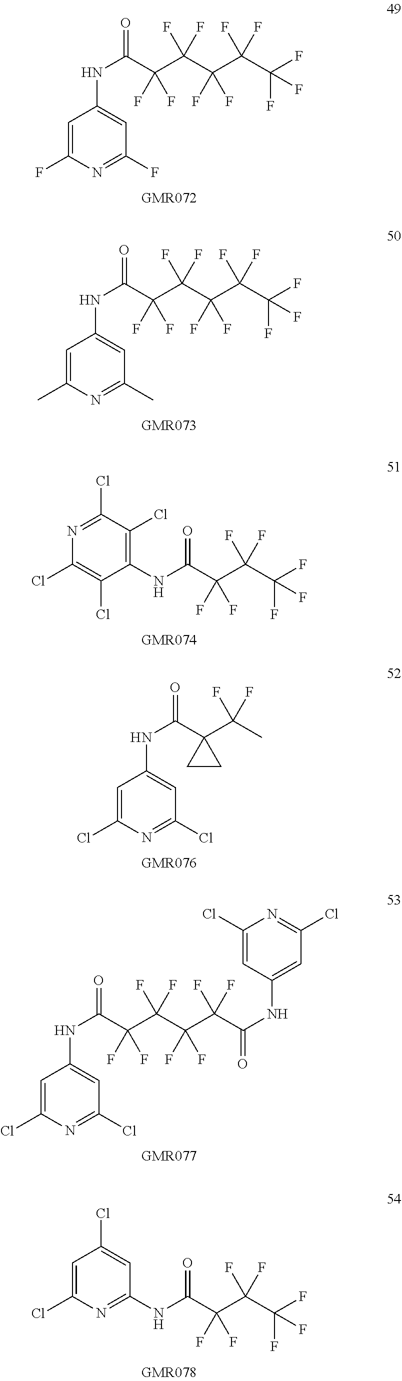 [SMILES: O=C(Nc1cc(F)nc(F)c1)C(F)(F)C(F)(F)C(F)(F)C(F)(F)C(F)(F)F ; Cc1cc(NC(=O)C(F)(F)C(F)(F)C(F)(F)C(F)(F)C(F)(F)F)cc(C)n1 ; O=C(Nc1c(Cl)c(Cl)nc(Cl)c1Cl)C(F)(F)C(F)(F)C(F)(F)F ; CC(F)(F)C1(C(=O)Nc2cc(Cl)nc(Cl)c2)CC1 ; O=C(Nc1cc(Cl)nc(Cl)c1)C(F)(F)C(F)(F)C(F)(F)C(F)(F)C(=O)Nc1cc(Cl)nc(Cl)c1 ; O=C(Nc1cc(Cl)cc(Cl)n1)C(F)(F)C(F)(F)C(F)(F)F]

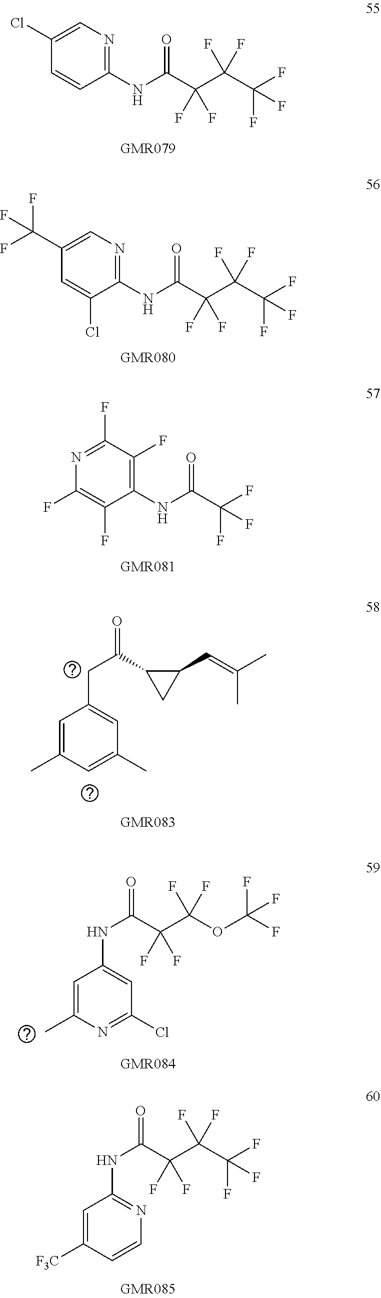

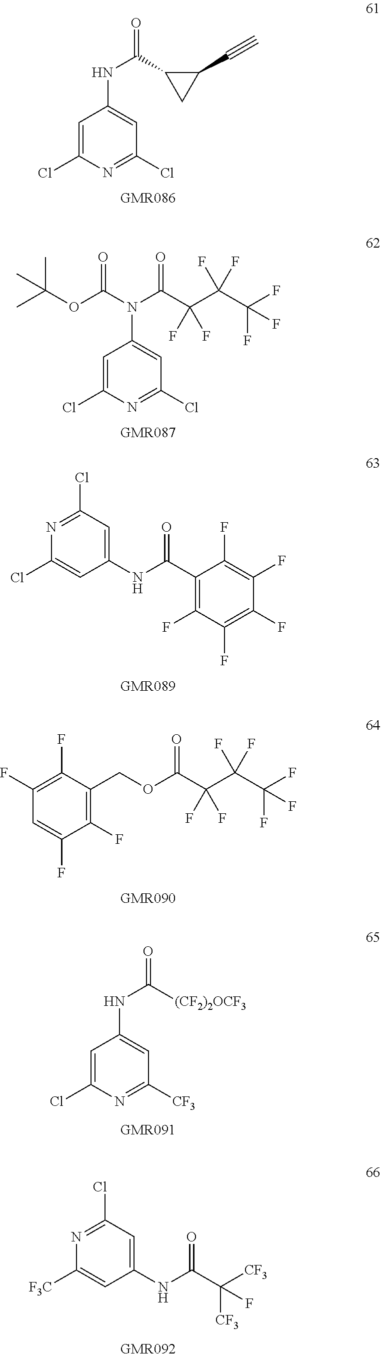

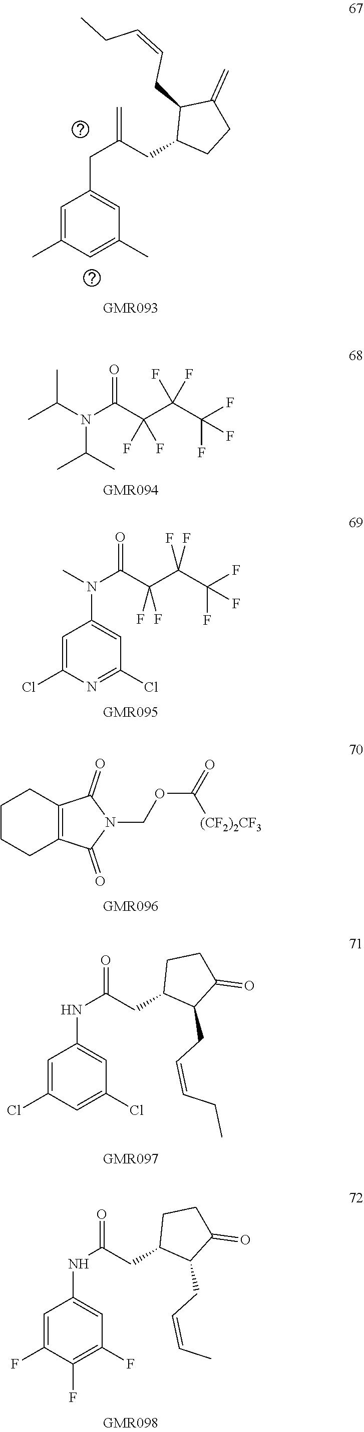

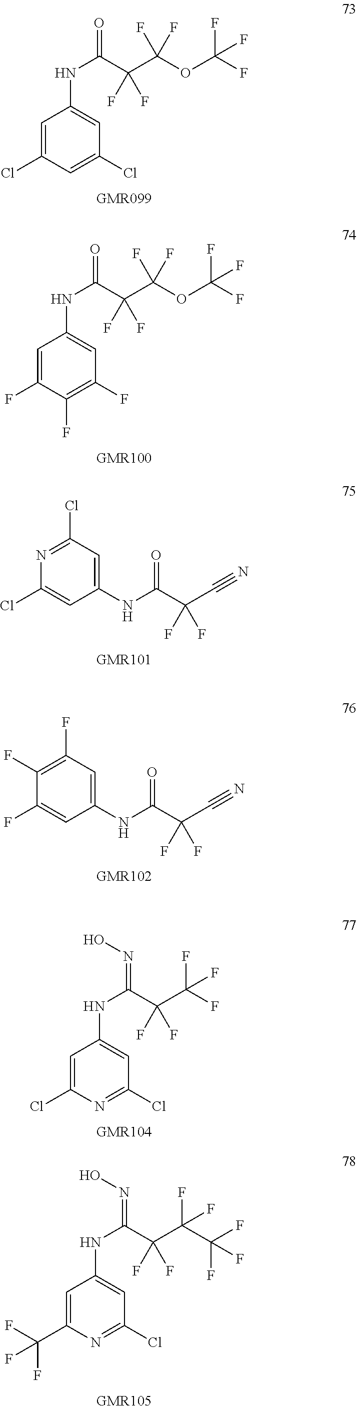 [SMILES: O=C(Nc1cc(Cl)cc(Cl)c1)C(F)(F)C(F)(F)OC(F)(F)F ; O=C(Nc1cc(F)c(F)c(F)c1)C(F)(F)C(F)(F)OC(F)(F)F ; N#CC(F)(F)C(=O)Nc1cc(Cl)nc(Cl)c1 ; N#CC(F)(F)C(=O)Nc1cc(F)c(F)c(F)c1 ; O/N=C(\Nc1cc(Cl)nc(Cl)c1)C(F)(F)C(F)(F)F ; O/N=C(\Nc1cc(Cl)nc(C(F)(F)F)c1)C(F)(F)C(F)(F)C(F)(F)F]

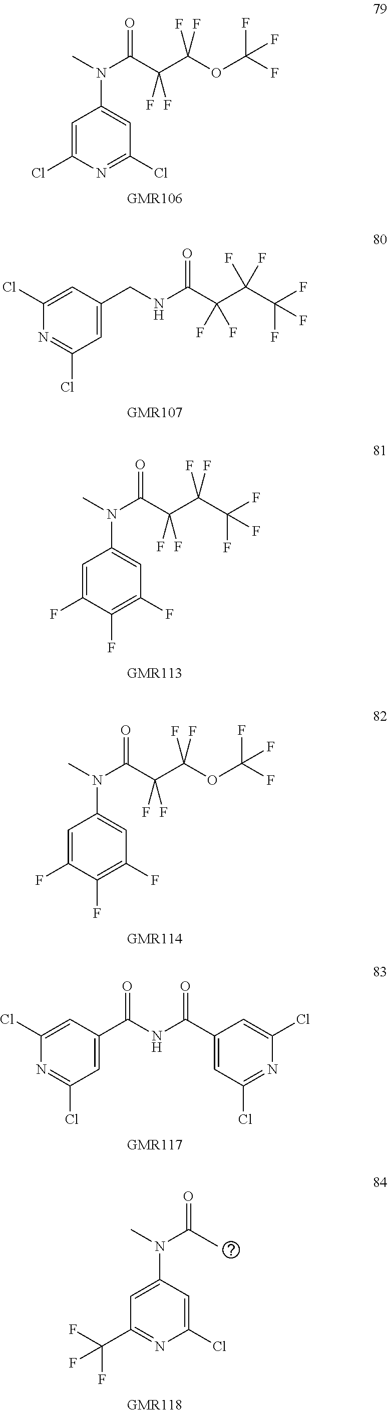 [SMILES: CN(C(=O)C(F)(F)C(F)(F)OC(F)(F)F)c1cc(Cl)nc(Cl)c1 ; O=C(NCc1cc(Cl)nc(Cl)c1)C(F)(F)C(F)(F)C(F)(F)F ; CN(C(=O)C(F)(F)C(F)(F)C(F)(F)F)c1cc(F)c(F)c(F)c1 ; CN(C(=O)C(F)(F)C(F)(F)OC(F)(F)F)c1cc(F)c(F)c(F)c1 ; O=C(NC(=O)c1cc(Cl)nc(Cl)c1)c1cc(Cl)nc(Cl)c1 ; CC(=O)N(C)c1cc(Cl)nc(C(F)(F)F)c1]

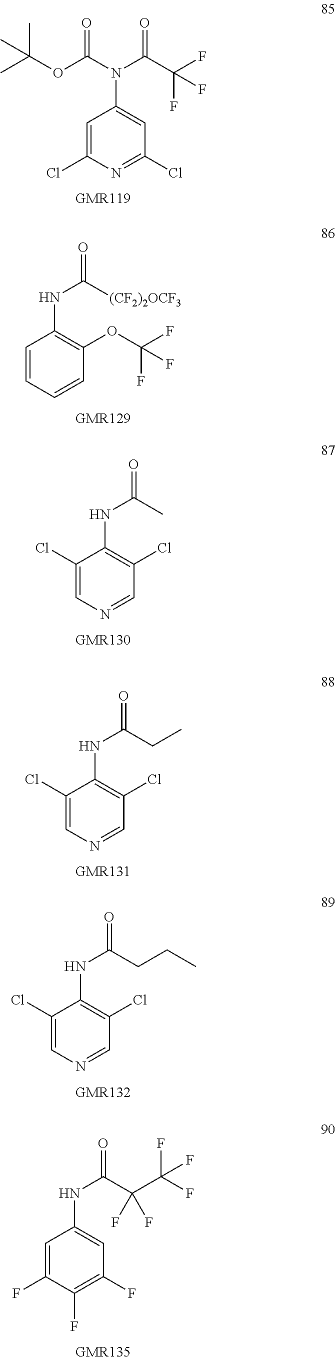

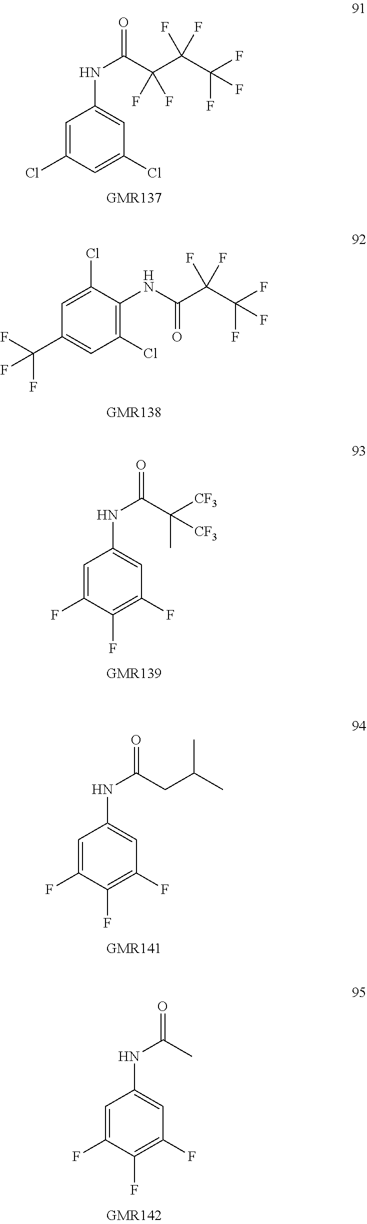 [SMILES: O=C(Nc1cc(Cl)cc(Cl)c1)C(F)(F)C(F)(F)C(F)(F)F ; O=C(Nc1c(Cl)cc(C(F)(F)F)cc1Cl)C(F)(F)C(F)(F)F ; CC(C(=O)Nc1cc(F)c(F)c(F)c1)(C(F)(F)F)C(F)(F)F ; CC(C)CC(=O)Nc1cc(F)c(F)c(F)c1 ; CC(=O)Nc1cc(F)c(F)c(F)c1]

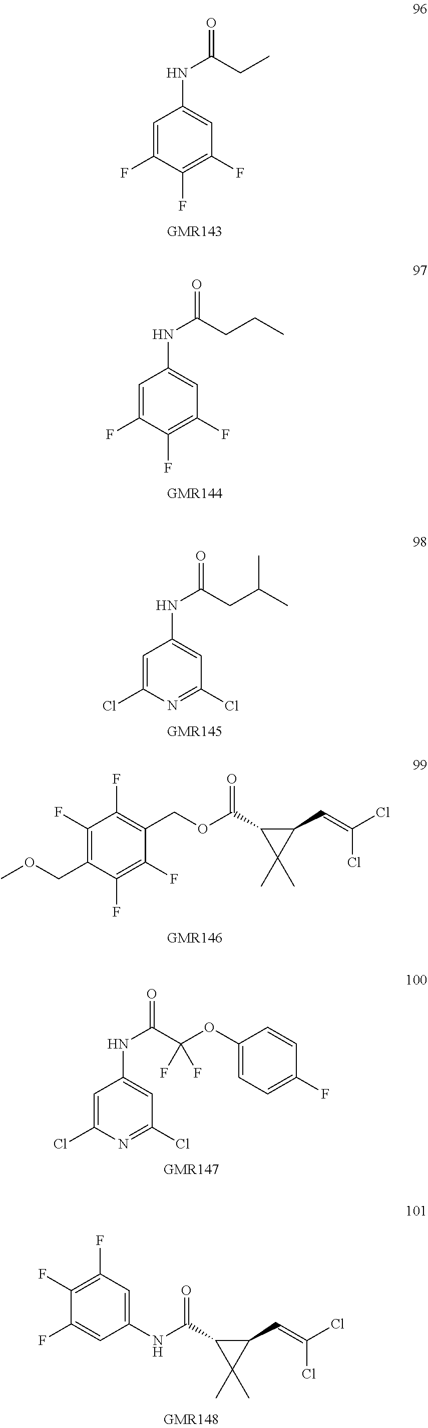 [SMILES: CCC(=O)Nc1cc(F)c(F)c(F)c1 ; CCCC(=O)Nc1cc(F)c(F)c(F)c1 ; CC(C)CC(=O)Nc1cc(Cl)nc(Cl)c1 ; COCc1c(F)c(F)c(COC(=O)[C@@H]2[C@@H](C=C(Cl)Cl)C2(C)C)c(F)c1F ; O=C(Nc1cc(Cl)nc(Cl)c1)C(F)(F)Oc1ccc(F)cc1 ; CC1(C)[C@H](C=C(Cl)Cl)[C@H]1C(=O)Nc1cc(F)c(F)c(F)c1]

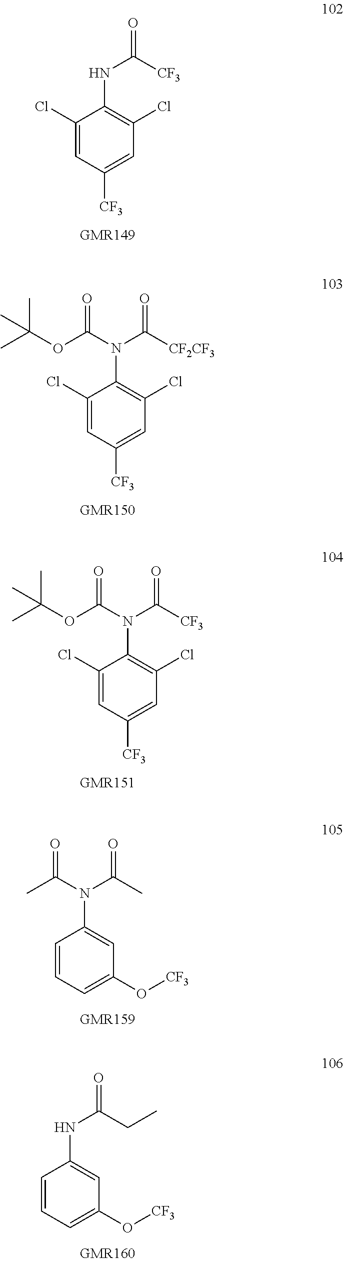

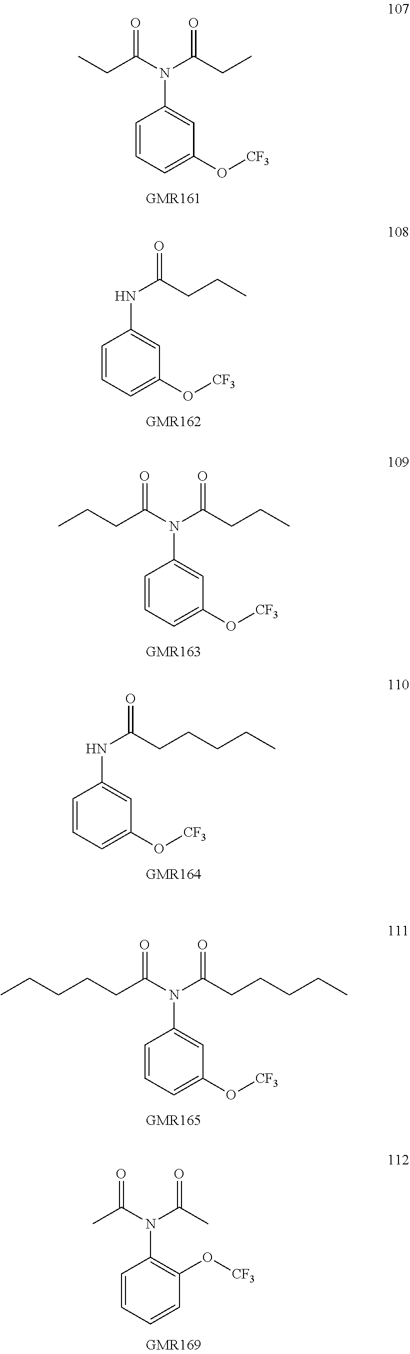

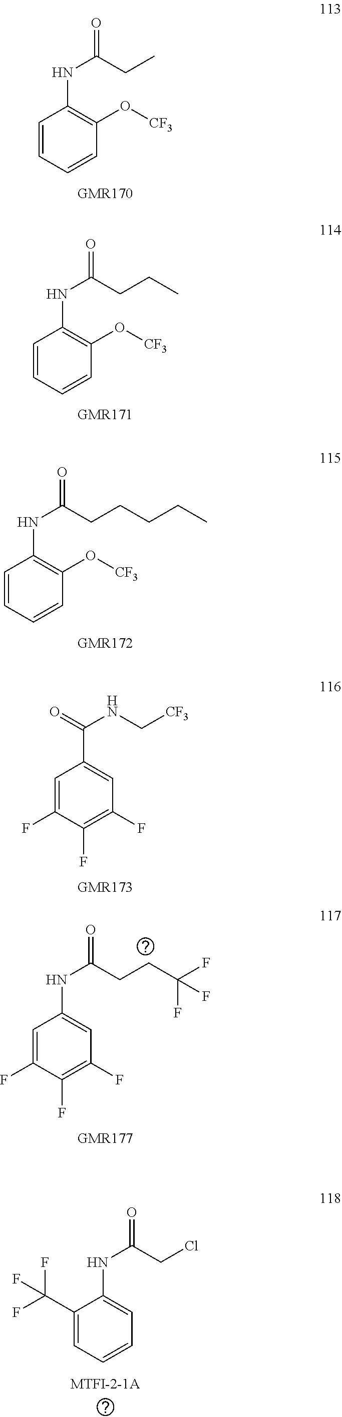 [SMILES: CCC(=O)Nc1ccccc1OC(F)(F)F ; CCCC(=O)Nc1ccccc1OC(F)(F)F ; CCCCCC(=O)Nc1ccccc1OC(F)(F)F ; O=C(NCC(F)(F)F)c1cc(F)c(F)c(F)c1 ; O=C(CCC(F)(F)F)Nc1cc(F)c(F)c(F)c1 ; O=C(CCl)Nc1ccccc1C(F)(F)F]

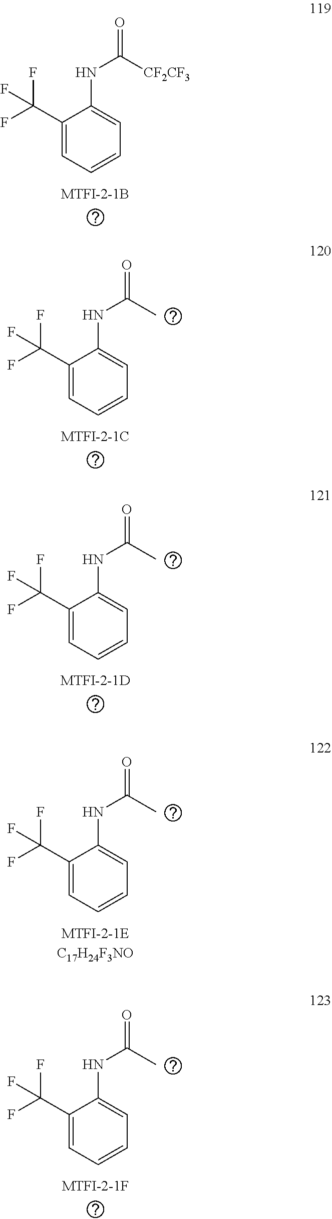

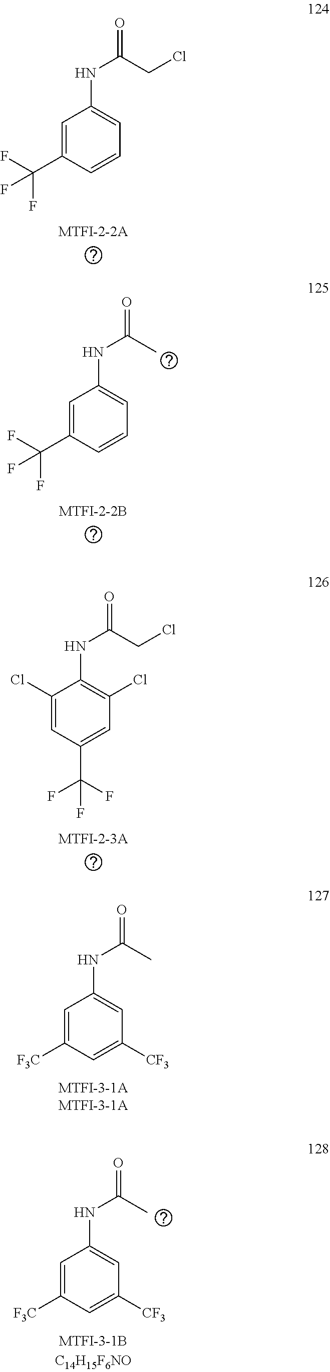 [SMILES: O=C(CCl)Nc1cccc(C(F)(F)F)c1 ; CC(=O)Nc1cccc(C(F)(F)F)c1 ; O=C(CCl)Nc1c(Cl)cc(C(F)(F)F)cc1Cl ; CC(=O)Nc1cc(C(F)(F)F)cc(C(F)(F)F)c1 ; CC(=O)Nc1cc(C(F)(F)F)cc(C(F)(F)F)c1]

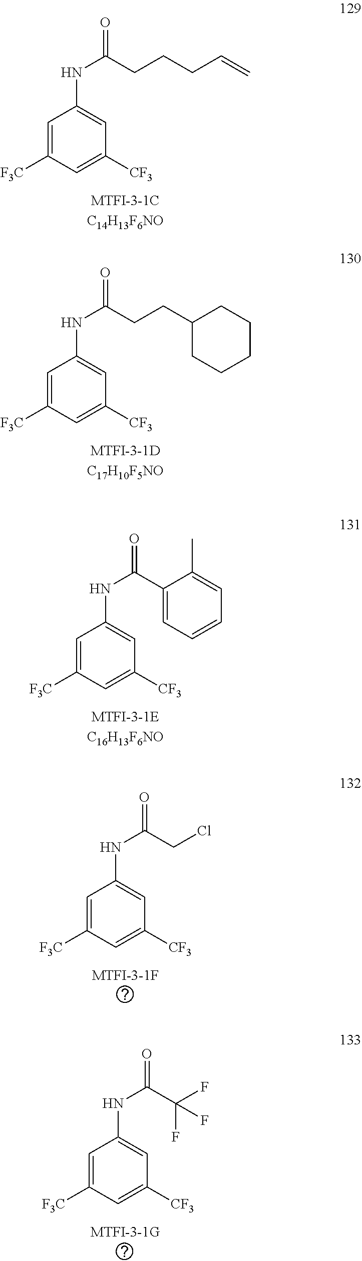 [SMILES: C=CCCCC(=O)Nc1cc(C(F)(F)F)cc(C(F)(F)F)c1 ; O=C(CCC1CCCCC1)Nc1cc(C(F)(F)F)cc(C(F)(F)F)c1 ; Cc1ccccc1C(=O)Nc1cc(C(F)(F)F)cc(C(F)(F)F)c1 ; O=C(CCl)Nc1cc(C(F)(F)F)cc(C(F)(F)F)c1 ; O=C(Nc1cc(C(F)(F)F)cc(C(F)(F)F)c1)C(F)(F)F]

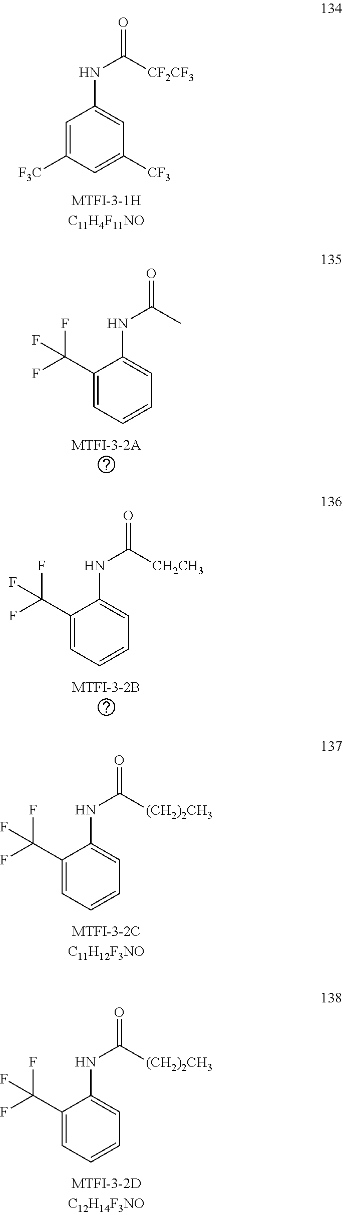

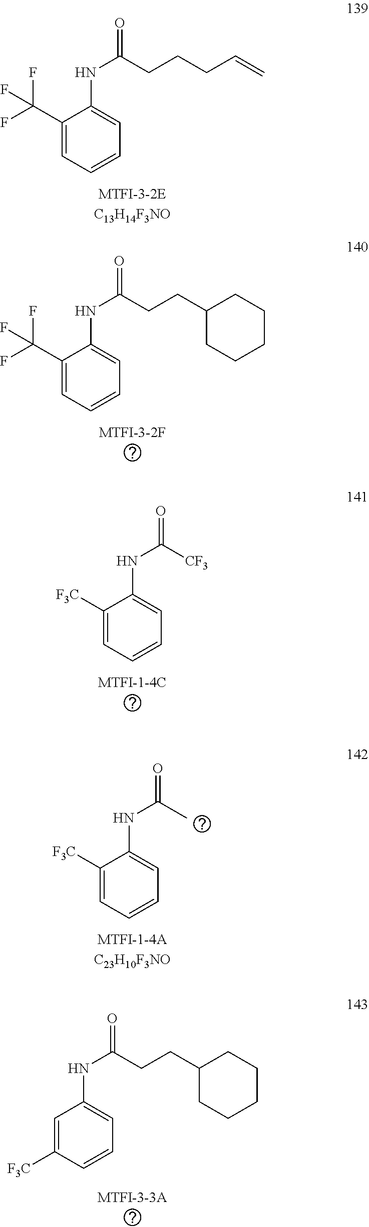 [SMILES: C=CCCCC(=O)Nc1ccccc1C(F)(F)F ; O=C(CCC1CCCCC1)Nc1ccccc1C(F)(F)F ; O=C(Nc1ccccc1C(F)(F)F)C(F)(F)F ; CC(=O)Nc1ccccc1C(F)(F)F ; O=C(CCC1CCCCC1)Nc1cccc(C(F)(F)F)c1]

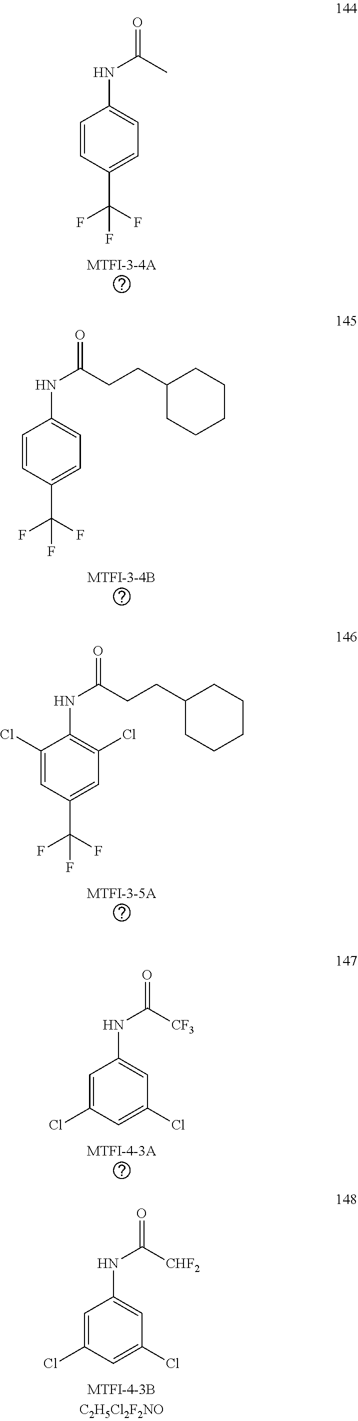 [SMILES: CC(=O)Nc1ccc(C(F)(F)F)cc1 ; O=C(CCC1CCCCC1)Nc1ccc(C(F)(F)F)cc1 ; O=C(CCC1CCCCC1)Nc1c(Cl)cc(C(F)(F)F)cc1Cl ; O=C(Nc1cc(Cl)cc(Cl)c1)C(F)(F)F ; O=C(Nc1cc(Cl)cc(Cl)c1)C(F)F]

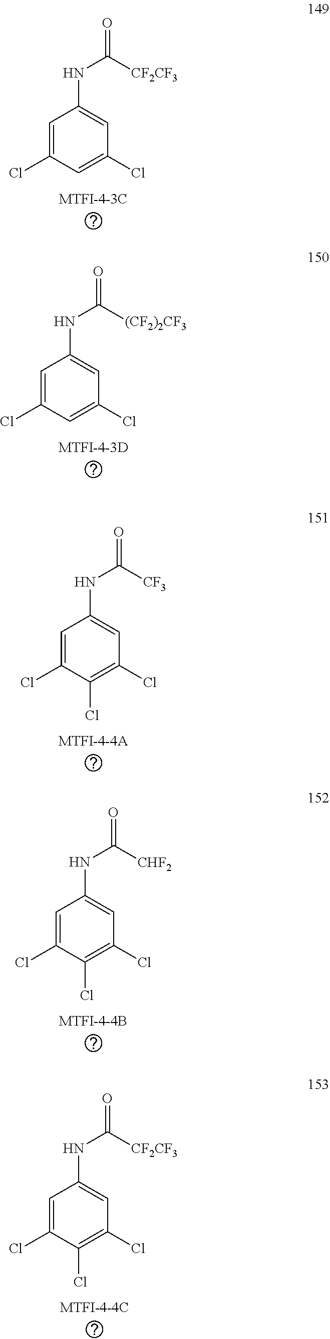

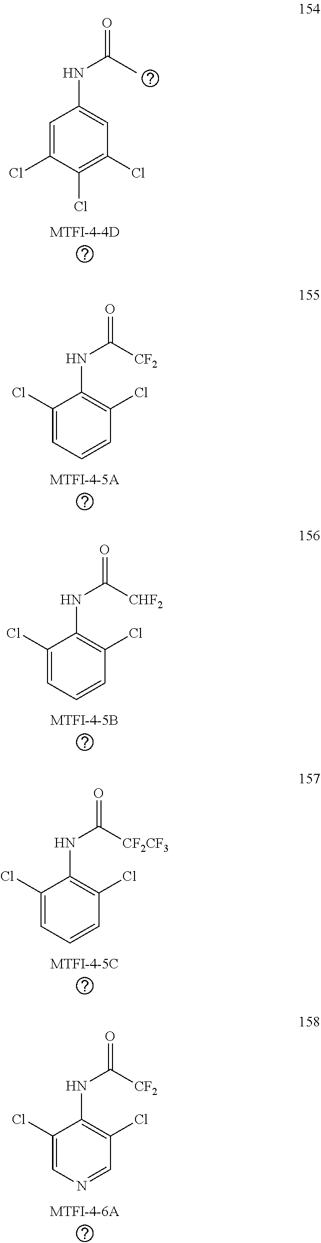

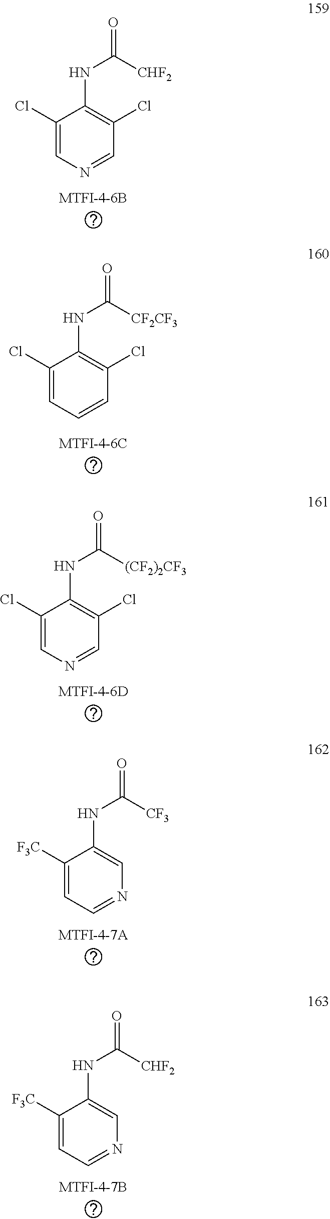

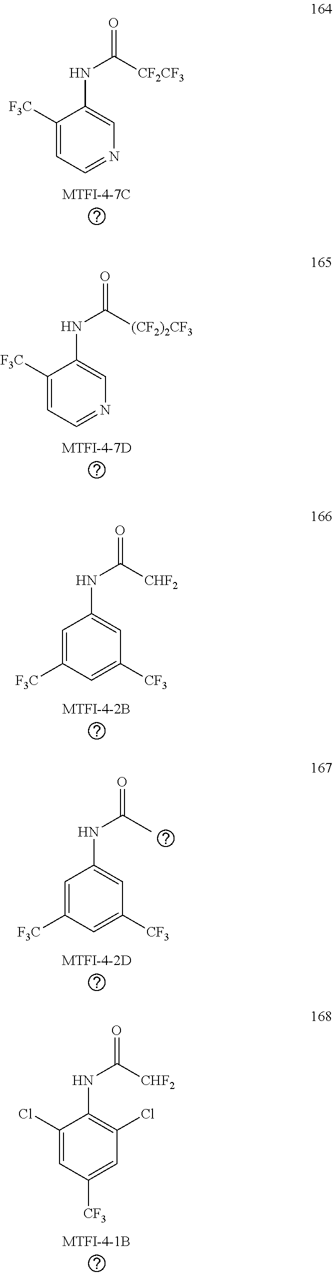

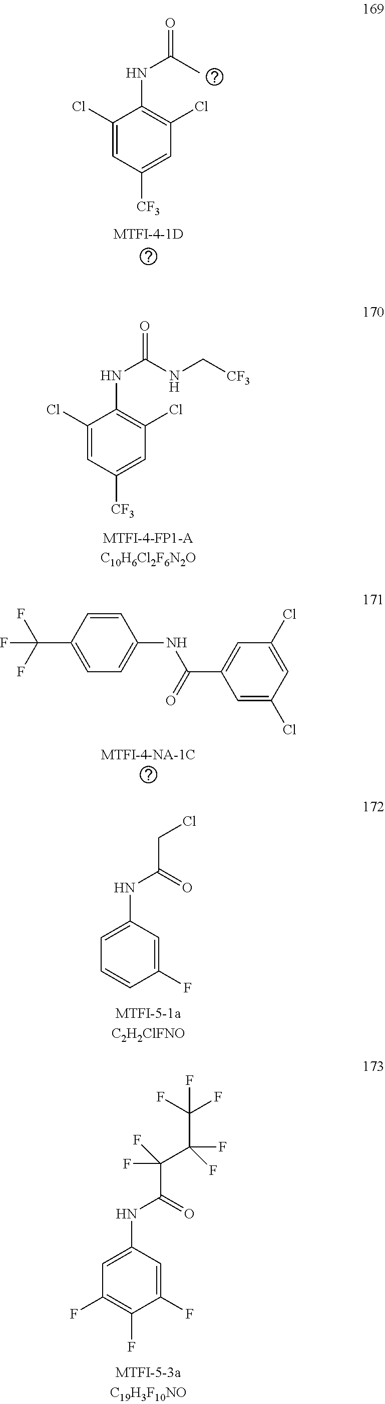 [SMILES: CC(=O)Nc1c(Cl)cc(C(F)(F)F)cc1Cl ; O=C(NCC(F)(F)F)Nc1c(Cl)cc(C(F)(F)F)cc1Cl ; O=C(Nc1ccc(C(F)(F)F)cc1)c1cc(Cl)cc(Cl)c1 ; O=C(CCl)Nc1cccc(F)c1 ; O=C(Nc1cc(F)c(F)c(F)c1)C(F)(F)C(F)(F)C(F)(F)F]

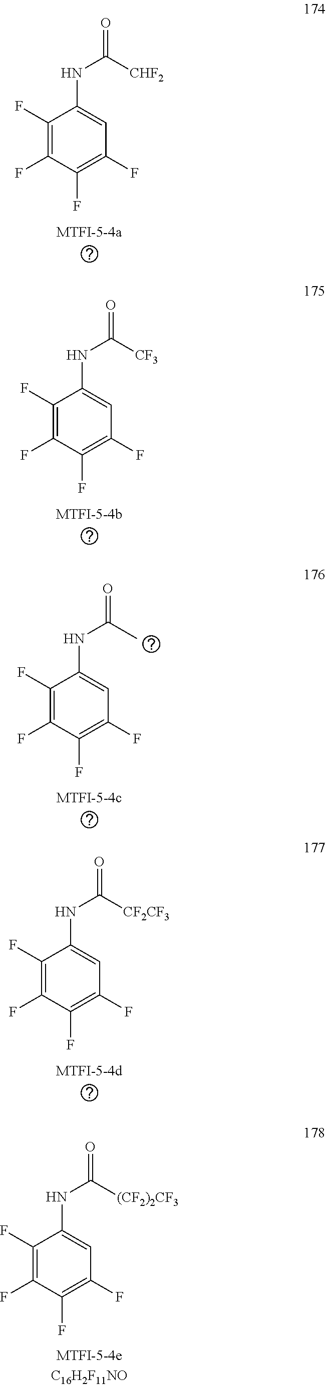

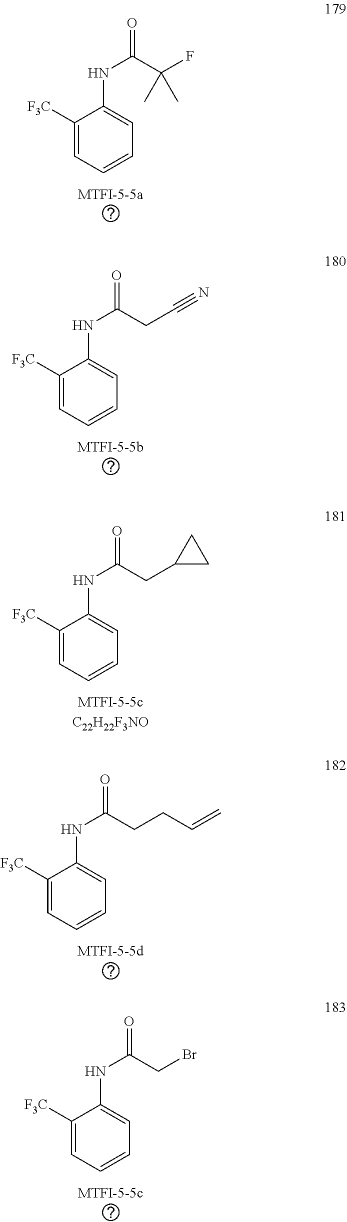 [SMILES: CC(C)(F)C(=O)Nc1ccccc1C(F)(F)F ; N#CCC(=O)Nc1ccccc1C(F)(F)F ; O=C(CC1CC1)Nc1ccccc1C(F)(F)F ; C=CCCC(=O)Nc1ccccc1C(F)(F)F ; O=C(CBr)Nc1ccccc1C(F)(F)F]

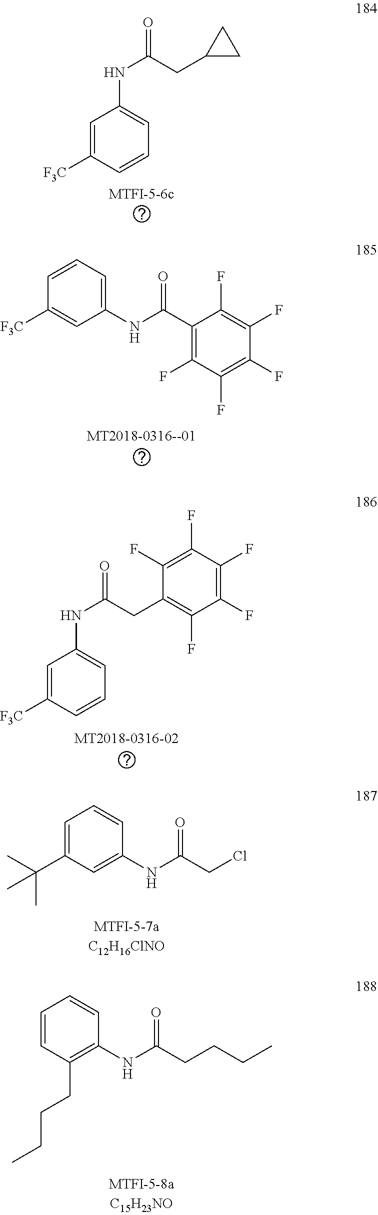 [SMILES: O=C(CC1CC1)Nc1cccc(C(F)(F)F)c1 ; O=C(Nc1cccc(C(F)(F)F)c1)c1c(F)c(F)c(F)c(F)c1F ; O=C(Cc1c(F)c(F)c(F)c(F)c1F)Nc1cccc(C(F)(F)F)c1 ; CC(C)(C)c1cccc(NC(=O)CCl)c1 ; CCCCC(=O)Nc1ccccc1CCCC]

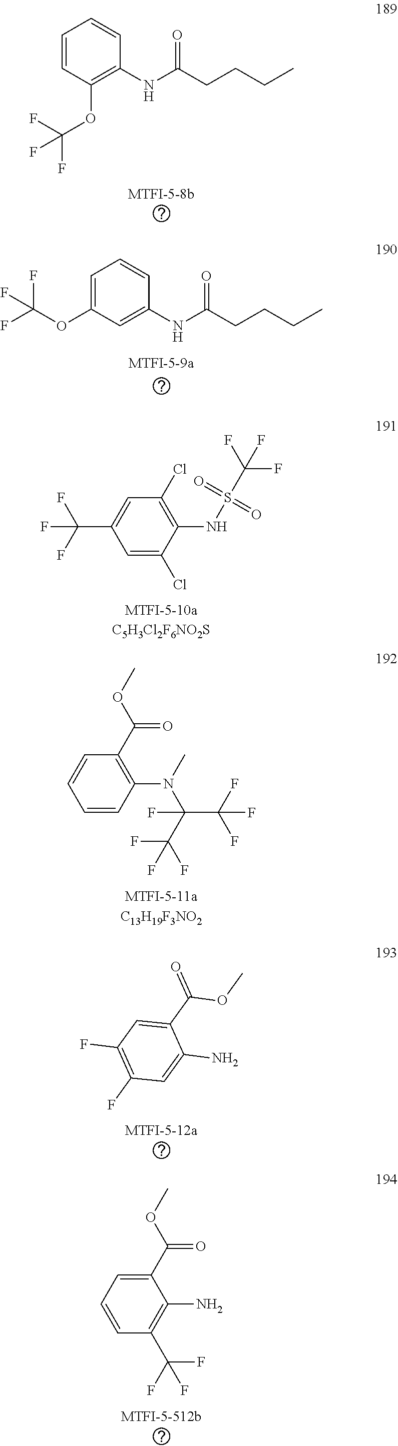 [SMILES: CCCCC(=O)Nc1ccccc1OC(F)(F)F ; CCCCC(=O)Nc1cccc(OC(F)(F)F)c1 ; O=S(=O)(Nc1c(Cl)cc(C(F)(F)F)cc1Cl)C(F)(F)F ; COC(=O)c1ccccc1N(C)C(F)(C(F)(F)F)C(F)(F)F ; COC(=O)c1cc(F)c(F)cc1N ; COC(=O)c1cccc(C(F)(F)F)c1N]

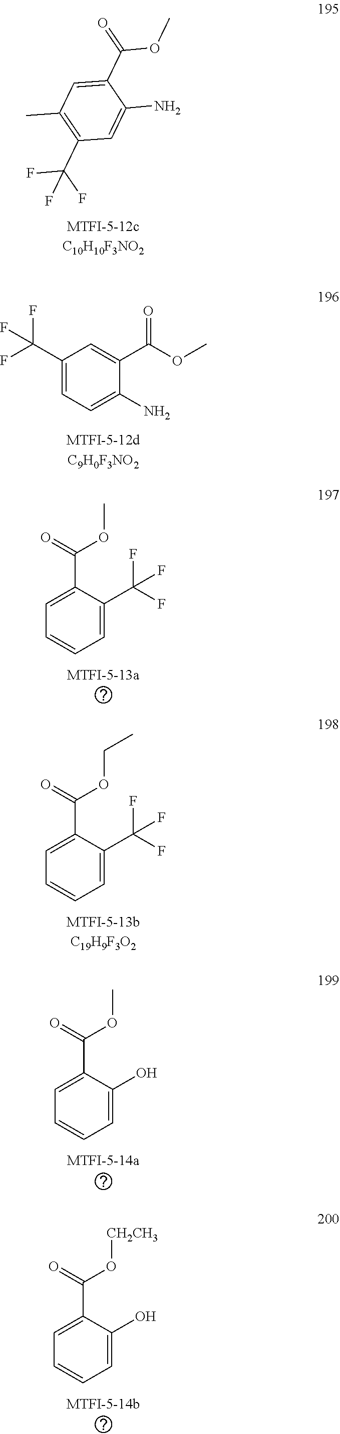

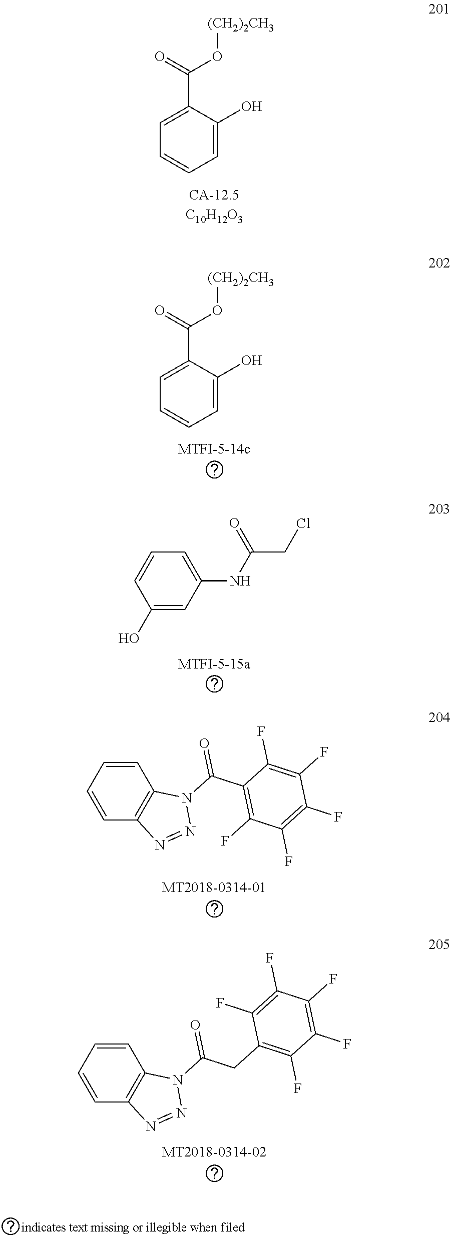

17. A composition comprising a compound of any one of embodiments 1-17 and a carrier.

18. A method of repelling insects, comprising contacting an object or locus with a compound of any one of embodiments 1-17, wherein said insects are repelled from an adjacent area to said object or locus, wherein said insects are not physically contacted with said locus.

19. A method of providing a compound of any one of embodiments 1-17 in an insect repellent medium from which a vaporized compound of any one of embodiments 1-17 can be dispersed.

20. A compound of Formula X

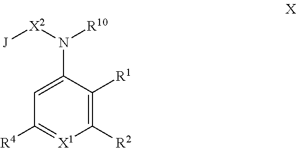

wherein,
when R¹ is hydrogen, X¹ is CR³ wherein R³ is fluorine, and R² and R⁴ are each fluorine;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₃CF₃, —CF₂C_{F}OCF₃, —CF₂CN, —(CF₂)₂CF₃, —CH₂SCF₃,

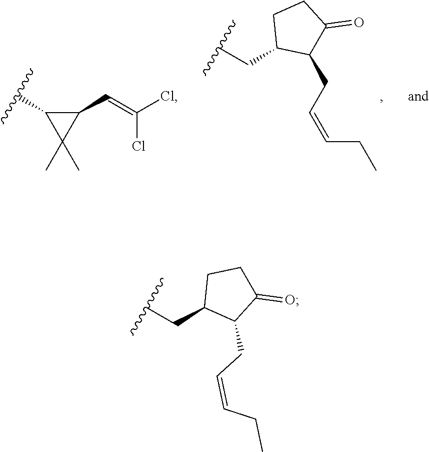

or,
when X¹ is CR³ wherein R³ is fluorine and R¹, R², and R⁴ are each fluorine; X² is selected from the group consisting of C(O), S(O), S(O)₂, —CH(OH), and —CHCF₃;
R¹⁰ is hydrogen or C₁-C₆ alkyl; and
J is selected from the group consisting of —CHF₂, —CF₃, —CF₂CHF₂, —CF₂CF₃, —(CF₂)₂CF₃;
or,
when R¹ is hydrogen, X¹ is N, and R⁴ and R² are each chlorine;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃, —(CF₂)₂CF₃, —(CF₂)₃CF₃,

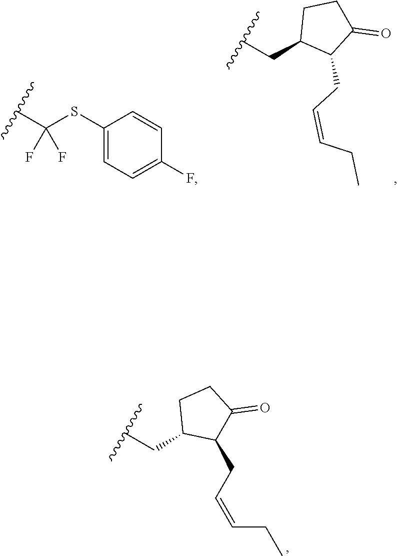 [SMILES: *C(F)(F)Sc1ccc(F)cc1 ; *C[C@H]1CCC(=O)[C@@H]1C/C=C\CC ; *C[C@@H]1CCC(=O)[C@H]1C/C=C\CC]

—CF₂CN, —(CH₂)₃CF₃, —C(CF₃)₂F, —CCl₃,

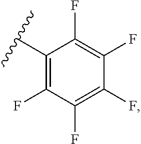 [SMILES: *c1c(F)c(F)c(F)c(F)c1F]

—(CH₂)₄CF₃,

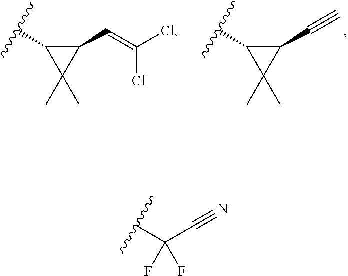

—CHF₂, —CF₃, and —CF₂CF₃;
or,
when R is hydrogen, X¹ is N, and R⁴ and R² are each fluorine;
X² is selected from the group consisting of C(O), S(O), S(O)₂, —CH(OH), and —CHCF₃;
R¹⁰ is hydrogen or C₁-C₆ alkyl; and
J is selected from the group consisting of —CHF₂, —CF₃, —CF₂CF₃, and —(CF₂)₄CF₃;
or
when R¹ is hydrogen, X¹ is CR³ wherein R³ is hydrogen, R⁴ is chlorine, and R² is chlorine,
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃,

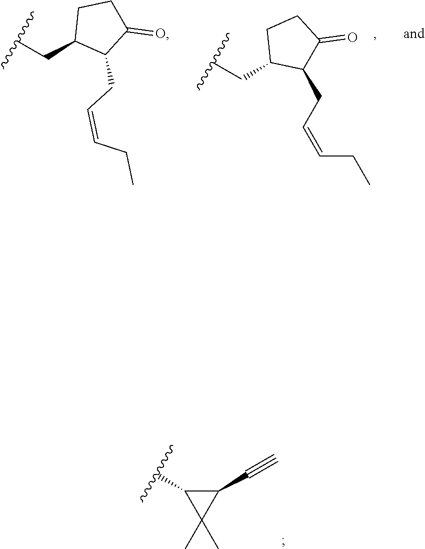

or,
when R¹ is hydrogen, X¹ is N, and R⁴ and R² are each bromine;
X² is selected from the group consisting of C(O), S(O), S(O)₂, —CH(OH), and —CHCF₃;
R¹⁰ is hydrogen or C₁-C₆ alkyl; and
J is selected from the group consisting of —CF₂H, —CF₃, —CF₂CF₃, —(CF₂)₂CF₃, and —(CF₂)₄CF₃;
or,
when R¹ is hydrogen, X¹ is CR³ wherein R³ is hydrogen, one of R⁴ and R² is chlorine and the other is —CF₃;
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₄CF₃, —CF(CF₃)₂, —(CF₂)₂OCF₃, —(CF₂)₂CF₃, —CHF₂, —CF₃, and —(CF₂)CF₃;
or,
when R¹ is hydrogen, X¹ is N, and R⁴ and R² are C₁-C₃ alkyl;
X² is selected from the group consisting of C(O), S(O), S(O)₂, —CH(OH), and —CHCF₃;
R¹⁰ is hydrogen or C₁-C₆ alkyl; and
J is selected from the group consisting of —CF₃, —CF₂CF₃, —CHF₂, and —(CF₂)₄CF₃; or,
when R¹ is hydrogen, X¹ is CR³ wherein R³ is hydrogen, R⁴ is —CF₃, and R² is —CF₃;
X² is selected from the group consisting of C(O), S(O), S(O)₂, —CH(OH), and —CHCF₃;
R¹⁰ is hydrogen or C₁-C₆ alkyl; and
J is —(CF₂)₂CF₃.

21. The compound of embodiment 20, wherein X² is C(O).

22. The compound of embodiment 20 or 21, wherein R¹⁰ is hydrogen.

23. The compound of embodiment 20 or 21, wherein R¹⁰ is C₁-C₃ alkyl.

24. The compound of embodiment 20, 21, or 23, wherein R¹⁰ is methyl.

25. The compound of any one of embodiments 20-24, wherein R¹ is hydrogen, X¹ is CR³ wherein R³ is fluorine, and R² and R⁴ are each fluorine, J is selected from the group consisting of —(CF₂)₂CF₃,

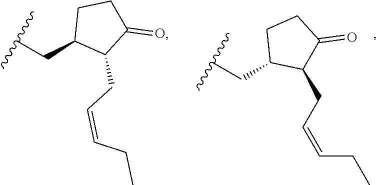 [SMILES: *C[C@H]1CCC(=O)[C@@H]1C/C=C\CC ; *C[C@@H]1CCC(=O)[C@H]1C/C=C\CC]

—CF₂CF₂OCF₃, —CH₂SCF₃, —CF₂CN, and

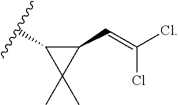 [SMILES: *[C@@H]1[C@@H](C=C(Cl)Cl)C1(C)C]

26. The compound of any one of embodiments 20-25, wherein R¹ is hydrogen, X is CR³ wherein R³ is fluorine, and R² and R⁴ are each fluorine, J is —(CF₂)₂CF₃.

27. The compound of any one of embodiments 20-26, wherein said compound has the structure

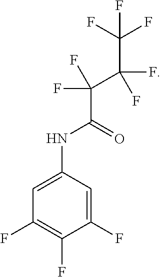 [SMILES: O=C(Nc1cc(F)c(F)c(F)c1)C(F)(F)C(F)(F)C(F)(F)F]

28. The compound of any one of embodiments 20-24, wherein R¹ is hydrogen, X¹ is CR³ wherein R³ is fluorine, and R² and R⁴ are each fluorine, J is selected from the group consisting of —(CF₂)₃CF₃, —(CF₂)₂CF₃, and —CF₂CF₂OCF₃.

29. The compound of any one of embodiments 20-22, wherein X¹ is CR³, R³ is fluorine and R¹, R², and R⁴ are each fluorine, and J is selected from the group consisting of —CHF₂, —CF₃, —CF₂CHF₂, —CF₂CF₃, —(CF₂)₂CF₃.

30. The compound of any one of embodiments 20-22, wherein R¹ is hydrogen, X¹ is N, and R⁴ and R² are each chlorine, and J is selected from the group consisting of —CHF₂, —CF₃, —CF₂CF₃, —(CF₂)₂CF₃,

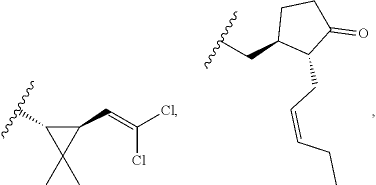

—(CF₂)₄CF₃, —(CF₂)₃CF₃, —(CH₂)₃CF₃, —C(CF₃)₂F, —(CH₂)₄CH₃, —(CF₂)₂OCF₃,

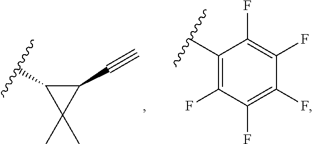

### —CCl₃,

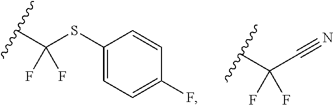 [SMILES: *C(F)(F)Sc1ccc(F)cc1 ; *C(F)(F)C#N]

31. The compound of any one of embodiments 20-24, wherein R¹ is hydrogen, X¹ is N, and R⁴ and R² are each chlorine, and J is —(CF₂)₂CF₃ or —(CF₂)₂OCF₃.

32. The compound of any one of embodiments 20-22, wherein R is hydrogen, X¹ is N, and R⁴ and R² are each fluorine, and J is selected from the group consisting of —CHF₂, —CF₃, —CF₂CF₃, and —(CF₂)₄CF₃.

33. The compound of any one of embodiments 20-22, wherein R¹ is hydrogen, X¹ is CR³ wherein R³ is hydrogen, R⁴ is chlorine, and R² is chlorine, and J is

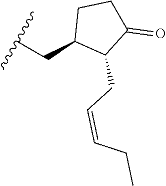 [SMILES: *C[C@H]1CCC(=O)[C@@H]1C/C=C\CC]

or —(CF₂)₂OCF₃.

34. The compound of any one of embodiments 20-22, wherein R¹ is hydrogen, X¹ is N, and R⁴ and R² are each bromine, and J is selected from the group consisting of —CF₂H, —CF₃, —CF₂CF₃, —(CF₂)₂CF₃, and —(CF₂)₄CF₃.

35. The compound of any one of embodiments 20-22, wherein R¹ is hydrogen, X¹ is CR³ wherein R³ is hydrogen, one of R⁴ and R² is chlorine and the other is —CF₃, and J is selected from the group consisting of —(CF₂)₂OCF₃, —CHF₂, —CF₃, —(CF₂)CF₃, —(CF₂)₂CF₃, —(CF₂)₄CF₃, —(CH₂)₄CH₃, and —CF(CF₃)₂.

36. The compound of any one of embodiments 20-24, wherein R¹ is hydrogen, X¹ is CR³ wherein R³ is hydrogen, one of R⁴ and R⁵ is chlorine and the other is —CF₃, and J is —(CF₂)₂CF₃.

37. The compound of any one of embodiments 20-22, wherein R¹ is hydrogen, X¹ is N, and R⁴ and R² are each methyl, and J is selected from the group consisting of —CF₃, —CF₂CF₃, —CHF₂, and —(CFC)₄CF₃.

38. The compound of any one of embodiments 20-22, wherein R¹ is hydrogen, X¹ is CR³ wherein R³ is hydrogen, R⁴ is —CF₃, and R² is —CF₃, and J is —(CF₂)₂CF₃.

39. The compound of any one of embodiments 20-38, wherein said compound has a structure selected from the group consisting of

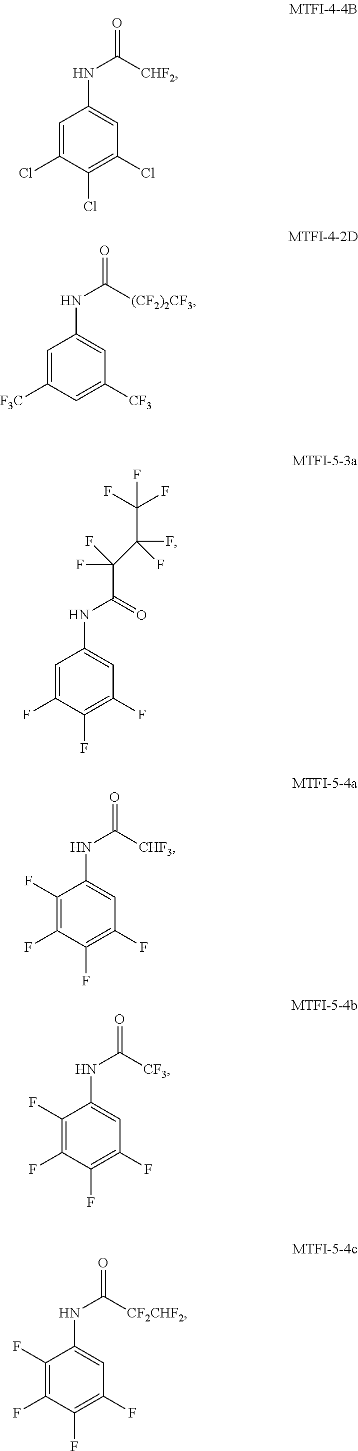

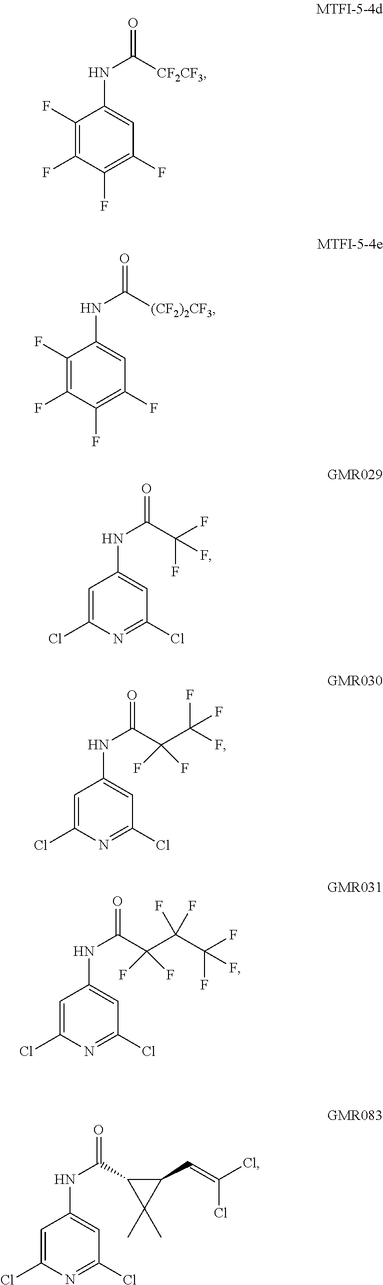

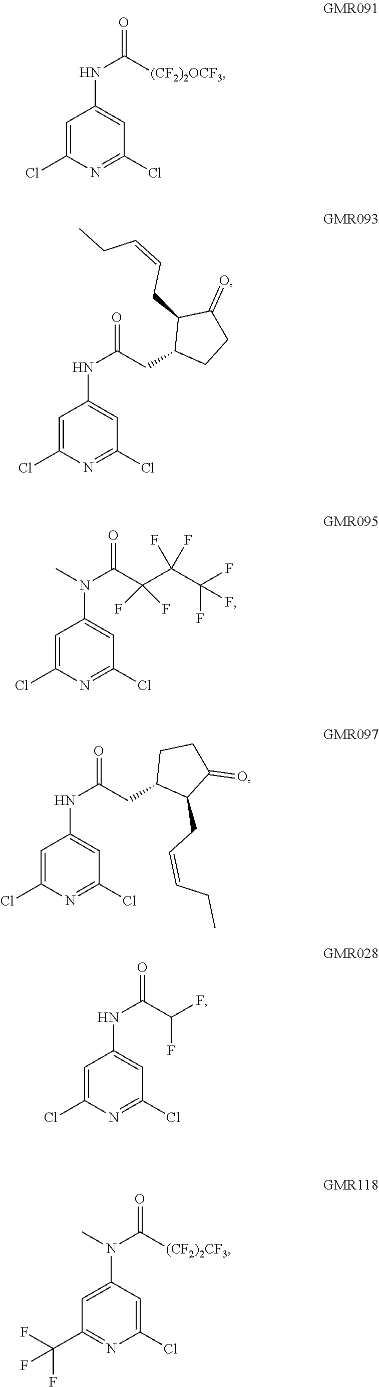

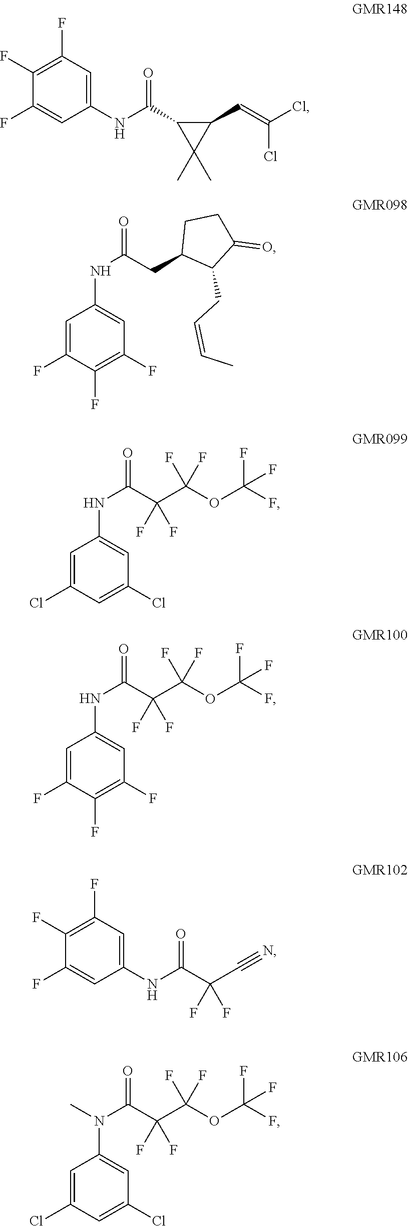

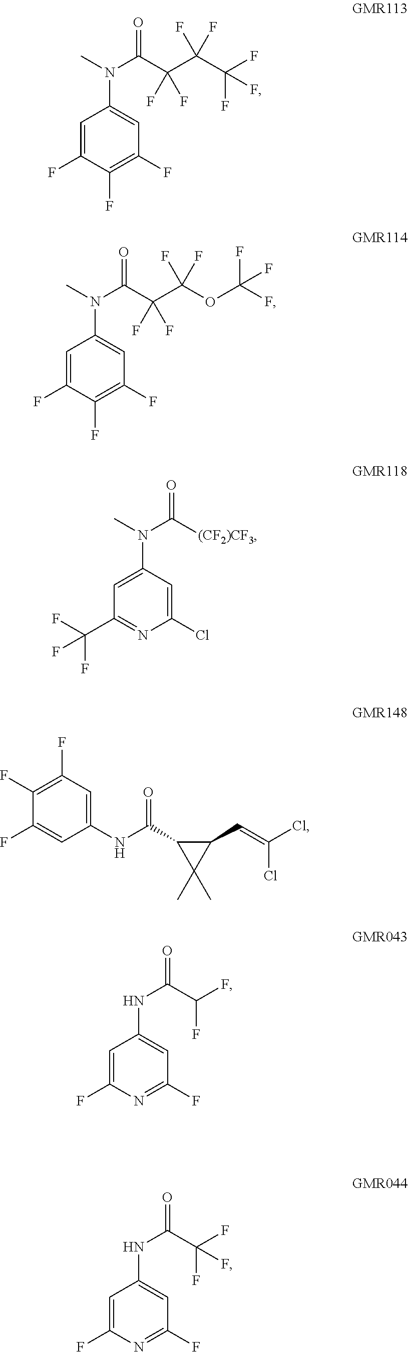

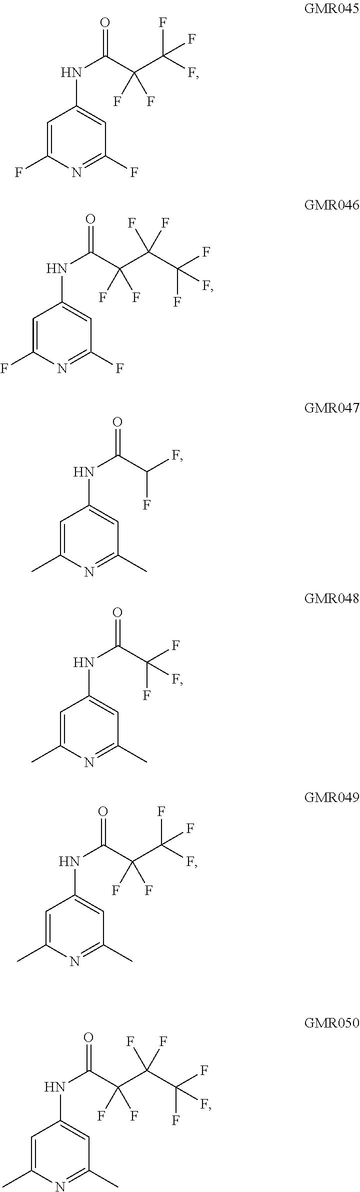 [SMILES: O=C(Nc1cc(F)nc(F)c1)C(F)(F)C(F)(F)F ; O=C(Nc1cc(F)nc(F)c1)C(F)(F)C(F)(F)C(F)(F)F ; Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1]

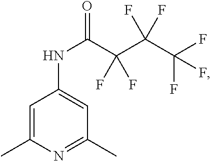 [SMILES: Cc1cc(NC(=O)C(F)(F)C(F)(F)C(F)(F)F)cc(C)n1]

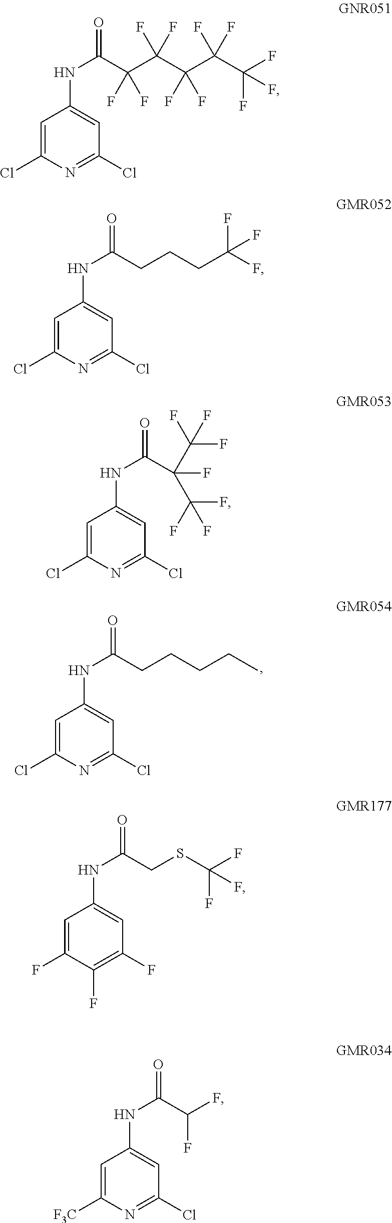 [SMILES: O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1 ; O=C(Nc1cc(Cl)nc(Cl)c1)C(F)(C(F)(F)F)C(F)(F)F ; CCCCCC(=O)Nc1cc(Cl)nc(Cl)c1 ; O=C(CSC(F)(F)F)Nc1cc(F)c(F)c(F)c1 ; O=C(Nc1cc(Cl)nc(C(F)(F)F)c1)C(F)F]

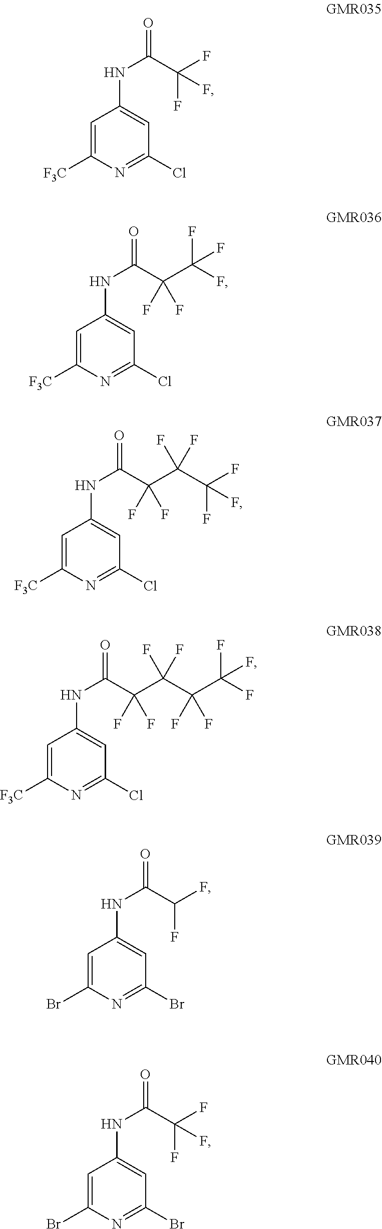 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(C(F)(F)F)c1)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

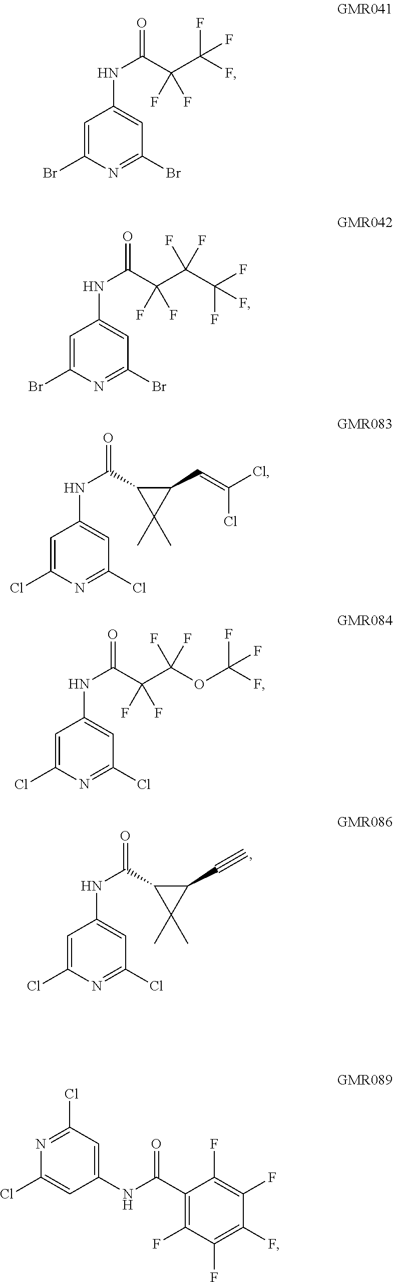 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; CC1(C)[C@H](C=C(Cl)Cl)[C@H]1C(=O)Nc1cc(Cl)nc(Cl)c1 ; O=C(Nc1cc(Cl)nc(Cl)c1)C(F)(F)C(F)(F)OC(F)(F)F ; C#C[C@@H]1[C@@H](C(=O)Nc2cc(Cl)nc(Cl)c2)C1(C)C ; O=C(Nc1cc(Cl)nc(Cl)c1)c1c(F)c(F)c(F)c(F)c1F]

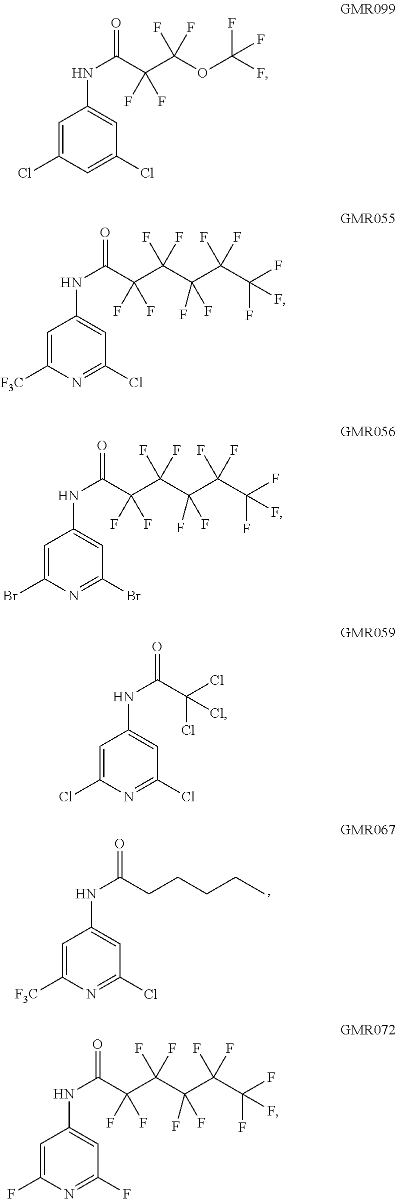 [SMILES: O=C(Nc1cc(Cl)cc(Cl)c1)C(F)(F)C(F)(F)OC(F)(F)F ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F ; O=C(Nc1cc(Cl)nc(Cl)c1)C(Cl)(Cl)Cl ; CCCCCC(=O)Nc1cc(Cl)nc(C(F)(F)F)c1 ; O=C(Nc1cc(F)nc(F)c1)C(F)(F)C(F)(F)C(F)(F)C(F)(F)C(F)(F)F]

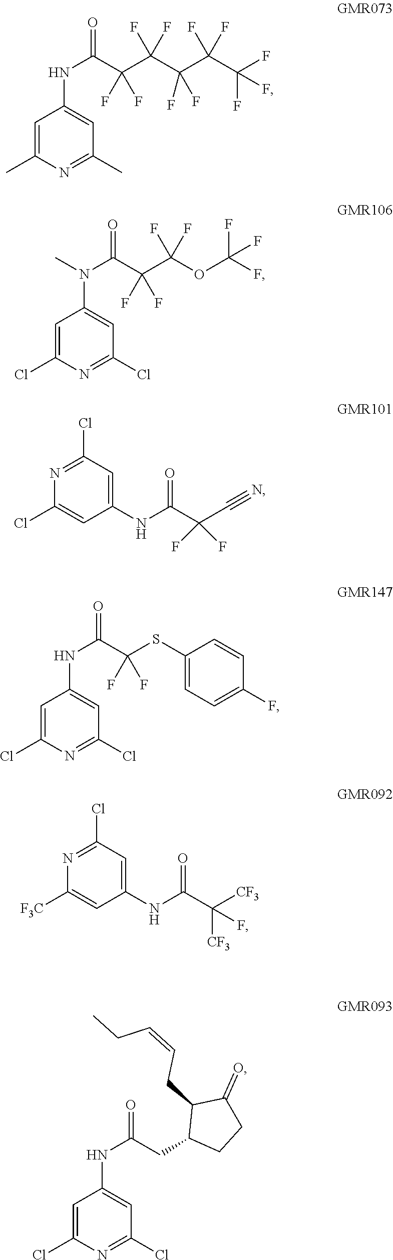 [SMILES: Cc1cc(NC(=O)C(F)(F)C(F)(F)C(F)(F)C(F)(F)C(F)(F)F)cc(C)n1 ; CN(C(=O)C(F)(F)C(F)(F)OC(F)(F)F)c1cc(Cl)nc(Cl)c1 ; N#CC(F)(F)C(=O)Nc1cc(Cl)nc(Cl)c1 ; O=C(Nc1cc(Cl)nc(Cl)c1)C(F)(F)Sc1ccc(F)cc1 ; O=C(Nc1cc(Cl)nc(C(F)(F)F)c1)C(F)(C(F)(F)F)C(F)(F)F ; CC/C=C\C[C@H]1C(=O)CC[C@@H]1CC(=O)Nc1cc(Cl)nc(Cl)c1]

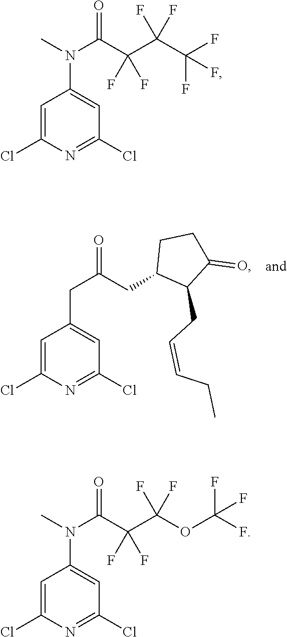 [SMILES: CN(C(=O)C(F)(F)C(F)(F)C(F)(F)F)c1cc(Cl)nc(Cl)c1 ; CC/C=C\C[C@@H]1C(=O)CC[C@H]1CC(=O)Cc1cc(Cl)nc(Cl)c1 ; "and" ; CN(C(=O)C(F)(F)C(F)(F)OC(F)(F)F)c1cc(Cl)nc(Cl)c1]

40. A composition comprising a compound of any one of embodiments 20-39 and a carrier.

41. A method of repelling a species of insect from a locus to which said species of insect is normally attracted, comprising contacting on said locus a non-toxic effective insect repellent of the composition of embodiment 40.

42. A method for controlling one or more insects, comprising contacting the insects with the composition of embodiment 40.

43. The method of any one of embodiments 41-42, wherein the composition is a solution, dust, granular formulation, or emulsion.

44. The method of any one of embodiments 41-43, wherein the composition is a liquid solution or emulsion.

45. The method of any one of embodiments 41-43, wherein said species of insect is a fly, spider, butterfly, crab, mosquito, centipede, tick, millipede, or scorpion.

46. The method of any one of embodiments 41-45, wherein said insect is a mosquito.

47. The method of any one of embodiments 41-46, where the locus is an area, an environment, or the skin of an animal.

48. A method of repelling insects, comprising contacting an object or locus with a compound of any one of embodiments 20-39, wherein said insects are repelled from an adjacent area to said object or locus, wherein said insects are not physically contacted with said locus.

49. A method of providing a compound of any one of embodiments 20-29 in an insect repellent medium from which a vaporized compound of any one of embodiments 20-29 can be dispersed.

50. A compound, selected from the group consisting of:

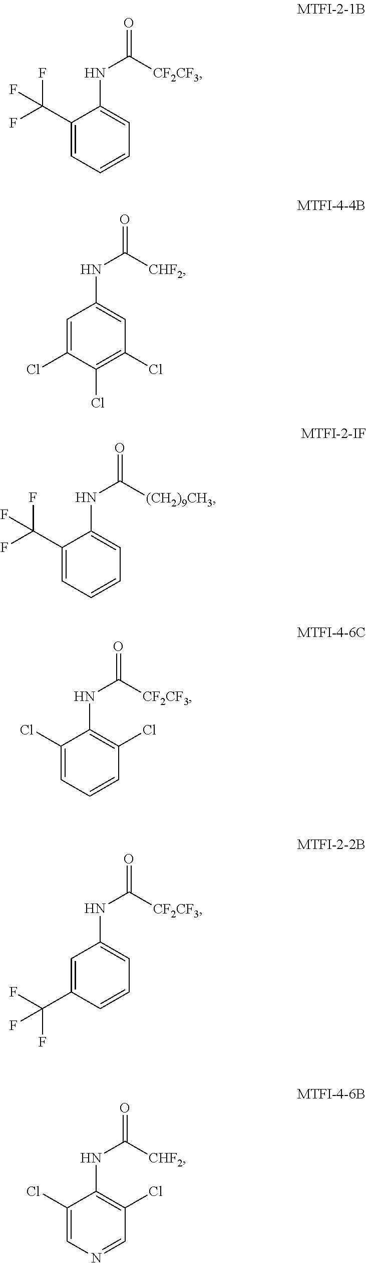

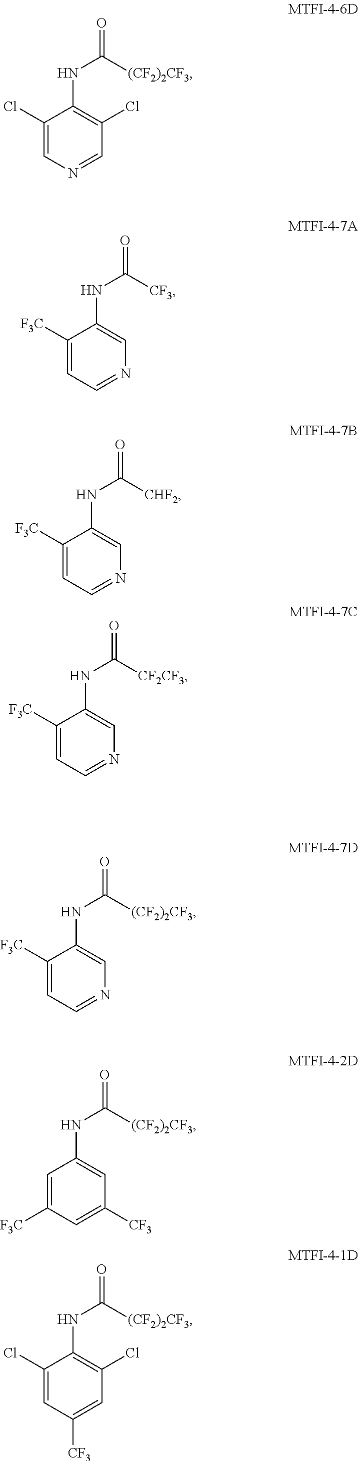

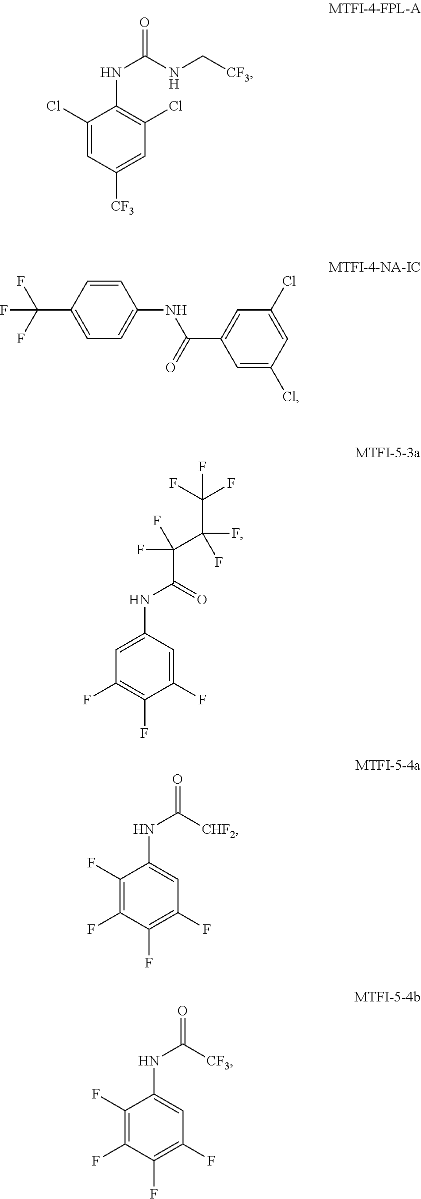 [SMILES: O=C(NCC(F)(F)F)Nc1c(Cl)cc(C(F)(F)F)cc1Cl ; O=C(Nc1ccc(C(F)(F)F)cc1)c1cc(Cl)cc(Cl)c1 ; O=C(Nc1cc(F)c(F)c(F)c1)C(F)(F)C(F)(F)C(F)(F)F ; O=C(Nc1cc(F)c(F)c(F)c1F)C(F)F ; O=C(Nc1cc(F)c(F)c(F)c1F)C(F)(F)F]

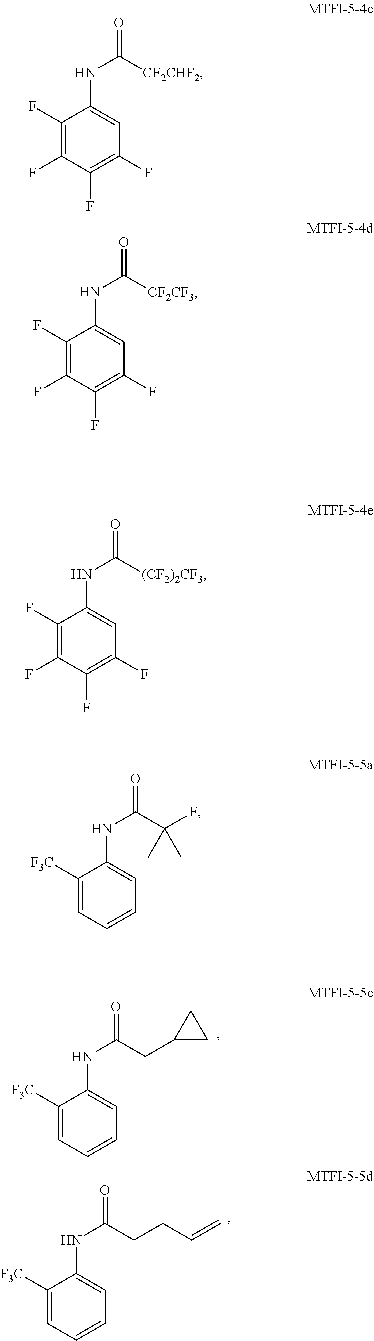

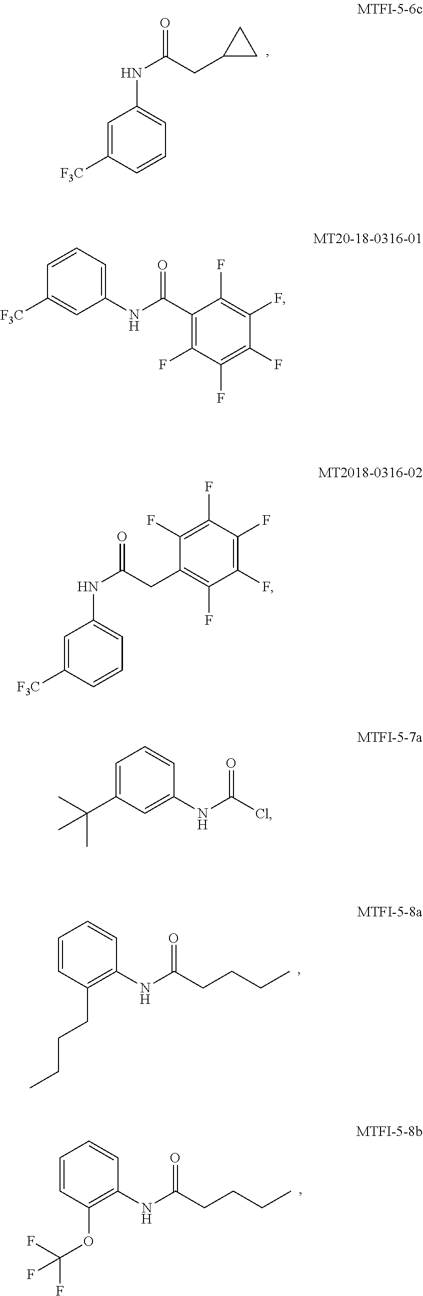 [SMILES: O=C(CC1CC1)Nc1cccc(C(F)(F)F)c1 ; O=C(Nc1cccc(C(F)(F)F)c1)c1c(F)c(F)c(F)c(F)c1F ; O=C(Cc1c(F)c(F)c(F)c(F)c1F)Nc1cccc(C(F)(F)F)c1 ; CC(C)(C)c1cccc(NC(=O)Cl)c1 ; CCCCC(=O)Nc1ccccc1CCCC ; CCCCC(=O)Nc1ccccc1OC(F)(F)F]

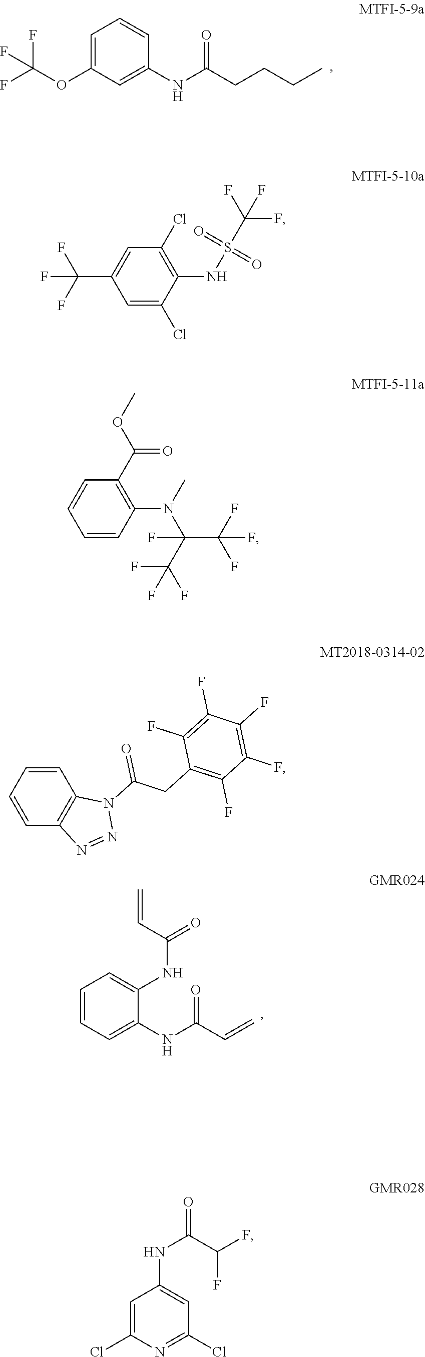 [SMILES: CCCCC(=O)Nc1cccc(OC(F)(F)F)c1 ; O=S(=O)(Nc1c(Cl)cc(C(F)(F)F)cc1Cl)C(F)(F)F ; COC(=O)c1ccccc1N(C)C(F)(C(F)(F)F)C(F)(F)F ; O=C(Cc1c(F)c(F)c(F)c(F)c1F)n1nnc2ccccc21 ; C=CC(=O)Nc1ccccc1NC(=O)C=C ; O=C(Nc1cc(Cl)nc(Cl)c1)C(F)F]

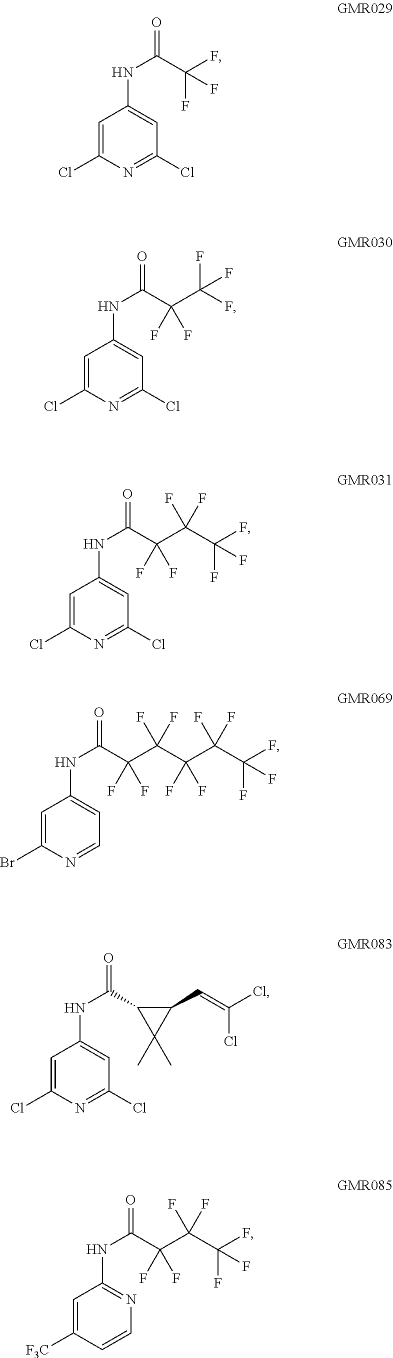 [SMILES: O=C(Nc1cc(Cl)nc(Cl)c1)C(F)(F)F ; O=C(Nc1cc(Cl)nc(Cl)c1)C(F)(F)C(F)(F)F ; O=C(Nc1cc(Cl)nc(Cl)c1)C(F)(F)C(F)(F)C(F)(F)F ; O=C(Nc1ccnc(Br)c1)C(F)(F)C(F)(F)C(F)(F)C(F)(F)C(F)(F)F ; CC1(C)[C@H](C=C(Cl)Cl)[C@H]1C(=O)Nc1cc(Cl)nc(Cl)c1 ; O=C(Nc1cc(C(F)(F)F)ccn1)C(F)(F)C(F)(F)C(F)(F)F]

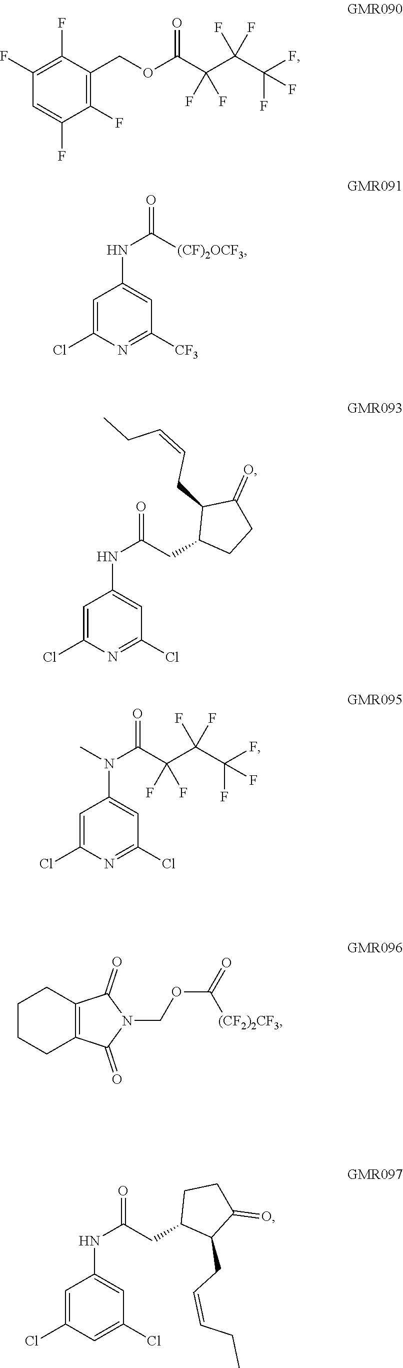

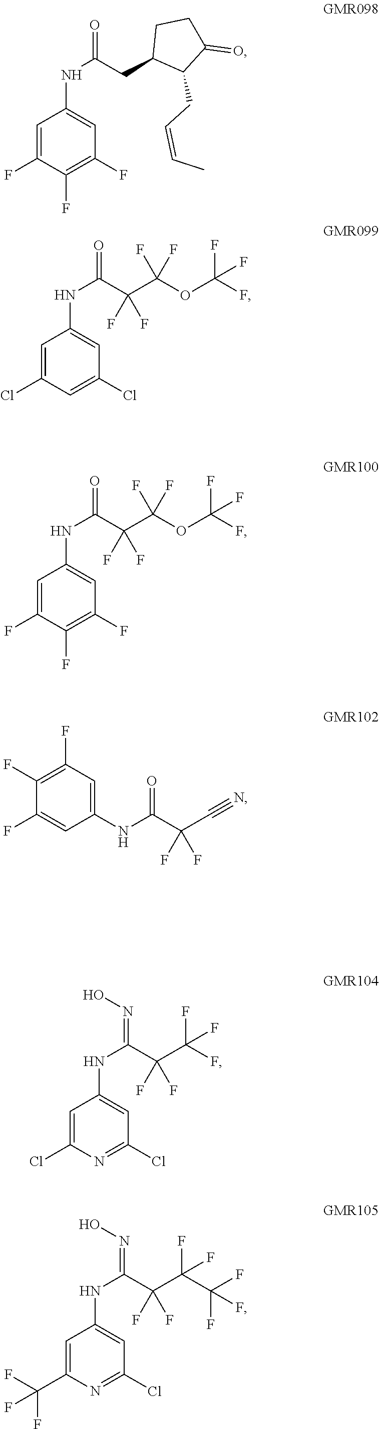 [SMILES: C/C=C\C[C@H]1C(=O)CC[C@@H]1CC(=O)Nc1cc(F)c(F)c(F)c1 ; O=C(Nc1cc(Cl)cc(Cl)c1)C(F)(F)C(F)(F)OC(F)(F)F ; O=C(Nc1cc(F)c(F)c(F)c1)C(F)(F)C(F)(F)OC(F)(F)F ; N#CC(F)(F)C(=O)Nc1cc(F)c(F)c(F)c1 ; O/N=C(\Nc1cc(Cl)nc(Cl)c1)C(F)(F)C(F)(F)F ; O/N=C(\Nc1cc(Cl)nc(C(F)(F)F)c1)C(F)(F)C(F)(F)C(F)(F)F]

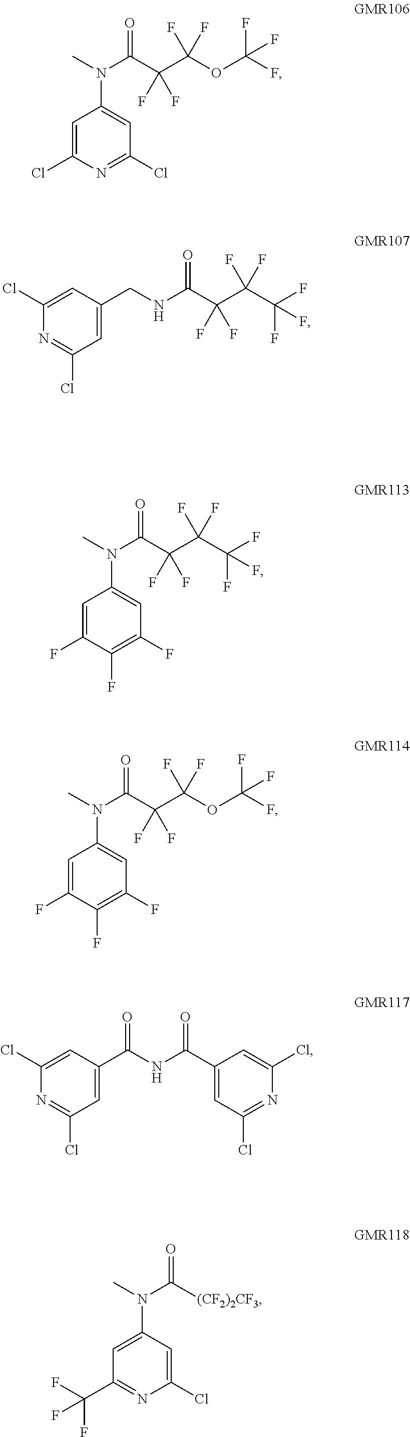

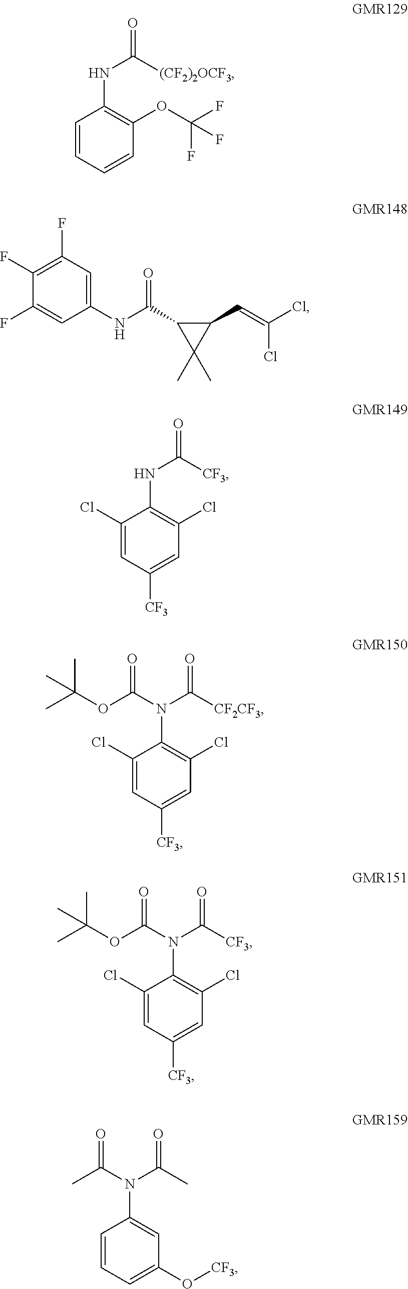

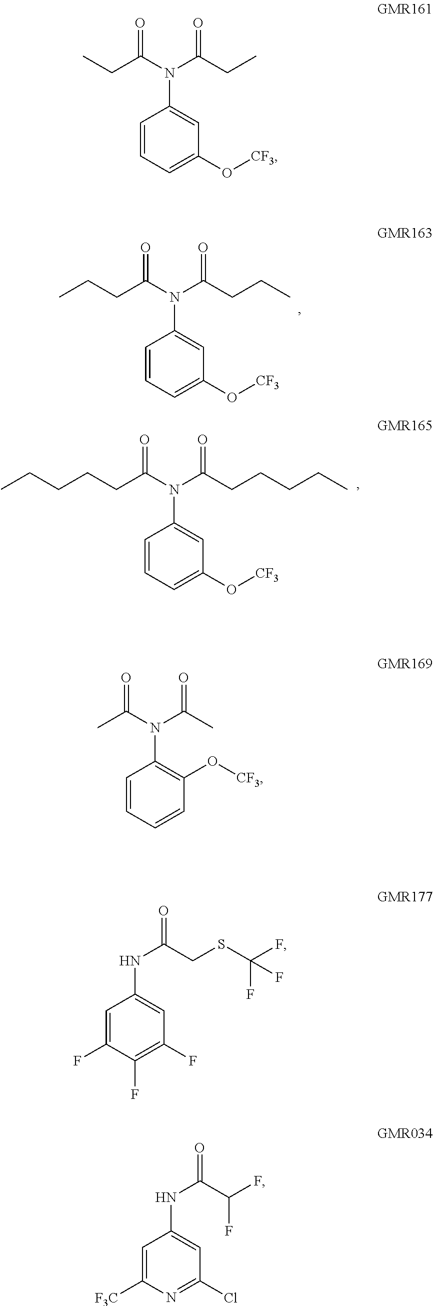

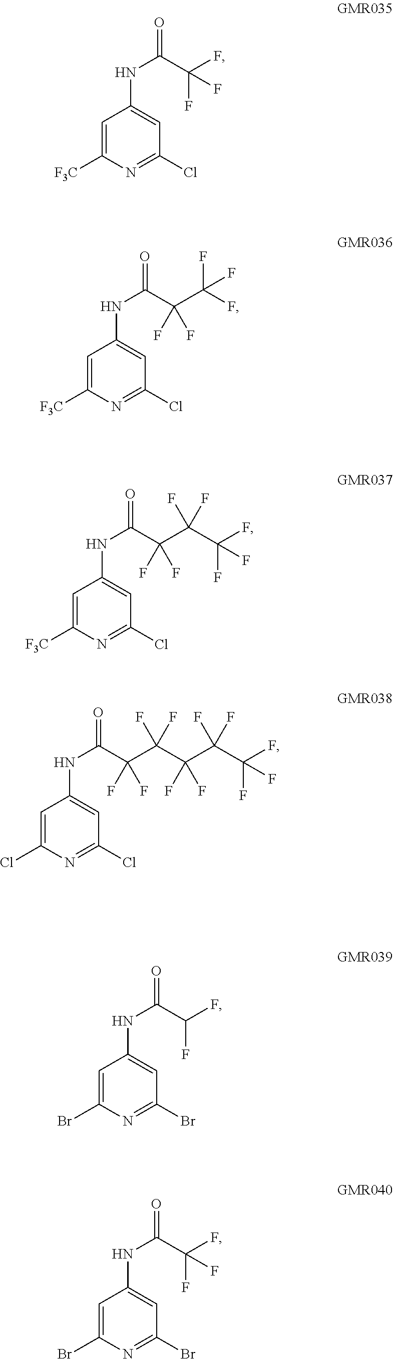 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(Cl)c1)C(F)(F)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

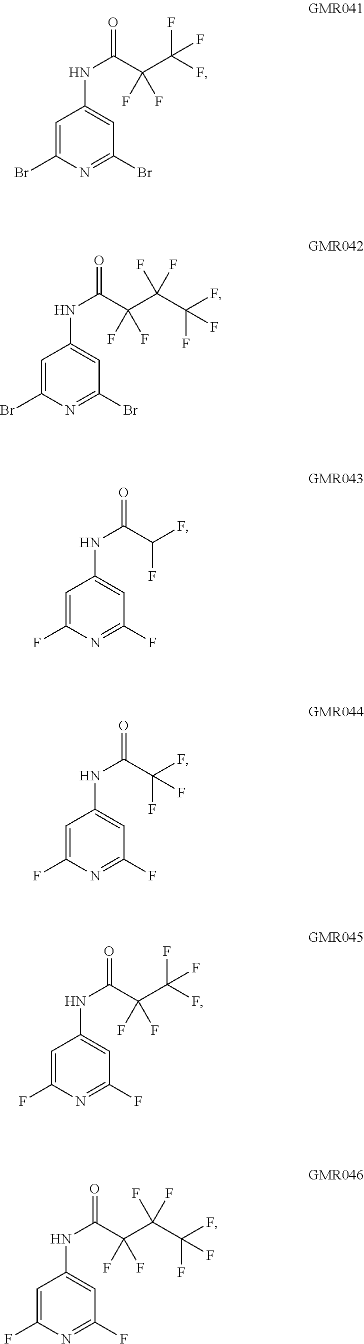 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; O=C(Nc1cc(F)nc(F)c1)C(F)F ; O=C(Nc1cc(F)nc(F)c1)C(F)(F)F ; O=C(Nc1cc(F)nc(F)c1)C(F)(F)C(F)(F)F ; O=C(Nc1cc(F)nc(F)c1)C(F)(F)C(F)(F)C(F)(F)F]

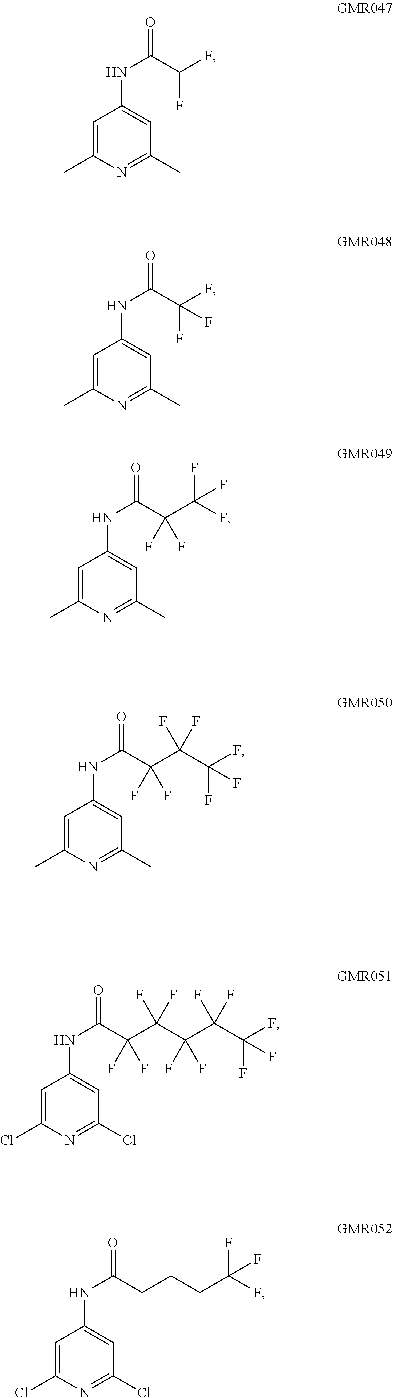 [SMILES: Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1 ; O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1]

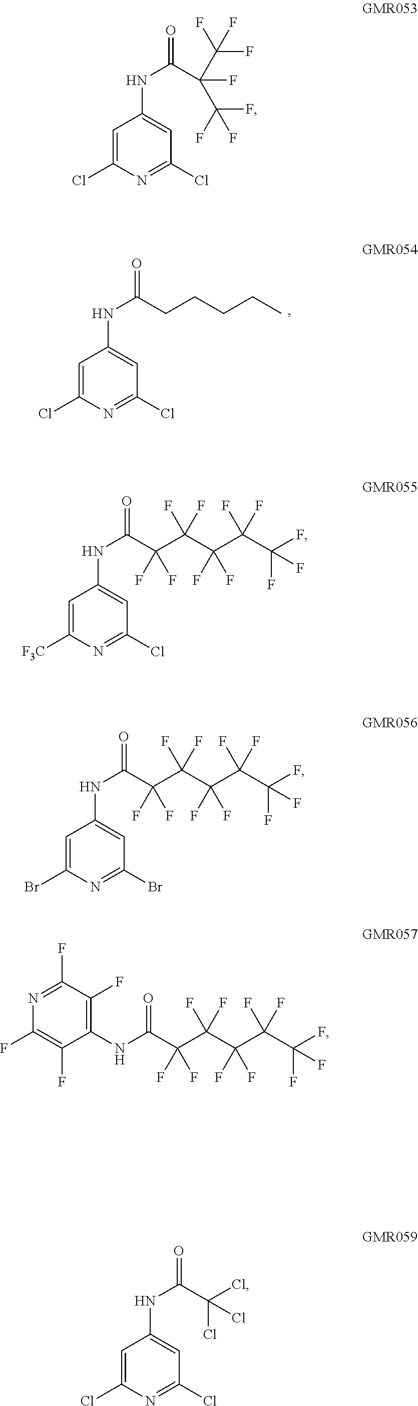 [SMILES: O=C(Nc1cc(Cl)nc(Cl)c1)C(F)(C(F)(F)F)C(F)(F)F ; CCCCCC(=O)Nc1cc(Cl)nc(Cl)c1 ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F ; O=C(Nc1c(F)c(F)nc(F)c1F)C(F)(F)C(F)(F)C(F)(F)C(F)(F)C(F)(F)F ; O=C(Nc1cc(Cl)nc(Cl)c1)C(Cl)(Cl)Cl]

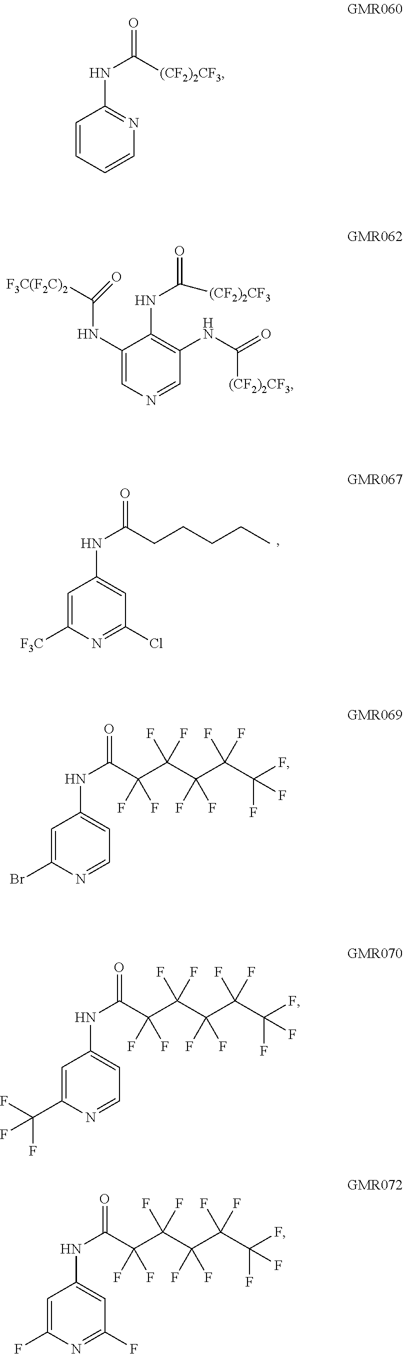

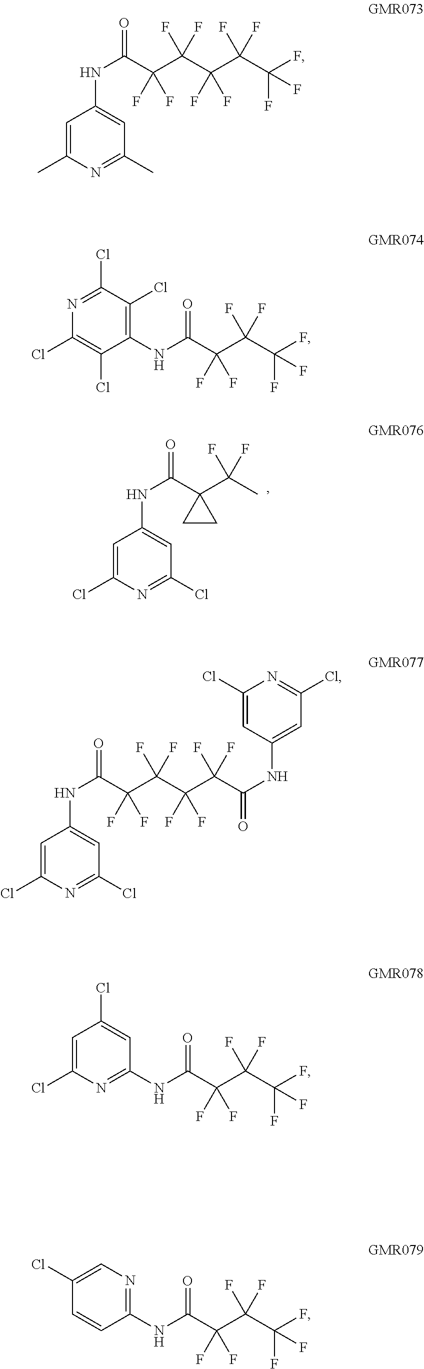 [SMILES: Cc1cc(NC(=O)C(F)(F)C(F)(F)C(F)(F)C(F)(F)C(F)(F)F)cc(C)n1 ; O=C(Nc1c(Cl)c(Cl)nc(Cl)c1Cl)C(F)(F)C(F)(F)C(F)(F)F ; CC(F)(F)C1(C(=O)Nc2cc(Cl)nc(Cl)c2)CC1 ; O=C(Nc1cc(Cl)nc(Cl)c1)C(F)(F)C(F)(F)C(F)(F)C(F)(F)C(=O)Nc1cc(Cl)nc(Cl)c1 ; O=C(Nc1cc(Cl)cc(Cl)n1)C(F)(F)C(F)(F)C(F)(F)F ; O=C(Nc1ccc(Cl)cn1)C(F)(F)C(F)(F)C(F)(F)F]

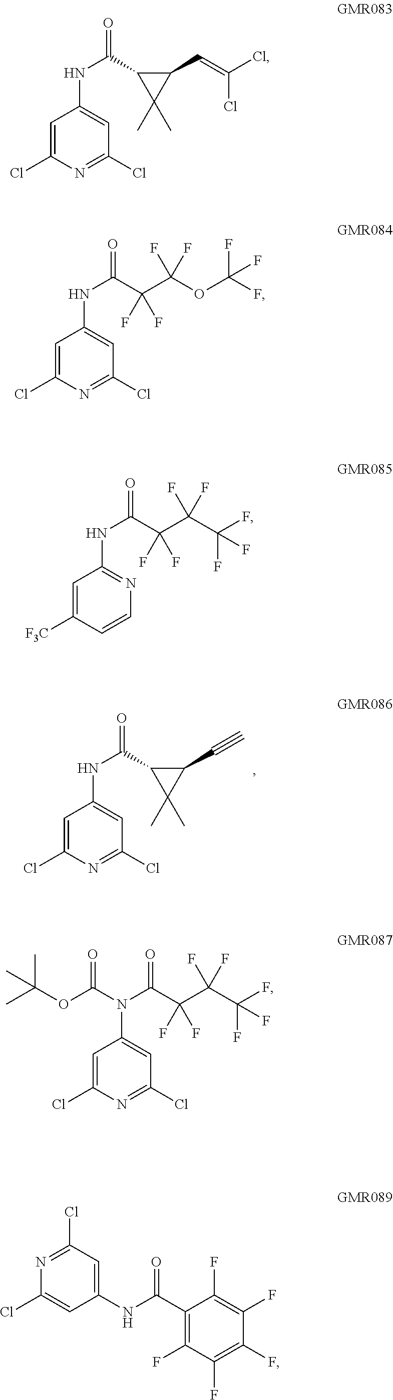

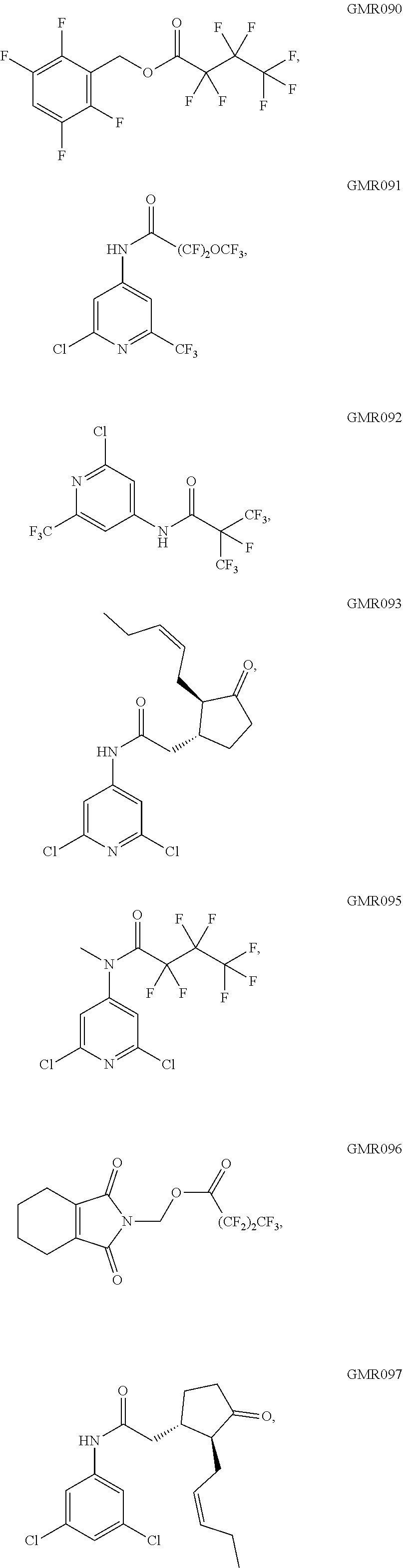

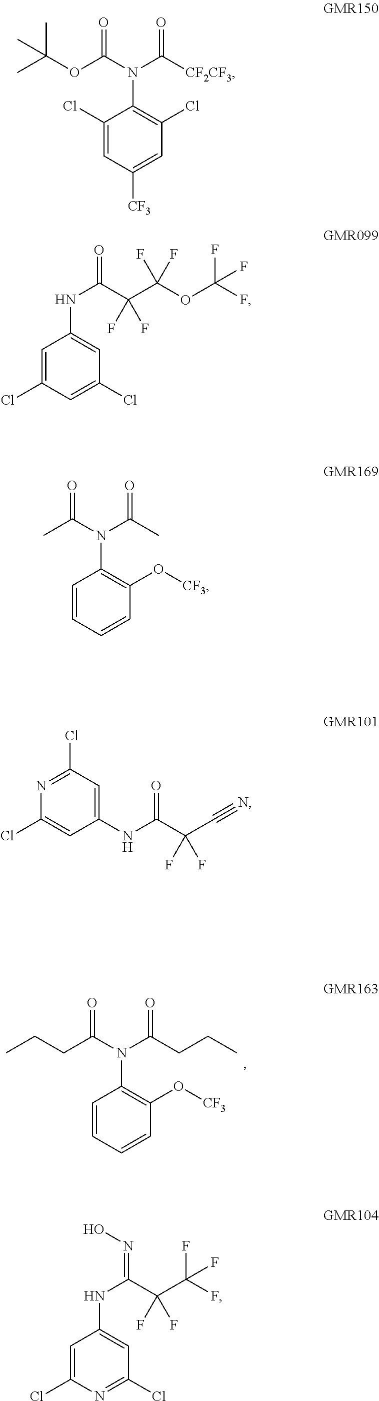

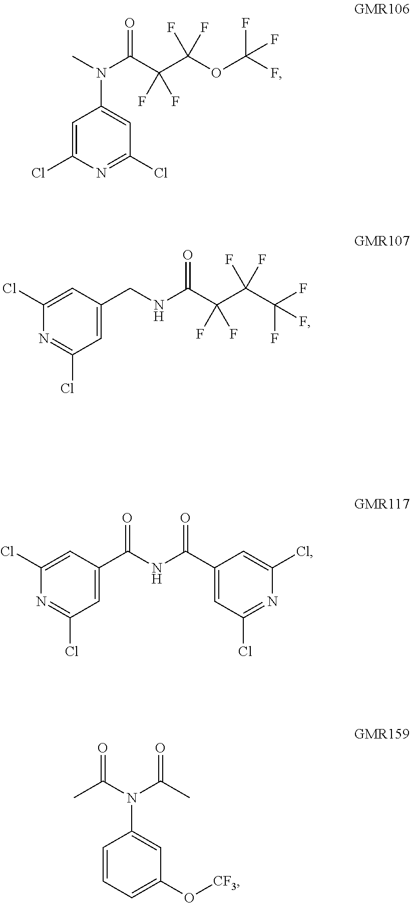 [SMILES: CN(C(=O)C(F)(F)C(F)(F)OC(F)(F)F)c1cc(Cl)nc(Cl)c1 ; O=C(NCc1cc(Cl)nc(Cl)c1)C(F)(F)C(F)(F)C(F)(F)F ; O=C(NC(=O)c1cc(Cl)nc(Cl)c1)c1cc(Cl)nc(Cl)c1 ; CC(=O)N(C(C)=O)c1cccc(OC(F)(F)F)c1]

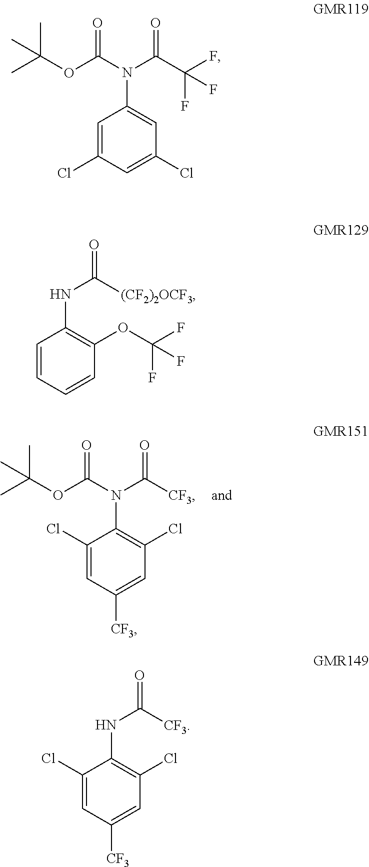

51. A composition comprising a compound of embodiment 50 and a carrier.

52. A method of repelling a species of insect from a locus to which said species of insect is normally attracted, comprising contacting on said locus a non-toxic effective insect repellent of the composition of embodiment 50.

53. A method for controlling one or more insects, comprising contacting the insects with the composition of embodiment 51.

54. The method of embodiment 52 or 53, wherein the composition is a solution, dust, granular formulation, or emulsion.

55. The method of any one of embodiments 52-54, wherein the composition is a liquid solution or emulsion.

56. The method of any one of embodiments 52-55, wherein said species of insect is a fly, spider, butterfly, crab, mosquito, centipede, tick, millipede, or scorpion.

57. The method of any one of embodiments 52-56, wherein said insect is a mosquito.

58. The method of any one of embodiments 52-57, where the locus is an area, an environment, or the skin of an animal.

59. A method of repelling insects, comprising contacting an object or locus with a compound of embodiment 50, wherein said insects are repelled from an adjacent area to said object or locus, wherein said insects are not physically contacted with said locus.

60. A method of providing a compound of embodiment 50 in an insect repellent medium from which a vaporized compound of embodiment 50 can be dispersed.

61. A composition comprising a compound of any one of embodiments 1-16, 20-39, or 50 and an essential oil, wherein the composition is synergistic, in intimate admixture with a carrier.

62. The composition of embodiment 61, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.

63. The composition of embodiment 61 or 62, wherein said essential oil is citronella oil.

64. The composition of any one of embodiments 61-63, wherein said compound of claim 1 is

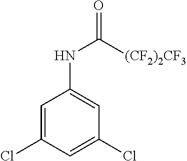

and said essential oil is citronella oil.

65. A method of repelling a species of insect from a locus to which said species of insect is normally attracted, comprising contacting on said locus a non-toxic effective insect repellent of a composition comprising a compound of any one of embodiments 1-16, 20-39, or 50 and an essential oil, wherein the composition is synergistic, in intimate admixture with a carrier.

66. The method of embodiment 65, wherein said composition comprises said compound of claim 1 in an amount from about 0.01-99.99% and an essential oil in an amount from about 99.99/0-0.01%.

67. The method of embodiment 65, wherein said composition contains about 0.0001 to 100000 parts per million of a compound of any one of embodiments 1-16, 20-39, or 50.

68. The method of any one of embodiments 65-67, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.

69. The method of any one of embodiments 65-68, wherein said essential oil is citronella oil.

70. The method of any one of embodiments 65-69, wherein said compound is

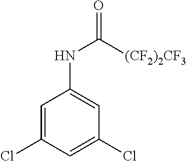

and said essential oil is citronella oil.

71. The method of any one of embodiments 65-70, wherein said insect is a mosquito.

72. A composition comprising a compound of any one of embodiments 1-16, 20-39, or 50 and second compound selected from a pyrethroid, a compound of Formula II′, or a compound of Formula III′

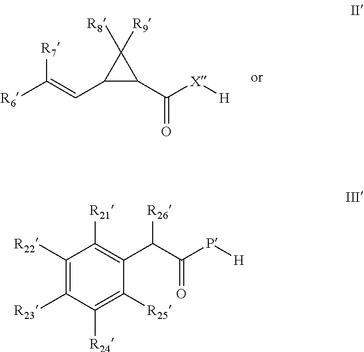

wherein,
X″ is O, NH, or S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl;
R_{21′}, R_{22′}, R_{23′}, R_{24′} and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl, and wherein the composition is synergistic, in intimate admixture with a carrier.

73. The composition of embodiment 72, wherein said composition comprises a compound of claim 1 and a compound of Formula II′

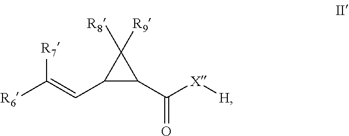

wherein,
X″ is O;
R_{6′} and R_{7′} are each independently selected from the group consisting of hydrogen, halo, haloalkyl, and C₁-C₃ alkyl; and
R_{8′} and R_{9′} are each methyl.

74. The composition of embodiment 72 or 73, wherein said composition comprises a compound of claim 1 having the structure

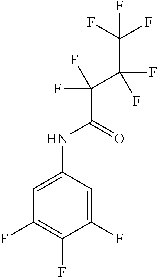 [SMILES: O=C(Nc1cc(F)c(F)c(F)c1)C(F)(F)C(F)(F)C(F)(F)F]

and a compound of Formula II′ having the structure

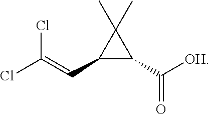

75. A method of repelling a species of insect from a locus to which said species of insect is normally attracted, comprising contacting on said locus a non-toxic effective insect repellent of a composition comprising a compound of any one of embodiments 1-16, 20-39, or 50 and a second compound selected from a pyrethroid, a compound of Formula II′, or compound of Formula III′

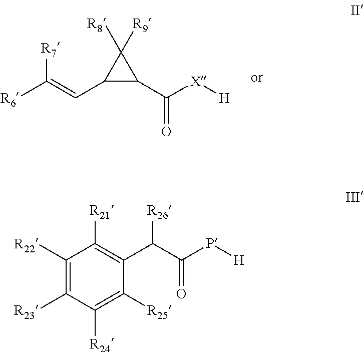

wherein,
X″ is O, NH, or S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl; R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl, and wherein the composition is synergistic, in intimate admixture with a carrier.

76. The method of embodiment 75, wherein said composition comprises said compound of claim 1 in an amount from about 0.01-99.99% and said second compound in an amount from about 99.99%-0.01%.

77. The method of embodiment 75 or 76, wherein said composition contains about 0.0001 to 100000 parts per million of a compound of any one of embodiments 1-16, 20-39, or 50.

78. The method of any one of embodiments 75-77, wherein said composition comprises a compound of claim 1 and a compound of Formula II′

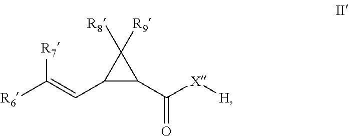

wherein,
X″ is O;
R_{6′} and R_{7′} are each independently selected from the group consisting of hydrogen, halo, haloalkyl, and C₁-C₃ alkyl; and
R_{8′} and R_{9′} are each methyl.

79. The method of any one of embodiments 75-78, wherein said composition comprises a compound of claim 1 having the structure

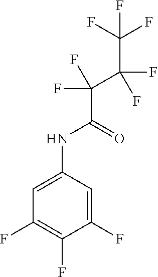 [SMILES: O=C(Nc1cc(F)c(F)c(F)c1)C(F)(F)C(F)(F)C(F)(F)F]

and a compound of Formula II′ having the structure

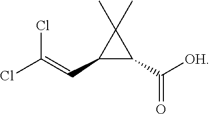

80. The method of any one of embodiments 75-79, wherein said species of insect is a fly, spider, butterfly, crab, mosquito, moth centipede, tick, millipede, or scorpion.

81. The method of any one of embodiments 75-80, wherein said insect is a mosquito.

82. The method of embodiment 17 or 48, wherein said insect is a fly, spider, butterfly, crab, mosquito, moth centipede, tick, millipede, or scorpion.

83. The method of any one of embodiments 17, 48, or 82, wherein said insect is a mosquito.

84. A method of repelling arthropods, comprising contacting an object or locus with a compound of Formula IA, IB, IC, ID, IE, IF, or IG

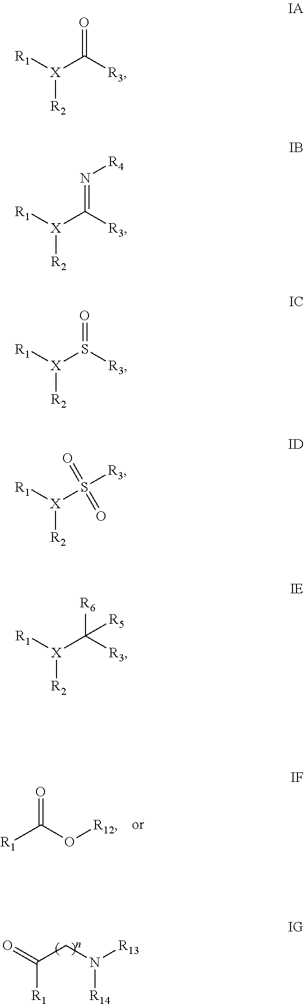

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, NR₂₁R₂₂, haloalkyl-haloalkoxy, cyano-alkyl, (C₃-C₇ cycloalkyl)-alkyl, C₃-C₇ cycloalkyl, (C₆-C₁₀ aryl)-C₁-C₃ alkyl, phenyl,

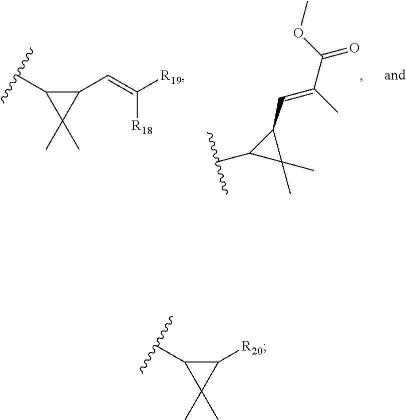

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
   or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
wherein R₁₃ and R₁₄, together with the nitrogen to which each is attached, form a 5 to 10-membered heterocyclyl or C₆-C₁₀ heteroaryl; and
   n is 0 or 1;
or a composition comprising a compound of Formula IA, IB, IC, ID, IE, IF, or IG and a carrier;
wherein said arthropods are repelled from an adjacent area to said object or locus, wherein said arthropods are not physically contacted with said object or locus.

85. A method of providing a compound of Formula IA, IB, IC, ID, IE, IF, or IG in an arthropod repellent medium from which a vaporized compound of Formula IA, IB, IC, ID, IE, IF, or IG can be dispersed

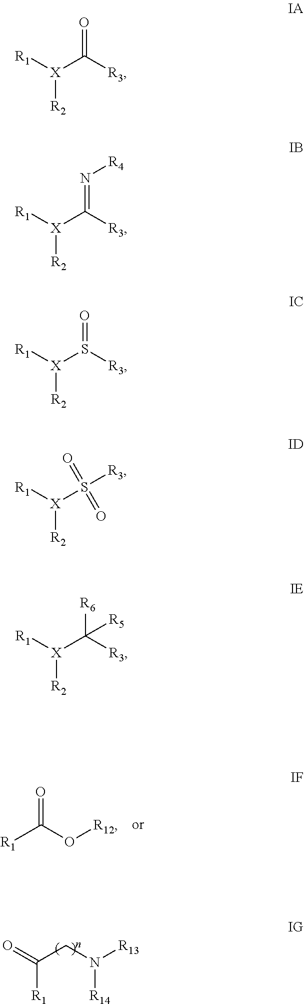

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl; R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

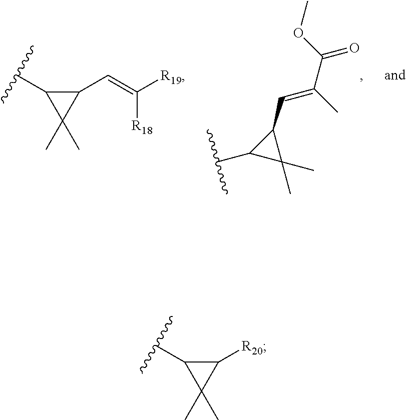

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
   or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
wherein R₁₃ and R₁₄, together with the nitrogen to which each is attached, form a 5 to 10-membered heterocyclyl or C₆-C₁₀ heteroaryl; and
   n is 0 or 1.

86. A method for controlling one or more arthropods, comprising exposing said one or more arthropods to a composition comprising a compound of Formula IA, IB, IC, ID, IE, IF, or IG and a carrier; wherein said composition is a vapor;

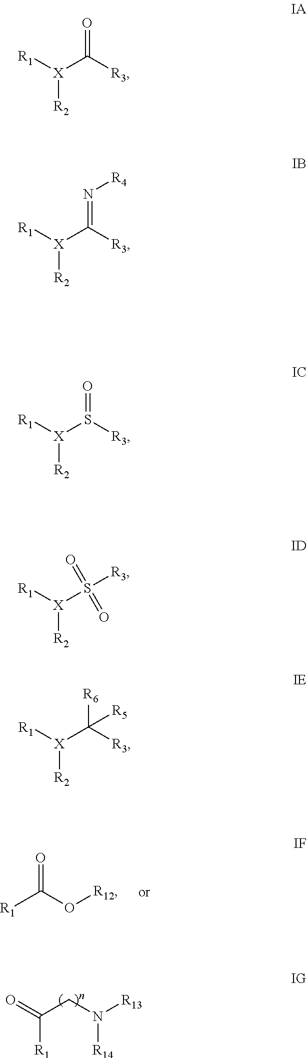

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

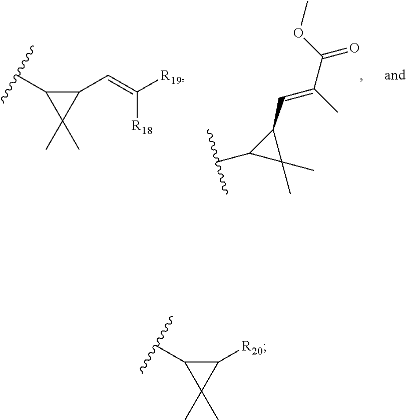

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally substituted with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently
selected from the group consisting of hydrogen, halogen, haloalkyl, and C₁-C₃ alkyl; R₆ and R₅ are each independently selected from the group consisting of hydrogen, hydroxy, haloalkyl, halogen, and C₁-C₃ alkyl;
   or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
wherein R₁₃ and R₁₄, together with the nitrogen to which each is attached, form a 5 to 10-membered heterocyclyl or C₆-C₁₀ heteroaryl; and
n is 0 or 1.

87. The method of any one of embodiments 84-86, wherein said compound is of Formula IA.

88. The method of any one of embodiments 84-87, wherein X is N.

89. The method of any one of embodiments 84-88, wherein R₁ is selected from the group consisting of 6-membered heteroaryl, 6-membered heteroaryl-C₁-C₃ alkyl, phenyl, benzyl, and (5- to 10-membered heterocyclyl)-C₁-C₃ alkyl wherein said heteroaryl, phenyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from the group consisting of hydrogen, C₁-C₆ alkyl, alkoxy, alkene, and haloalkyl.

90. The method of any one of embodiments 84-89, wherein R₁ is phenyl or pyridinyl optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁, where R₇, R₈, R₉, R₁₀, and R₁₁ are each independently selected from the group consisting of hydrogen, C₁-C₆ alkyl, alkoxy, alkene, and haloalkyl.

91. The method of embodiments 84-90, wherein halogen is fluorine, chlorine, or bromine.

92. The method of any one of embodiments 84-91, wherein R₁ is selected from the group consisting of

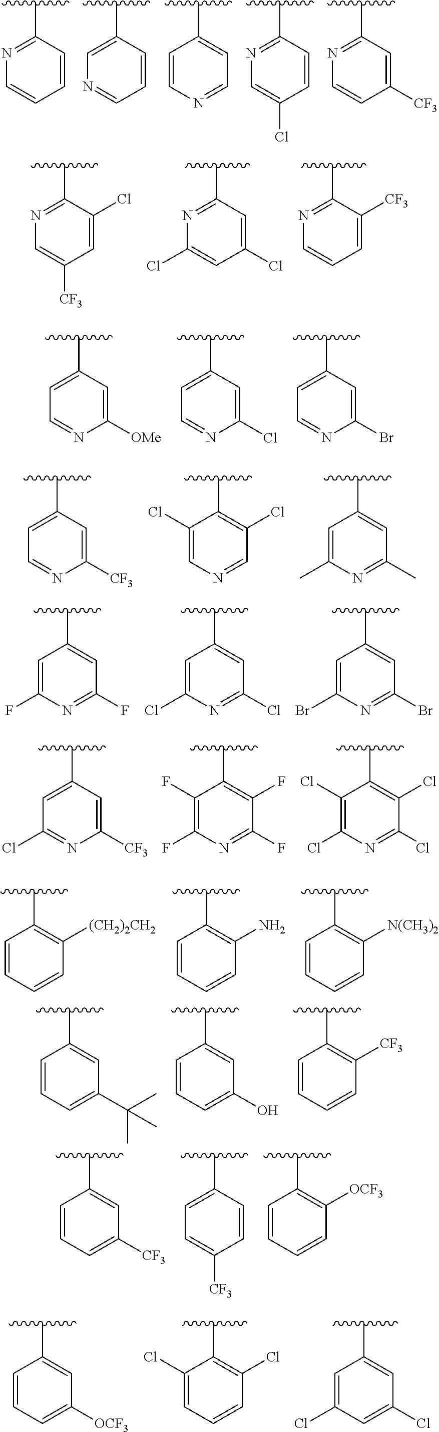

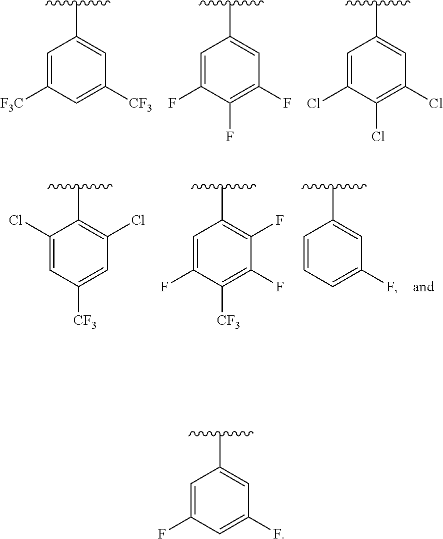

93. The method of an one of embodiments 84-92, wherein R₁ is selected from the group consisting of

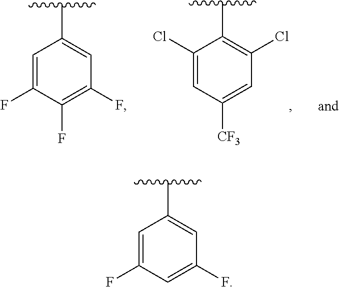 [SMILES: *c1cc(F)c(F)c(F)c1 ; *c1c(Cl)cc(C(F)(F)F)cc1Cl ; *c1cc(F)cc(F)c1]

94. The method of any one of embodiments 84-92, wherein R₁ is

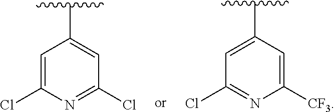 [SMILES: *c1cc(Cl)nc(Cl)c1 ; *c1cc(Cl)nc(C(F)(F)F)c1]

95. The method of any one of embodiments 84-94, wherein R₂ is selected from the group consisting of

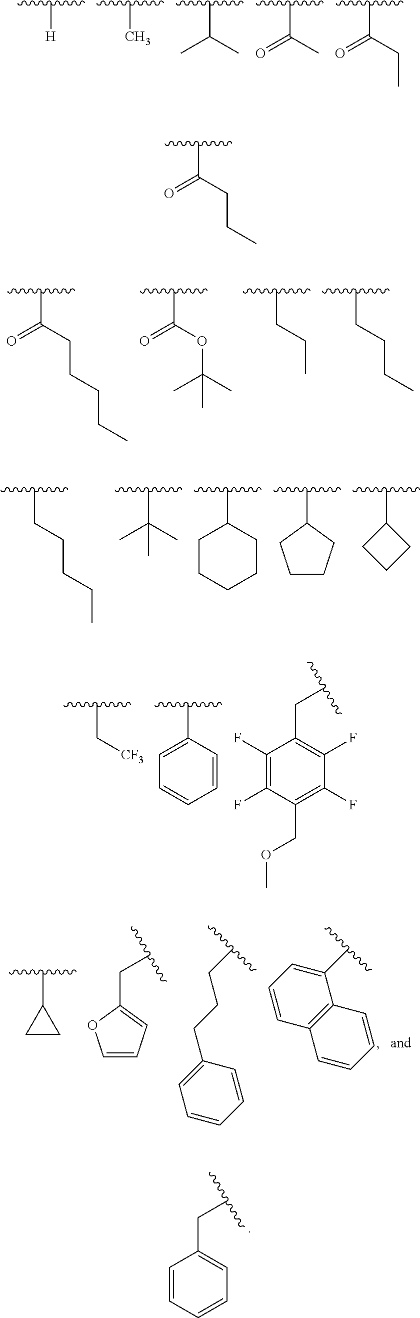

96. The method of any one of embodiments 84-95, wherein R₂ is hydrogen.

97. The method of any one of embodiments 84-96, wherein R₃ is selected from the group consisting of

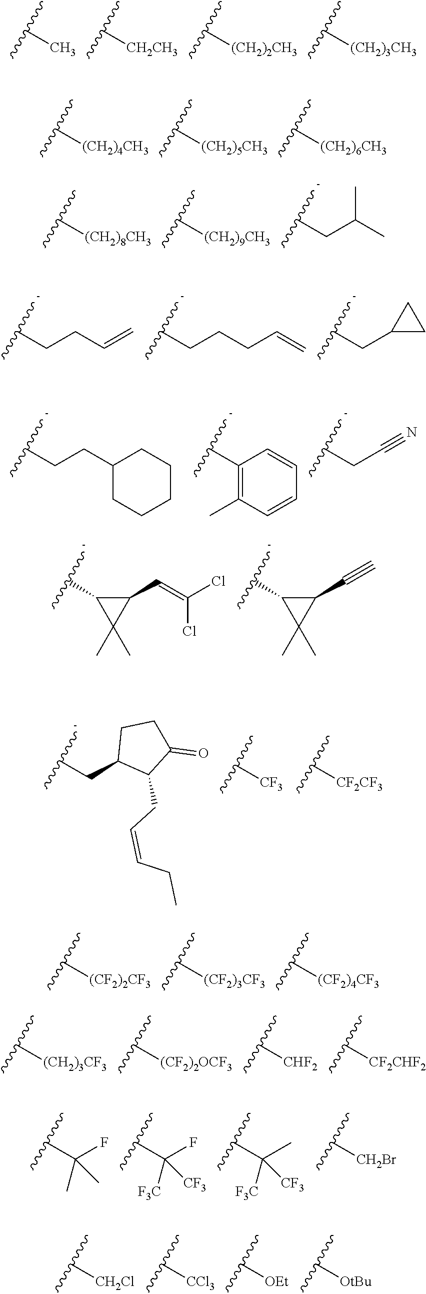

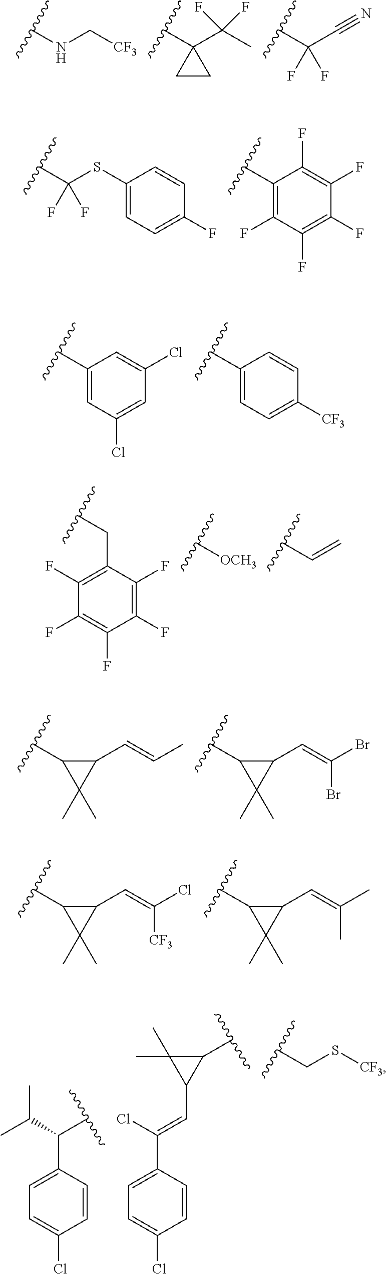

and —C(CF₃)₂H.

98. The method of any one of embodiments 84-97, wherein R₃ is selected from the group consisting of

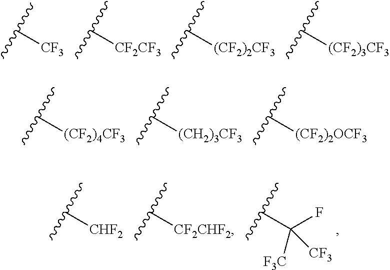

and C₁-C₆ alkyl.

99. The method of any one of embodiments 84-98, wherein R₃ is selected from the group consisting of

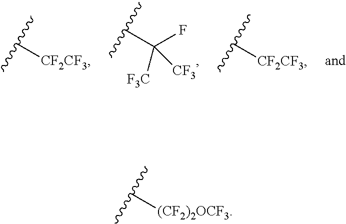

100. The method of any one of embodiments 84-99, wherein said compound is of Formula IA and is selected from the group consisting of

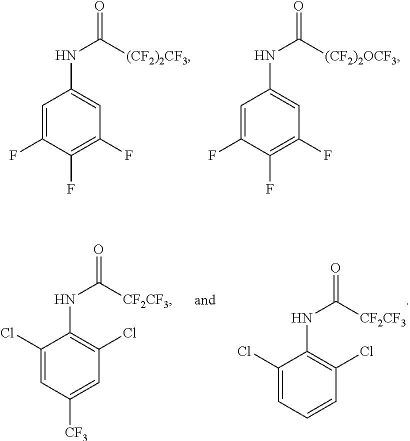

101. The method of any one of embodiments 84-99, wherein said compound is of Formula IA and is selected from the group consisting of

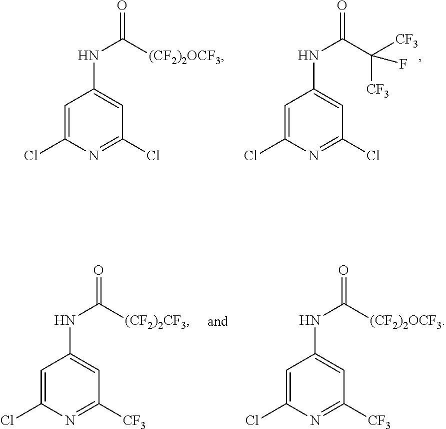

102. The method of embodiment 84 or 86, wherein said arthropods are insects.

103. The method of embodiment 85, wherein said arthropod repellent medium is an insect repellent medium.

104. The method of any one of embodiments 84-86, wherein said compound is selected from the group consisting of

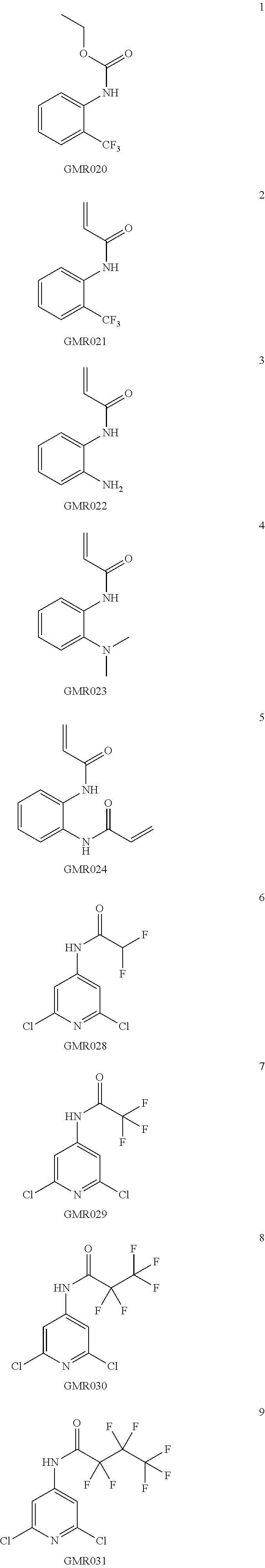

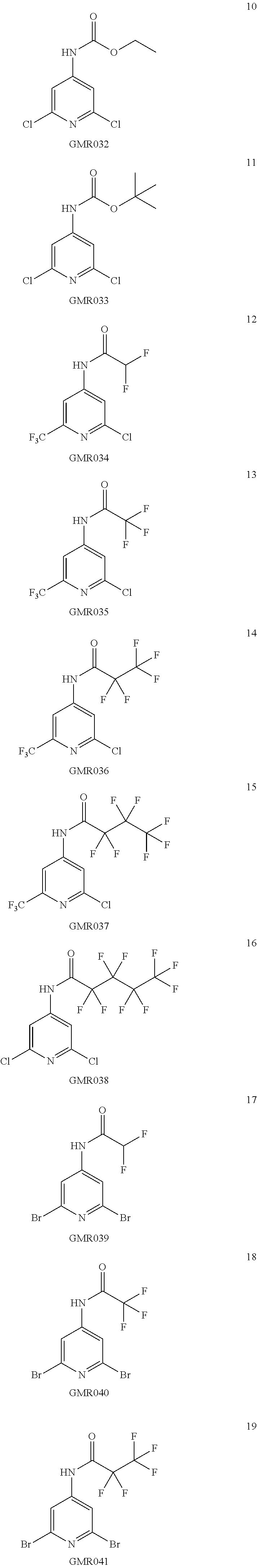

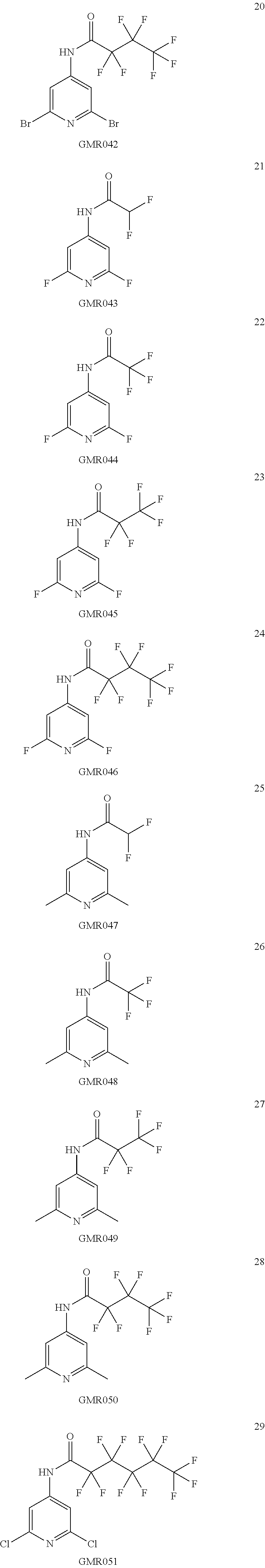

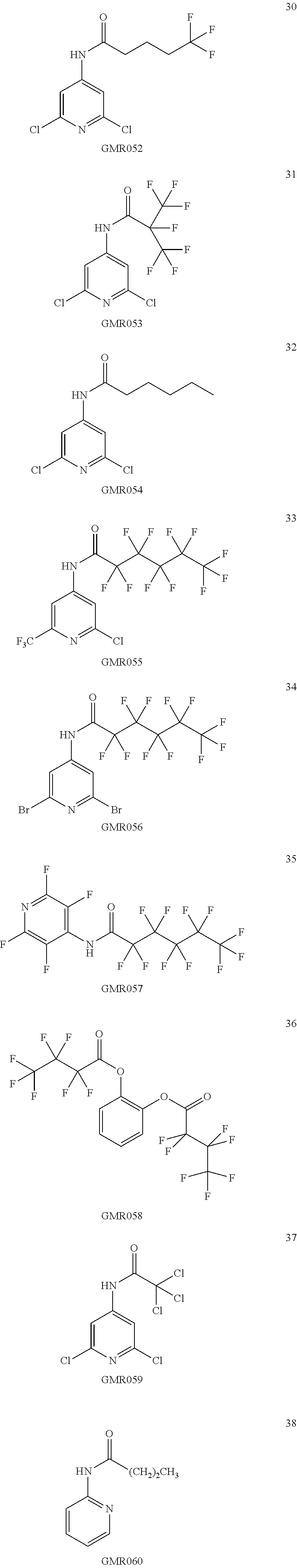

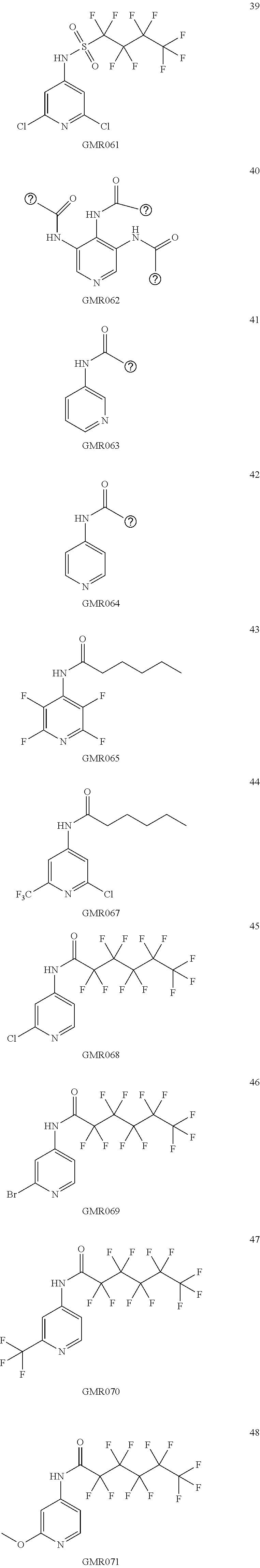

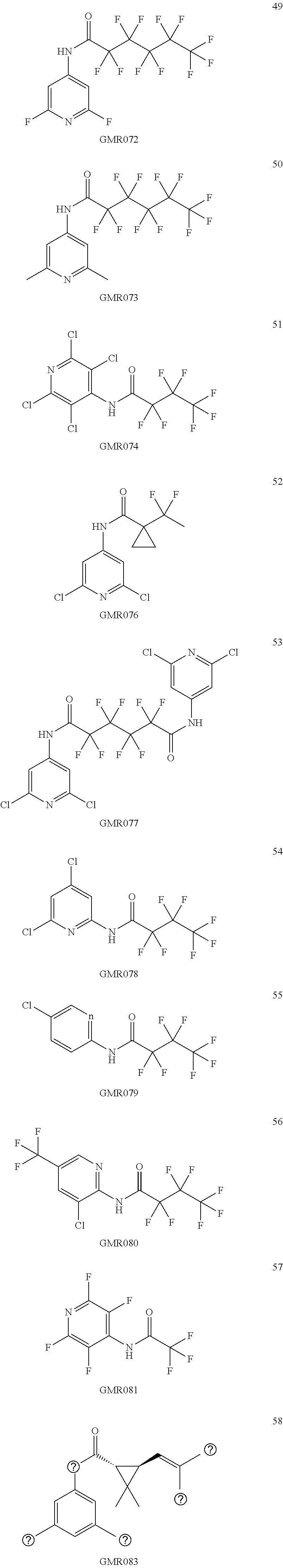

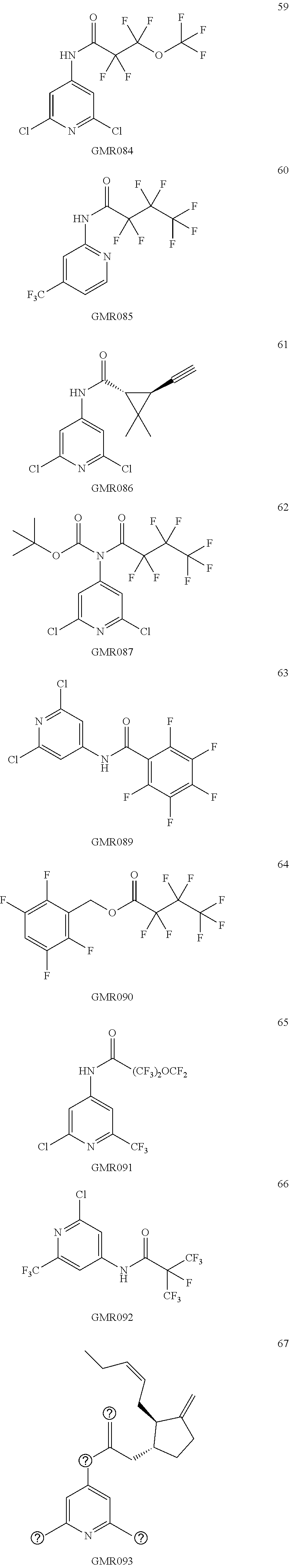

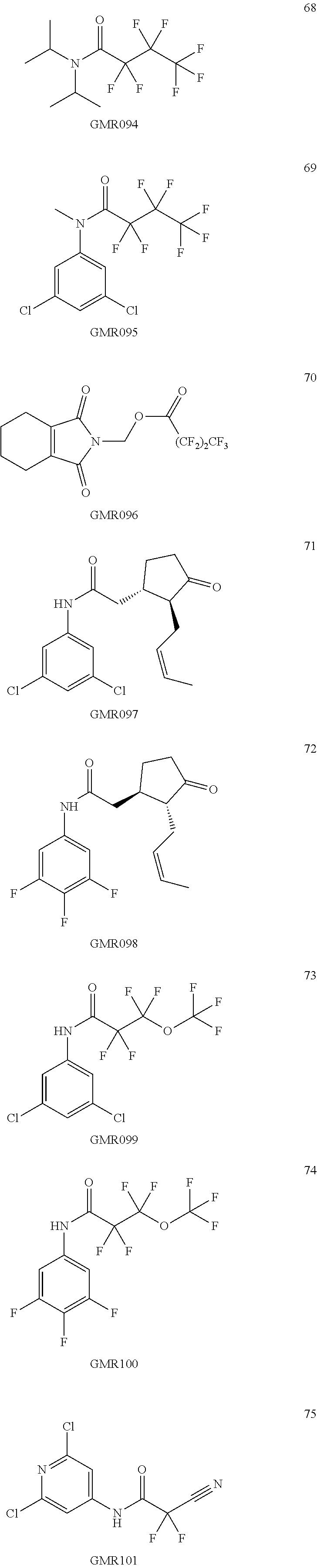

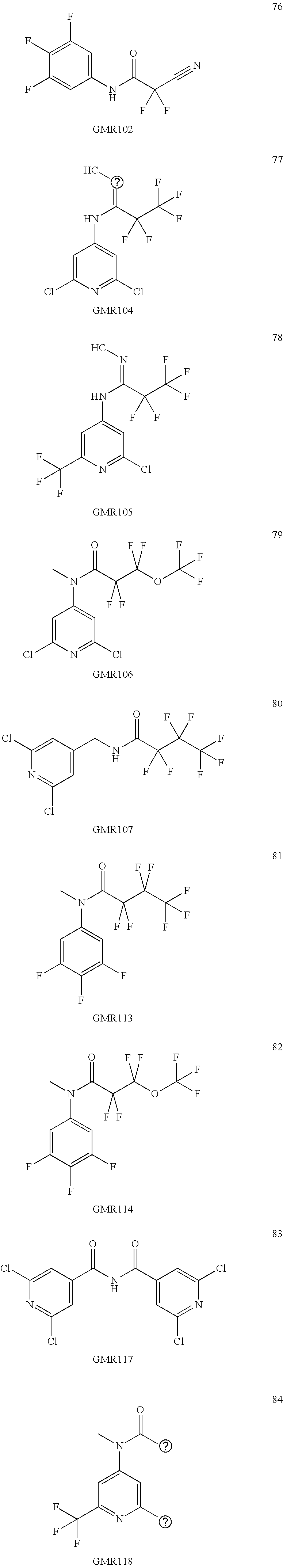

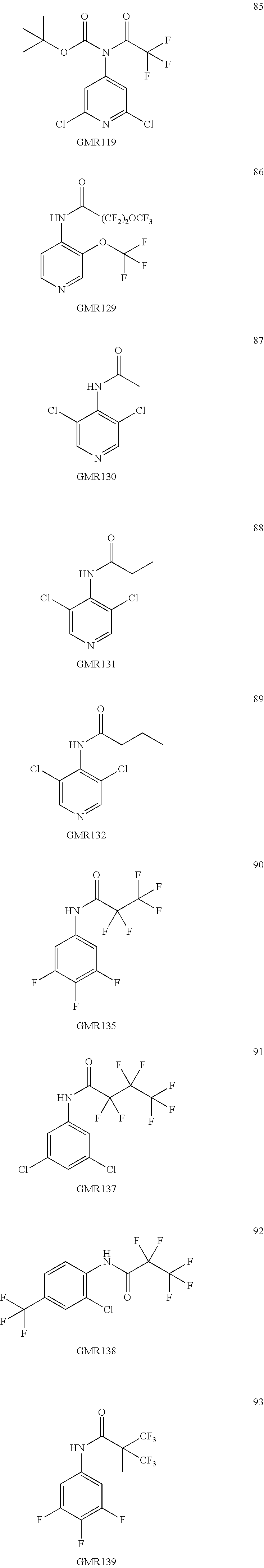

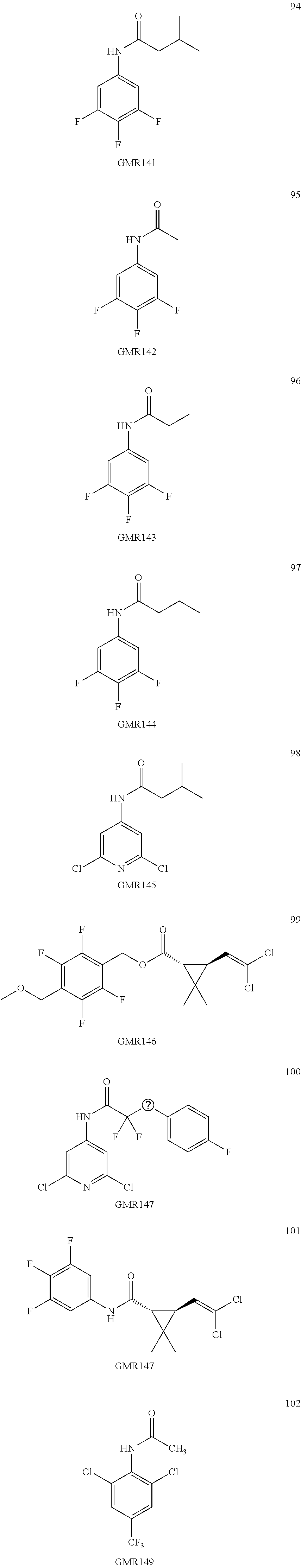

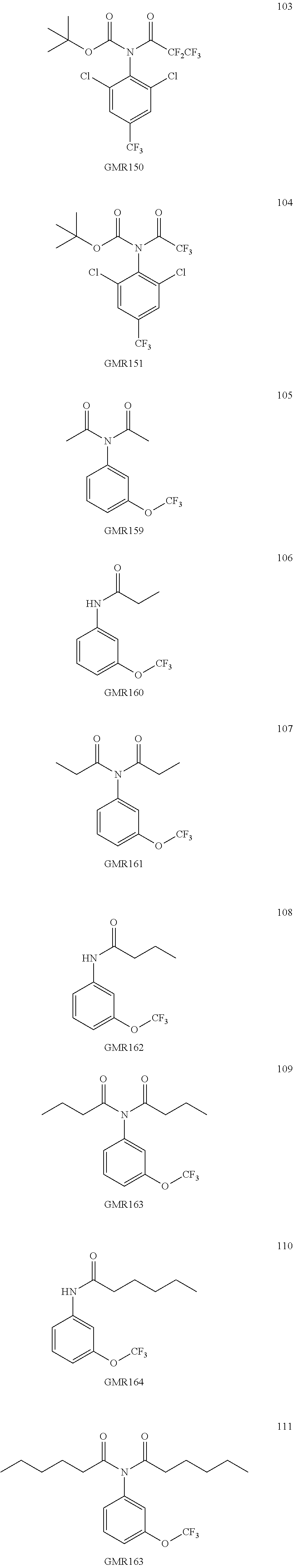

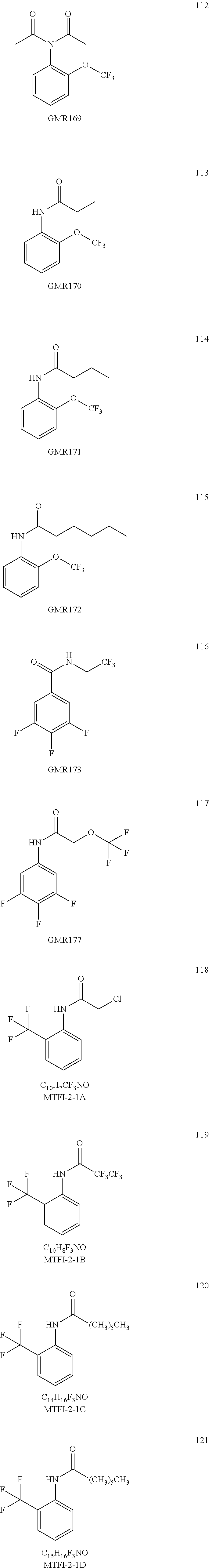

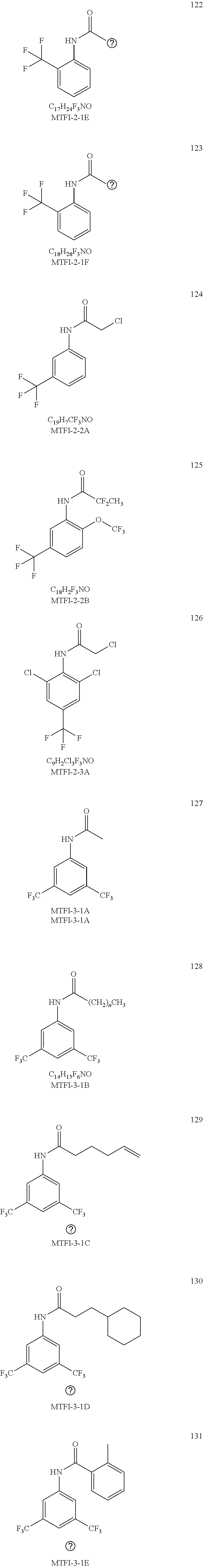

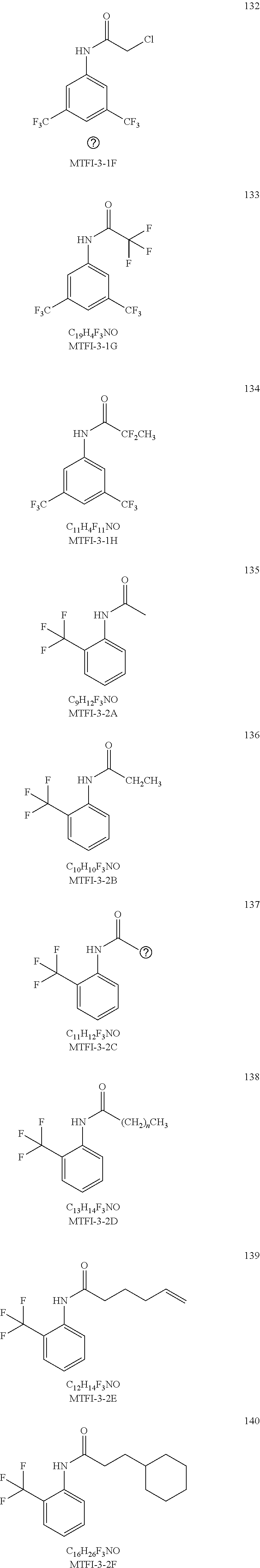

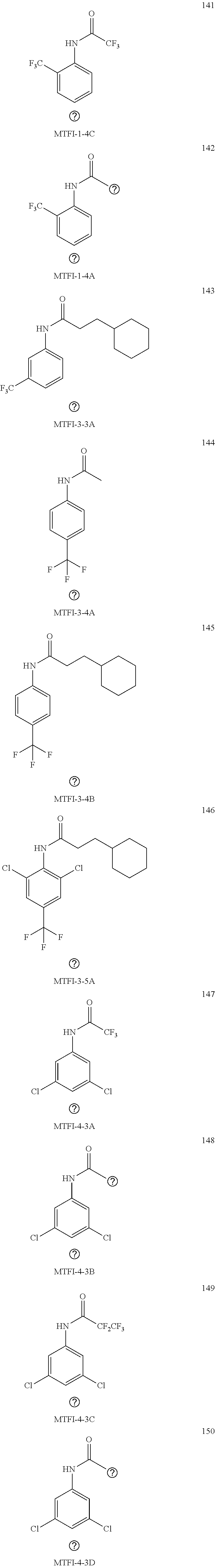

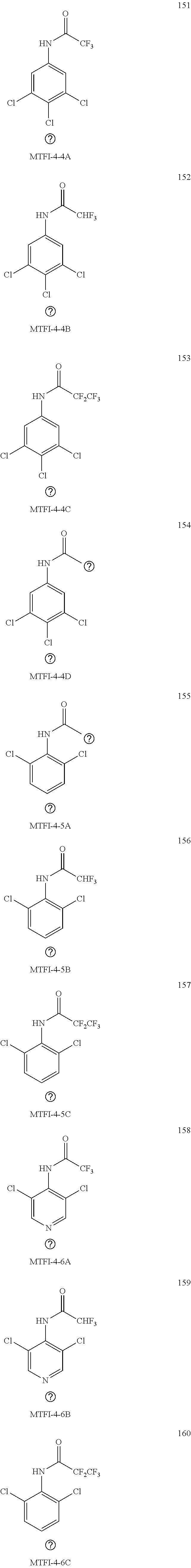

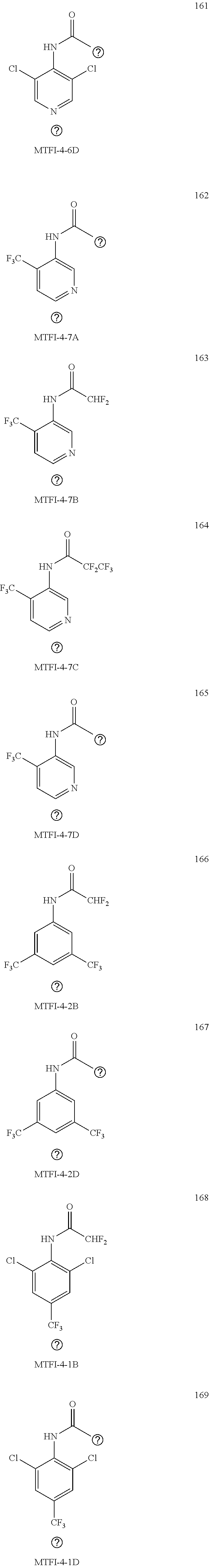

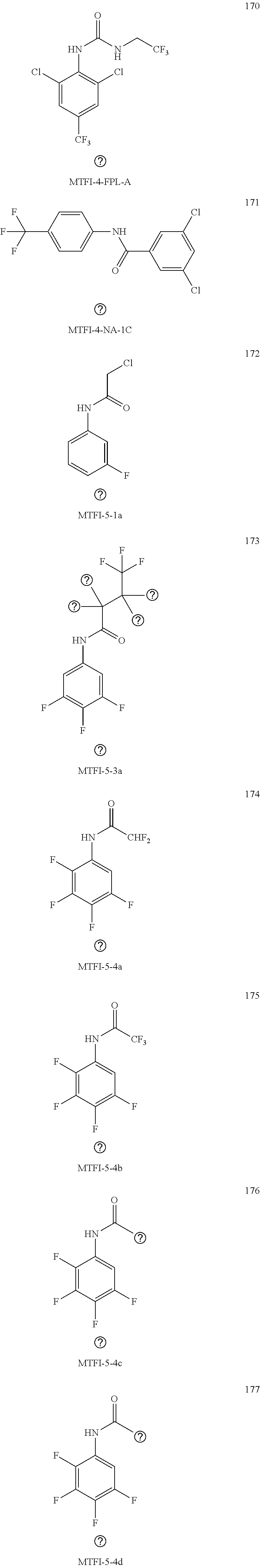

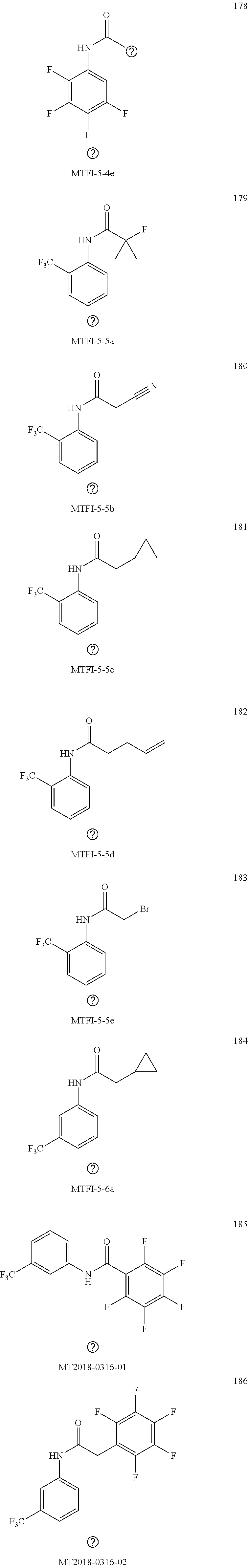

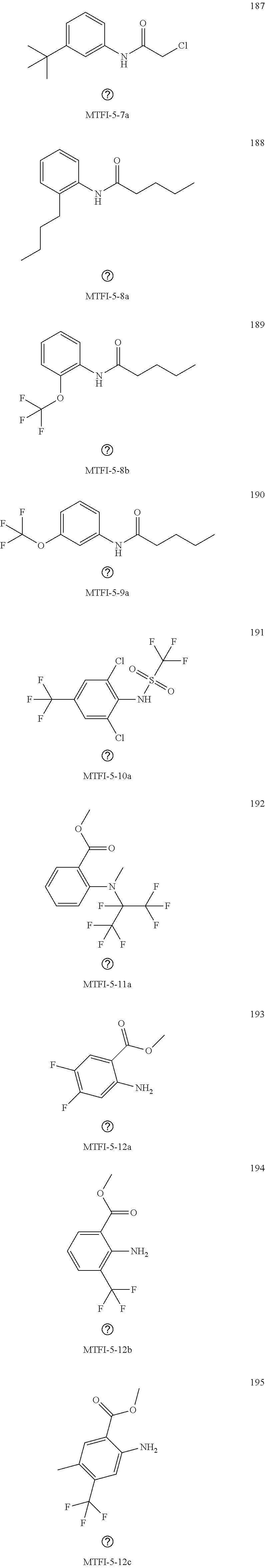

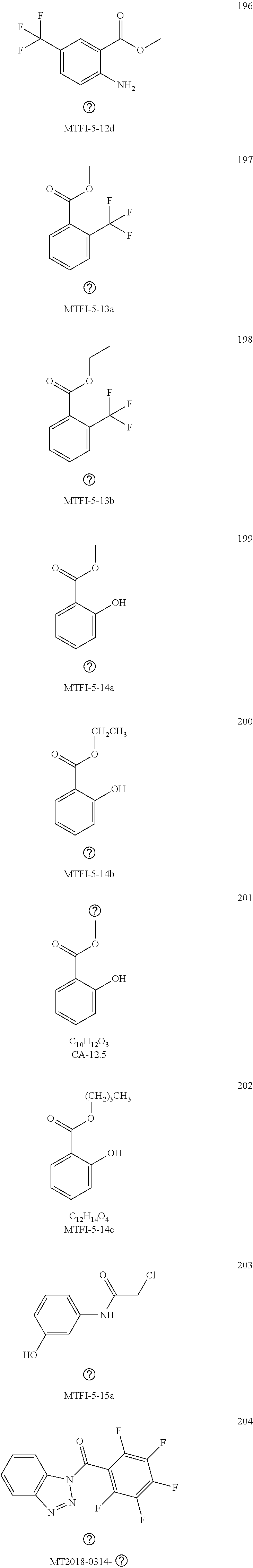

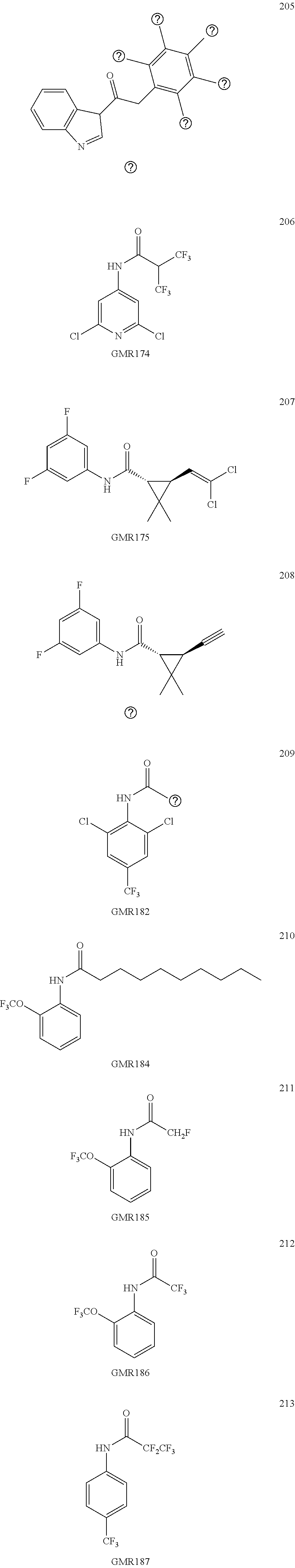

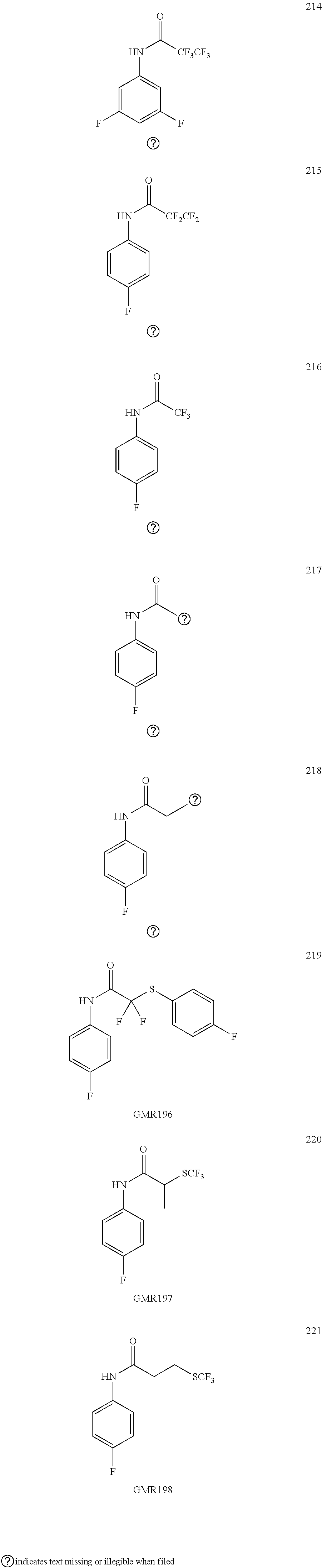

105. A compound of Formula X

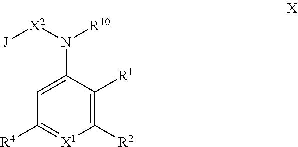

wherein,
   when R¹ is hydrogen, X is CR³ wherein R³ is fluorine, and R² and R⁴ are each fluorine, then:
X² is selected from the group consisting of C(O), S(O), S(O)₂, —CH(OH), and —CHCF₃;
R¹⁰ is hydrogen or C₁-C₆ alkyl; and J is selected from the group consisting of —(CF₂)₃CF₃, —CF₂CF₂OCF₃, —CF₂CN, —(CF₂)₂CF₃, —CH₂SCF₃,

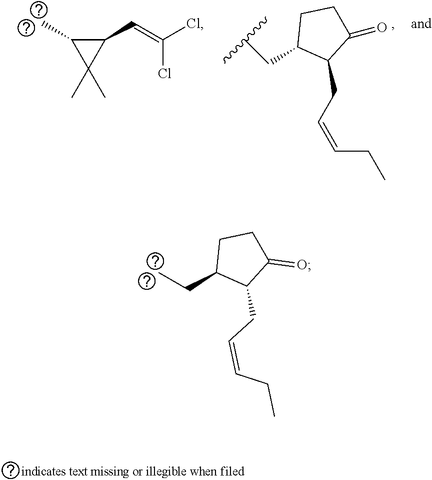

or,
when X¹ is CR³ wherein R³ is fluorine and R¹, R², and R⁴ are each fluorine, then:
X² is selected from the group consisting of C(O), S(O), S(O)₂, —CH(OH), and —CHCF₃; R¹⁰ is hydrogen or C₁-C₆ alkyl; and
J is selected from the group consisting of —CHF₂, —CF₃, —CF₂CHF₂, —CF₂CF₃, —(CF₂)₂CF₃;
or,
when R¹ is hydrogen, X¹ is N, and R⁴ and R² are each chlorine, then:
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃, —(CF₂)₂CF₃, —(CF₂)₃CF₃, —C(CF₃)₂H,

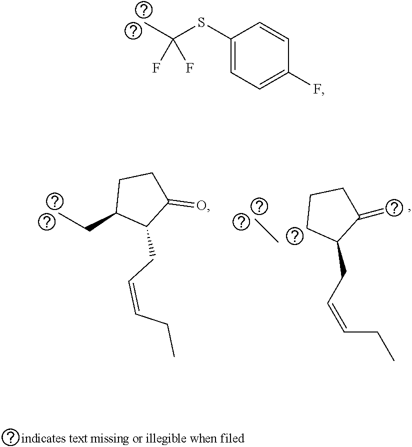

—CF₂CN, —(CH₂)₃CF₃, —C(CF₃)₂F, —CCl₃,

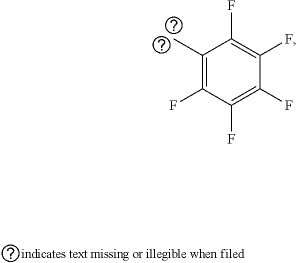

—(CH₂)₄CF₃,

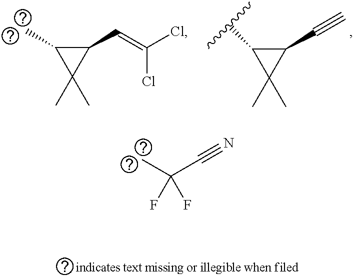

—CHF₂, —CF₃, and —CF₂CF₃;
or,
when R¹ is hydrogen, X¹ is N, and R⁴ and R² are each fluorine, then:
X² is selected from the group consisting of C(O), S(O), S(O)₂, —CH(OH), and —CHCF₃;
R¹⁰ is hydrogen or C₁-C₆ alkyl; and
J is selected from the group consisting of —CHF₂, —CF₃, —CF₂CF₃, and —(CF₂)₄CF₃;
or,
when R¹ is hydrogen, X¹ is CR³ wherein R³ is hydrogen, R⁴ is chlorine, and R² is chlorine, then:
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₂OCF₃,

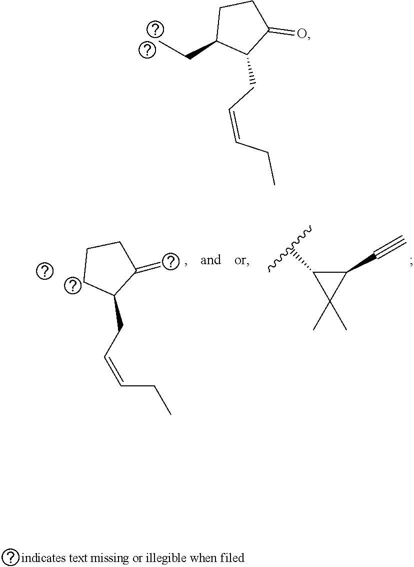

or,
when R¹ is hydrogen, X¹ is N, and R⁴ and R² are each bromine, then:
X² is selected from the group consisting of C(O), S(O), S(O)₂, —CH(OH), and —CHCF₃; R¹⁰ is hydrogen or C₁-C₆ alkyl; and
J is selected from the group consisting of —CF₂H, —CF₃, —CF₂CF₃, —(CF₂)₂CF₃, and —(CF₂)₄CF₃;
or,
when R¹ is hydrogen, X¹ is CR³ wherein R³ is hydrogen, one of R⁴ and R² is chlorine and the other is —CF₃, then:
X² is selected from the group consisting of C(O), S(O), S(O)₂, —CH(OH), and —CHCF₃;
R¹⁰ is hydrogen or C₁-C₆ alkyl; and
J is selected from the group consisting of —(CF₂)₄CF₃, —CF(CF₃)₂, —(CF₂)₂OCF₃, —(CF₂)₂CF₃, —CHF₂, —CF₃, and —(CF₂)CF₃;
or,
when R¹ is hydrogen, X¹ is N, and R⁴ and R² are C₁-C₃ alkyl, then:
X² is selected from the group consisting of C(O), S(O), S(O)₂, —CH(OH), and —CHCF₃;
R¹⁰ is hydrogen or C₁-C₆ alkyl; and
J is selected from the group consisting of —CF₃, —CF₂CF₃, —CHF₂, and —(CF₂)₄CF₃;
or,
when R¹ is hydrogen, X¹ is CR³ wherein R³ is hydrogen, R⁴ is —CF₃, and R² is —CF₃, then:
X² is selected from the group consisting of C(O), S(O), S(O)₂, —CH(OH), and —CHCF₃;
R¹⁰ is hydrogen or C₁-C₆ alkyl; and
J is —(CF₂)₂CF₃.

106. The compound of embodiment 105, wherein X² is C(O).

107. The compound of embodiment 106, wherein R¹⁰ is hydrogen.

108. The compound of embodiment 106, wherein R¹⁰ is C₁-C₃ alkyl.

109. The compound of embodiment 108, wherein R¹⁰ is methyl.

110. The compound of any one of embodiments 105-107, wherein R¹ is hydrogen, X¹ is CR³ wherein R³ is fluorine, and R² and R⁴ are each fluorine, J is selected from the group consisting of —(CF₂)₂CF₃,

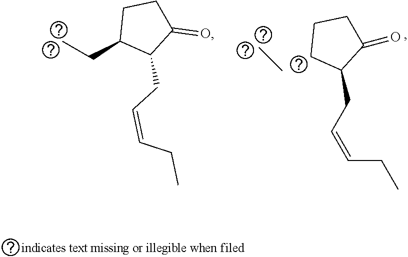

—CF₂CF₂OCF₃, —CH₂SCF₃, —CF₂CN, and

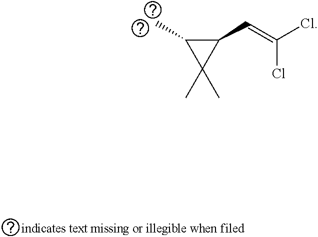

111. The compound of any one of embodiments 105-107, wherein J is —(CF₂)₂CF₃.

112. The compound of any one of embodiments 105-111, wherein said compound has the structure

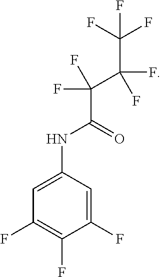 [SMILES: O=C(Nc1cc(F)c(F)c(F)c1)C(F)(F)C(F)(F)C(F)(F)F]

113. The compound of any one of embodiments 105-109, wherein R¹ is hydrogen, X¹ is CR³ wherein R³ is fluorine, and R² and R⁴ are each fluorine, and J is selected from the group consisting of —(CF₂)₃CF₃, —(CF₂)₂CF₃, and —CF₂CF₂OCF₃.

114. The compound of any one of embodiments 105-109, wherein X¹ is CR³, R³ is fluorine and R¹, R², and R⁴ are each fluorine, and J is selected from the group consisting of —CHF₂, —CF₃, —CF₂CHF₂, —CF₂CF₃, —(CF₂)₂CF₃.

115. The compound of any one of embodiments 105-109, wherein R¹ is hydrogen, X¹ is N, and R⁴ and R² are each chlorine, and J is selected from the group consisting of —CHF₂, —CF₃, CF₂CF₃, —(CF₂)₂CF₃,

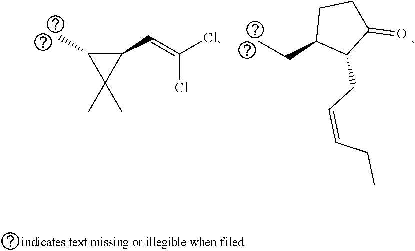

—(CF₂)₄CF₃, —(CF₂)₃CF₃, —(CH₂)₃CF₃, —C(CF₃)₂F, —(CH₂)₄CH₃, —(CF₂)₂OCF₃,

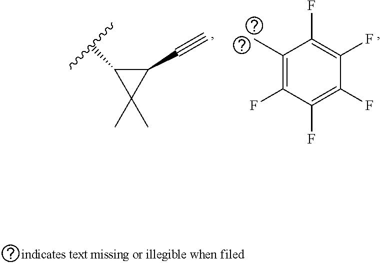

### —CCl₃,

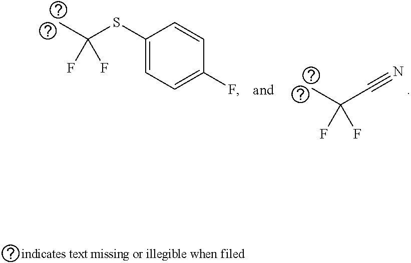

116. The compound of any one of embodiments 105-109, wherein R¹ is hydrogen, X¹ is N, and R⁴ and R² are each chlorine, and J is —(CF₂)₂CF₃ or —(CF₂)₂OCF₃.

117. The compound of any one of embodiments 105-109, wherein R¹ is hydrogen, X¹ is N, and R⁴ and R² are each fluorine, and J is selected from the group consisting of —CHF₂, —CF₃, —CF₂CF₃, and —(CF₂)₄CF₃.

118. The compound of any one of embodiments 105-109, wherein R¹ is hydrogen, X is CR³ wherein R³ is hydrogen, R⁴ is chlorine, and R² is chlorine, and J is

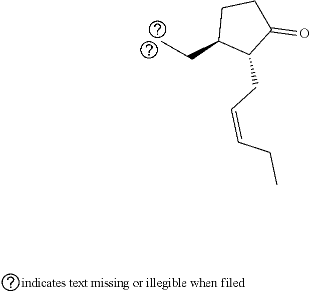

or —(CF₂)₂OCF₃.

119. The compound of any one of embodiments 105-109, wherein R¹ is hydrogen, X¹ is N, and R⁴ and R² are each bromine, and J is selected from the group consisting of —CF₂H, —CF₃, —CF₂CF₃, —(CF₂)₂CF₃, and —(CF₂)₄CF₃.

120. The compound of any one of embodiments 105-109, wherein R¹ is hydrogen, X¹ is CR³ wherein R³ is hydrogen, one of R⁴ and R² is chlorine and the other is —CF₃, and J is selected from the group consisting of —(CF₂)₂OCF₃, —CHF₂, —CF₃, —(CF₂)CF₃, —(CF₂)₂CF₃, —(CF₂)₄CF₃, —(CH₂)₄CH₃, and —CF(CF₃)₂.

121. The compound of any one of embodiments 105-109, wherein R¹ is hydrogen, X¹ is CR³ wherein R³ is hydrogen, one of R⁴ and R² is chlorine and the other is —CF₃, and J is —(CF₂)₂CF₃.

122. The compound of any one of embodiments 105-109, wherein R¹ is hydrogen, X¹ is N, and R⁴ and R² are each methyl, and J is selected from the group consisting of —CF₃, —CF₂CF₃, —CHF₂, and —(CF₂)₄CF₃.

123. The compound of any one of embodiments 105-109, wherein R¹ is hydrogen, X is CR³ wherein R³ is hydrogen, R⁴ is —CF₃, and R² is —CF₃, and J is —(CF₂)₂CF₃.

124. The compound of embodiment 105, wherein said compound has a structure selected from the group consisting of

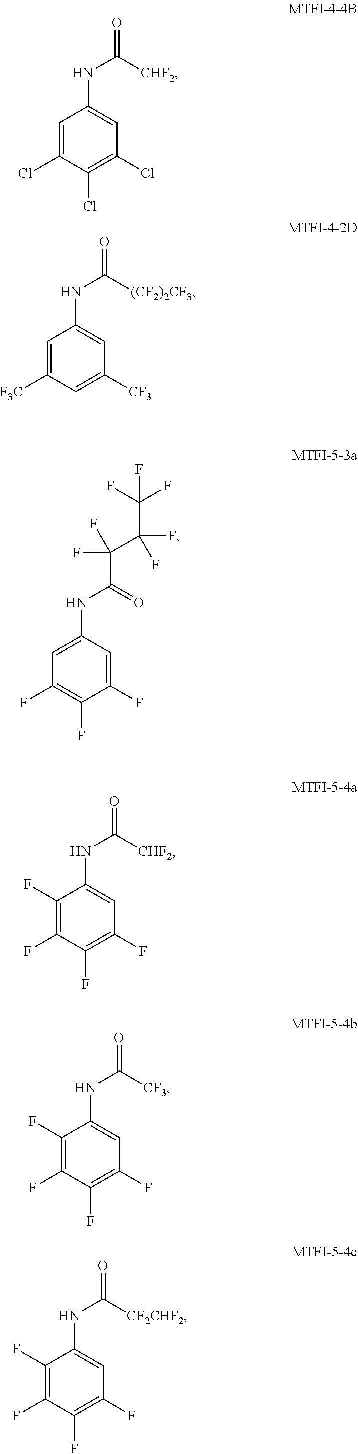

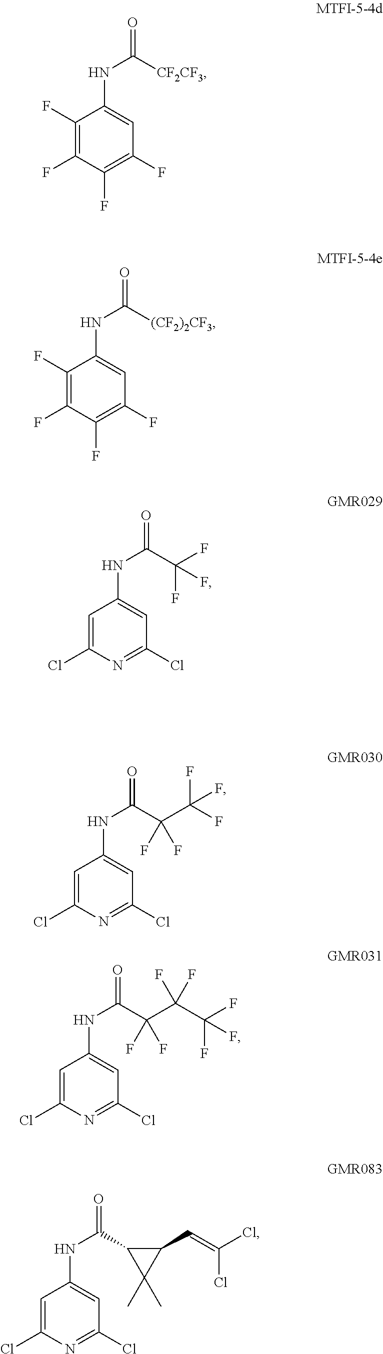

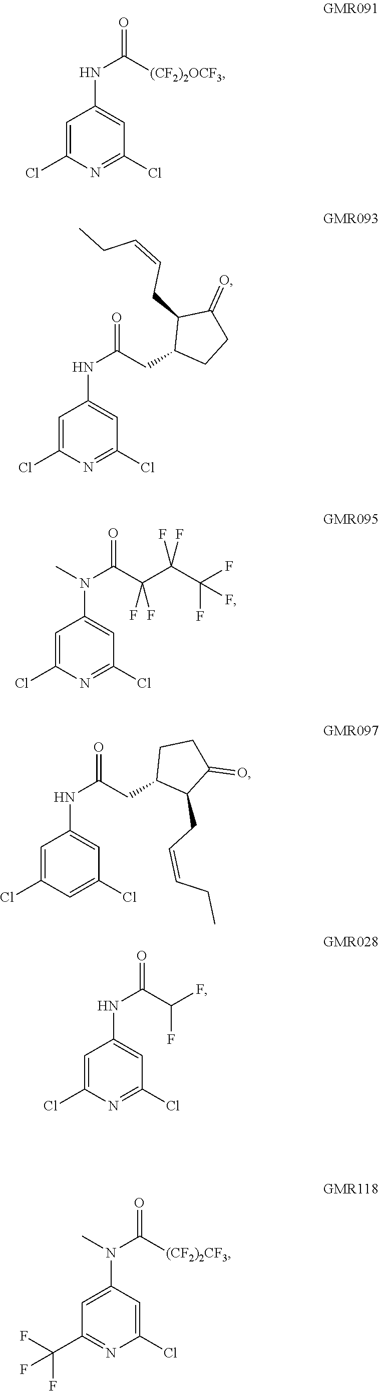

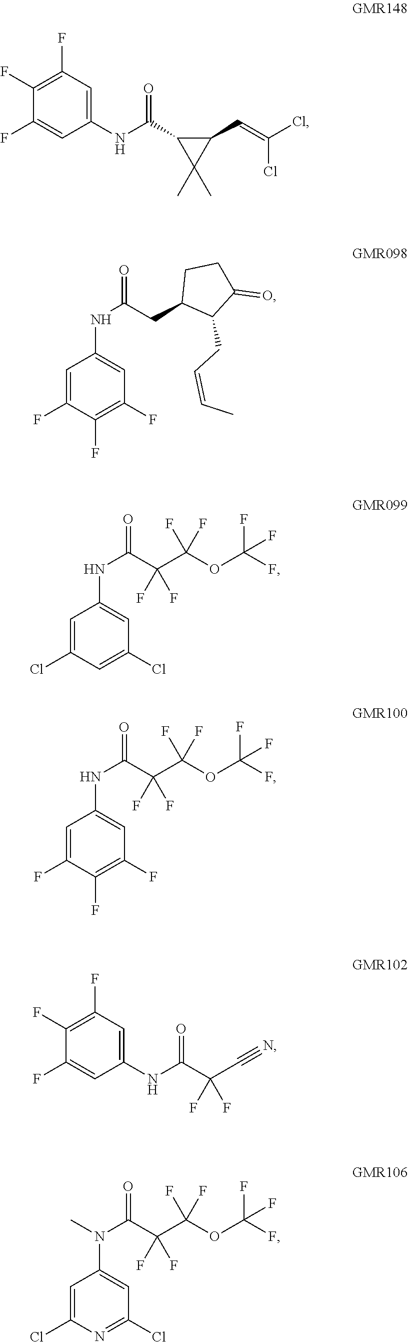 [SMILES: CC1(C)[C@H](C=C(Cl)Cl)[C@H]1C(=O)Nc1cc(F)c(F)c(F)c1 ; C/C=C\C[C@H]1C(=O)CC[C@@H]1CC(=O)Nc1cc(F)c(F)c(F)c1 ; O=C(Nc1cc(Cl)cc(Cl)c1)C(F)(F)C(F)(F)OC(F)(F)F ; O=C(Nc1cc(F)c(F)c(F)c1)C(F)(F)C(F)(F)OC(F)(F)F ; N#CC(F)(F)C(=O)Nc1cc(F)c(F)c(F)c1 ; CN(C(=O)C(F)(F)C(F)(F)OC(F)(F)F)c1cc(Cl)nc(Cl)c1]

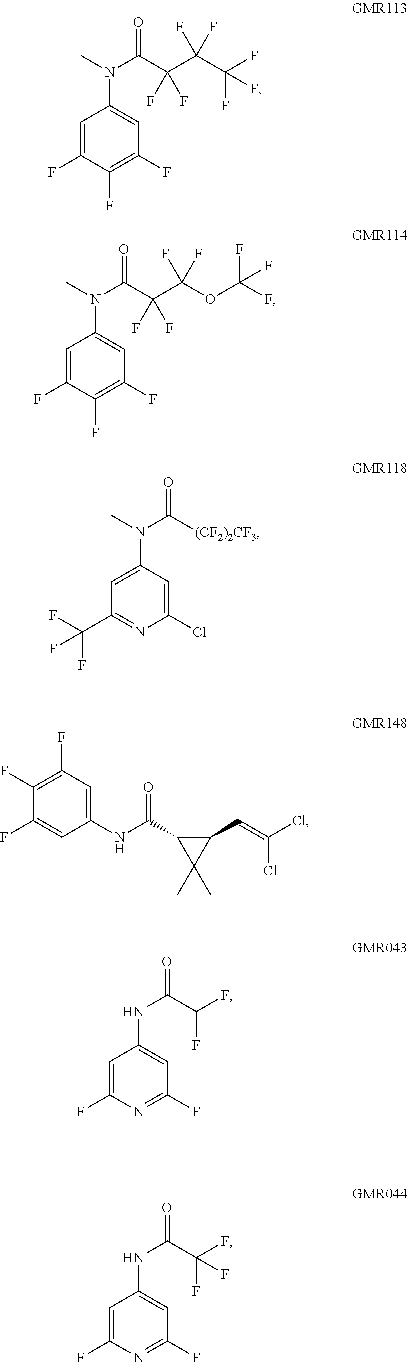

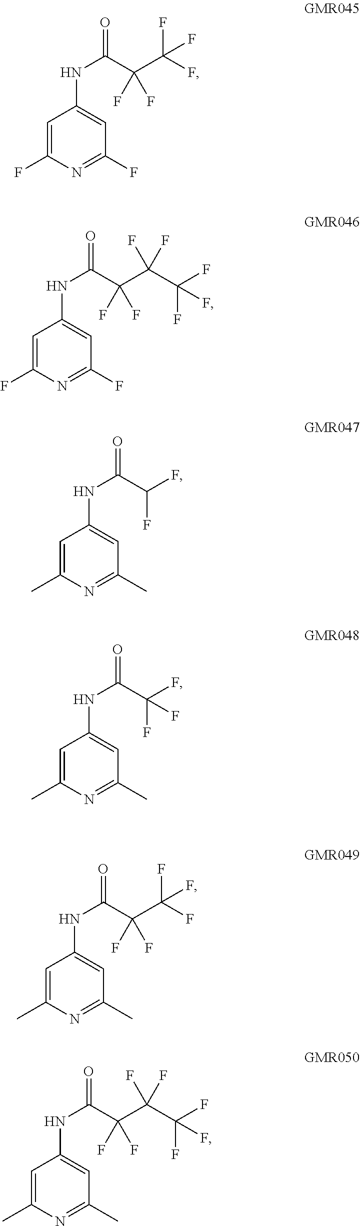 [SMILES: O=C(Nc1cc(F)nc(F)c1)C(F)(F)C(F)(F)F ; O=C(Nc1cc(F)nc(F)c1)C(F)(F)C(F)(F)C(F)(F)F ; Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1]

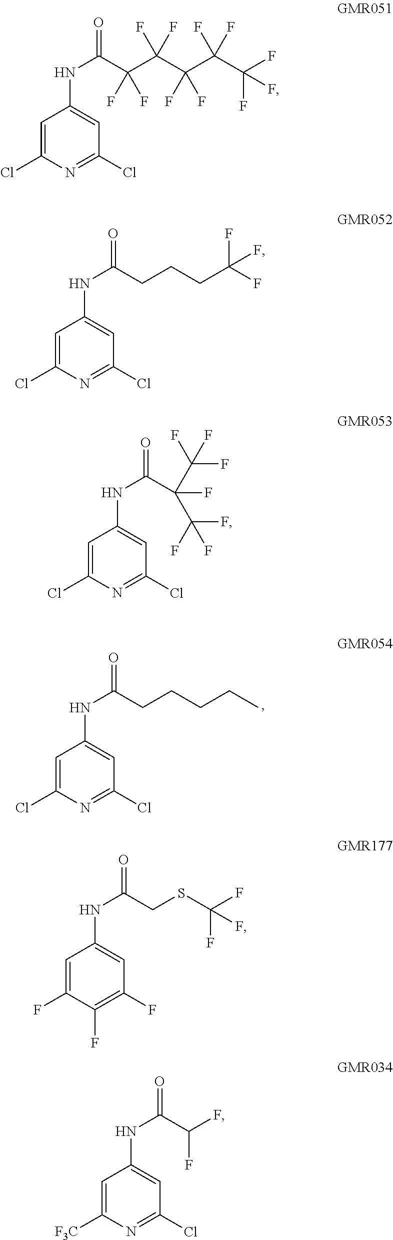 [SMILES: O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1 ; O=C(Nc1cc(Cl)nc(Cl)c1)C(F)(C(F)(F)F)C(F)(F)F ; CCCCCC(=O)Nc1cc(Cl)nc(Cl)c1 ; O=C(CSC(F)(F)F)Nc1cc(F)c(F)c(F)c1 ; O=C(Nc1cc(Cl)nc(C(F)(F)F)c1)C(F)F]

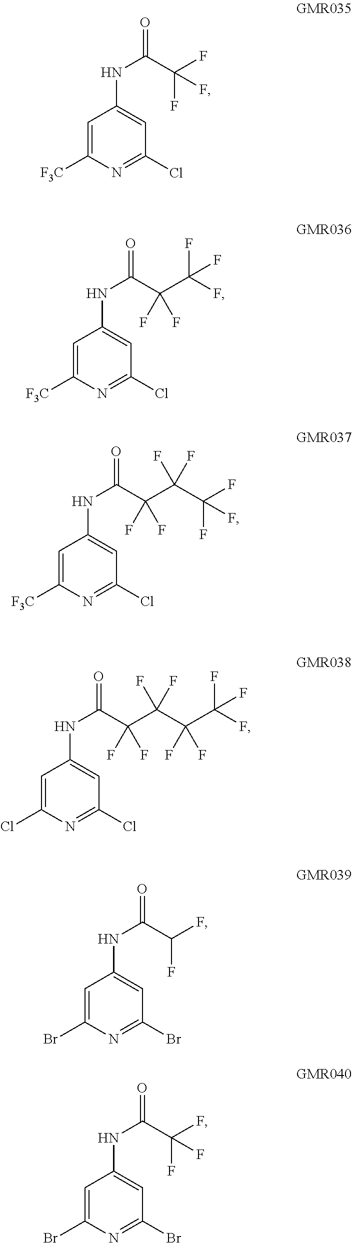 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(Cl)c1)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

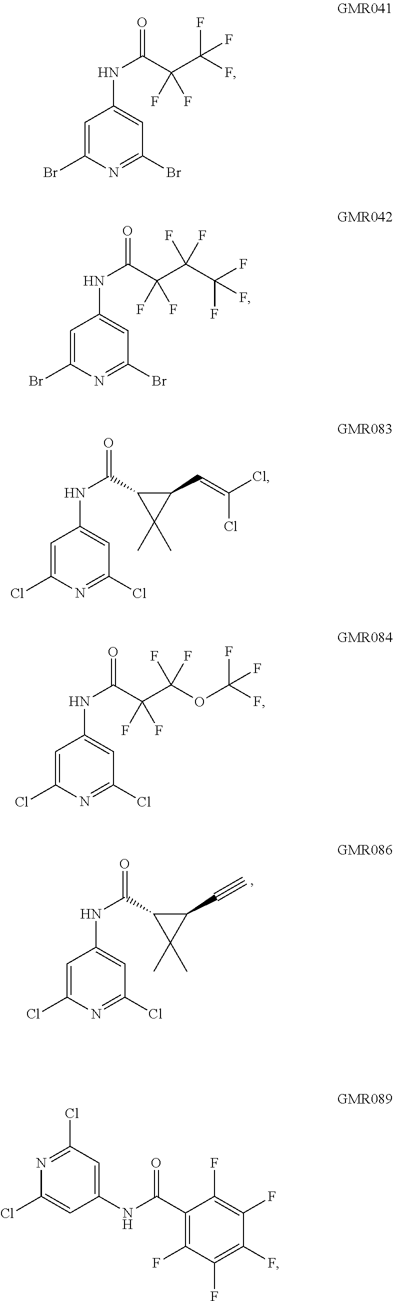 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; CC1(C)[C@H](C=C(Cl)Cl)[C@H]1C(=O)Nc1cc(Cl)nc(Cl)c1 ; O=C(Nc1cc(Cl)nc(Cl)c1)C(F)(F)C(F)(F)OC(F)(F)F ; C#C[C@@H]1[C@@H](C(=O)Nc2cc(Cl)nc(Cl)c2)C1(C)C ; O=C(Nc1cc(Cl)nc(Cl)c1)c1c(F)c(F)c(F)c(F)c1F]

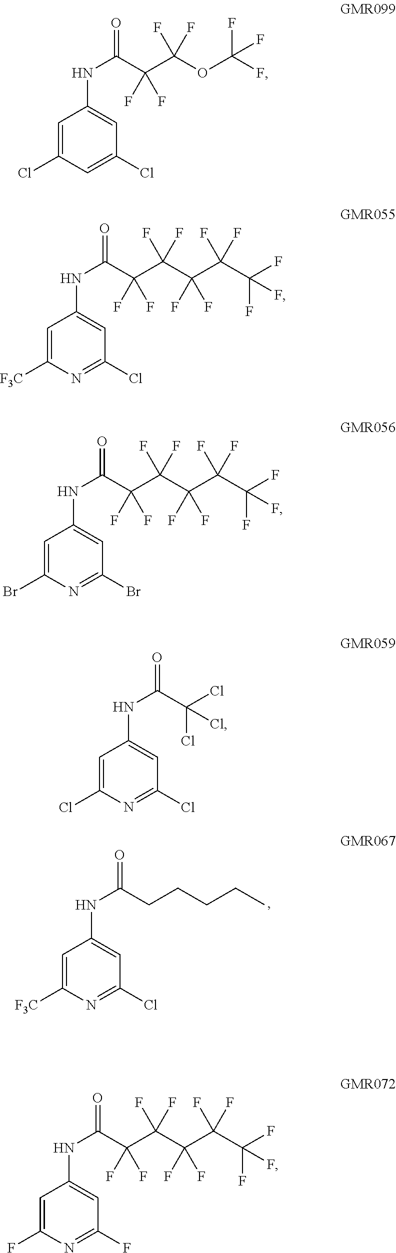 [SMILES: O=C(Nc1cc(Cl)cc(Cl)c1)C(F)(F)C(F)(F)OC(F)(F)F ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F ; O=C(Nc1cc(Cl)nc(Cl)c1)C(Cl)(Cl)Cl ; CCCCCC(=O)Nc1cc(Cl)nc(C(F)(F)F)c1 ; O=C(Nc1cc(F)nc(F)c1)C(F)(F)C(F)(F)C(F)(F)C(F)(F)C(F)(F)F]

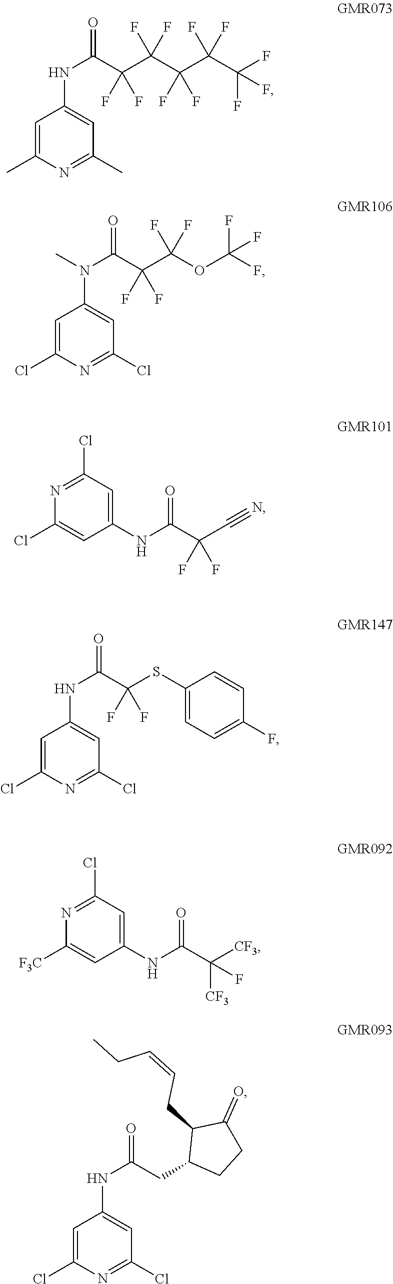 [SMILES: Cc1cc(NC(=O)C(F)(F)C(F)(F)C(F)(F)C(F)(F)C(F)(F)F)cc(C)n1 ; CN(C(=O)C(F)(F)C(F)(F)OC(F)(F)F)c1cc(Cl)nc(Cl)c1 ; N#CC(F)(F)C(=O)Nc1cc(Cl)nc(Cl)c1 ; O=C(Nc1cc(Cl)nc(Cl)c1)C(F)(F)Sc1ccc(F)cc1 ; O=C(Nc1cc(Cl)nc(C(F)(F)F)c1)C(F)(C(F)(F)F)C(F)(F)F ; CC/C=C\C[C@H]1C(=O)CC[C@@H]1CC(=O)Nc1cc(Cl)nc(Cl)c1]

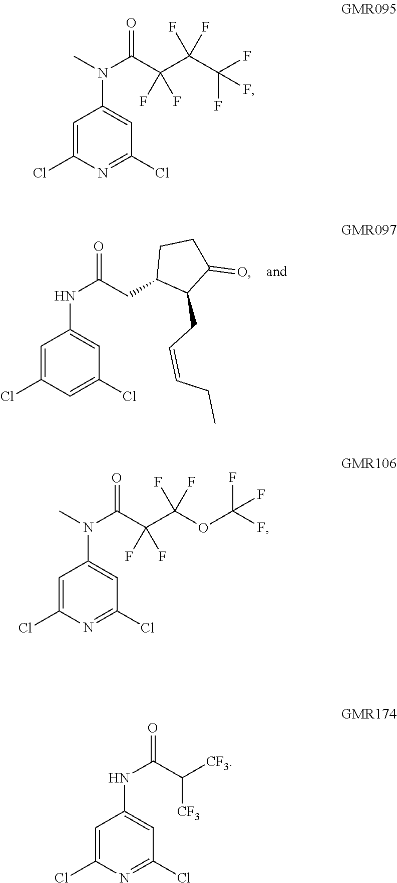 [SMILES: CN(C(=O)C(F)(F)C(F)(F)C(F)(F)F)c1cc(Cl)nc(Cl)c1 ; CC/C=C\C[C@@H]1C(=O)CC[C@H]1CC(=O)Nc1cc(Cl)cc(Cl)c1 ; CN(C(=O)C(F)(F)C(F)(F)OC(F)(F)F)c1cc(Cl)nc(Cl)c1 ; O=C(Nc1cc(Cl)nc(Cl)c1)C(C(F)(F)F)C(F)(F)F]

125. A composition comprising a compound of embodiment 104 and a carrier.

126. A method of repelling an arthropod from a locus, comprising contacting said locus with the composition of embodiment 125.

127. A method for controlling one or more arthropods, comprising contacting said one or more arthropods with the composition of embodiment 125.

128. The method of embodiment 126 or 127, wherein the composition is a solution, dust, granular formulation, or emulsion.

129. The method of embodiment 126 or 127, wherein the composition is a liquid solution or emulsion.

130. The method of embodiment 126, wherein said arthropod is a fly, spider, butterfly, crab, mosquito, centipede, tick, millipede, or scorpion.

131. The method of embodiment 126, wherein said arthropod is an insect.

132. The method of embodiment 131, wherein said insect is a mosquito.

133. The method of embodiment 126, where the locus is an area, an environment, or the skin of an animal.

134. A method of repelling arthropods, comprising contacting an object or locus with a compound of embodiment 104, wherein said arthropods are repelled from an adjacent area to said object or locus, wherein said arthropods are not physically contacted with said object or locus.

135. The method of embodiment 134, wherein said arthropods are insects.

136. A method of providing a compound of embodiment 104 in an arthropod repellent medium from which a vaporized compound of embodiment 104 can be dispersed.

137. The method of embodiment 136, wherein said arthropod repellent medium is an insect repellent medium.

138. A compound, selected from the group consisting of:

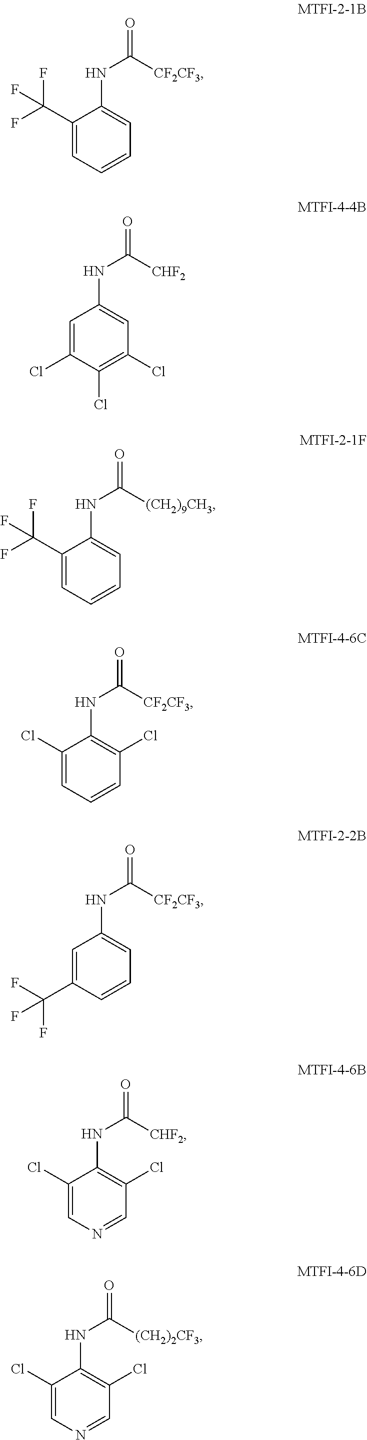

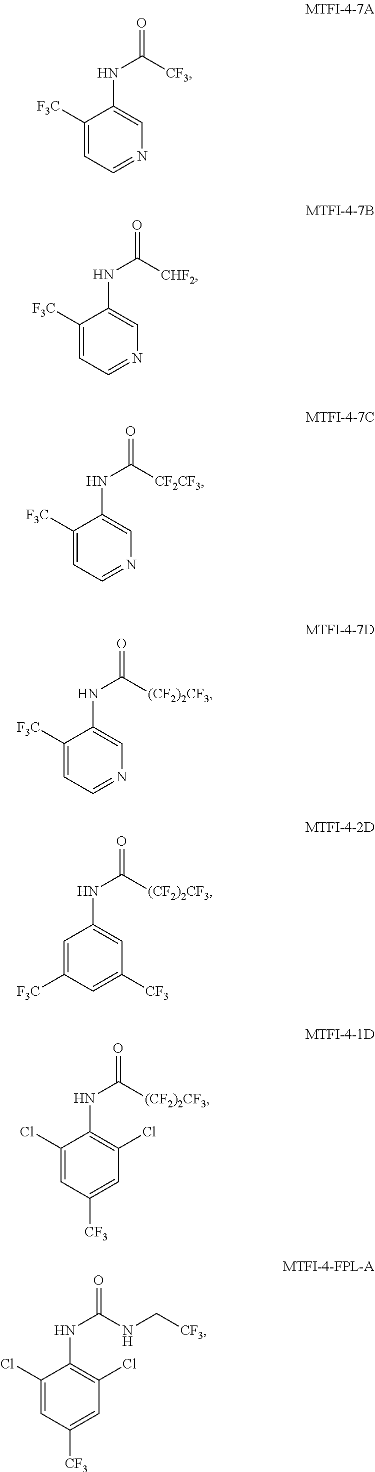

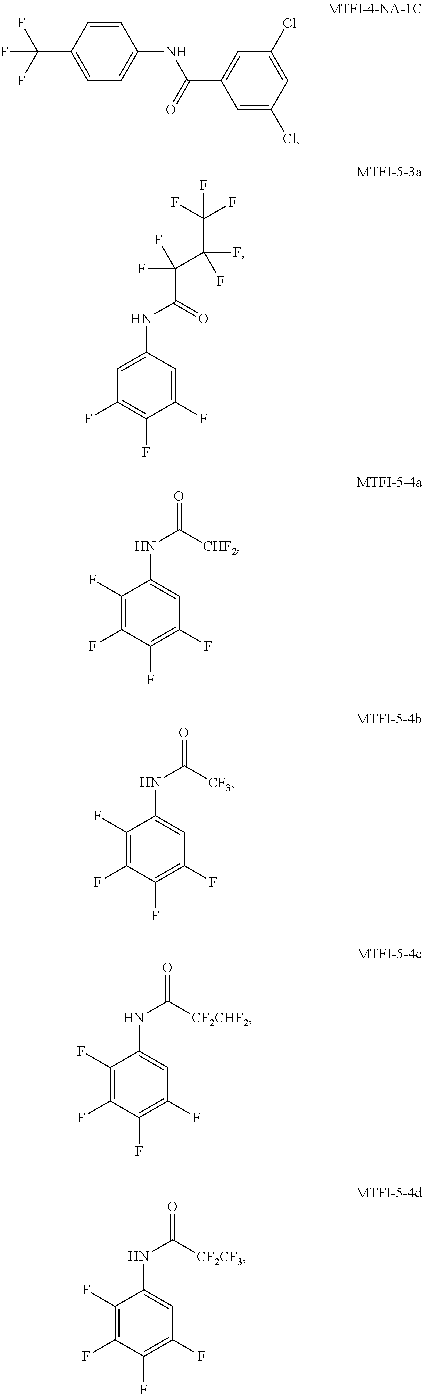

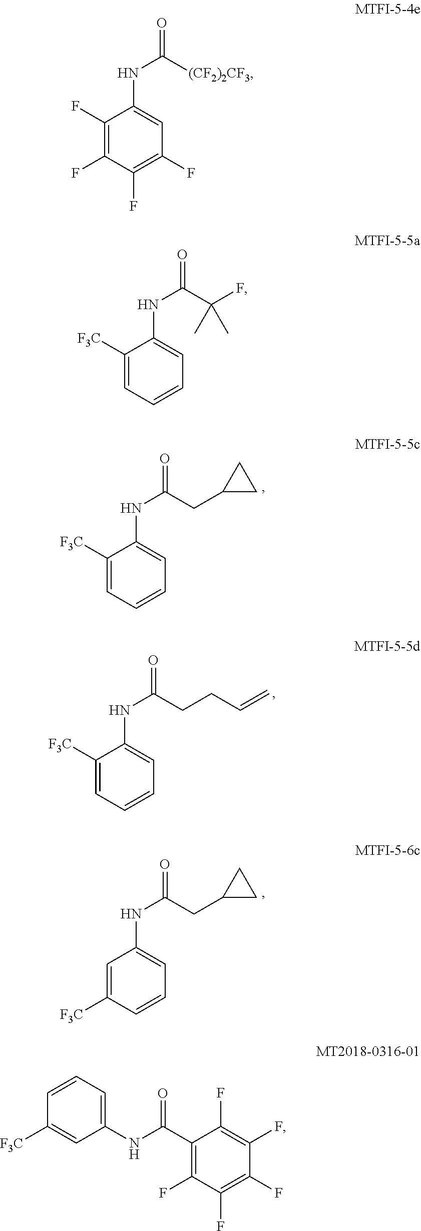

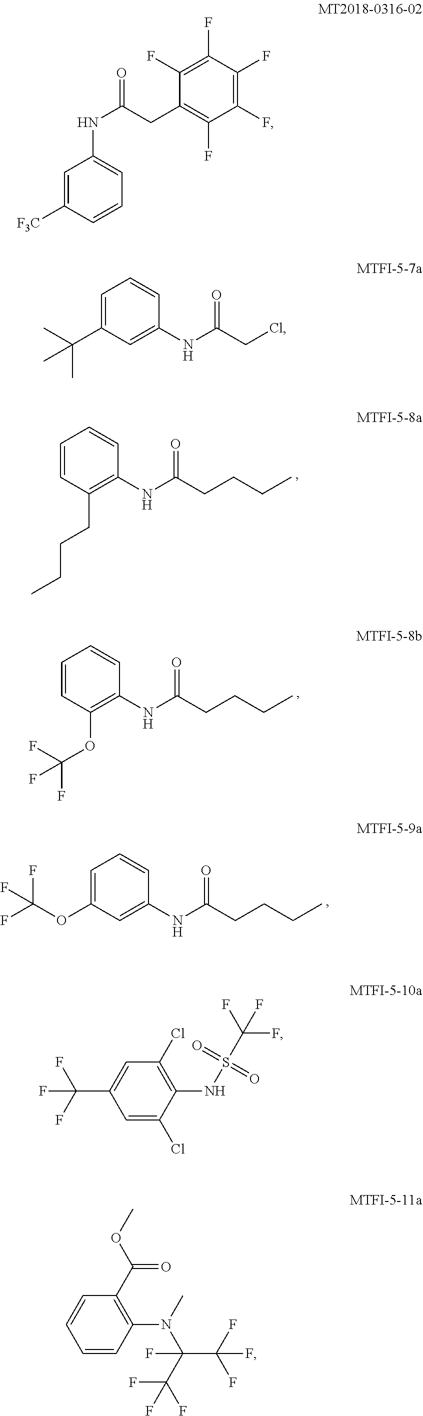

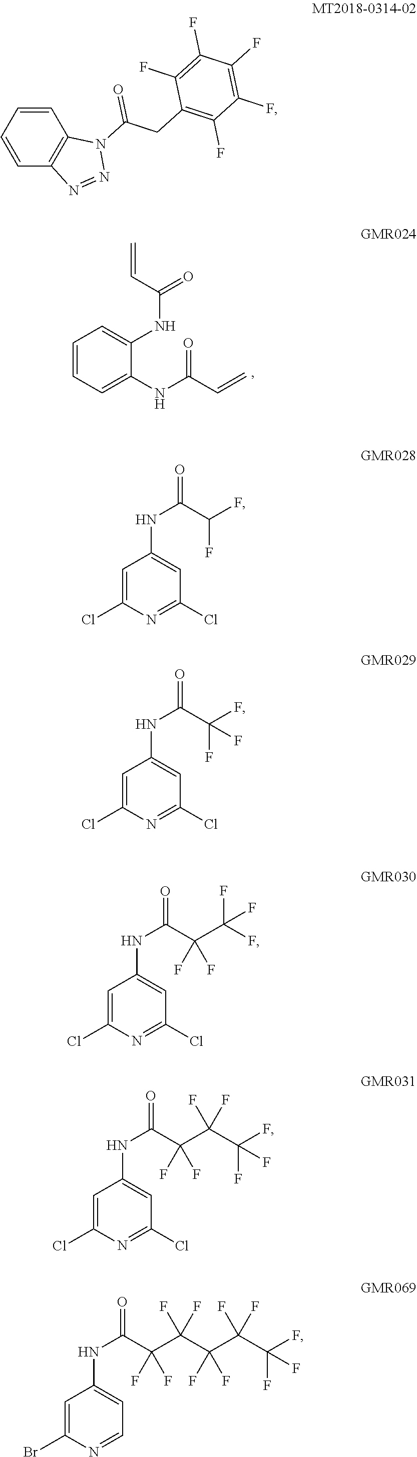

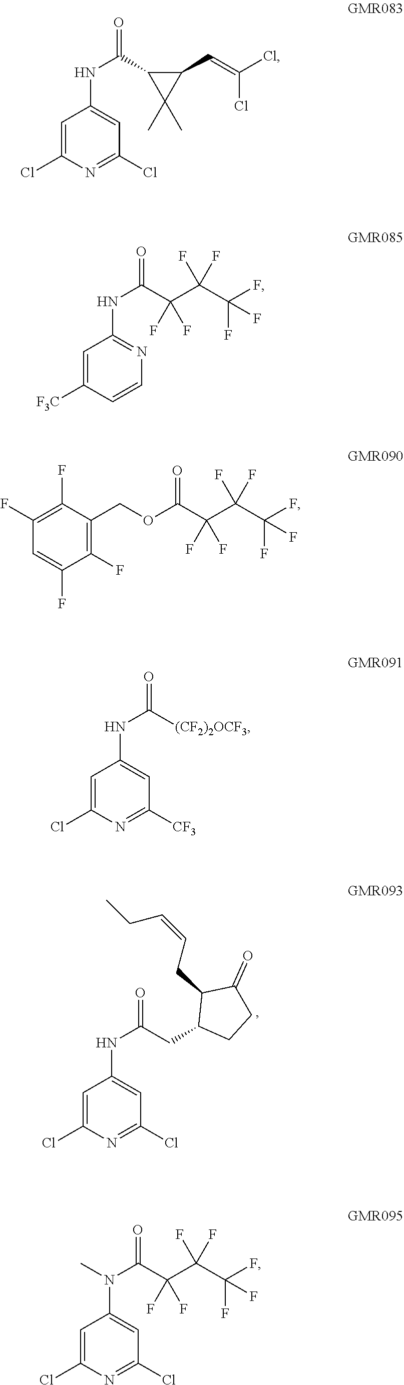

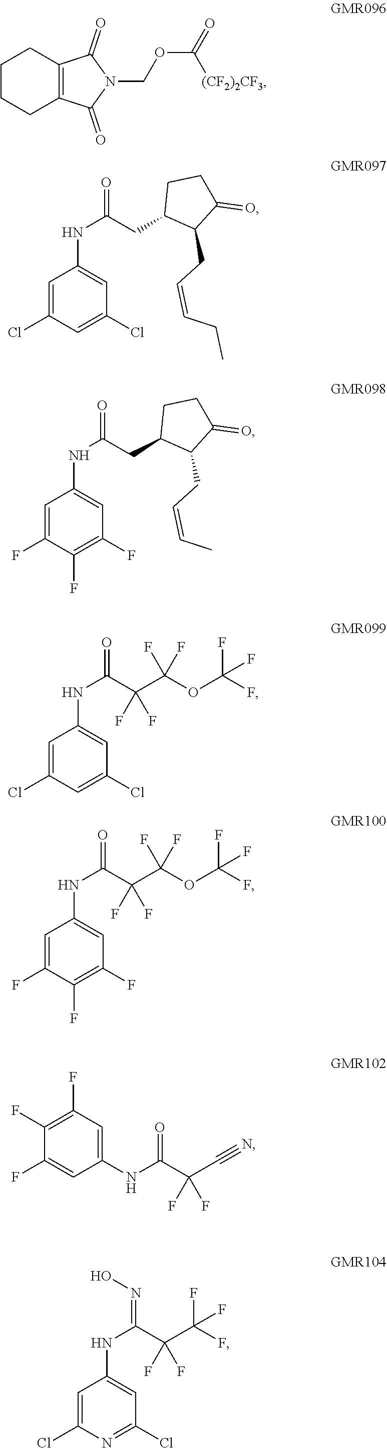

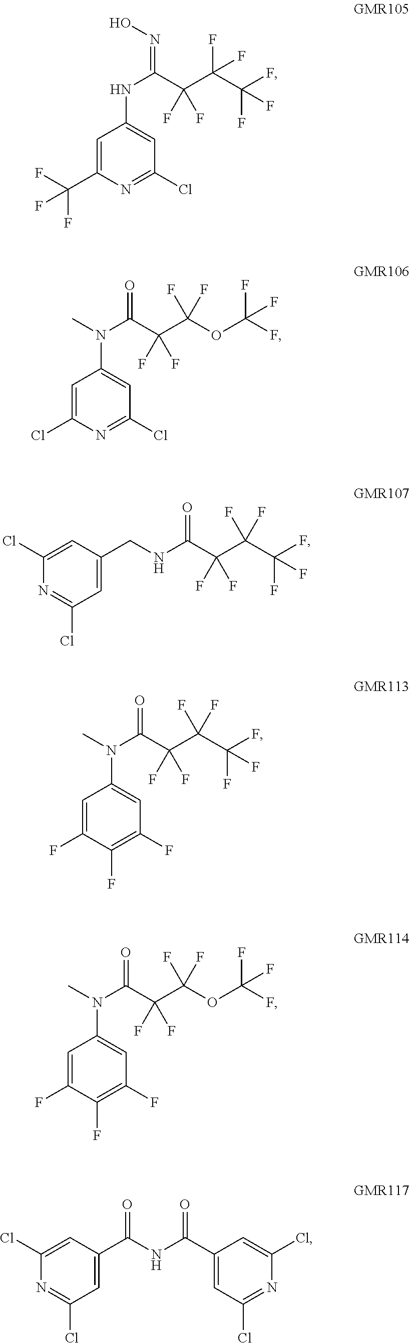 [SMILES: O/N=C(\Nc1cc(Cl)nc(C(F)(F)F)c1)C(F)(F)C(F)(F)C(F)(F)F ; CN(C(=O)C(F)(F)C(F)(F)OC(F)(F)F)c1cc(Cl)nc(Cl)c1 ; O=C(NCc1cc(Cl)nc(Cl)c1)C(F)(F)C(F)(F)C(F)(F)F ; CN(C(=O)C(F)(F)C(F)(F)C(F)(F)F)c1cc(F)c(F)c(F)c1 ; CN(C(=O)C(F)(F)C(F)(F)OC(F)(F)F)c1cc(F)c(F)c(F)c1 ; O=C(NC(=O)c1cc(Cl)nc(Cl)c1)c1cc(Cl)nc(Cl)c1]

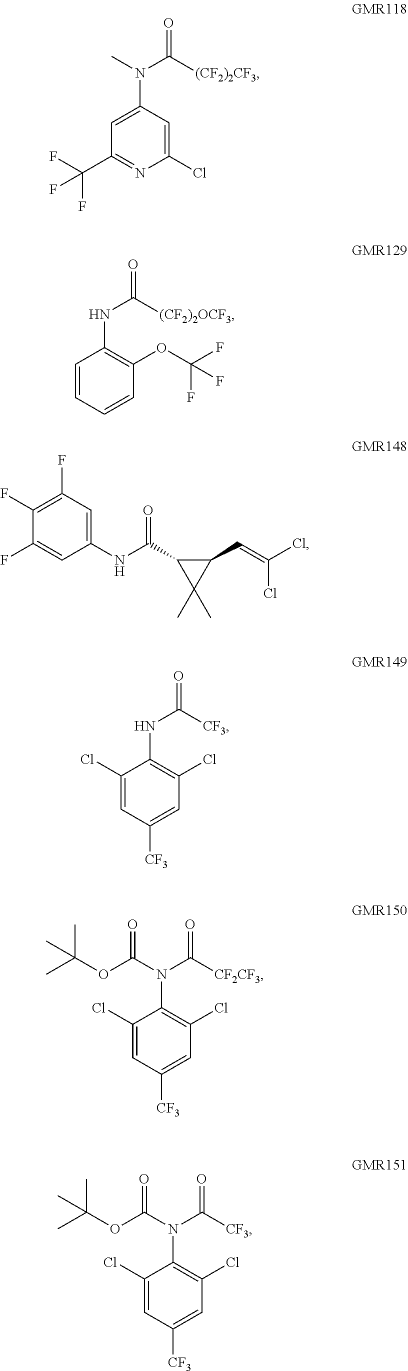

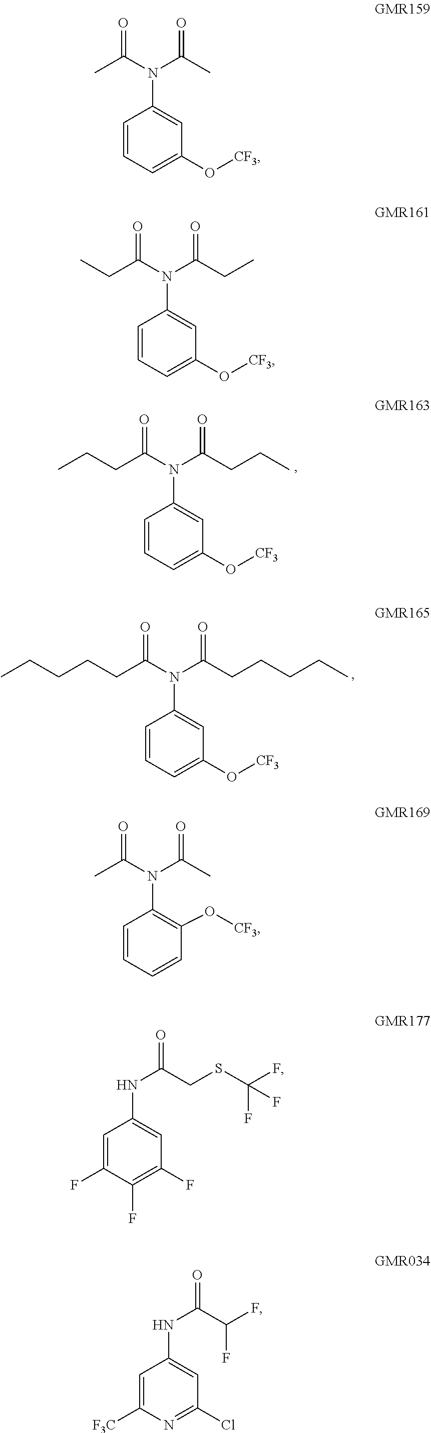

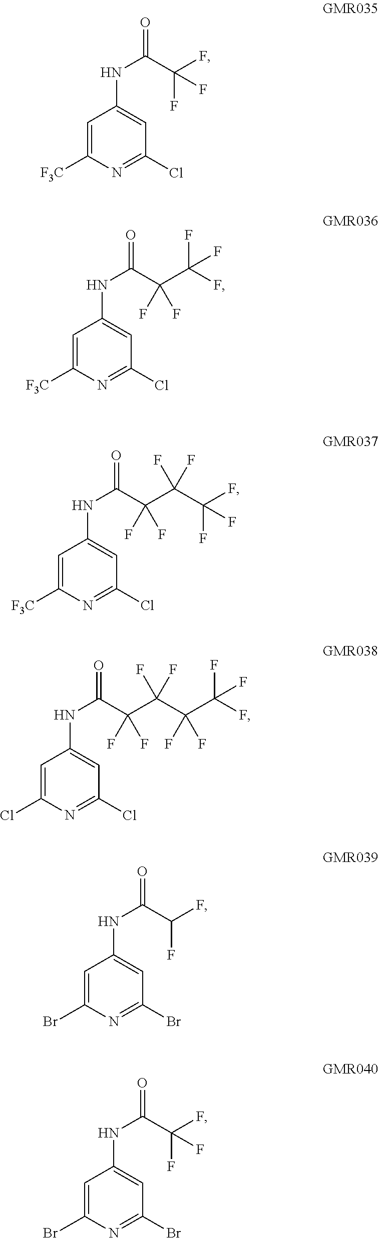 [SMILES: O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(Cl)c1)C(F)(F)C(F)(F)C(F)(F)C(F)(F)F ; O=C(Nc1cc(Br)nc(Br)c1)C(F)F ; O=C(Nc1cc(Br)nc(Br)c1)C(F)(F)F]

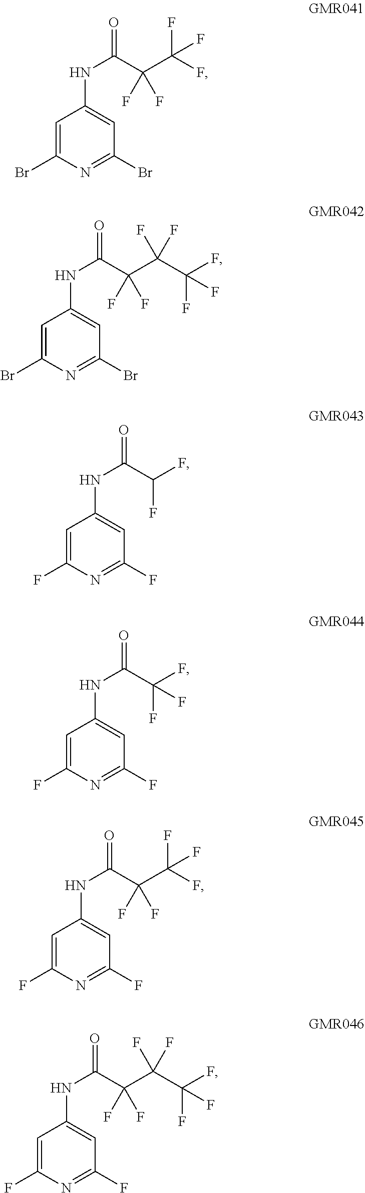 [SMILES: O=C(Nc1cc(Br)nc(Br)c1)C(F)(F)C(F)(F)F ; O=C(Nc1cc(Br)nc(Br)c1)C(F)(F)C(F)(F)C(F)(F)F ; O=C(Nc1cc(F)nc(F)c1)C(F)F ; O=C(Nc1cc(F)nc(F)c1)C(F)(F)F ; O=C(Nc1cc(F)nc(F)c1)C(F)(F)C(F)(F)F ; O=C(Nc1cc(F)nc(F)c1)C(F)(F)C(F)(F)C(F)(F)F]

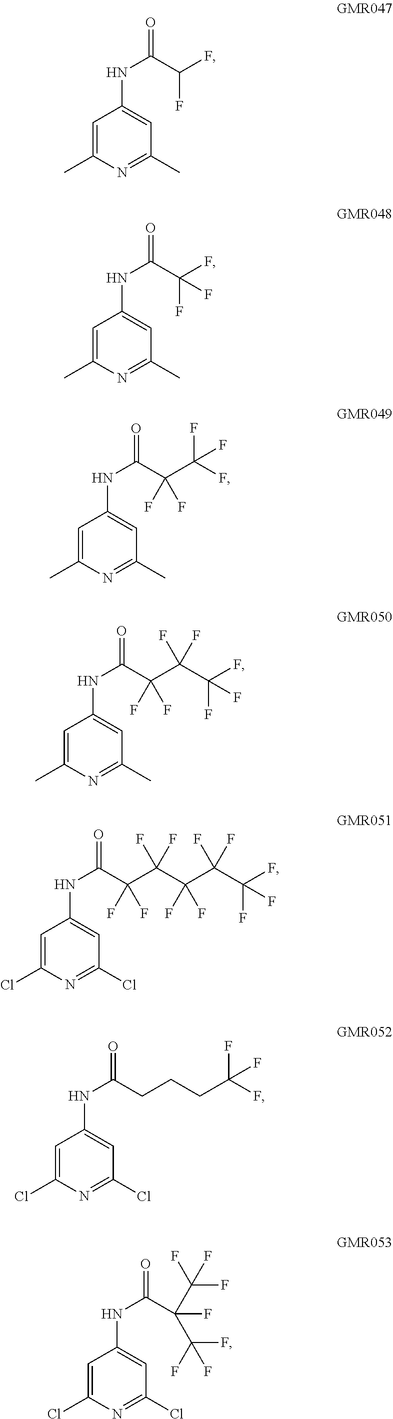

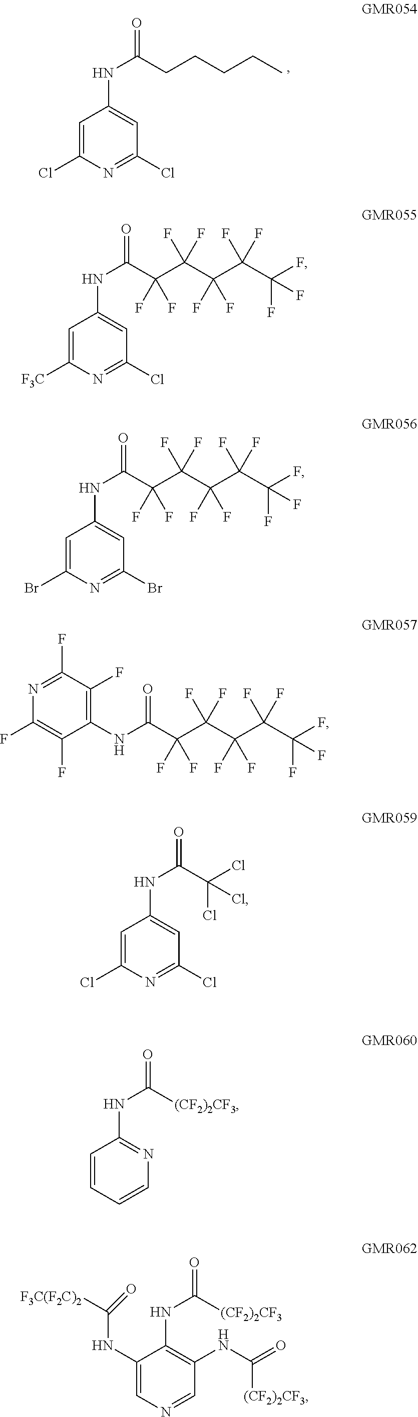

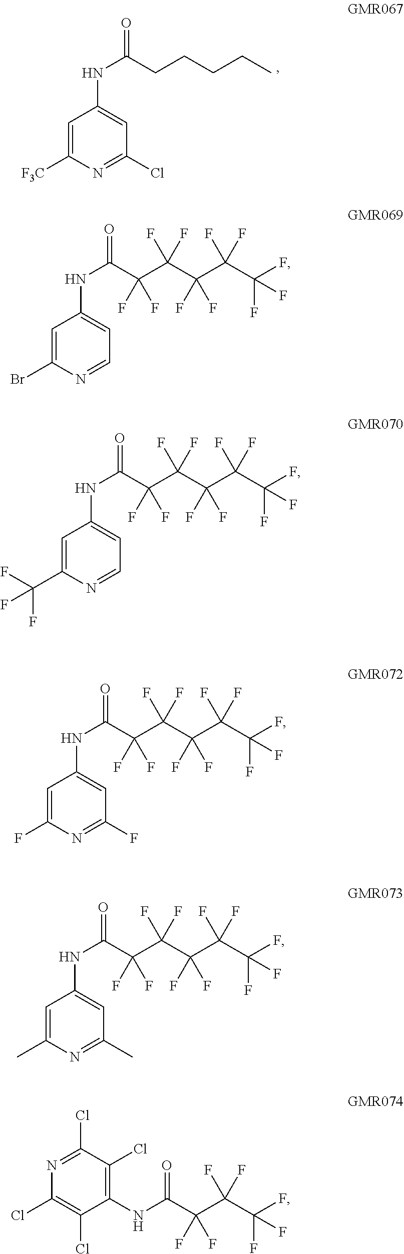

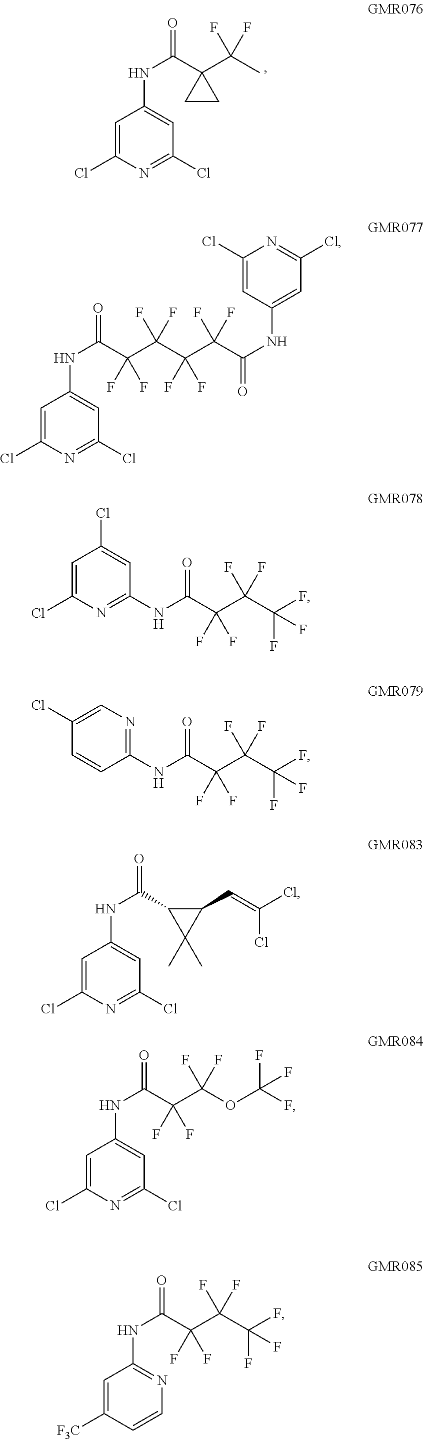

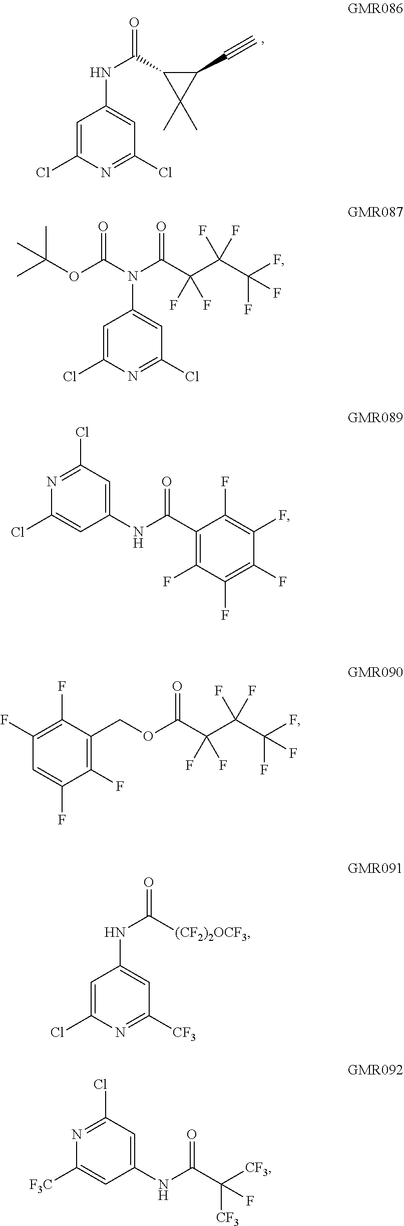

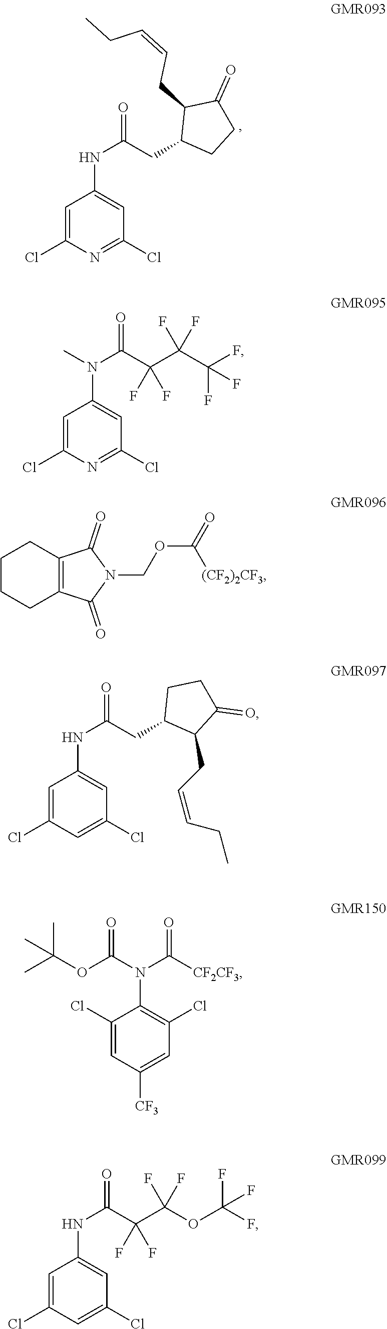

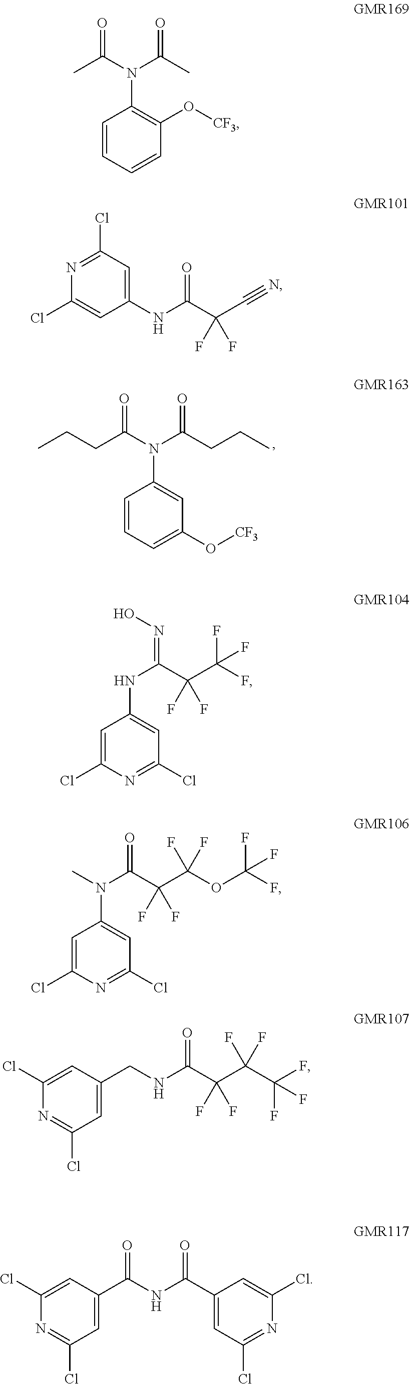

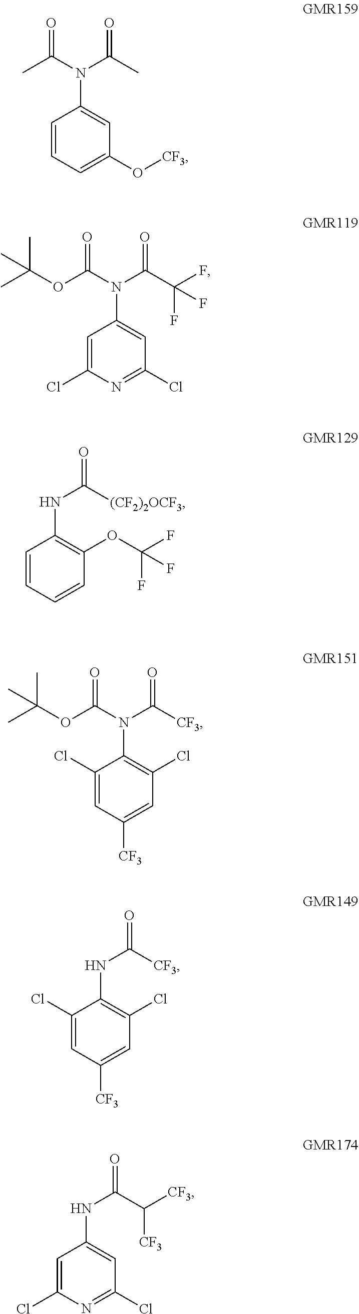

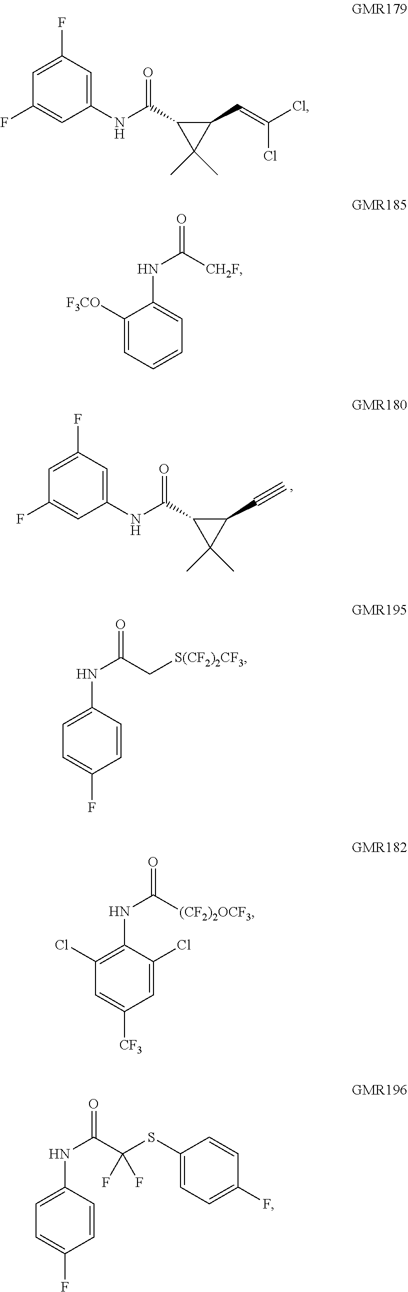

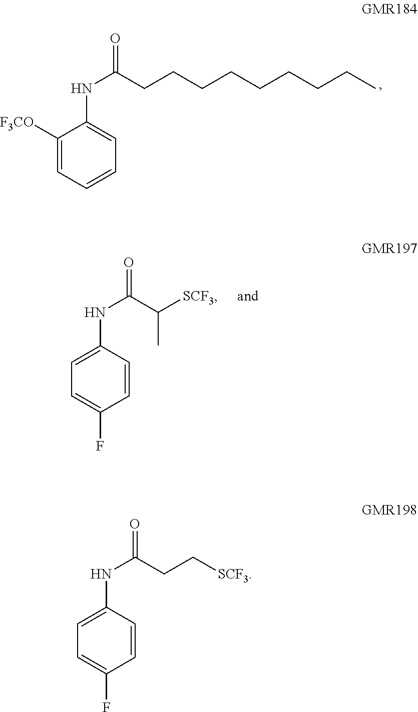

139. A composition comprising a compound of embodiment 138 and a carrier.

140. A method of repelling an arthropod from a locus, comprising contacting said locus with the composition of embodiment 139.

141. A method for controlling one or more arthropods, comprising contacting said one or more arthropods with the composition of embodiment 139.

142. The method of embodiment 140 or 141, wherein the composition is a solution, dust, granular formulation, or emulsion.

143. The method of embodiment 140 or 141, wherein the composition is a liquid solution or emulsion.

144. The method of embodiment 140, wherein said arthropod is a fly, spider, butterfly, crab, mosquito, centipede, tick, millipede, or scorpion.

145. The method of embodiment 141, wherein said one or more arthropods are one or more insects.

146. The method of embodiment 144, wherein said arthropod is an insect.

147. The method of embodiment 146, wherein said insect is a mosquito.

148. The method of embodiment 140, where the locus is an area, an environment, or the skin of an animal.

149. A method of repelling arthropods, comprising contacting an object or locus with a compound of embodiment 138, wherein said arthropods are repelled from an adjacent area to said object or locus, wherein said arthropods are not physically contacted with said object or locus.

150. The method of embodiment 149, wherein said arthropods are insects.

151. A method of providing a compound of embodiment 138 in an arthropod repellent medium from which a vaporized compound of embodiment 138 can be dispersed.

152. The method of embodiment 151, wherein said arthropod repellent medium is an insect repellent medium.

153. A composition comprising an essential oil and a compound of Formula IA, IB, IC, ID, IE, IF, or IG

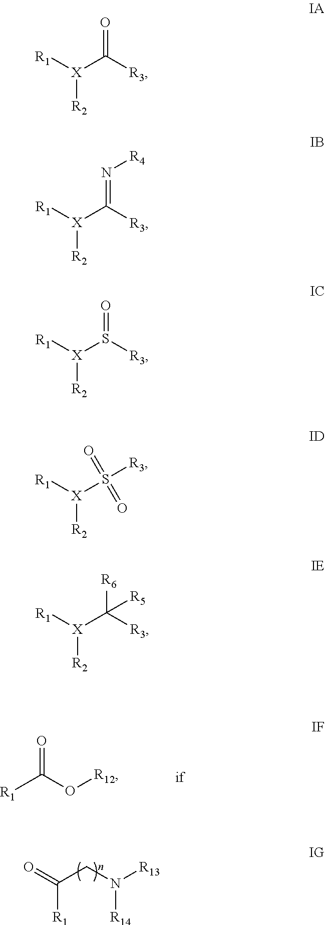

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃. C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl, Rao

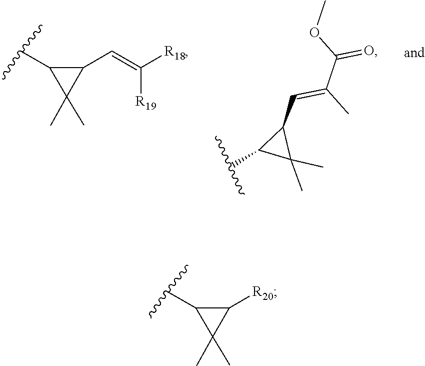

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
   wherein R₁₃ and R₁₄, together with the nitrogen to which each is attached, form a 5 to 10-membered heterocyclyl or C₆-C₁₀ heteroaryl; and
n is 0 or 1;
wherein the composition is synergistic, in admixture with a carrier.

154. The composition of claim embodiment 153, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.

155. The composition of embodiment 153 or 154, wherein said essential oil is citronella oil.

156. The composition of any one of embodiments 153-155, wherein said compound is of Formula IA.

157. The composition of any one of embodiments 153-156, wherein X is N.

158. The composition of any one of embodiments 153-157, wherein R₁ is selected from the group consisting of

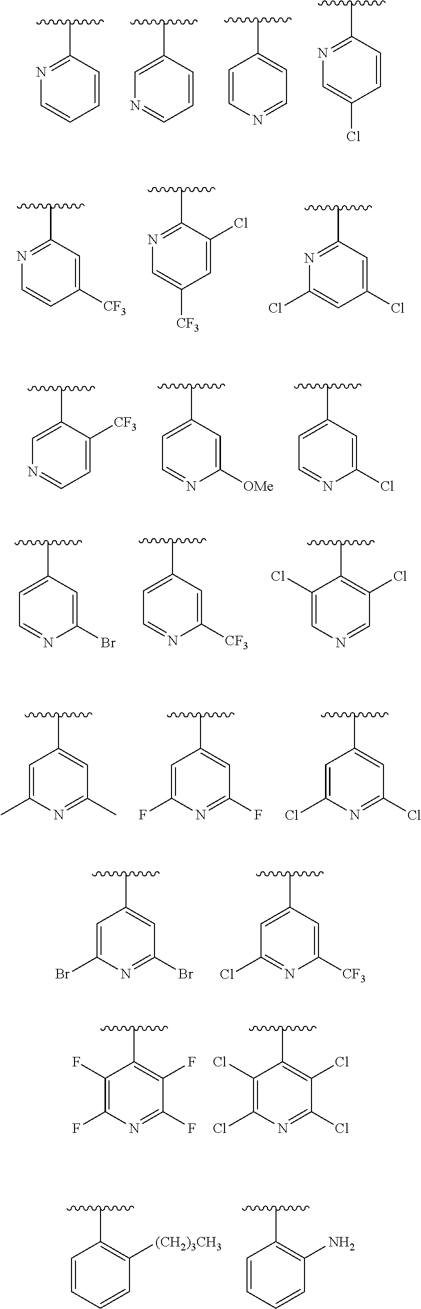

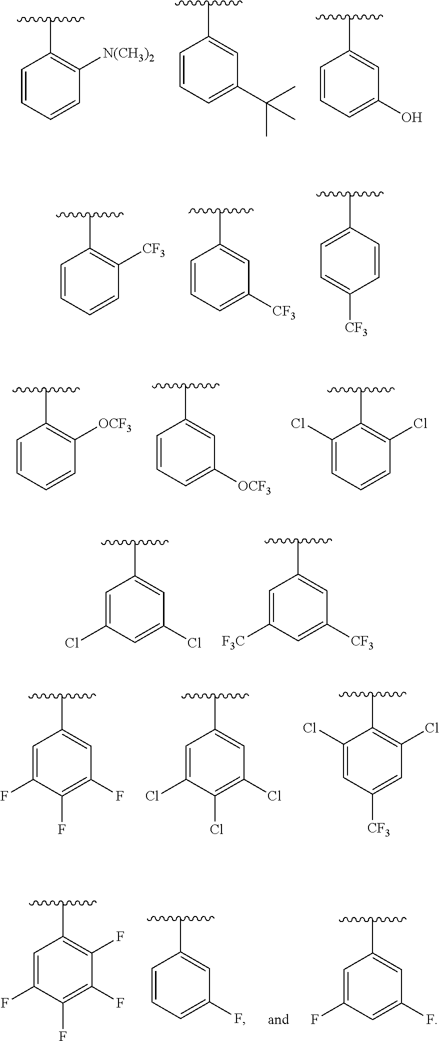

159. The composition of any one of embodiments 153-158, wherein R₂ is H.

160. The composition of any one of embodiments 153-159, wherein R₃ is selected from the group consisting of

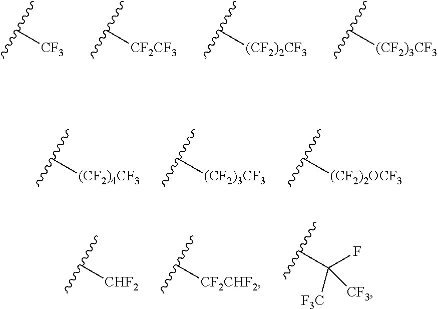

and C₁-C₆ alkyl.

161. The composition ofany one of embodiments 153-160, wherein said compound is

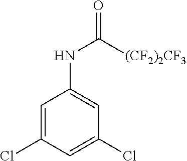

and said essential oil is citronella oil.

162. A method of repelling arthropods, comprising contacting an object or locus with a composition comprising an essential oil and a compound of Formula IA, 11B, IC, ID, IE, IF, or IG

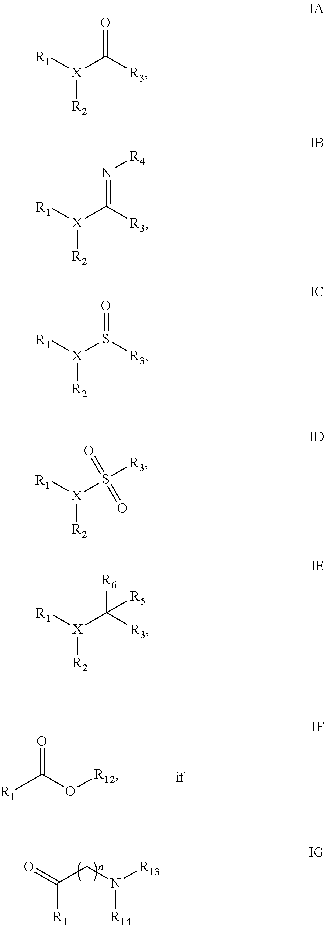

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃. C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

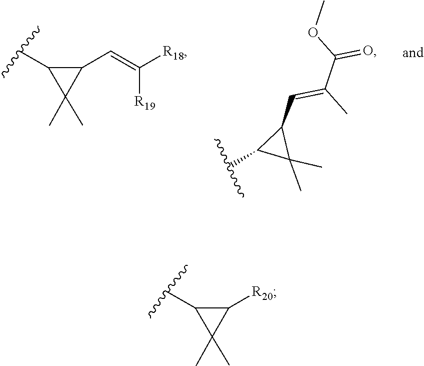

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
   wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
wherein R₁₃ and R₁₄, together with the nitrogen to which each is attached, form a 5 to 10-membered heterocyclyl or C₆-C₁₀ heteroaryl; and
n is 0 or 1;
wherein said arthropods are repelled from an adjacent area to said object or locus, wherein said arthropods are not physically contacted with said object or locus; and, wherein the composition is synergistic, in admixture with a carrier.

163. The method of claim embodiment 162, wherein said composition comprises said compound in an amount from about 0.01-99.99% and said essential oil in an amount from about 99.99%-0.01%.

164. The method of embodiment 162, wherein said composition contains about 0.0001 to 100000 parts per million of said compound.

165. The method of any one of embodiments 162-164, wherein said essential oil is selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil.

166. The method of any one of embodiments 162-165, wherein said essential oil is citronella oil.

167. The method of any one of embodiments 162-166, wherein said compound is of Formula IA.

168. The method of any one of embodiments 162-167, wherein X is N.

169. The method of any one of embodiments 162-168, wherein R₁ is selected from the group consisting of

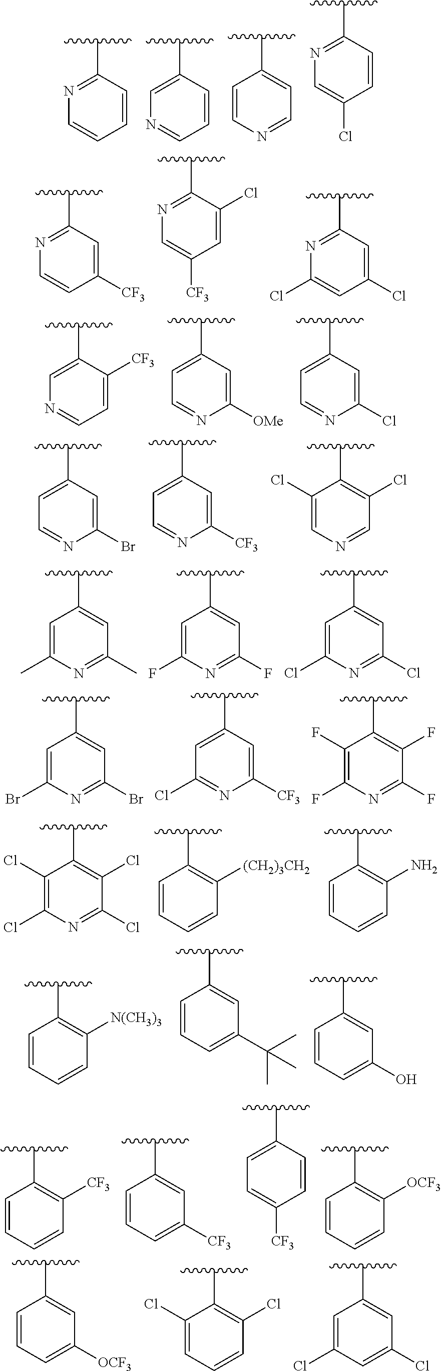

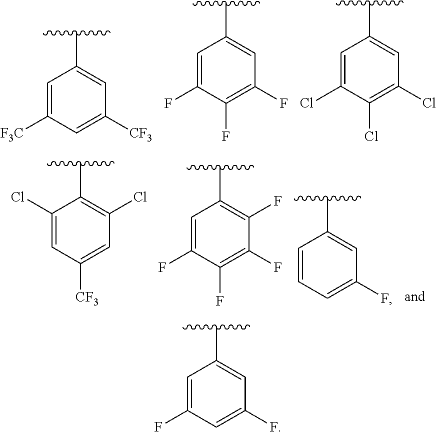

170. The method of any one of embodiments 162-169, wherein R₂ is H.

171. The method of any one of embodiments 162-170, wherein R₃ is selected from the group consisting of

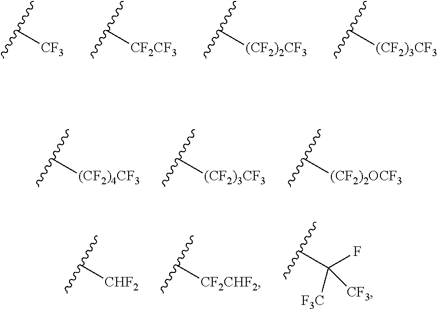

and C₁-C₆ alkyl.

172. The method of embodiments 162-170, wherein said compound is

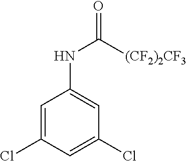

and said essential oil is citronella oil.

173. The method of embodiment 162, wherein said arthropods are insects.

174. The method of embodiment 173, wherein said insects are mosquitos.

175. A composition comprising a compound of Formula IA, IB, IC, ID, IE, IF, or IG

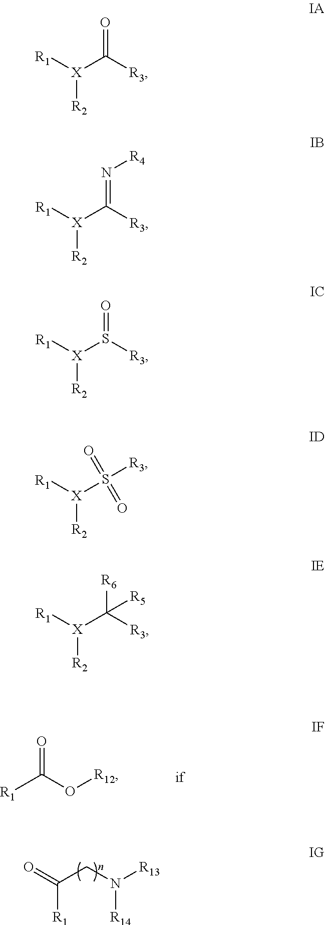

wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃. C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

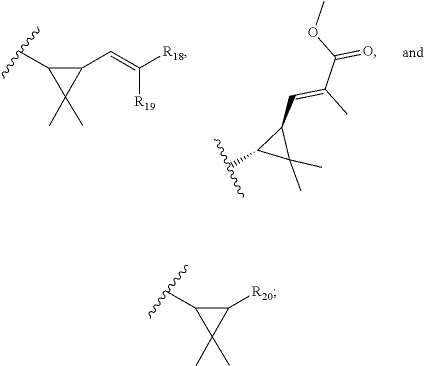

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl;
or,
wherein R₁₃ and R₁₄, together with the nitrogen to which each is attached, form a 5 to 10-membered heterocyclyl or C₆-C₁₀ heteroaryl; and
n is 0 or 1;
   and second compound selected from the group consisting of a pyrethroid, a compound of Formula II′, and a compound of Formula III′

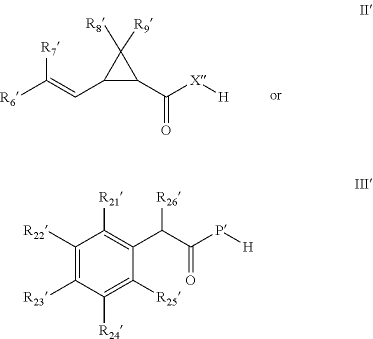

wherein,
X″ is O, NH, or S;
   R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or
   wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
   R_{8′} and R_{9′} are each independently selected from hydrogen, haloalkyl, alkenyl, alkynyl, and C₁. C₆ alkyl;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl, and
wherein the composition is synergistic, in intimate admixture with a carrier.

176. The composition of embodiment 175, wherein said composition comprises a compound of Formula II′

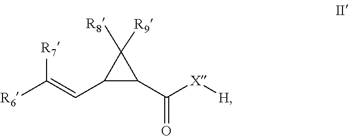

wherein,
X″ is O;
   R_{6′} and R_{7′} are each independently selected from the group consisting of hydrogen, halo, haloalkyl, and C₁-C₃ alkyl; and
R_{8′} and R_{9′} are each methyl.

177. The composition of embodiment 175 or 176, wherein said compound is of Formula IA.

178. The composition of any one of embodiments 175-177, wherein X is N.

179. The composition of any one of embodiments 175-178, wherein R₁ is selected from the group consisting of

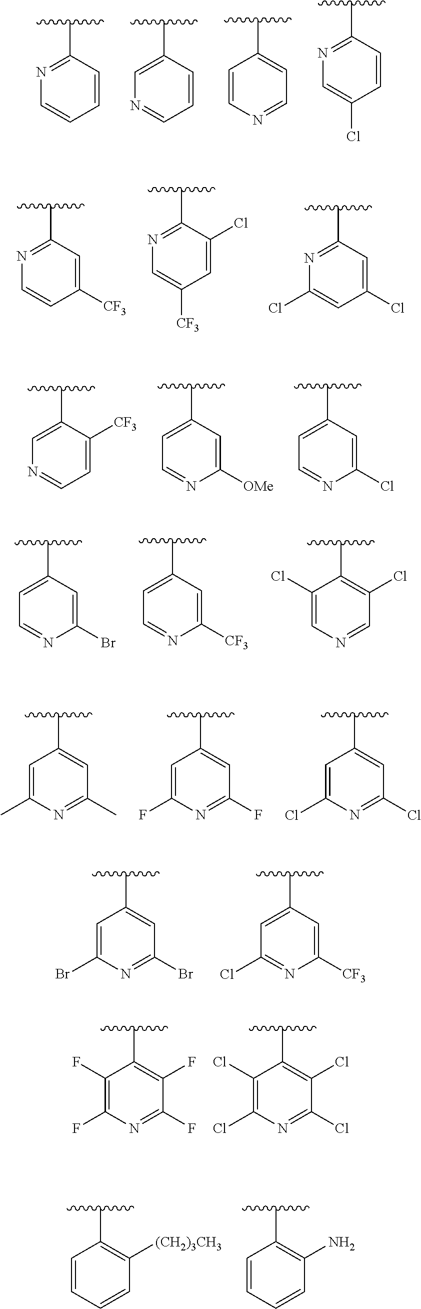

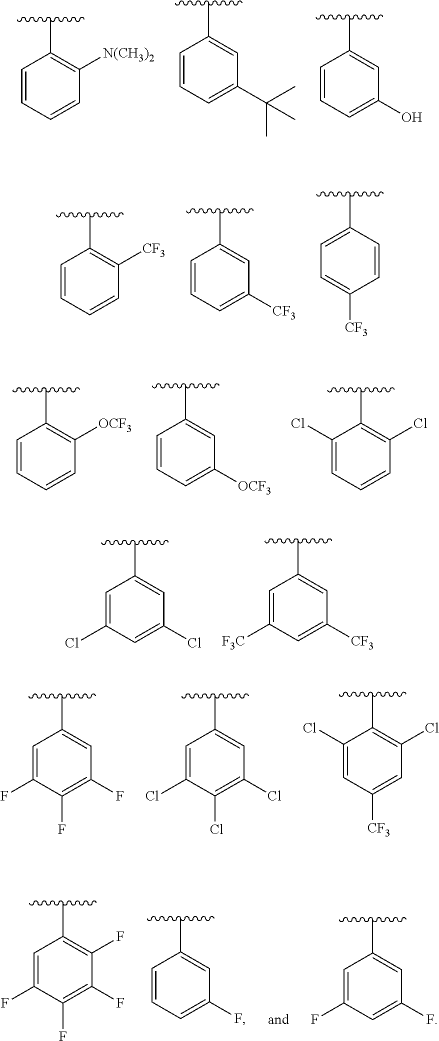

180. The composition ofany one of embodiments 175-179, wherein R₂ is H.

181. The composition of any one of embodiments 175-180, wherein R₃ is selected from the group consisting of

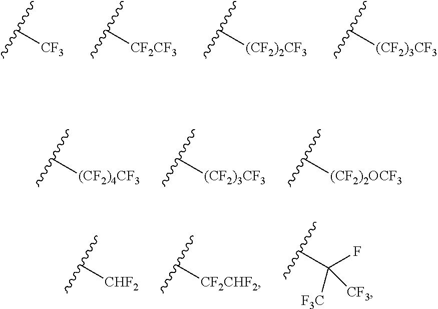

and C₁-C₆ alkyl.

182. The composition of any one of embodiments 175-181, wherein said composition comprises a compound having the structure

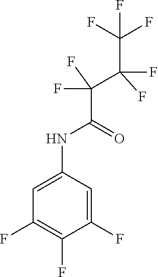 [SMILES: O=C(Nc1cc(F)c(F)c(F)c1)C(F)(F)C(F)(F)C(F)(F)F]

and a compound of Formula II′ having the structure

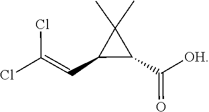

183. A method of repelling arthropods, comprising contacting an object or locus with a composition comprising a compound of Formula IA, IB, IC, ID, IE, IF, or IG

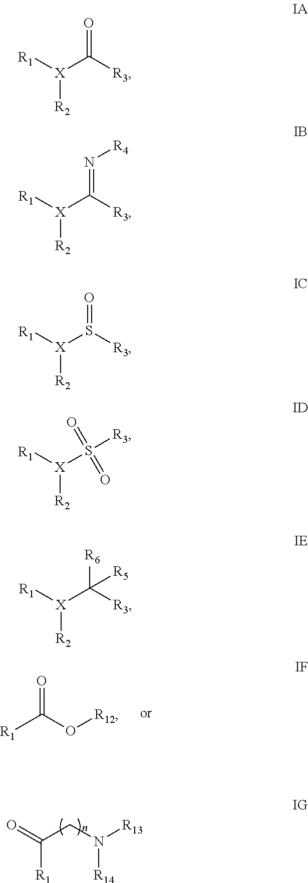

wherein,
X is O, N, or C; R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl; wherein said heteroaryl, phenyl, cycloalkyl, or heterocycyl is optionally substituted with one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, benzyl, C₃-C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is optionally substituted with one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, haloalkyl-haloalkoxy, NR₂₁R₂₂, cyano-alkyl, (C₃-C₇ cycloalkyl)-alkyl, C₃-C₇ cycloalkyl, (C₆-C₁₀ aryl)-C₁-C₃ alkyl, phenyl,

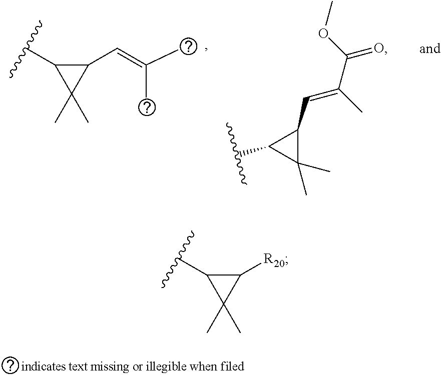

where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is optionally substituted one, two, or three times with halogen;
wherein said “alkyl” in cyano-alkyl is optionally substituted with halogen;
one or more carbons in said “haloalkyl” is optionally replaced with S; and
said cycloalkyl, phenyl, (C₆-C₁₀ aryl)-C₁-C₃ alkyl, or (C₃-C₇ cycloalkyl)-alkyl is optionally substituted with one, two, three, four, or five substituents, each independently selected from the group consisting of alkene, haloalkyl, and halogen; and
wherein the “alkyl” in (C₃-C₇ cycloalkyl)-alkyl or (C₆-C₁₀ aryl)-C₁-C₃ alkyl is optionally substituted one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is selected from hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from 5- or 6-membered heterocyclyl, phenyl, hydrogen, and C₁-C₃ alkyl, wherein said heterocyclyl or phenyl is optionally substituted with one or two substituents each independently selected from the group consisting of halogen, haloalkyl, and linear or branched C₁-C₃ alkyl; or,
wherein R₁₃ and R₁₄, together with the nitrogen to which each is attached, form a 5 to 10-membered heterocyclyl or C₆-C₁₀ heteroaryl; and
n is 0 or 1;
   and, a second compound selected from the group consisting of a pyrethroid, a compound of Formula II′, and a compound of Formula III′

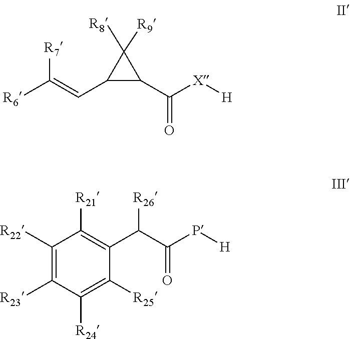

wherein,
X″ is O, NH, or S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or
wherein R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl, and
wherein said arthropods are repelled from an adjacent area to said object or locus, wherein said arthropods are not physically contacted with said locus; and, wherein the composition is synergistic, in admixture with a carrier.

184. The method of embodiment 183, wherein said composition comprises said compound of Formula IA, IB, IC, ID, IE, IF, or IG in an amount from about 0.01-99.99% and said second compound in an amount from about 99.99/6-0.01%.

185. The method of embodiment 183, wherein said composition contains about 0.0001 to 100000 parts per million of a compound of Formula IA, IB, IC, ID, IE, IF, or IG.

186. The method of any one of embodiments 183-185, wherein said composition comprises a compound of Formula II′

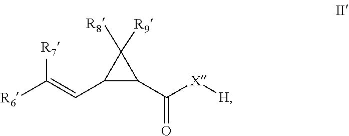

wherein,
X″ is O;
R_{6′} and R_{7′} are each independently selected from the group consisting of hydrogen, halo, haloalkyl, and C₁-C₃ alkyl; and
R_{8′} and R_{9′} are each methyl.

187. The method of any one of embodiments 183-186, wherein said compound is of Formula IA.

188. The method of any one of embodiments 183-187, wherein X is N.

189. The method of any one of embodiments 183-188, wherein R₁ is selected from the group consisting of

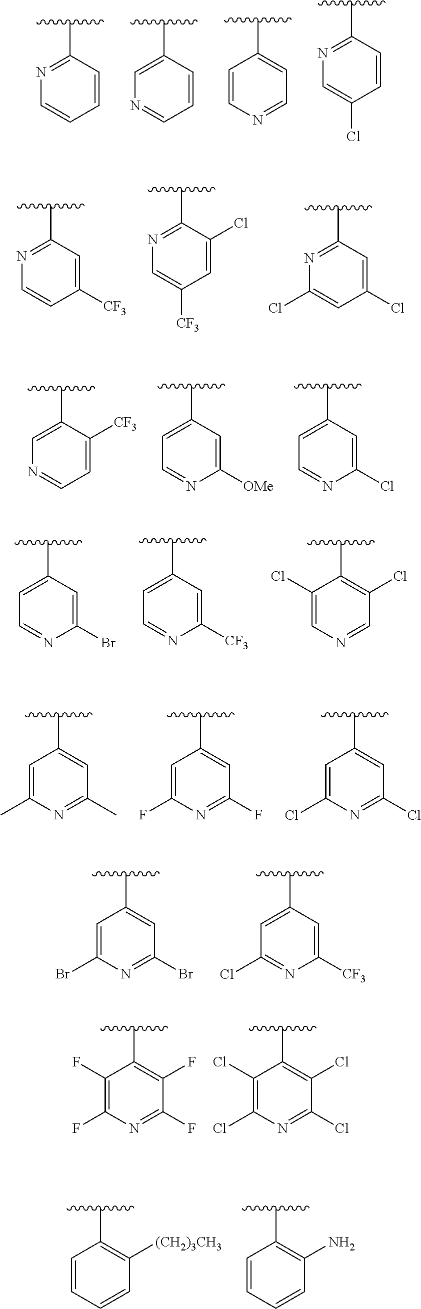

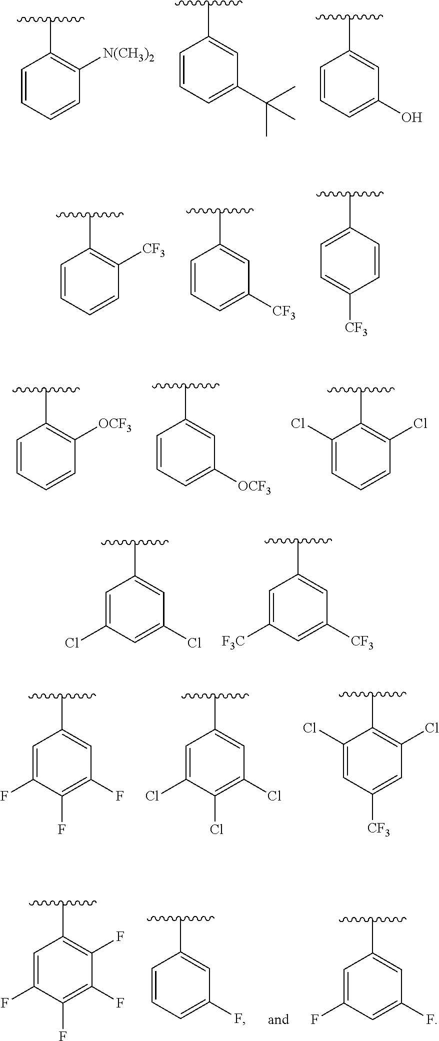

190. The method of any one of embodiments 183-189, wherein R₂ is H.

191. The method of any one of embodiments 183-190, wherein R₃ is selected from the group consisting of

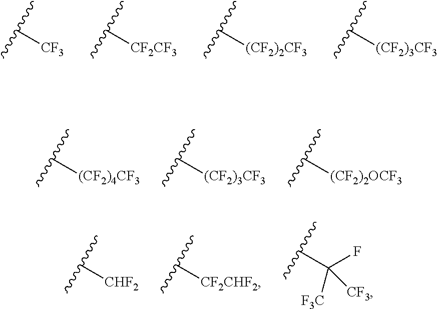

and C₁-C₆ alkyl.

192. The method of embodiments 183-191, wherein said composition comprises

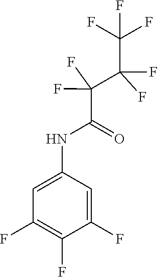 [SMILES: O=C(Nc1cc(F)c(F)c(F)c1)C(F)(F)C(F)(F)C(F)(F)F]

and a compound of Formula II′ having the structure

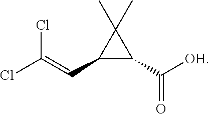

193. The method of embodiments 183-192, wherein said arthropod is a fly, spider, butterfly, crab, mosquito, moth centipede, tick, millipede, or scorpion.

194. The method of embodiments 183-193, wherein said arthropod is an insect.

195. The method of embodiment 194, wherein said insect is a mosquito.

The following examples are offered by way of illustration and not by way of limitation.

### EXAMPLES

### Example 1: Preparation of 2,2,3,3,4,4,4-heptafluoro-N-(3,4,5-trifluorophenyl)butanamide (MTFI-5-3A)

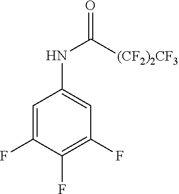

Prepared using General Procedure Method A from triethylamine (0.95 mL, 6.8 mmol), 3,4,5-trifluoroaniline (0.5 mg, 3.4 mmol), dichloromethane (17 mL), heptafluorobutyric anhydride (0.93 mL, 3.7 mmol), and 4-Dimethylaminopyridine (83 mg, 0.7 mmol). Automated flash chromatography yielded the product as a white solid (1.07 g, 92% yield). ¹H NMR (500 MHz, Acetone-d₆) δ 10.61 (s, 1H), 7.64 (dd, J=9.1, 6.5 Hz, 2H). ¹³C NMR (126 MHz, Acetone-d₆) δ 155.76 (t, J=26.2 Hz), 150.80 (ddd, J=246.5, 10.3, 5.0 Hz), 137.19 (dt, J=248.6, 15.5 Hz), 132.52 (td, J=11.8, 4.3 Hz), 117.19 (qt, J=287.0, 33.8, 32.6 Hz), 110.73-105.87 (m), 105.75 (ddd, J=19.6, 6.4, 0.8 Hz).

### Example 2: Preparation of N-(2,6-dichloro-4-(trifluoromethyl)phenyl)-2,2,3,3,3-pentafluoropropanamide (GMR138)

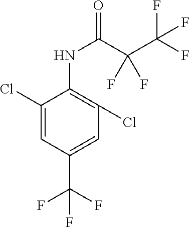 [SMILES: O=C(Nc1c(Cl)cc(C(F)(F)F)cc1Cl)C(F)(F)C(F)(F)F]

Prepared using General Procedure Method A from triethylamine (0.24 mL, 1.74 mmol), 2,6-dichloro-4-(trifluoromethyl)aniline (0.189 mg, 0.96 mmol), dichloromethane (4.35 mL), pentafluoropropionic anhydride (0.19 mL, 0.956 mmol), and 4-Dimethylaminopyridine (21.3 mg, 0.17 mmol). Automated flash chromatography yielded the product as a white solid (194.7 mg, 62% yield). ¹H NMR (500 MHz, Chloroform-d) δ 8.83 (s, 1H), 7.66 (s, 2H). ¹³C NMR (126 MHz, Chloroform-d) δ 156.10 (t, J=26.9 Hz), 134.69, 132.68, 132.33 (q, J=34.4 Hz), 125.66 (q, J=3.7 Hz), 122.16 (q, J=285.5, 273.3, 272.3 Hz), 116.75 (tt, J=285.6, 34.4, 33.8 Hz), 106.89 (tq, J=266.6, 39.5 Hz).

### Example 3: Preparation of N-(2-chloro-6-(trifluoromethyl)pyridin-4-yl)-2,3,3,3-tetrafluoro-2-(trifluoromethyl)propanamide (GMR092)

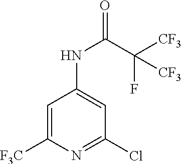 [SMILES: O=C(Nc1cc(Cl)nc(C(F)(F)F)c1)C(F)(C(F)(F)F)C(F)(F)F]

Prepared using General Procedure Method B from 2.5M nBuLi (1.65 mL, 4.11 mmol), 2-chloro-6-(trifluoromethyl)pyridin-4-amine (1.5 g, 7.48 mmol), THF (37.4 mL), and methyl 2,3,3,3-tetrafluoro-2-(trifluoromethyl)propanoate (0.9 g, 3.74 mmol). Automated flash chromatography yielded the product as a clear oil (287.6 mg, 20% yield). 1H NMR (500 MHz, Chloroform-d) δ 9.59 (s, 1H), 8.05 (d, 4J=1.8 Hz, 1H), 7.99 (d, 4J=1.8 Hz, 1H). 13C NMR (126 MHz, Chloroform-d) δ 156.47 (d, 2J=19.7 Hz), 153.41, 149.38 (q, 2J=36.2 Hz), 146.14, 120.34 (q, 1J=274.5 Hz), 117.15, 115.20 (qd, 1J=287.7, 2J=26.5 Hz), 110.54 (q, 3J=3.1 Hz), 88.53 (dhept, 1J=221.7, 2J=32.8 Hz).

### Example 4: N-(2-chloro-6-(trifluoromethyl)pyridin-4-yl)-2,2,3,3,4,4,4-heptafluorobutanamide (GMR037)

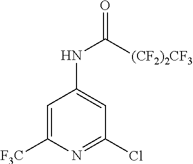

Prepared using General Procedure Method A from triethylamine (0.14 mL, 0.99 mmol), 2-chloro-6-(trifluoromethyl)pyridin-4-amine (0.1 g, 0.49 mmol), dichloromethane (1 mL), heptafluorobutyric anhydride (0.12 mL, 0.49 mmol), and 4-Dimethylaminopyridine (12 mg, 0.1 mmol). Automated flash chromatography yielded the product as a clear oil (0.19 mg, >99% yield). ¹H NMR (500 MHz, Methanol-d₄) δ 8.08 (d, ⁴J=1.5 Hz, 1H), 8.06 (d, ⁴J=1.5 Hz, 1H). “C NMR (126 MHz, Methanol-d₄) δ 156.93 (t, ²J=27.5 Hz), 152.75, 148.58 (q, ²J=35.8 Hz), 147.52, 120.56 (q, ¹J=273.5 Hz), 117.43 (qt, J¹=286.7, 286.7, ²J=33.5 Hz), 116.88, 110.40 (q, ³J=3.2 Hz), 110.63-105.58 (m).

### Example 5: Assay for Determining Repelleney and Vapor Toxicity

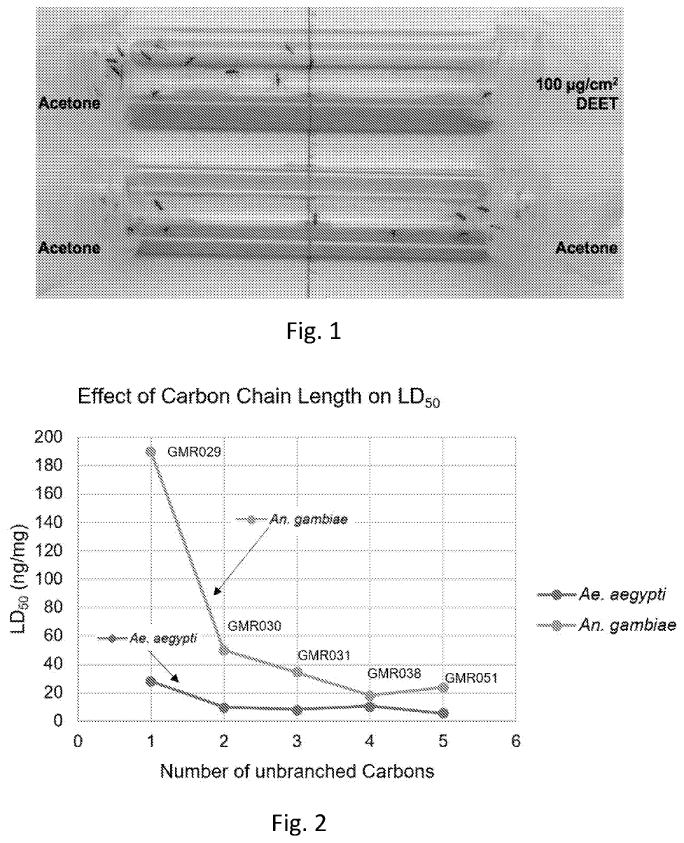

For assessing behavioral repellency (FIG. 1), Aedes aegypti mosquitoes were used from the insecticide-susceptible Orlando strain and the pyrethroid-resistant Puerto Rico strain. Sixteen female mosquitoes were transferred to a glass tube (12.5 cm long, 2.5 cm outer diameter) with the ends covered with netting. Each compound was dissolved in 1 mL of acetone and serial dilutions ranging from 30-0.01 μg/ml were made. Circular filter papers (5 cm²) were saturated with 50 μL of each concentration and allowed 10 min for solvent evaporation. The filter papers were then placed in clear conical polypropylene caps and fixed to the end of the glass tubes. The netting prevented mosquito contact with the filter paper. The tubes were then aligned with a mark denoting the midline and the proportion of mosquitoes on the chemically treated side was scored for up to 60 min. At the end of the test, the tube was placed in a cage and the ability of the mosquitoes to fly out of the tube was assessed for 30 min. For controls, after a 60 min observation period, all the mosquitoes left the tube within 10 min. This post-test evaluated whether any non-responding mosquitoes (not repelled) resulted from intoxication or disorientation from the test chemical. For assessment of lethality, insects were held for 24 hr under constant exposure in glass tubes having treated filter papers. The compounds analyzed in the following examples correspond with the general structures provided in Table 1.

TABLE 1

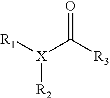

R₁

| | |
|---|---|
| 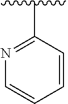 [SMILES: *c1ccccn1] | A |
| 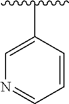 [SMILES: *c1cccnc1] | B |
| 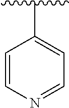 [SMILES: *c1ccncc1] | C |
| 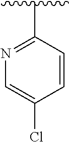 [SMILES: *c1ccc(Cl)cn1] | D |
| 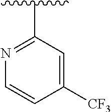 [SMILES: *c1cc(C(F)(F)F)ccn1] | E |
| 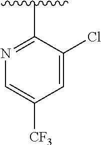 [SMILES: *c1ncc(C(F)(F)F)cc1Cl] | F |
| 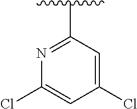 [SMILES: *c1cc(Cl)cc(Cl)n1] | G |
| 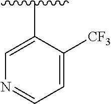 [SMILES: *c1cnccc1C(F)(F)F] | H |
| 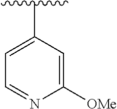 [SMILES: *c1ccnc(OC)c1] | I |
| 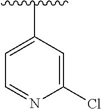 [SMILES: *c1ccnc(Cl)c1] | J |
| 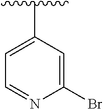 [SMILES: *c1ccnc(Br)c1] | K |
| 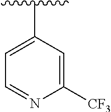 [SMILES: *c1ccnc(C(F)(F)F)c1] | L |
| 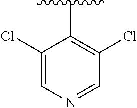 [SMILES: *c1c(Cl)cncc1Cl] | M |
| 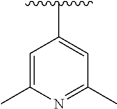 | N |
| 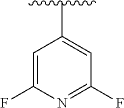 [SMILES: *c1cc(F)nc(F)c1] | O |
| 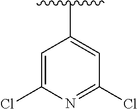 [SMILES: *c1cc(Cl)nc(Cl)c1] | P |
| 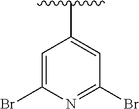 [SMILES: *c1cc(Br)nc(Br)c1] | Q |
| 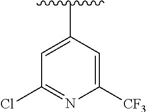 [SMILES: *c1cc(Cl)nc(C(F)(F)F)c1] | R |
| 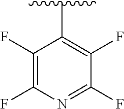 [SMILES: *c1c(F)c(F)nc(F)c1F] | S |
| 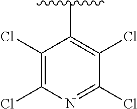 [SMILES: *c1c(Cl)c(Cl)nc(Cl)c1Cl] | T |
| 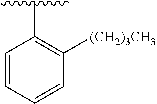 | U |
| 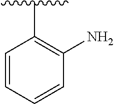 [SMILES: *c1ccccc1N] | V |
| 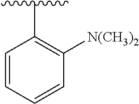 [SMILES: *c1ccccc1N(C)C] | W |
| 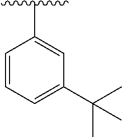 [SMILES: *c1cccc(C(C)(C)C)c1] | X |
| 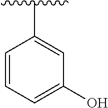 [SMILES: *c1cccc(O)c1] | Y |
| 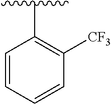 [SMILES: *c1ccccc1C(F)(F)F] | Z |
| 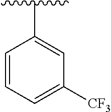 [SMILES: *c1cccc(C(F)(F)F)c1] | A′ |
| 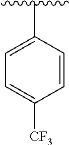 [SMILES: *c1ccc(C(F)(F)F)cc1] | B′ |
| 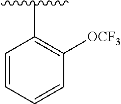 [SMILES: *c1ccccc1OC(F)(F)F] | C′ |
| 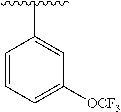 [SMILES: *c1cccc(OC(F)(F)F)c1] | D′ |
| 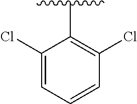 [SMILES: *c1c(Cl)cccc1Cl] | E′ |
| 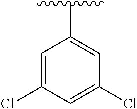 [SMILES: *c1cc(Cl)cc(Cl)c1] | F′ |
| 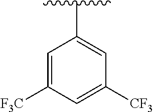 [SMILES: *c1cc(C(F)(F)F)cc(C(F)(F)F)c1] | G′ |
| 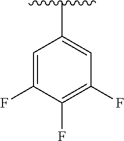 [SMILES: *c1cc(F)c(F)c(F)c1] | H′ |
| 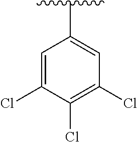 [SMILES: *c1cc(Cl)c(Cl)c(Cl)c1] | I′ |
| 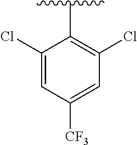 [SMILES: *c1c(Cl)cc(C(F)(F)F)cc1Cl] | J′ |
| 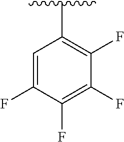 [SMILES: *c1cc(F)c(F)c(F)c1F] | K′ |
| 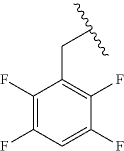 [SMILES: *Cc1c(F)c(F)cc(F)c1F] | L′ |
| 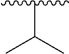 [SMILES: *C(C)C] | M′ |
| 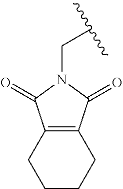 [SMILES: *CN1C(=O)C2=C(CCCC2)C1=O] | N′ |
| 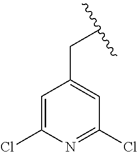 [SMILES: *Cc1cc(Cl)nc(Cl)c1] | O′ |
| 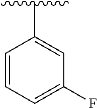 [SMILES: *c1cccc(F)c1] | P′ |
| 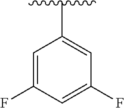 [SMILES: *c1cc(F)cc(F)c1] | Q′ |

R₂

| | |
|---|---|
| 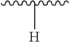 [SMILES: *[H]] | 1 |
| 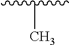 | 2 |
| 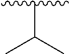 [SMILES: *C(C)C] | 3 |
| 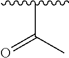 [SMILES: *C(C)=O] | 4 |
| 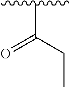 [SMILES: *C(=O)CC] | 5 |
| 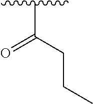 [SMILES: *C(=O)CCC] | 6 |
| 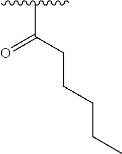 [SMILES: *C(=O)CCCCC] | 7 |
| 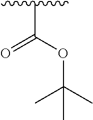 [SMILES: *C(=O)OC(C)(C)C] | 8 |
| 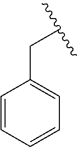 [SMILES: *Cc1ccccc1] | 9 |
| 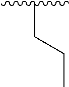 [SMILES: *CCC] | 10 |
| 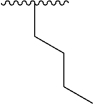 [SMILES: *CCCC] | 11 |
| 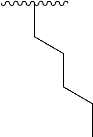 [SMILES: *CCCCC] | 12 |
| 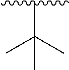 [SMILES: *C(C)(C)C] | 13 |
| 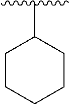 [SMILES: *C1CCCCC1] | 14 |
| 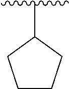 [SMILES: *C1CCCC1] | 15 |
| 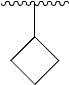 [SMILES: *C1CCC1] | 16 |
| 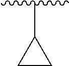 [SMILES: *C1CC1] | 17 |
| 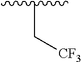 [SMILES: *CC(F)(F)F] | 18 |
| 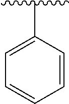 [SMILES: *c1ccccc1] | 19 |
| 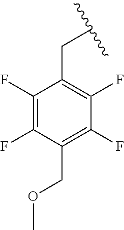 [SMILES: *Cc1c(F)c(F)c(COC)c(F)c1F] | 20 |
| 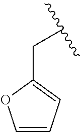 [SMILES: *Cc1ccco1] | 21 |
| 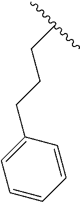 [SMILES: *CCCc1ccccc1] | 22 |
| 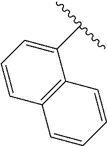 [SMILES: *c1cccc2ccccc12] | 23 |

R₃

| | |
|---|---|
| 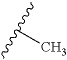 | a |
| 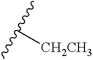 [SMILES: *CC] | b |
| 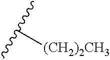 | c |
| 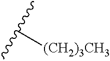 | d |
| 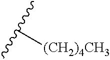 | e |
| 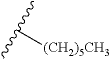 | f |
| 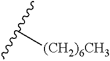 | g |
| 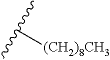 | h |
| 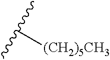 | i |
| 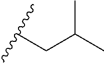 [SMILES: *CC(C)C] | j |
| 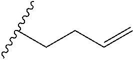 [SMILES: *CCC=C] | k |
| 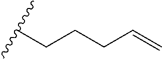 [SMILES: *CCCC=C] | l |
| 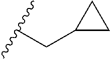 [SMILES: *CC1CC1] | m |
| 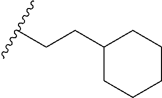 [SMILES: *CCC1CCCCC1] | n |
| 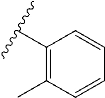 [SMILES: *c1ccccc1C] | o |
| 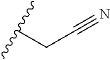 [SMILES: *CC#N] | p |
| 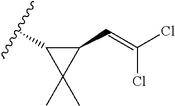 | q |
| 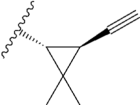 [SMILES: *[C@@H]1[C@@H](C#C)C1(C)C] | r |
| 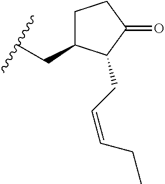 [SMILES: *C[C@H]1CCC(=O)[C@@H]1C/C=C\CC] | s |
| 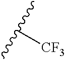 [SMILES: *C(F)(F)F] | t |
| 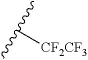 | u |
| 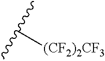 | v |
| 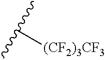 | w |
| 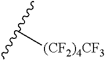 | x |
| 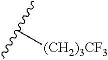 | y |
| 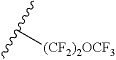 | z |
| 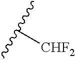 [SMILES: *C(F)F] | a′ |
| 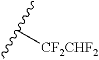 | b′ |
| 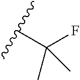 [SMILES: *C(C)(C)F] | c′ |
| 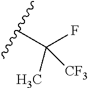 [SMILES: *C(C)(F)C(F)(F)F] | d′ |
| 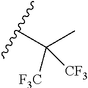 [SMILES: *C(C)(C(F)(F)F)C(F)(F)F] | e′ |
| 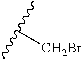 | f′ |
| 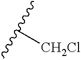 | g′ |
| 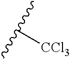 | h′ |
| 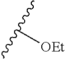 [SMILES: *OCC] | i′ |
| 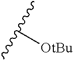 [SMILES: *OC(C)(C)C] | j′ |
| 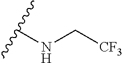 [SMILES: *NCC(F)(F)F] | k′ |
| 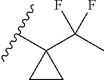 [SMILES: *C1(C(C)(F)F)CC1] | l′ |
| 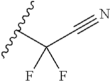 [SMILES: *C(F)(F)C#N] | m′ |
| 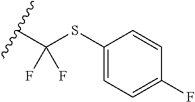 [SMILES: *C(F)(F)Sc1ccc(F)cc1] | n′ |
| 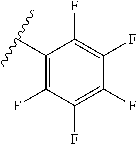 [SMILES: *c1c(F)c(F)c(F)c(F)c1F] | o′ |
| 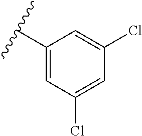 [SMILES: *c1cc(Cl)cc(Cl)c1] | p′ |
| 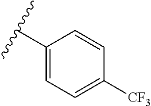 [SMILES: *c1ccc(C(F)(F)F)cc1] | q′ |
| 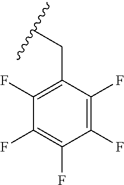 [SMILES: *Cc1c(F)c(F)c(F)c(F)c1F] | r′ |
| 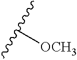 [SMILES: *OC] | s′ |
| 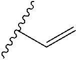 [SMILES: *C=C] | t′ |
| 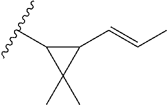 [SMILES: *C1C(/C=C/C)C1(C)C] | u′ |
| 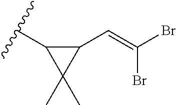 [SMILES: *C1C(C=C(Br)Br)C1(C)C] | v′ |
| 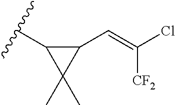 | w′ |
| 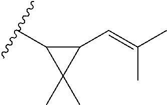 [SMILES: *C1C(C=C(C)C)C1(C)C] | x′ |
| 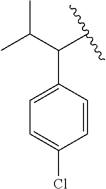 [SMILES: *C(c1ccc(Cl)cc1)C(C)C] | y′ |
| 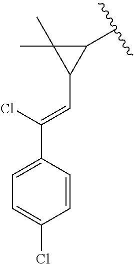 [SMILES: *C1C(/C=C(\Cl)c2ccc(Cl)cc2)C1(C)C] | z′ |
| 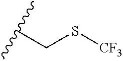 [SMILES: *CSC(F)(F)F] | a″ |
| 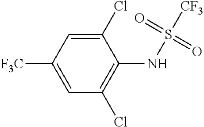 [SMILES: O=S(=O)(Nc1c(Cl)cc(C(F)(F)F)cc1Cl)C(F)(F)F] | MTFI-5-10a |
| 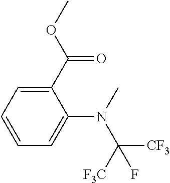 [SMILES: COC(=O)c1ccccc1N(C)C(F)(C(F)(F)F)C(F)(F)F] | MTFI-5-11a |
| 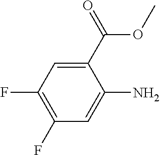 | MTFI-5-12a |
| 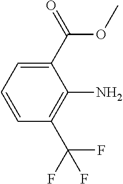 | MTFI-5-12b |
| 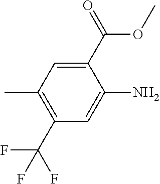 | MTFI-5-12c |
| 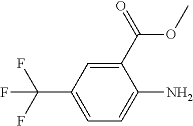 | MTFI-5-12d |
| 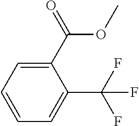 | MTFI-5-13a |
| 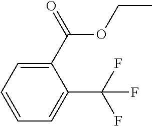 | MTFI-5-13b |
| 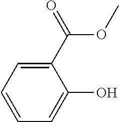 | MTFI-5-14a |
| 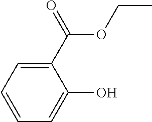 | MTFI-5-14b |
| 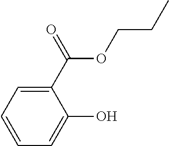 | CA-12.5 |
| 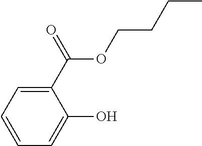 | MTFI-5-14c |
| 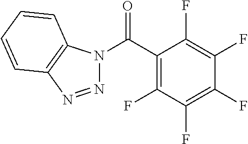 [SMILES: O=C(c1c(F)c(F)c(F)c(F)c1F)n1nnc2ccccc21] | MT2018-0314-01 |
| 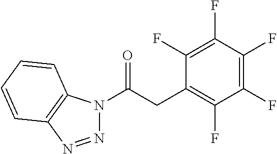 [SMILES: O=C(Cc1c(F)c(F)c(F)c(F)c1F)n1nnc2ccccc21] | MT2018-0314-02 |
| 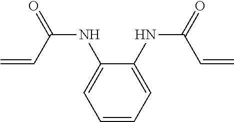 [SMILES: C=CC(=O)Nc1ccccc1NC(=O)C=C] | GMR024 |
| 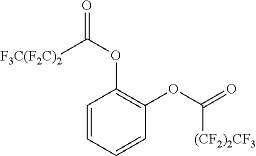 | GMR058 |
| 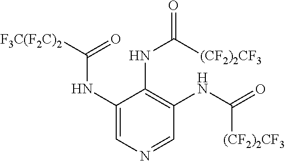 | GMR062 |
| 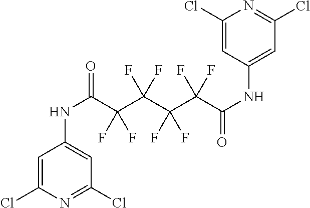 [SMILES: O=C(Nc1cc(Cl)nc(Cl)c1)C(F)(F)C(F)(F)C(F)(F)C(F)(F)C(=O)Nc1cc(Cl)nc(Cl)c1] | GMR077 |
| 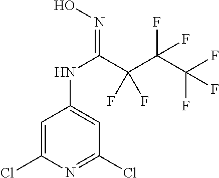 [SMILES: O/N=C(\Nc1cc(Cl)nc(Cl)c1)C(F)(F)C(F)(F)C(F)(F)F] | GMR104 |
| 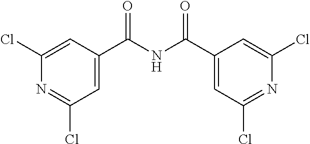 [SMILES: O=C(NC(=O)c1cc(Cl)nc(Cl)c1)c1cc(Cl)nc(Cl)c1] | GMR117 |

### Example 6: Vapor Repellency, Knockdown, and Toxicity

Compounds were first screened at 100 μg/cm² for repellency and 24 h mortality. The results are provided in Table 2. When knockdown was observed in 1 hr repellency tests, the notation, “XXkd” denotes the number of insects incapacitated out of 16 females exposed in the tube. The presence of knockdown precluded a good measurement of repellency at 100 μg/cm². Resistance ratio (RR)=24 h LC₅₀ PR/24 h LC₅₀ OR. Compounds showing negative repellency values were observed to be slightly attractive.

TABLE 2

| | | | | | | | | | 1 h | | | |
|---|---|---|---|---|---|---|---|---|---|---|---|---|
| | | | | | | | 1 h | | Repellency | | 24 h | |
| | | | | | 1 h % | 24 h % | Repellency | 24 h | EC₅₀, | | LC₅₀, | |
| | | | | | Repellency, | Mortality, | EC₅₀, | LC₅₀, | μg/cm² | | μg/cm² | |
| Name | X | R₁ | R₂ | R₃ | 100 μg/cm² | 100 μg/cm² | μg/cm² | μg/cm² | (RR) | | (RR) | |
| Metofluthrin | — | — | — | — | | | 0.3 | 0.84 | 12 | (40) | 43 | (51) |
| Transfluthrin | — | — | — | — | | | 0.5 | 0.16 | 2.3 | (4.6) | 7.6 | (48) |

| | | | | | | | |
|---|---|---|---|---|---|---|---|
| DEET | — | — | — | — | 35 | 79 | (2.3) |

| MTFI-2-1A | N | Z | 1 | g′ | 94 | | 100 | 14 | 14 | | | |
|---|---|---|---|---|---|---|---|---|---|---|---|---|
| MTFI-2-1B | N | Z | 1 | u | 38 | | 0 | | | | | |
| MTFI-2-1C | N | Z | 1 | f | 88 | | 3 | | | | | |
| MTFI-2-1D | N | Z | 1 | g | 42 | | 6 | | | | | |
| MTFI-2-1E | N | Z | 1 | h | 0 | | 0 | | | | | |
| MTFI-2-1F | N | Z | 1 | i | 6 | | 0 | | | | | |
| MTFI-2-2A | N | A′ | 1 | g′ | 50 | | 3 | | | | | |
| MTFI-2-2B | N | A′ | 1 | u | 38 | | | | | | | |
| MTFI-2-3A | N | J′ | 1 | g′ | 18 | | 12 | | | | | |
| MTFI-3-1A | N | G′ | 1 | a | 0 | | 21 | | | | | |
| MTFI-3-1B | N | G′ | 1 | e | 12 | | 29 | | | | | |
| MTFI-3-1C | N | G′ | 1 | l | 38 | | 0 | | | | | |
| MTFI-3-1D | N | G′ | 1 | n | 6 | | 7 | | | | | |
| MTFI-3-1E | N | G′ | 1 | o | 6 | | 7 | | | | | |
| MTFI-3-1F | N | G′ | 1 | g′ | 88 | | 100 | 11 | 35 | | | |
| MTFI-3-1G | N | G′ | 1 | t | 3 | kd | 100 | | 16 | | | |
| MTFI-3-1H | N | G′ | 1 | u | 4 | kd | 100 | | | | | |
| MTFI-3-2A | N | Z | 1 | a | 80 | | 22 | | | | | |
| MTFI-3-2B | N | Z | 1 | b | 84 | | | 25 | | 45 | | |
| MTFI-3-2C | N | Z | 1 | c | 88 | | | 25 | | 34 | | |
| MTFI-3-2D | N | Z | 1 | d | 76 | | | 31 | | 50 | | |
| MTFI-3-2E | N | Z | 1 | l | 76 | | | | | | | |
| MTFI-3-2F | N | Z | 1 | n | 18 | | 25 | | | | | |
| MTFI-1-4C | N | Z | 1 | t | −6 | | | | | | | |
| MTFI-1-4A | N | Z | 1 | e | 78 | | | 26 | | 81 | | |
| MTFI-3-3A | N | A′ | 1 | n | 16 | | | | | | | |
| MTFI-3-4A | N | B′ | 1 | a | 8 | | | | | | | |
| MTFI-3-4B | N | B′ | 1 | n | 8 | | | | | | | |
| MTFI-3-5A | N | J′ | 1 | n | 0 | | | | | | | |
| MTFI-4-3A | N | F′ | 1 | t | 60 | | 44 | | | | | |
| MTFI-4-3B | N | F′ | 1 | a′ | −6 | | | | | | | |
| MTFI-4-3C | N | F′ | 1 | u | 44 | | | | | | | |
| MTFI-4-3D | N | F′ | 1 | v | 66 | | | 14 | | 16 | | |
| MTFI-4-4A | N | I′ | 1 | t | 6 | | | | | | | |
| MTFI-4-4B | N | I′ | 1 | a′ | 16 | | | | | | | |
| MTFI-4-4C | N | I′ | 1 | u | 46 | | | | | | | |
| MTFI-4-4D | N | I′ | 1 | v | 30 | | | | | | | |
| MTFI-4-5A | N | E′ | 1 | t | 88 | | | 17 | | 33 | | |
| MTFI-4-5B | N | E′ | 1 | a′ | 60 | | | | | | | |
| MTFI-4-5C | N | E′ | 1 | u | 78 | | 75 | | | 34 | | |
| MTFI-4-6A | N | M | 1 | t | 30 | | | | | | | |
| MTFI-4-6B | N | M | 1 | a′ | 34 | | | | | | | |
| MTFI-4-6C | N | E′ | 1 | u | 13 | kd | 100 | 9 | 7.6 | 19 | 17 | (2.2) |
| MTFI-4-6D | N | M | 1 | v | 5.3 | kd | 100 | 19 | 21 | 29 | 24 | (1.1) |

| MTFI-4-7A | N | H | 1 | t | 24 | | 0 | 67 | | | |
|---|---|---|---|---|---|---|---|---|---|---|---|
| MTFI-4-7B | N | H | 1 | a′ | 66 | | | | | | |
| MTFI-4-7C | N | H | 1 | u | 46 | | | | | | |
| MTFI-4-7D | N | H | 1 | v | 80 | | 72 | | | | |
| MTFI-4-2B | N | G′ | 1 | a′ | 100 | | 88 | 15 | | 24 | |
| MTFI-4-2D | N | G′ | 1 | w | 12 | | | | | | |
| MTFI-4-1B | N | J′ | 1 | a′ | 54 | | | | | | |
| MTFI-4-1D | N | J′ | 1 | v | 6 | | | | | | |
| MTFI-4-FPL-A | N | J′ | 1 | k′ | 0 | | | | | | |
| MTFI-4-NA-1C | N | B′ | 1 | p′ | 8 | | | | | | |
| MTFI-5-1A | N | P′ | 1 | g′ | 6 | | | | | | |
| MTFI-5-3A | N | H′ | 1 | v | 68 | | 100 | 2.9 | 13 | 4.5 | 11 |
| MTFI-5-4A | N | H′ | 1 | a′ | 68 | | 0 | | | | |
| MTFI-5-4B | N | K′ | 1 | t | 82 | | 0 | | | | |
| MTFI-5-4C | N | K′ | 1 | b′ | 0 | | 0 | | | | |
| MTFI-5-4D | N | K′ | 1 | u | 0 | | 90 | | | | |
| MTFI-5-4E | N | K′ | 1 | v | 56 | | 6 | | | | |
| MTFI-5-5A | N | Z | 1 | c′ | 82 | | 3 | | | | |
| MTFI-5-5B | N | Z | 1 | p | 0 | | 0 | | | | |
| MTFI-5-5C | N | Z | 1 | m | 82 | | 0 | 34 | | | |
| MTFI-5-5D | N | Z | 1 | k | 56 | | 6 | | | | |
| MTFI-5-5E | N | Z | 1 | f′ | 62 | | 60 | | | | |
| MTFI-5-6C | N | A′ | 1 | m | 50 | | 0 | | | | |
| MT2018-0316-01 | N | A′ | 1 | o′ | 24 | | 0 | | | | |
| MT2018-0316-02 | N | A′ | 1 | r′ | 6 | | 0 | | | | |
| MTFI-5-7A | N | X | 1 | g′ | 6 | | 0 | | | | |
| MTFI-5-8A | N | U | 1 | d | 38 | | 6 | | | | |
| MTFI-5-8B | N | C′ | 1 | d | 96 | | 13 | 11 | | | |
| MTFI-5-9A | N | D′ | 1 | d | 50 | | 0 | | | | |
| MTFI-5-10A | — | — | — | — | 0 | | 0 | | | | |
| MTFI-5-11A | — | — | — | — | — | | — | | | | |
| MTFI-5-12A | — | — | — | — | 92 | | | 23 | | | |
| MTFI-5-512B | — | — | — | — | 42 | | | | | | |
| MTFI-5-12C | — | — | — | — | 30 | | | | | | |
| MTFI-5-12D | — | — | — | — | 84 | | | | | | |
| MTFI-5-13A | — | — | — | — | 6 | | | | | | |
| MTFI-5-13B | — | — | — | — | 12 | | | | | | |
| MTFI-5-14A | — | — | — | — | 70 | | | | | | |
| MTFI-5-14B | — | — | — | — | 30 | | | | | | |
| CA-12.5 | — | — | — | — | — | | — | | | | |
| MTFI-5-14C | — | — | — | — | 8 | kd | 69 | | | | |
| MTFI-5-15A | N | Y | 1 | g′ | 36 | | | | | | |
| MT2018-0314-01 | — | — | — | — | 32 | | 0 | | | | |
| MT2018-0314-02 | — | — | — | — | 6 | | 0 | | | | |
| GMR020 | N | Z | 1 | s′ | 16 | | 9 | | | | |
| GMR021 | N | Z | 1 | t′ | 92 | | | 32 | | | |
| GMR022 | N | V | 1 | t′ | −8 | | 16 | | | | |
| GMR023 | N | W | 1 | t′ | — | | — | | | | |
| GMR024 | — | — | — | — | 0 | | 3 | | | | |
| GMR028 | N | P | 1 | a′ | 12 | | 59 | | | | |
| GMR029 | N | P | 1 | t | 42 | | 24 | | 181 | | |
| GMR030 | N | P | 1 | u | 28 | | 85 | | 35 | | |
| GMR031 | N | P | 1 | v | 34 | | 98 | | 25 | | 22 |
| GMR069 | N | K′ | 1 | x | −12 | | 13 | | | | |
| GMR083 | N | P | 1 | q | 6 | | 0 | | | | |
| GMR085 | N | E | 1 | v | 70 | | 13 | 49 | | | |
| GMR090 | O | L′ | — | v | −12 | | 9 | | | | |
| GMR091 | N | R | 1 | z | 8 | | 100 | | | | |
| GMR093 | N | P | 1 | s | 30 | | 0 | | | | |
| GMR094 | N | M′ | 3 | v | — | | — | | | | |
| GMR095 | N | P | 2 | v | 84 | | 100 | 10 | 14 | | |
| GMR096 | N | N′ | 1 | v | | | | | | | |
| GMR097 | N | F′ | 1 | s | 16 | | 0 | | | | |
| GMR098 | N | H′ | 1 | s | 18 | | 0 | | | | |
| GMR099 | N | F′ | 1 | z | 74 | | 81 | | | | |
| GMR100 | N | H′ | 1 | z | 10 | kd | 21 | 3 | 15 | | |
| (screened at 10 | | | | | | | | | | | |
| μg/cm²) | | | | | | | | | | | |
| GMR102 | N | H′ | 1 | m′ | 74 | | 22 | 28 | | | |
| GMR104 | — | — | — | — | 12 | | 0 | | | | |
| GMR105 | N | R | 1 | v | 24 | | 21 | | | | |
| GMR106 | N | P | 2 | z | 2 | kd | 100 | | | | |
| GMR107 | N | O′ | 1 | v | 50 | | 0 | | | | |
| GMR113 | N | H′ | 2 | v | 0 | | 6 | | | | |
| GMR114 | N | H′ | 2 | z | 0 | | 6 | | | | |
| GMR117 | — | — | — | — | 62 | | 100 | | | | |
| GMR118 | N | R | 2 | v | 84 | | 100 | | | | |
| GMR129 | N | C′ | 1 | z | 34 | | | | | | |
| GMR130 | N | M | 1 | b′ | 34 | | 81 | | | | |
| GMR131 | N | M | 1 | b′ | −4 | | 6 | | | | |
| GMR132 | N | M | 1 | c′ | 4 | | 28 | | | | |
| GMR135 | N | H′ | 1 | u | 74 | | 100 | 22.1 | 21.6 | | |
| GMR136 | — | — | — | — | 32 | | 19 | | | | |
| GMR138 | N | J′ | 1 | u | 66 | | 100 | 5.3 | 0.3 | | |
| GMR139 | N | H′ | 1 | e′ | 24 | | 100 | 6.9 | 16.6 | | |
| GMR140 | O | — | 18 | q | 80 | | 100 | | | | |
| GMR141 | N | H′ | 1 | j | 16 | | 19 | | | | |
| GMR142 | N | H′ | 1 | a | 50 | | 66 | | | | |
| GMR143 | N | H′ | 1 | b | 88 | | 53 | 23 | 95 | | |
| GMR144 | N | H′ | 1 | c | 62 | | 38 | | | | |
| GMR146 | O | — | 20 | q | 15 | kd | 100 | | 1 | | |
| GMR148 | N | H′ | 1 | q | 12 | | 6 | | | | |
| GMR149 | N | J′ | 1 | t | 44 | | 81 | | | | |
| GMR150 | N | J′ | 8 | u | 11 | kd | 100 | | 12 | | |

| | | | | | | | | |
|---|---|---|---|---|---|---|---|---|
| GMR151 | N | J′ | 8 | t | 32 | 0 | | |
| GMR159 | N | D′ | 4 | a | −26 | 19 | | |
| GMR160 | N | D′ | 1 | b | 42 | 16 | 123 | |
| GMR161 | N | D′ | 5 | b | 74 | 72 | 36 | 52 |
| GMR162 | N | D′ | 1 | c | 50 | | | |
| GMR163 | N | D′ | 6 | c | 50 | | | |
| GMR164 | N | D′ | 1 | e | 38 | | | |
| GMR165 | N | D′ | 7 | e | 62 | | | |
| GMR169 | N | C′ | 4 | a | 82 | | | |
| GMR170 | N | C′ | 1 | b | 94 | | 15 | |
| GMR171 | N | C′ | 1 | c | 100 | | 29 | |
| GMR172 | N | C′ | 1 | e | 80 | | 14 | |
| GMR173 | — | — | — | — | 44 | | | |
| GMR177 | N | H′ | 1 | a″ | 30 | | | |
| GMR179 | | | | | 30 | — | — | — |
| GMR180 | | | | | −24 | 14.3 | | |
| GMR182 | | | | | − | 100 | 3.5 | 3 |
| GMR184 | | | | | −8 | 2 | — | — |
| GMR185 | | | | | − | 100 | — | 3.5 |
| GMR186 | | | | | −4 | 52.1 | — | — |
| GMR187 | | | | | 78 | 46 | — | 9.8 |
| GMR188 | | | | | − | 81 | — | 4.9 |
| GMR189 | | | | | 42 | 94 | 4.5 | 35.3 |
| GMR193 | | | | | 68 | 0 | — | — |
| GMR194 | | | | | 100 | 0 | 6.5 | — |
| GMR195 | | | | | −4 | 0 | — | — |
| GMR196 | | | | | 18 | 0 | — | — |
| GMR197 | | | | | 30 | 0 | — | — |
| GMR198 | | | | | 8 | 0 | — | — |

### Example 7: Mammalian Toxicity

Mice (inbred strain ICR, body weight between 20 and 26 g, 35 days old+/−4 days), were force fed with compounds solubilized in olive oil to a volume of 10 μL per gram of mouse via 1 mL disposable syringes attached to 22 gauge metal canulas directly in the stomach following the Institutional Animal Care and Use Committee (IACUC) standard procedure. One hour after gavage, mice were returned to food and water access ad libitum. Dose tested ranged from 60 up to 600 mg/kg of mice, following the method recommended by the US EPA to reduce the number of mice used to determine the LD₅₀ doses. Mice were monitored after drug administration during the first hour, and then every 4 hours up to 24 hours. The surviving mice were humanely sacrificed. Any mice displaying a high state of pain/distress would also be considered as not surviving the treatment and were sacrificed before the end of the investigation. The EC₅₀ was determined using the AOT425StatPgm software; the study was registered and approved by IACUC under number 201810416.

### Example 8: Analysis of Vapor Toxicity and Repellency

Representative compounds with high vapor repellency and selective toxicity are provided in Table 3A. Preliminary mouse screening studies based on the protocol of Example 7 indicated good selectivity.

TABLE 3A

| | 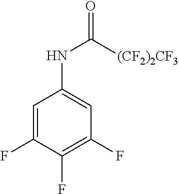 | 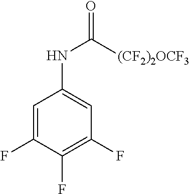 | 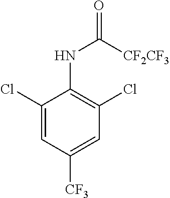 | 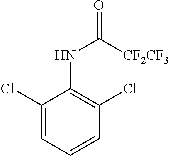 |
|---|---|---|---|---|
| | 52 | 51 | 41 | 53 |
| 1h % repellency^{a} | 68 | 100 | 66 | 13kd |
| 24h % mortalityª | 100 | 100 | 100 | 100 |
| 1h EC₅₀ (μg/cm²) | 2.92 | 3.09 | 5.30 | 8.90 |
| 24h LC₅₀ (μg/cm²) | 13.30 | 14.94 | 0.27 | 7.60 |
| Mouse LD₅₀ | >2000 | — | 200 | >2000 |

^{a}Screened at μg/cm²

Repellency did not directly correlate with EWG vs. EDG, as indicated in Table 3B. Hammett substitution constants (a) were based on the ionization of benzoic acids.

TABLE 3B

| | 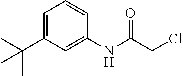 | 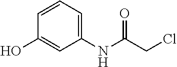 [SMILES: O=C(CCl)Nc1cccc(O)c1] | 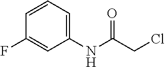 [SMILES: O=C(CCl)Nc1cccc(F)c1] | 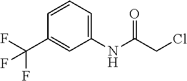 [SMILES: O=C(CCl)Nc1cccc(C(F)(F)F)c1] |
|---|---|---|---|---|
| | 1 | 2 | 3 | 4 |
| σₘₑₜₐ | −10 | 0.12 | 0.337 | 0.52 |
| 1h % repellency^{a} | 6 | 36 | 6 | 50 |
| 24h % mortality^{a} | 0 | — | — | 3.13 |
| 1h EC₅₀ (μg/cm²) | — | — | — | — |
| 24h LC₅₀ (μg/cm²) | — | — | — | — |

^{a}Screened at μg/cm²

### R₁

### Substituent Effects

Generally, an increase in electron withdrawing effect of groups meta to the amino group increased repellency activity. This was observed in the case of MTFI-5-7a vs. MTFI-5-15a vs. MTFI-5-1a MTFI-2-2a.

For the electron donating effect meta, electron donors typically gave increased repellency and mortality. This was observed in the case of MTFI-5-7A vs. MTFI-31F vs. MTFI-2-2A.

Generally, an increase in electron withdrawing groups ortho to the amino group increased repellency activity. This was observed in the case of GMR022 vs. GMR021.

The presence of a lone pair and increased volatility on the ortho group appeared to increase activity. This was observed in the case of MTFI-5-8B vs. MTFI-3-2D and MTFI-5-8D.

For phenyl substituents meta to the amino group, CF₃ was more effective than Cl. This was observed, for example, in the case of MTFI-4-3B vs. MTFI-4-2B, MTFI-4-3A vs. MTFI-3-1G, and MTFI-4-3C vs. MTFI-3-1H.

For phenyl substituents ortho to the amino group, electron withdrawing increased activity, as demonstrated by GMR022 vs. GMR021.

Substituents Ortho Vs. Meta to Amino Group

Generally, for phenyl derivatives, ortho substituted compounds exhibited better repellency. This was demonstrated by MTFI-4-5B vs. MTFI-4-3B, MTFI-4-5A vs. MTFI-4-3A, MTFI-4-6C vs. MTFI-4-3C, and MTFI-5-8B vs. MTFI-5-9A.

For pyridyl derivatives, ortho substitution generally exhibited slightly better repellency and mortality, as shown by MTFI-4-6B vs. GMR028, MTFI-4-6A vs. GMR029, and MTFI-4-6D vs. GMR031.

Degree of Ring Substitution not Including Amino Group

Derivatives with increased fluorine substitution generally exhibited greater activity, whereas increased substitution with other groups had the opposite effect. This was demonstrated by MTFI-2-2A vs. MTFI-3-1F, MTFI-3-3A vs. MTFI-3-1D, MTFI-4-3A vs. MTFI-4-4A, MTFI-4-3C vs. MTFI-4-4C, MTFI-4-3D vs. MTFI-4-4D, and MTFI-5-3A vs. MTFI-5-5D

Replacement of Pyridyl-N

In many cases, the introduction of an aromatic nitrogen to change phenyl to pyridyl decreased repellency, but in some cases increased toxicity. This was seen by MTFI-4-7A vs. MTFI-1-4C, and MTFI-4-7C vs. MTFI-MTFI-2-1B, MTFI-4-5B vs. MTFI-4-6B, MTFI-4-5A vs MTFI-4-6A, MTFI-4-3D vs. GMR031, MTFI-4-3B vs. GMR028, MTFI-4-3A vs. GMR029, MTFI-4-3C vs. GMR030, and GMR099 vs. GMR106.

Generally, the substitution of pyridyl-N with C—Cl afforded little to no change in repellency, but led to loss of mortality. This was observed, for example by MTFI-4-4B vs. GMR028, MTFI-4-4A vs. GMR029, MTFI-4-4C vs. GMR030, and MTFI-4-4D vs. GMR031.

The substitution of pyridyl-N with C—CF₃ led to a slight increase in repellency and no change in mortality, as demonstrated by MTFI-4-1B vs. MTFI-4-6B and GMR149 vs. MTFI-4-6A.

The extension of the aromatic ring away from X with the insertion of saturated carbon had little effect on repellency, but drastically reduced toxicity, as demonstrated by GMR031 vs. GMR107.

### R2

The replacement of H with Di-tert-butyl dicarbonate (Boc) led to a reduction of repellency and mortality, as shown by GMR149 vs. GMR151 and GMR138 vs. GMR150.

The replacement of H with short a alkyl group increased both repellency and mortality, as shown by GMR031 vs. GMR095 and GMR105 vs. GMR118.

The di-amide exhibited little to no change in repellency, as demonstrated by GMR158 through GMR165.

### R3

Extension of the Unbranched Substituted Alkyl Chain

For cases where R₁ had a lone pair or hydrogen para to the amino group, the optimal length appeared to be a 3-carbon long chain. In instances where other groups were present para to the amino group attachment, steric hinderance was less tolerated and the optimal R₃ chain length was shorter. This was demonstrated by GMR169 vs. GMR170 vs. GMR171 vs. MTFI-5-8B vs. GMR172; GMR158 vs. GMR160 vs. GMR162 vs. MTFI-5-9A vs. GMR164; GMR142 vs. GMR143 vs. GMR144; and MTFI-3-2A vs. MTFI-3-2B vs. MTFI-3-2C vs. MTFI-3-2D vs. MTFI-1-4A.

Branched Derivatives

Branching appeared to reduce repellency and mortality, as demonstrated by GMR144 vs. GMR141, MTFI-3-2C vs MTFI-5-5C, and MTFI-2-1C vs. MTFI-3-2F.

Short alkyl cycles provided greater repellency than longer/larger groups, as shown by MTFI-5-6C and MTFI-5-5C vs. MTFI-3-3A.

Several examples with aromatic rings produced poor results, as shown by MTFI-3-1E, MT2018-0316-01, MTFI-4-NA-1C, and MT201-0316-02.

The introduction of non-aromatic unsaturated bonds led to a very slight loss of repellency in most cases, as shown by MTFI-3-2D vs. MTFI-5-5D, MTFI-3-1B vs. MTFI-3-1C, MTFI-1-4A vs. MTFI-3-2E, and MTFI-3-2B vs. GMR021.

Extension of Unbranched Halogenated Alkyl Chain

In many cases, Cl>Br, as shown by MTFI-5-5E vs. MTFI-2-1A.

Increasing the length of C and the degree of fluorination increased repellency and mortality. In some cases, a slight decrease in repellency was observed after C=3, as shown by MTFI-4-5B vs. MTFI-4-5A vs. MTFI-4-6C; MTFI-4-3B vs. MTFI-4-3A vs. MTFI-4-3C vs. MTFI-4-3D; MTFI-4-2B vs. MTFI-3-1G vs. MTFI-3-1H; MTFI-5-4A vs. GMR135; MTFI-4-4B vs. MTFI-4-4A vs. MTFI-4-4C vs. MTFI-4-4D; MTFI-4-1B vs. GMR149 vs. GMR138 vs. MTFI-4-1D; MTFI-4-6B vs. MTFI-4-6A vs. MTFI-4-6D; and GMR028 vs. GMR029 vs. GMR030 vs. GMR031.

Branched Fluorinated Derivatives

The branched chain derivatives with varying degrees of fluorination demonstrated reduced repellency, as seen by MTFI-5-5A and GMR139.

Introduction of Heteroatoms

The carbamate derivative exhibited less repellency and mortality, as shown by MTFI-3-2B vs. GMR020.

The ester linkage of fluorinated carbon chains did not diminish activity and it increased mortality in certain cases, as shown by MTFI-4-3D vs. GMR099 and GMR105 vs. GMR091.

Generally, fluorinated Urea derivatives exhibited reduced repellency, as shown by MTFI-4-FPL-A.

Fluorinated cyano derivatives exhibited moderate repellency and toxicity but non fluorinated derivatives demonstrated little to no activity, as shown by GMR102 vs. MTFI-5-5A.

Generally, the non-fully fluorinated thioether exhibited poor activity, as shown by GMR177.

Separate Related Structures

The di-amide structure exhibited reduced repellency and poor toxicity, as shown by GMR024.

The conversion of the amide to amide oxime led to a reduction in activity, as shown by GMR031 vs GMR104.

Several derivatives with reversed placement of nitrogen and carbonyl positions exhibited some repellency and mortality, as indicated by GMR117 and GMR173.

The sulfonamide led to a reduction of repellency and mortality, as demonstrated by GMR149 vs. MTFI-5-10A.

The ester and fluorine containing aniline derivatives exhibited moderate repellency in some instances, as shown by MTFI-5-11A, MTFI-5-12A, MTFI-5-12B, MTFI-5-12C, and MTFI-5-12D.

The long chain hydroxy benzoate derivatives exhibited moderate repellency and toxicity, as demonstrated by MTFI-5-14A, MTFI-5-14B, MTFI-5-14C.

### Example 9: Topical Toxicity

The Aedes aegypti strains Orlando (sensitive) as well as the Puerto Rico (PR, resistant to permethrin) were used. The Anopheles gambiae (G3 sensitive strain, as well as Akron kdr (Akdr) strain, resistant to pyrethroids were also assessed. The larvae were provided by the USDA, Gainesville Fl, and raised in a pan with tap water maintained at 28° C. Larvae were fed a ground liver and yeast mixture, and pupae were placed in emergence cages. Adults were held at 28° C. and provided with cotton balls soaked in 10% sugar water for rearing and for all bioassays.

Female adults from 3 to 6 days were used for the experiments. The drug was dissolved in ethanol and then applied with a micro-applicator on the thorax, between the legs: 200 nL of drug per female mosquito, with different concentrations of the drug; 10 mosquitos were treated together per concentration per replicate, and put together in a small container with free access to sweet water. Mortality, i.e. no visible movement, was assessed 24 hours after treatment. Every compound was assessed on at least 3 different cohorts of mosquitoes, and the LD₅₀ values and slopes were determined with Probit analysis, corrected with Abbott's formula.

The topical toxicity (24 hour mortality) of substituted aryl and pyridyl amides and related analogs is provided in Table 4. Resistance ratio (RR)=24 h LD₅₀ resistant strain/24 h LD₅₀ susceptible strain.

TABLE 4

| | | | | | An. gambiae | | | Ae. aegypti | | |
|---|---|---|---|---|---|---|---|---|---|---|
| | | | | | G3 | AKDR | | OR | PR | |
| | | | | | LD₅₀ | LD₅₀ | | LD₅₀ | LD₅₀ | |
| Name | X | R₁ | R₂ | R₃ | (ng/mg) | (ng/mg) | RR | (ng/mg) | (ng/mg) | RR |
| Propoxur | — | — | — | — | 3.2 | 201 | 63 | 3.0 | | 0.0 |
| GMR028 | N | P | 1 | a′ | 43.1 | 28.2 | 0.7 | 266 | 854 | 3.2 |
| GMR029 | N | P | 1 | t | 27.9 | 45.3 | 1.6 | 190 | 238 | 1.3 |
| GMR030 | N | P | 1 | u | 9.6 | 14.2 | 1.5 | 49 | 50 | 1.0 |
| GMR031 | N | P | 1 | v | 8.0 | 6.7 | 0.8 | 34 | 31 | 0.9 |
| GMR032 | N | P | 1 | I′ | 18.3 | 66.0 | 3.6 | 892 | | |
| GMR033 | N | P | 1 | j′ | 21.4 | 46.7 | 2.2 | 1199 | | |
| GMR034 | N | R | 1 | a′ | 20.8 | 23.8 | 1.1 | 1454 | | |
| GMR035 | N | R | 1 | t | 30.5 | 52.1 | 1.7 | 106 | | |
| GMR036 | N | R | 1 | u | 13.5 | 10.0 | 0.7 | 25 | | |
| GMR037 | N | R | 1 | v | 6.5 | 5.3 | 0.8 | 17 | | |
| GMR038 | N | P | 1 | w | 10.4 | 6.7 | 0.6 | 18 | | |
| GMR039 | N | Q | 1 | a′ | 20.8 | 23.8 | 1.1 | 2253 | | |
| GMR040 | N | Q | 1 | t | >100 | >100 | | 1223 | | |
| GMR041 | N | Q | 1 | u | ~100 | ~100 | | 50 | | |
| GMR042 | N | Q | 1 | v | 11.4 | 4.7 | 0.4 | 24 | | |
| GMR043 | N | O | 1 | a′ | >100 | >100 | | 2480 | | |
| GMR044 | N | O | 1 | t | >100 | >100 | | 1996 | | |
| GMR045 | N | O | 1 | u | >100 | >100 | | 302 | | |
| GMR046 | N | O | 1 | v | >100 | >100 | | 300 | | |
| GMR047 | N | N | 1 | a′ | >100 | >100 | | >3000 | | |
| GMR048 | N | N | 1 | t | >100 | >100 | | >1500 | | |
| GMR049 | N | N | 1 | u | >100 | >100 | | >3000 | | |
| GMR050 | N | N | 1 | v | >100 | >100 | | >3000 | | |
| GMR051 | N | P | 1 | x | 5.2 | 3.7 | 0.7 | 23.4 | | |
| GMR052 | N | P | 1 | y | >100 | >100 | | 2743 | | |
| GMR053 | N | P | 1 | d′ | 5.8 | 2.7 | 0.5 | 17 | 7.10 | 0.4 |
| GMR054 | N | P | 1 | e | >100 | >100 | | >3000 | | |
| GMR055 | N | R | 1 | x | 11.0 | 4.0 | 0.4 | 10.5 | 5.90 | 0.6 |
| GMR056 | N | Q | 1 | x | 8.6 | 3.2 | 0.4 | 15 | 12.10 | 0.8 |
| GMR057 | N | S | 1 | x | 28.4 | 15.7 | 0.6 | 20 | 8.90 | 0.4 |
| GMR058 | — | — | — | — | | | | 6169 | | |
| GMR059 | N | P | 1 | h′ | | | | 866 | | |
| GMR060 | N | A | 1 | v | | | | 1758 | | |
| GMR062 | — | — | — | — | | >100 ng | | >3000 | | |
| GMR063 | N | B | 1 | v | | >100 | | >3000 | | |
| GMR064 | N | C | 1 | v | 1800 | >100 | | 1911 | | |
| GMR065 | N | S | 1 | e | | 1225 | | 1225 | | |
| GMR067 | N | R | 1 | e | | 3506 | | 3506 | | |
| GMR068 | N | J | 1 | x | | 3425 | | 3425 | | |
| GMR069 | N | K | 1 | x | | 3425 | | 2261 | | |
| GMR070 | N | L | 1 | x | | 1261 | | 1261 | | |
| GMR071 | N | I | 1 | x | | | | >2000 | | |
| GMR072 | N | O | 1 | x | 11.00 | 17 | 1.5 | 114 | | |
| GMR073 | N | N | 1 | x | | | | >3000 | | |
| GMR074 | N | T | 1 | v | 11.00 | 8.6 | 0.8 | 29 | | |
| GMR076 | N | P | 1 | l′ | | >100 | | 719 | | |
| GMR077 | — | — | — | — | | >100 | | 5373 | | |
| GMR078 | N | G | 1 | v | | >100 | | 5668 | | |
| GMR079 | N | D | 1 | v | | >100 | | >1000 | | |
| GMR080 | N | F | 1 | v | | >100 | | >1000 | | |
| GMR081 | N | S | 1 | t | | >100 | | 698 | | |
| GMR083 | N | P | 1 | q | >100 | >50 | | >6000 | | |
| GMR084 | N | P | 1 | z | 6.04 | 11.00 | 1.8 | 17.8 | | |
| GMR085 | N | E | 1 | v | >100 | >50 | | >6000 | | |
| GMR086 | N | P | 1 | r | >50 | >50 | | >6000 | | |
| GMR087 | N | P | 8 | v | 23.00 | 20.50 | 0.9 | 54.6 | | |
| GMR089 | N | P | 1 | o′ | >100 | >100 | | >6000 | | |
| GMR090 | O | L′ | — | v | >100 | >100 | | >1000 | | |
| GMR091 | N | P | 1 | z | 13.70 | 3.60 | 0.3 | 14.9 | | |
| GMR092 | N | R | 1 | d′ | 9.60 | 3.30 | 0.3 | 8.8 | | |
| GMR093 | N | P | 1 | s | >100 | >100 | | >6000 | | |
| GMR094 | N | M′ | 3 | v | >100 | | | 655 | | |
| GMR095 | N | P | 2 | v | 1300.00 | 775.00 | 0.6 | 1576 | | |
| GMR096 | O | N′ | — | v | >100 | | | 2538 | | |
| GMR097 | N | F′ | 1 | s | | | | >6000 | | |
| GMR098 | N | H′ | 1 | s | >1000 | >1000 | | >6000 | | |
| GMR099 | N | F′ | 1 | z | >1000 | >1000 | | 3279 | | |
| GMR100 | N | H′ | 1 | z | >1000 | >1000 | | 2374 | | |
| GMR101 | N | P | 1 | m′ | >100 | >100 | | 1841 | | |
| GMR102 | N | H′ | 1 | m′ | >100 | >100 | | 2164 | | |
| GMR103 | N | R | 1 | m′ | | | | | | |
| GMR104 | — | — | — | — | >100 | | | 393 | | |
| GMR106 | N | P | 2 | z | >1000 | | | 846 | | |
| GMR107 | N | O′ | 1 | v | | | | 2173 | | |
| GMR117 | — | — | — | — | | | | >1500 | | |
| GMR118 | N | R | 2 | v | | | | 321 | | |
| GMR119 | N | P | 8 | t | | | | 343 | | |
| GMR129 | N | C′ | 1 | z | >1000 | ~1000 | | >6000 | | |
| GMR135 | N | H′ | 1 | u | | | | 1792 | | |
| GMR137 | N | F′ | 1 | v | | | | | | |
| GMR138 | N | J′ | 1 | u | 120.00 | 90.00 | 0.8 | 57.6 | | |
| GMR145 | N | P | 1 | j | | | | 775 | | |
| GMR147 | N | P | 1 | n′ | | | | 1983 | | |
| GMR149 | N | J′ | 1 | t | | | | 251 | | |
| GMR150 | N | J′ | 8 | u | | | | 629 | | |
| GMR151 | N | J′ | 8 | t | | | | >5000 | | |
| GMR159 | N | D′ | 4 | a | >1000 | ~1000 | | | | |
| GMR162 | N | D′ | 1 | c | >1000 | >1000 | | | | |
| GMR163 | N | D′ | 6 | c | >1000 | >1000 | | | | |
| GMR169 | N | C′ | 4 | a | >1000 | >1000 | | | | |
| MTFI-4-6D | N | M | 1 | v | | | | >1000 | | |
| GMR174 | | | | | | | | 500 | | |
| GMR177 | | | | | | | | 220 | | |

### Example 10: Analysis of Topical Toxicity

Representative compounds for the topical toxicity analysis are provided in Table 5. Preliminary mouse screening studies indicate good selectivity.

TABLE 5

| | | 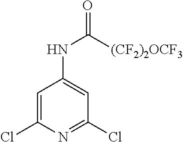 | 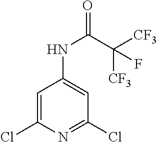 [SMILES: O=C(Nc1cc(Cl)nc(Cl)c1)C(F)(C(F)(F)F)C(F)(F)F] | 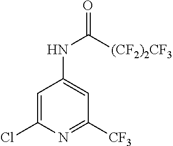 | 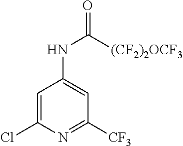 |
|---|---|---|---|---|---|
| | | 54 | 55 | 56 | 57 |
| An. | G3 LD₅₀ (ng/mg) | 6.04 | 5.80 | 6.50 | 13.70 |
| gambiae | AKDR LD₅₀ | 11.00 | 2.70 | 5.30 | 3.60 |
| | (ng/mg) | | | | |
| Ae. | OR LD₅₀ (ng/mg) | 17.80 | 17.20 | 17.30 | 14.90 |
| aegypti | LD₅₀ (ng/mg) | — | 7.10 | — | — |
| Mouse | LD₅₀ (ng/mg) | >2000 | ~50 | — | >200 |

### R₁

Pyridyl-N; the exclusion of nitrogen showed a decrease in mortality, as demonstrated by GMR097 vs. GMR93 and GMR099 vs. GMR084.

Position of the Nitrogen Around 6-Membered Ring

For preferential positioning of the amine ortho and para to pyridyl-N, para typically demonstrated better activity, as shown by GMR60 vs. GMR63 vs. GMR64; GMR078 vs. GMR031; and GMR080 & GMR078 vs GMR031.

Substituent Effects

Ortho vs. para substitution in respect to the 4-amino group demonstrated reduced toxicity, as shown by MTFI-4-5D vs. GMR031.

The degree of ring substitution other than the amino group: di-substituted, afforded good activity, as shown by GMR068 vs. GMR051, GMR031 vs GMR074, and GMR069 vs. GMR056.

In cases where pyridyl-N was para to the amino group, the strongly electron withdrawing substituents led to higher mortality. Potentially, steric crowding could offer more of a disadvantage in Ae. aegypti vs. An. Gambiae. This was shown in the case of GMR071 vs. GMR068 vs. GMR069 vs. GMR070; GMR047 vs. GMR043 vs. GMR028 vs. GMR039 vs. GMR034; GMR048 vs. GMR044 vs. GMR029 vs. GMR040 vs. GMR035; GMR049 vs. GMR045 vs. GMR030 vs. GMR041 vs. GMR046; GMR050 vs. GMR046 vs. GMR031 vs. GMR042 vs. GMR037; GMR072 vs. GMR057; and GMR031 vs GMR074.

The substitution of pyridyl-N with a C-heteroatom led to a reduction in activity, as shown by GMR045 vs GMR135.

Connectivity of R₁ to X

The extension of the aromatic group led to a reduction in activity, as shown by GMR107 vs. GMR031.

The alkyl R₁ attached to X led to a reduction in activity, as demonstrated by GMR094

R₂

H vs. alkyl; a reduction in activity was observed with removal of H, as demonstrated by GMR031 vs. GMR095, GMR084 vs. GMR086, and GMR037 vs GMR118.

H vs. ester; about a 2-fold loss in activity was observed with the introduction of ester. This was shown in the case of GMR149 vs. GMR151, GMR138 vs. GMR150, GMR029 vs GMR119, and GMR031 vs GMR087.

R₃

Chain Substituents

Increased fluorination increased mortality, as shown by GMR054 vs. GMR051, GMR067 vs. GMR055, GMR065 vs. GMR057, GMR028 vs. GMR029, and GMR038 vs. GMR052.

Chain Length

An increase in the fluorinated carbon chain length generally increased mortality (FIG. 2).

Branched Chains

Branched fully fluorinated chains retained high mortality, with non-fluorine saturated branched chains showing reduced activity. This was demonstrated by GMR053 and GMR092 vs. GMR054 and GMR067.

Chlorination vs. Fluorination

Chlorine derivatives appeared to exhibit poorer mortality than fluorinated derivatives, as shown by GMR029 vs. GMR059.

Non-Fluorine Saturated Chains and Introduction of Heteroatoms

Generally, a decrease in fluorine on the α-carbon decreased activity for An. gambiae and in many Ae. Aegypti, as shown by GMR047 vs. GMR048, GMR043 vs. GMR044, GMR028 vs. GMR029, GMR039 vs. GMR040, and GMR034 vs. GMR035.

Branching with limited fluorination derivative exhibited reduced toxicity, as shown in the case of GMR076.

Introduction of Heteroatoms and Unsaturated Carbon Bonds

TFA-like structures exhibited reduced toxicity, as seen by GMR083 and GMR086.

The methyl jasmonate derivative exhibited reduced toxicity, as shown by GMR093, GMR097, and GMR098.

The cyano moiety led to a decrease in activity, as demonstrated by GMR028 vs. GMR101.

The ether linkage led to an increase in activity, as shown by GMR084 vs. GMR031 and GMR038.

Nonfluorinated carbamates showed promising activity, as demonstrated by GMR032 and GMR033.

Aromatic group introduction led to a reduction in toxicity, as demonstrated by GMR089 and GMR147.

Separate Related Structures

Potential Multiple Bind Sites

The di-ester fluorinated derivative exhibited reduced activity, as shown by GMR058.

The tri-amide pyridine had reduced activity, as shown by GMR062.

The di-amide pyridyl exhibited reduced activity, as shown by GMR077 and GMR117.

Amidine led to reduced activity, as demonstrated by GMR031 vs. GMR104.

### Example 11: Synergistic Combinations with Pyrethroid Acids

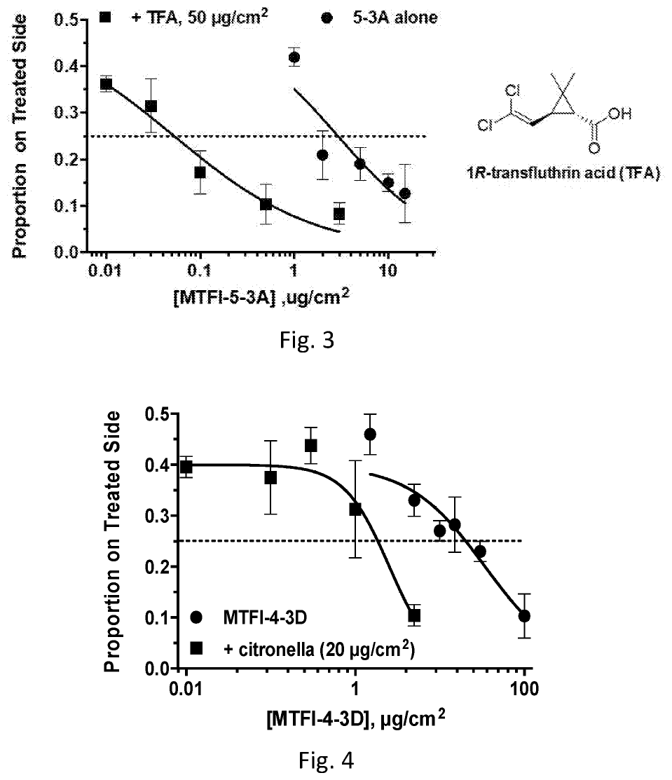

Additional experiments documented a synergistic repellent effect between certain derivatives of phenyl amides, such as MTFI-5-3A and 1R-transfluthrin acid (TFA), as shown in FIG. 3. MTFI-5-3A alone exhibited an EC₅₀ value of 2.9 μg/cm² (Table 1). With the addition of the pyrethroid acid TFA, which is inactive alone, MTFI-5-3A had an EC₅₀=0.05, an increase in potency of 60-fold.

### Example 12: Synergistic Combinations with Essential Oils

Synergistic repellent effects were also observed between certain phenyl amides, such as MTFI-4-3D and the essential oil citronella, as shown in FIG. 4. MTFI-4-3D alone had an EC₅₀ value of 18 μg/cm² (Table 1). With the addition of citronella at an EC₂₀ level, the EC₅₀ was 2.5, a 7-fold increase in potency.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of controlling and/or repelling one or more arthropods, comprising exposing said one or more arthropods to a composition containing a compound of Formula IA, IB, IC, ID, IE, IF, or IG and a carrier, wherein the compound of Formula IA, IB, IC, ID, IE, IF, or IG comprises: 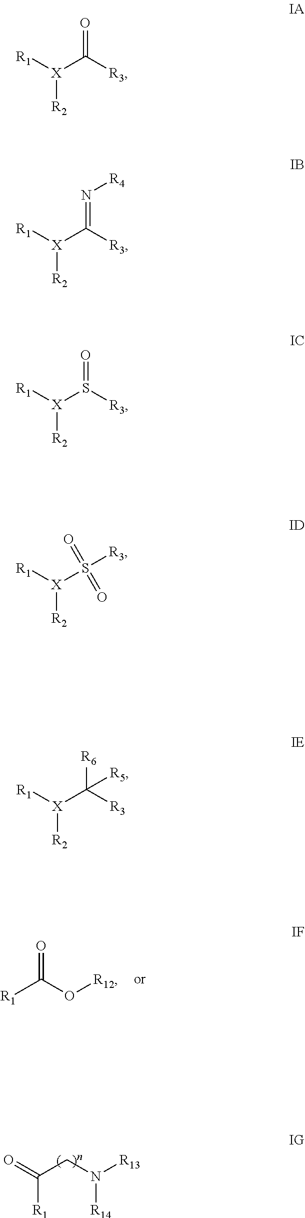
wherein,
X is O, N, or C;
R₁ is selected from the group consisting of linear or branched C₁-C₆ alkyl, 5- to 7-membered heteroaryl, (5- to 7-membered heteroaryl)-C₁-C₃ alkyl, phenyl, benzyl, C₃-C₇ cycloalkyl, and 5- to 10-membered heterocyclyl, (5- to 10-membered heterocyclyl)-C₁-C₃ alkyl;
wherein said heteroaryl, phenyl, benzyl, cycloalkyl, or heterocycyl is substituted with zero, one, two, three, four, or five substituents, each independently selected from the group consisting of halogen, C₁-C₆ alkyl, alkoxy, haloalkyl, hydroxy, haloalkoxy, cyano, —C(═O)R₇, —OC(═O)R₈, —NHC(═O)R₉, and —NR₁₀R₁₁; where R₇, R₈, R₉, R₁₀, and R₁₁ are each independently selected from the group consisting of hydrogen, C₁-C₆ alkyl, alkoxy, alkene, and haloalkyl;
R₂ is present or absent, and when present, is selected from the group consisting of hydrogen, linear or branched C₁-C₁₂ alkyl, alkene, —C(O)R₁₆, —C(O)OR₁₇, haloalkyl, C₃-C₇ cycloalkyl, C₆-C₁₀ aryl, (C₆-C₁₀ aryl)-C₁-C₃ alkyl, 5- to 10-membered heterocyclyl, and (5- to 10-membered heterocyclyl)-C₁-C₃ alkyl, wherein R₁₆ and R₁₇ are each independently selected from the group consisting of hydrogen, linear or branched C₁-C₁₂ alkyl, and alkene; and said C₆-C₁₀ aryl or (C₆-C₁₀ aryl)-C₁-C₃ alkyl is substituted with zero, one or two substituents, each independently selected from the group consisting of halogen, C₁-C₆ alkyl, alkylalkoxy, and haloalkyl;
R₃ is selected from the group consisting of C₁-C₁₂ alkyl, haloalkyl, alkoxy, haloalkoxy alkene, cyano, alkyl-haloalkoxy, NR₂₁R₂₂, haloalkyl-haloalkoxy, cyano-alkyl, (C₃-C₇ cycloalkyl)-alkyl, C₃-C₇ cycloalkyl, (C₆-C₁₀ aryl)-C₁-C₃ alkyl, phenyl, 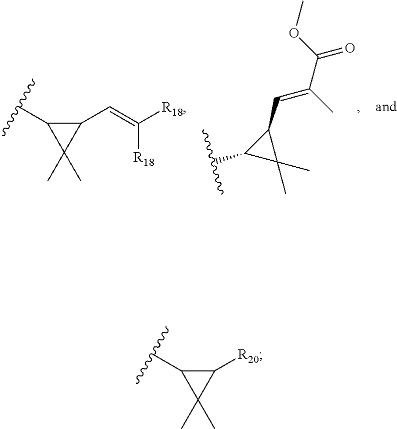
and where R₁₈, R₁₉, R₂₀, R₂₁, and R₂₂ are each independently selected from the group consisting of cyano, C₁-C₃ alkyl, C₂-C₈ alkyne, halogen, haloalkyl, hydrogen, and phenyl, wherein said phenyl is substituted zero, one, two, or three times with halogen;
wherein said “alkyl” in cyano-alkyl is substituted with halogen or not substituted;
0-3 carbons in said “haloalkyl” is are replaced with S; and
said cycloalkyl, phenyl, (C₆-C₁₀ aryl)-C₁-C₃ alkyl, or (C₃-C₇ cycloalkyl)-alkyl is substituted with zero, one, two, three, four, or five substituents, each independently selected from the group consisting of alkene, haloalkyl, and halogen; and
wherein the “alkyl” in (C₃-C₇ cycloalkyl)-alkyl or (C₆-C₁₀ aryl)-C₁-C₃ alkyl is substituted zero, one, two, or three times, in each instance, with linear or branched C₁-C₃ alkyl, sulfur, oxygen, or halogen;
R₄ is selected from the group consisting of hydrogen, C₁-C₆ alkyl, haloalkyl, alkene, alkyne, hydroxy, alkoxy, haloalkoxy, and NR₂₄R₂₅, wherein R₂₄ and R₂₅ are each independently selected from the group consisting of hydrogen, halogen, haloalkyl, and C₁-C₃ alkyl;
R₆ and R₅ are each independently selected from the group consisting of hydrogen, hydroxy, haloalkyl, halogen, and C₁-C₃ alkyl;
or,
R₆ and R₅, together with the carbon to which each is attached, form a C₃-C₇ cycloalkyl or a C₅-C₁₀ heterocycyl;
R₁₂ is hydrogen or linear or branched C₁-C₁₂ alkyl;
R₁₃ and R₁₄ are each independently selected from the group consisting of hydrogen, linear or branched C₁-C₃ alkyl, haloalkyl, alkyl, alkene, —C(═O)R₂₆, where R₂₆ is selected from the group consisting of 5- or 6-membered heterocyclyl, phenyl, hydrogen, and C₁-C₃ alkyl, wherein said heterocyclyl or phenyl is substituted with zero, one or two substituents each independently selected from the group consisting of halogen, haloalkyl, and linear or branched C₁-C₃ alkyl;
or,
wherein R₁₃ and R₁₄, together with the nitrogen to which they are attached, form a 5 to 10-membered heterocyclyl or C₆-C₁₀ heteroaryl; and
n is 0 or 1.

2. The method of claim 1, wherein the composition is dispersed in a vaporized form.

3. (canceled)

4. The method of claim 1, wherein said compound comprises a compound of Formula IA and X is N.

5-8. (canceled)

9. The method of claim 4, wherein;
(a) R₁ is selected from the group consisting of; 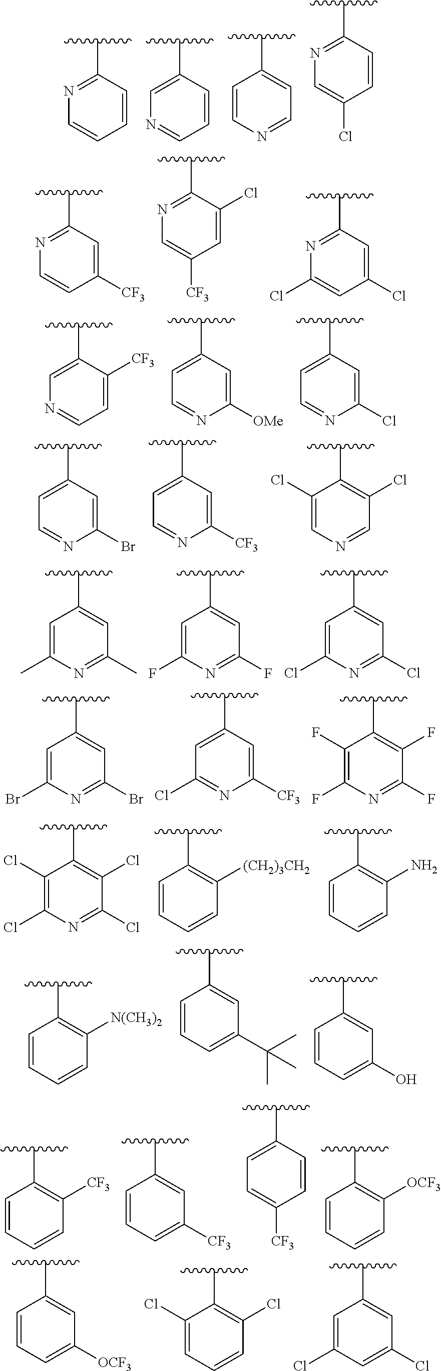 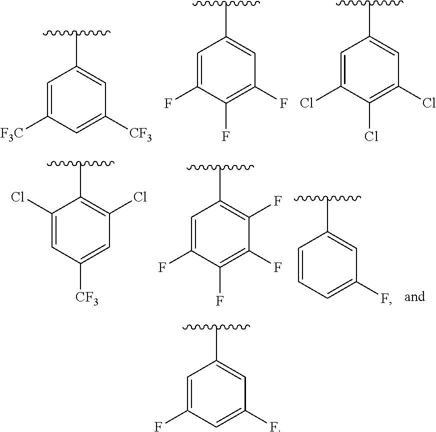
(b) R₂ is selected from the group consisting of: 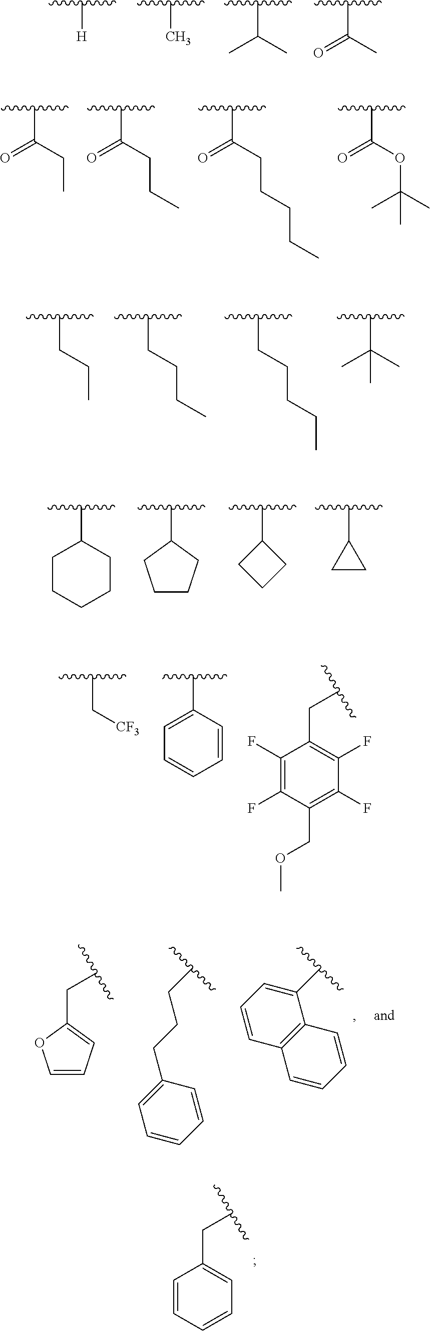
and
(c) R₃ is selected from the group consisting of: 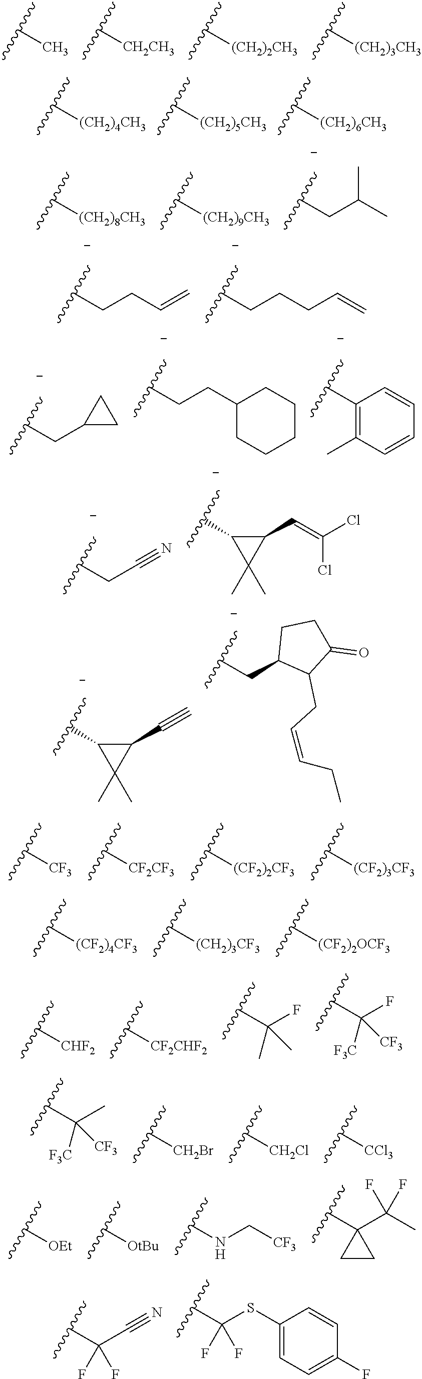 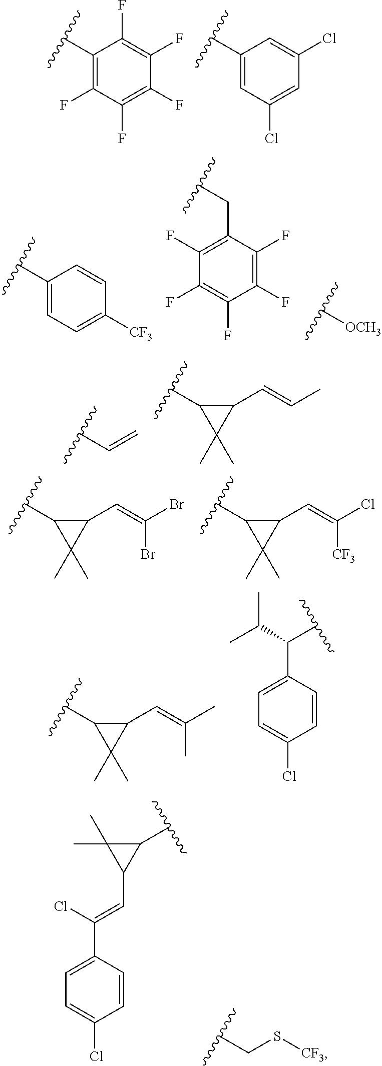
and —C(CF₃)₂H.

10. (canceled)

11. The method of claim 9, wherein;
(a) R₁ is 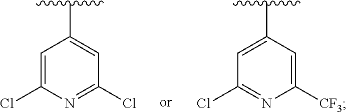 [SMILES: *c1cc(Cl)nc(Cl)c1 ; "or" ; *c1cc(Cl)nc(C(F)(F)F)c1]
(b) R₂ is hydrogen; and
(c) R₃ is

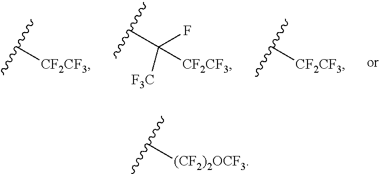

12-16. (canceled)

17. The method of claim 11, wherein said compound is selected from the group consisting of

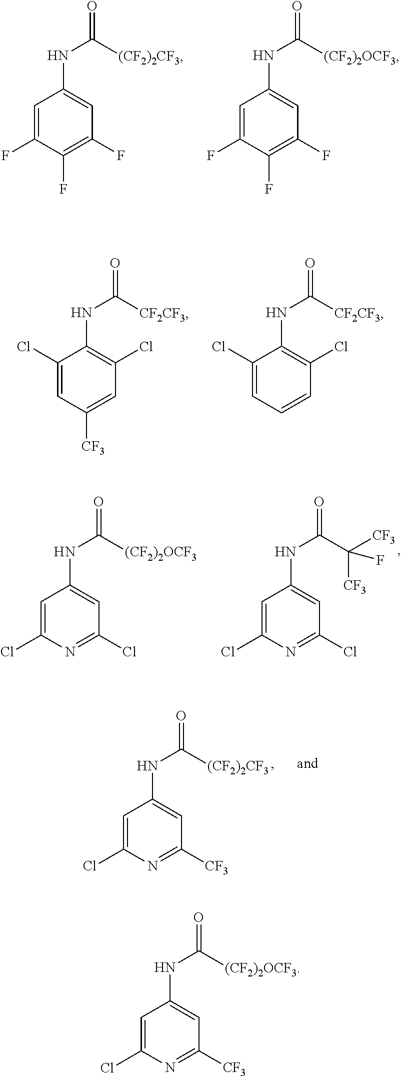

18. (canceled)

19. The method of claim 1, wherein said arthropods are insects.

20. (canceled)

21. The method of claim 1, wherein said compound is selected from the group consisting of:

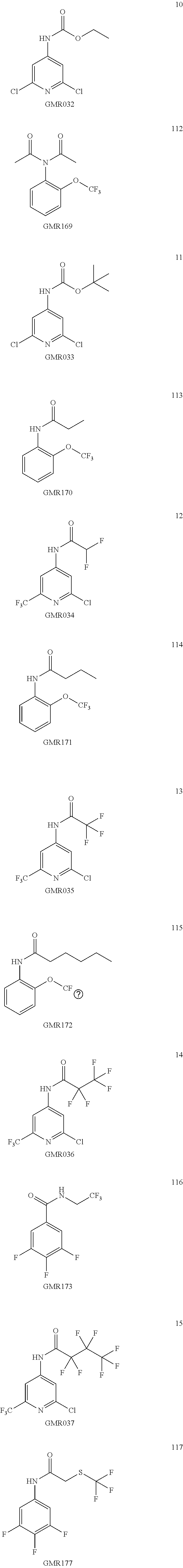

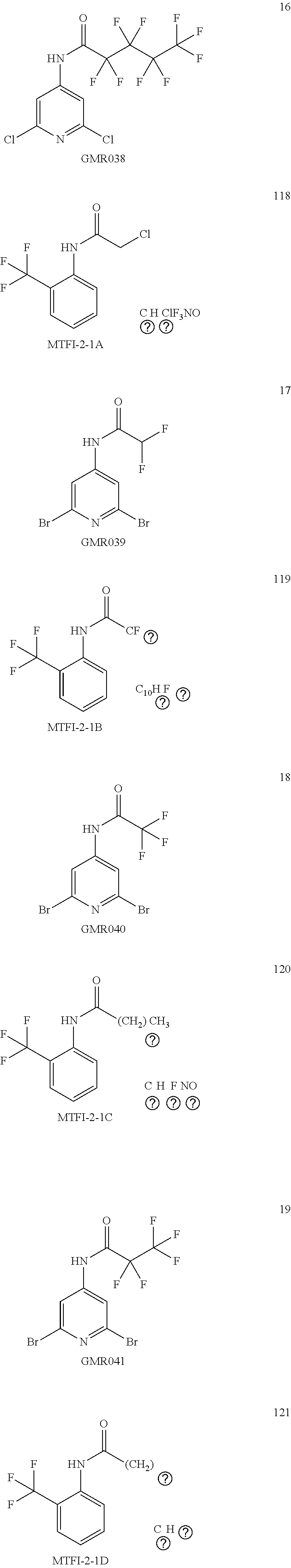

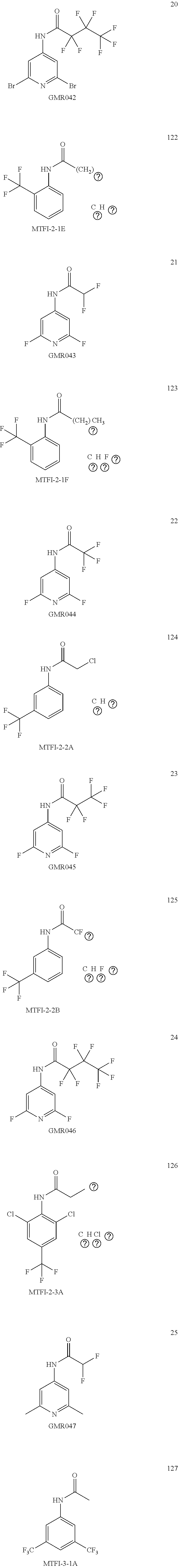

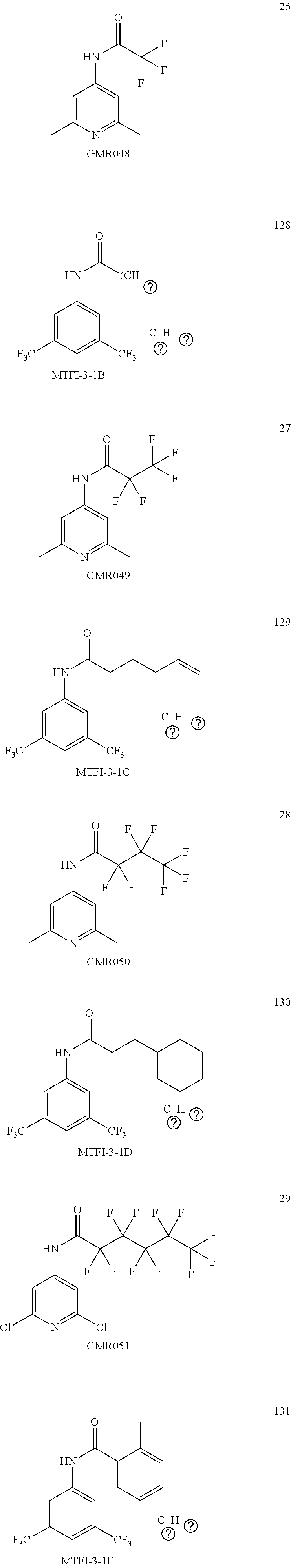

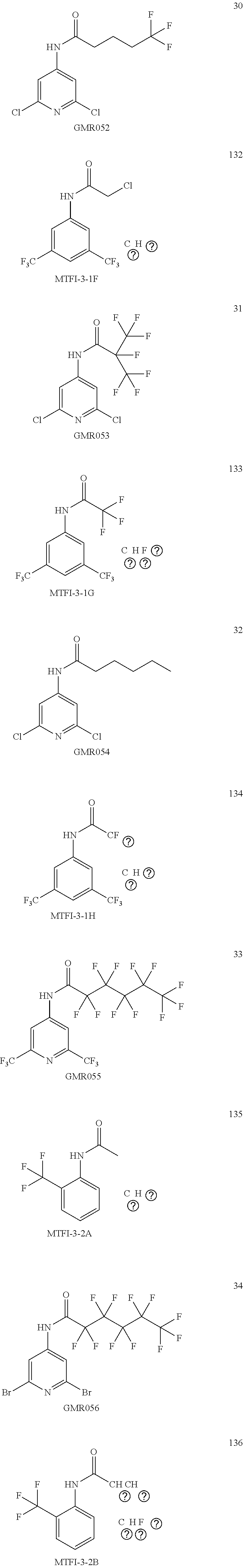

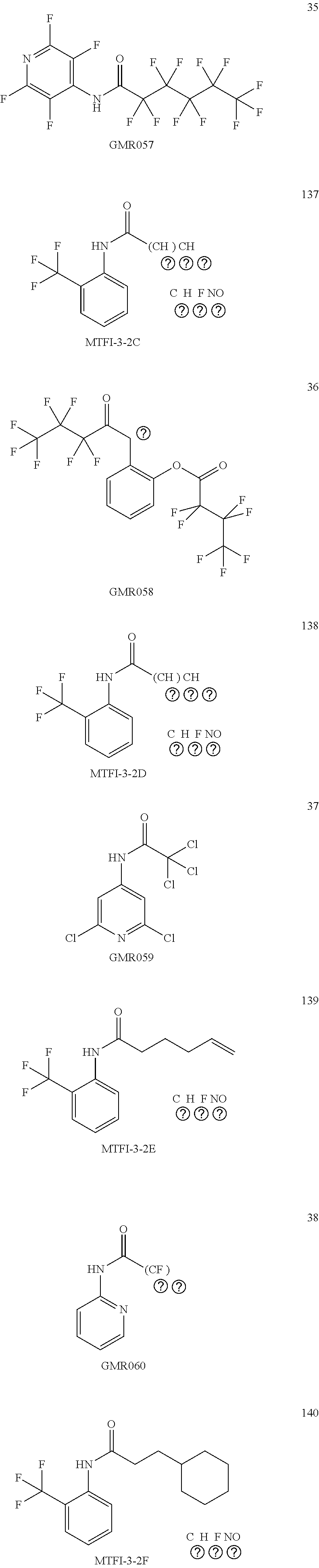

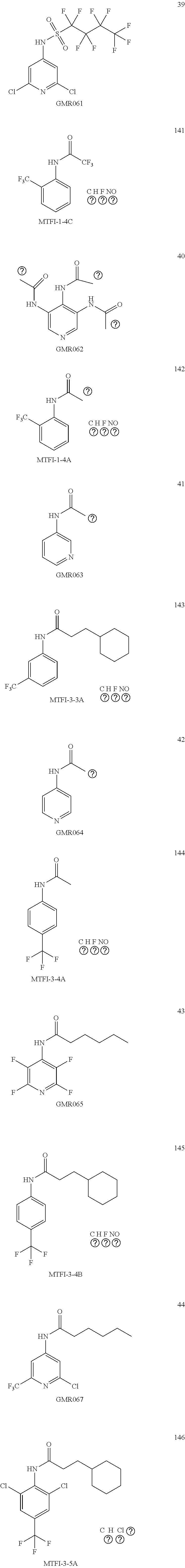

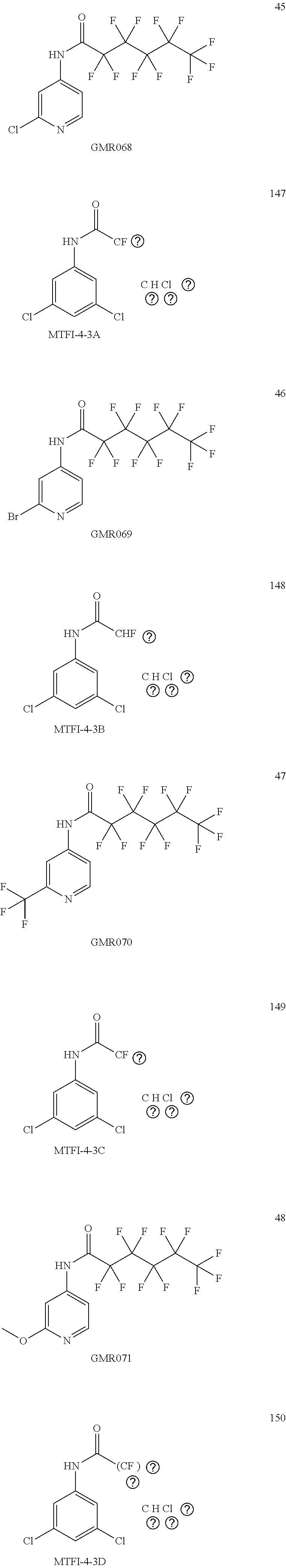

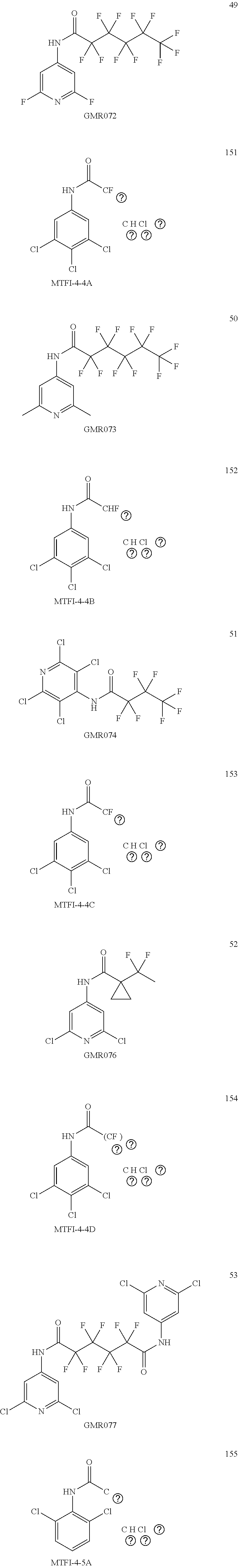

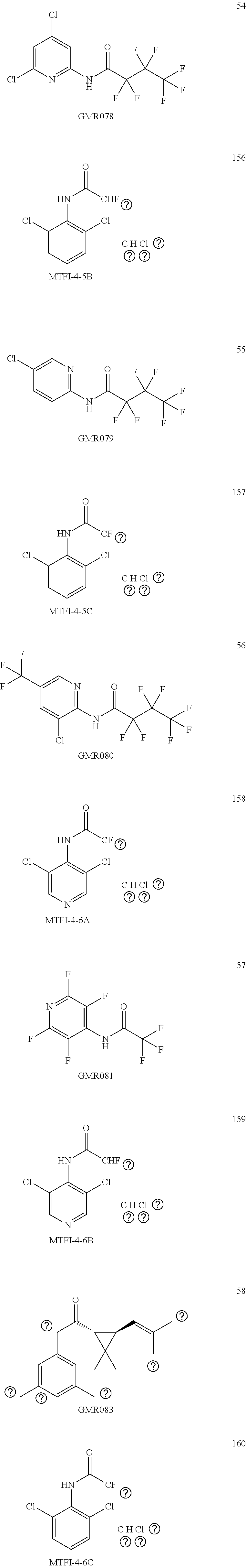

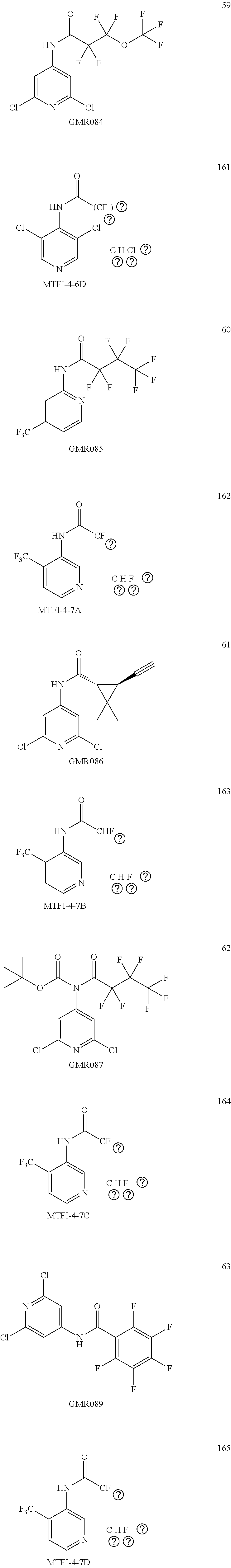

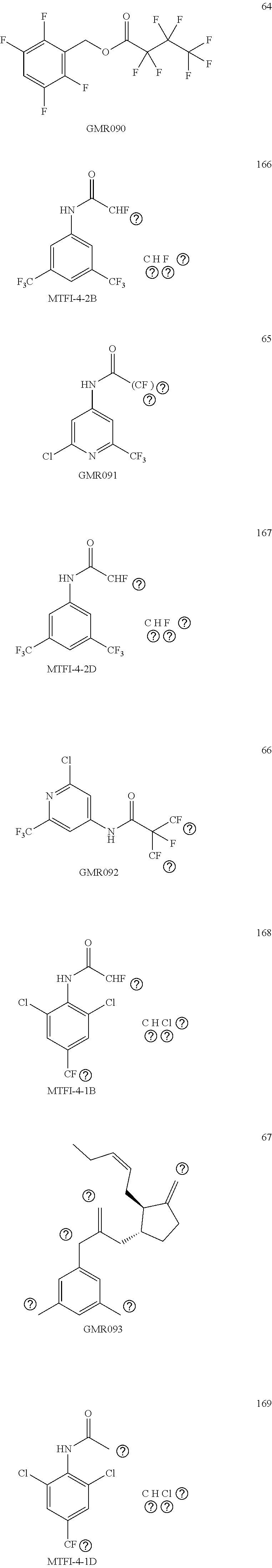

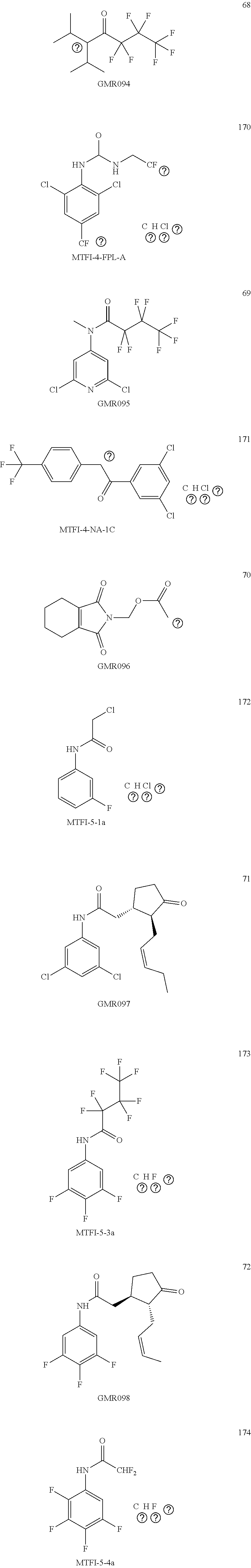

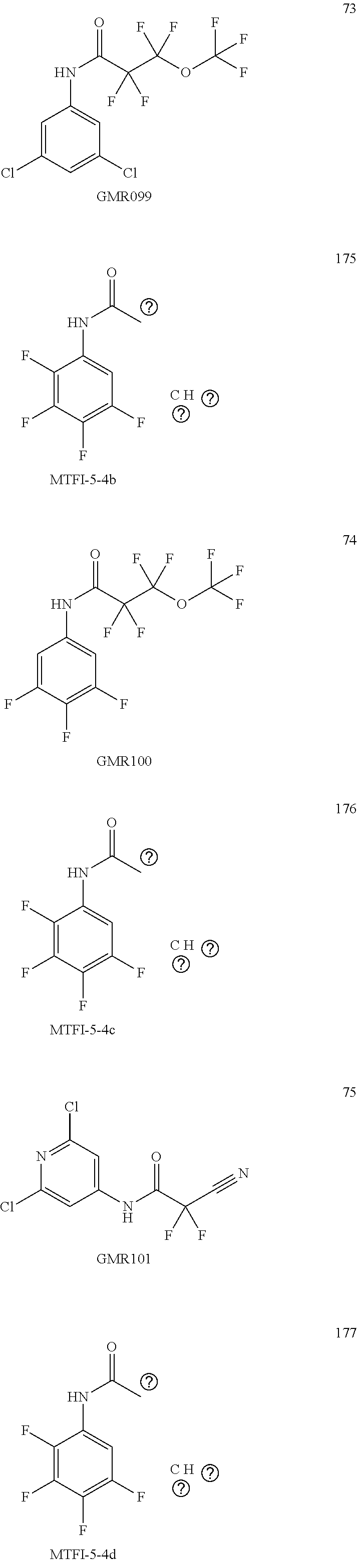

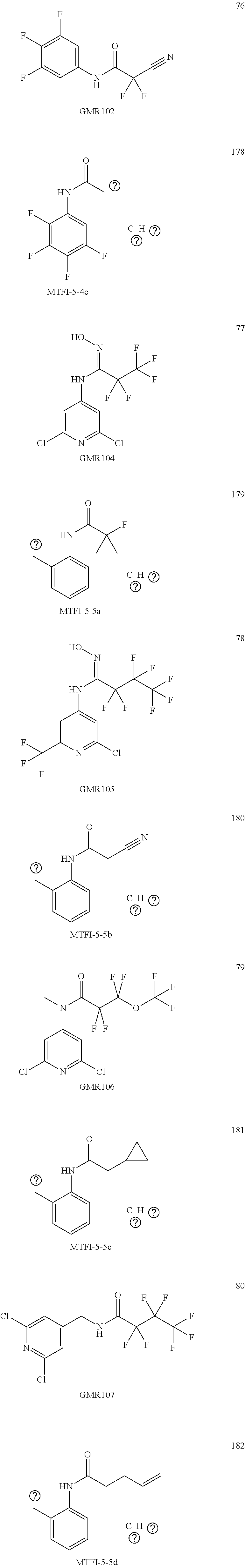

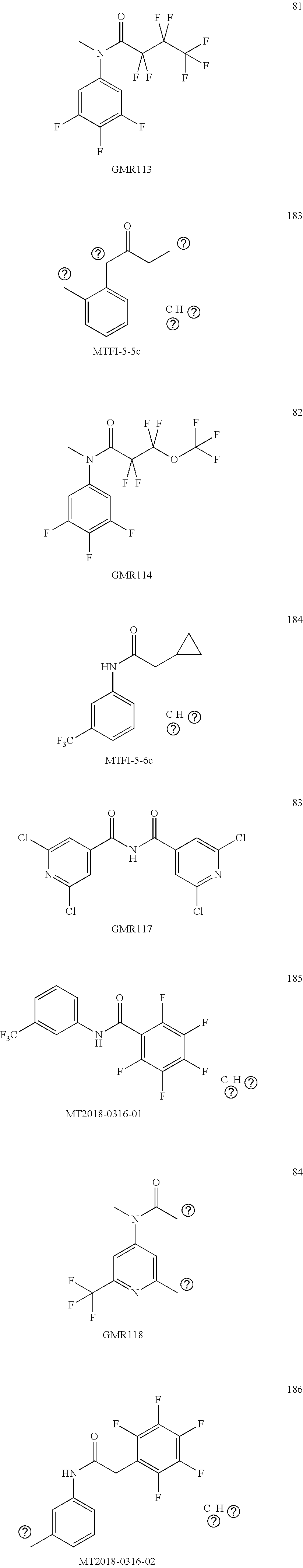

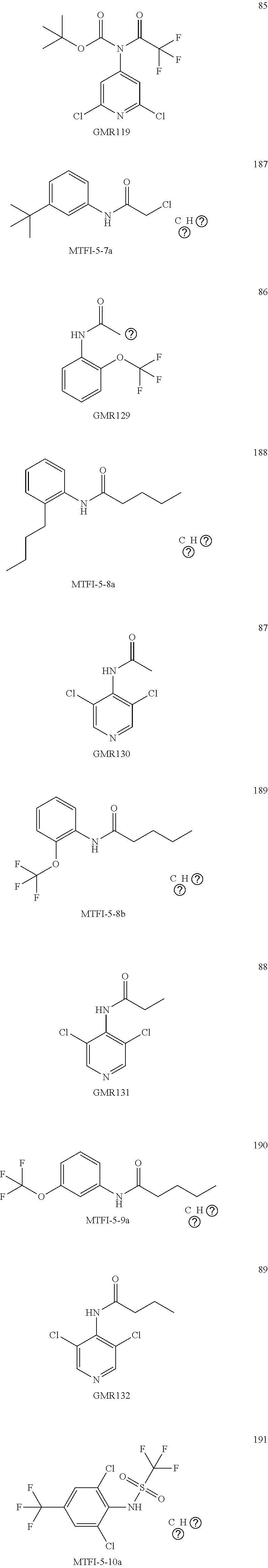

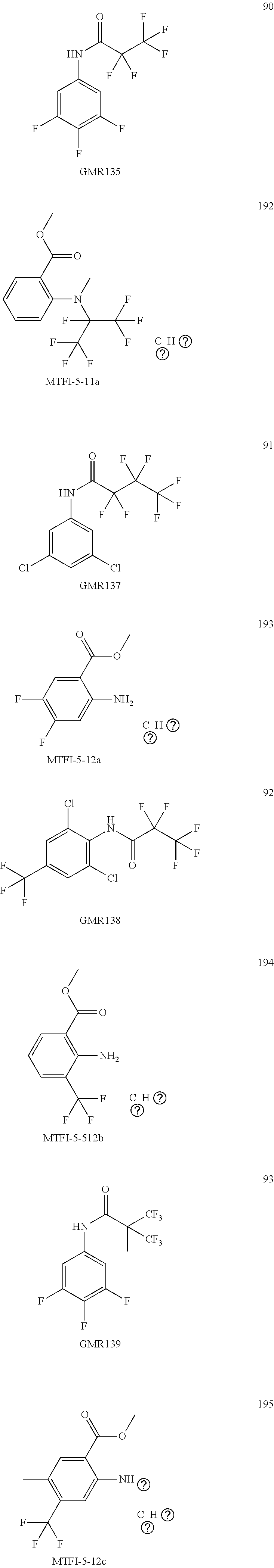

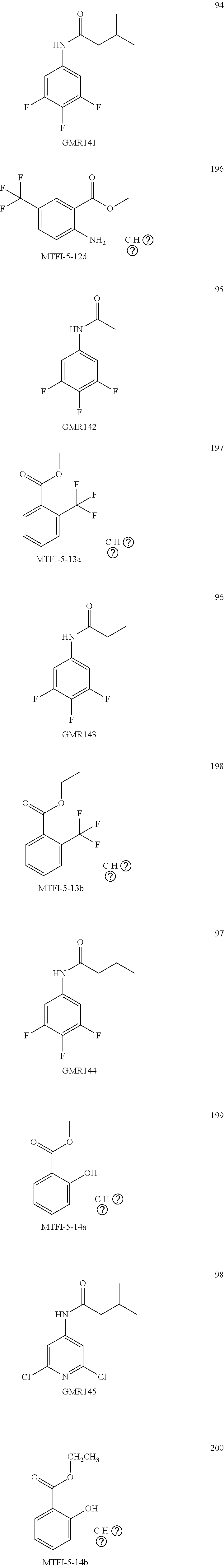

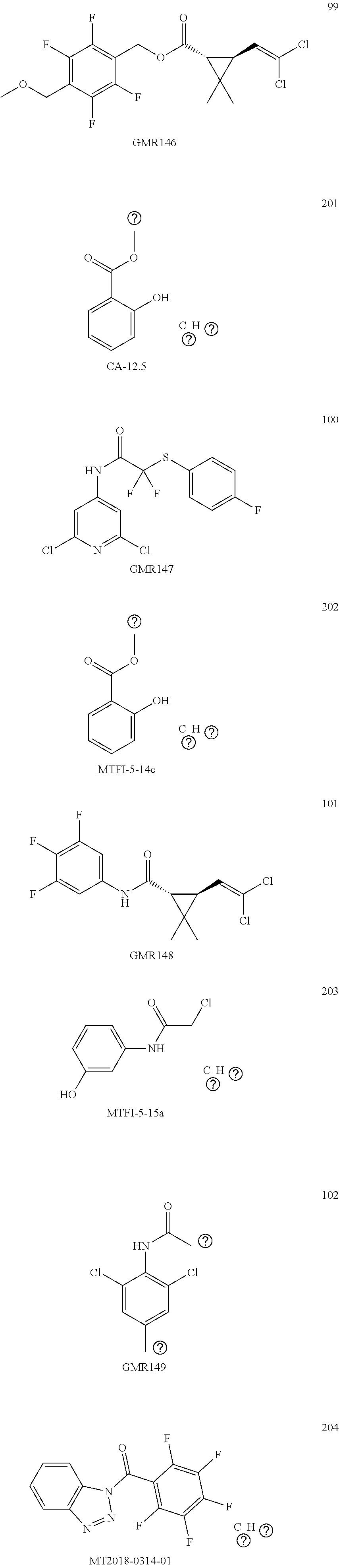

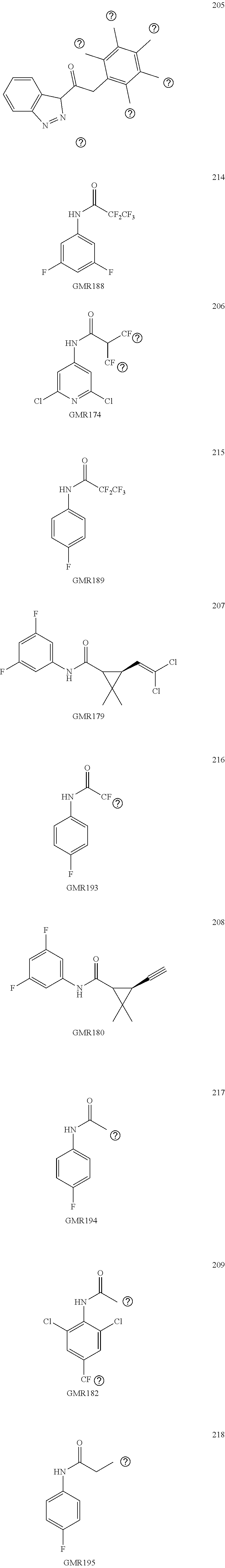

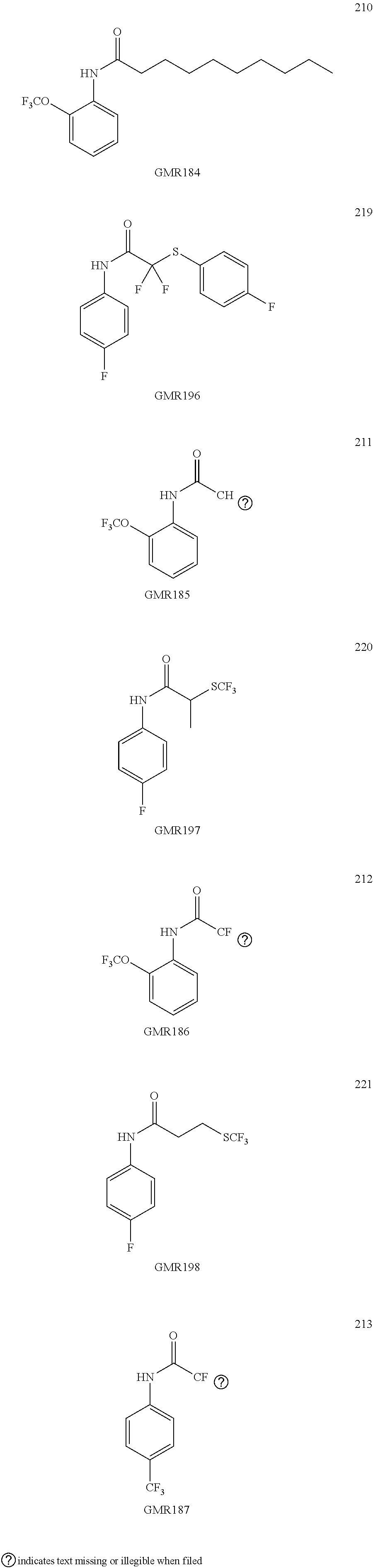

22. A compound of Formula X 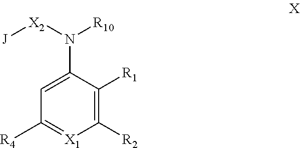
wherein,
X₁ is N or CR₃, wherein R₃ is selected from the group consisting of hydrogen, halogen, and —(C₁-C₃)alkyl, wherein the —(C₁-C₃)alkyl is substituted with zero, one or more hydrogens or halogens;
R₁ is hydrogen or a halogen;
R₂ and R₄ are each independently selected from the group consisting of hydrogen, halogen, and —(C₁-C₆)alkyl, wherein the —(C₁-C₆)alkyl is substituted with zero, one or more hydrogens or halogens;
X₂ is selected from the group consisting of C(O), S(O), S(O)₂, —CH(OH), and —CHCF₃;
R₁₀ is hydrogen or C₁-C₆ alkyl; and
J is selected from the group consisting of —(CF₂)OCF₃, —(CF₂)₂CF₃, —(CF₂)₃CF₃, —(CF₂)₄CF₃, —C(CF₃)₂H, —CH₂SCF₃, 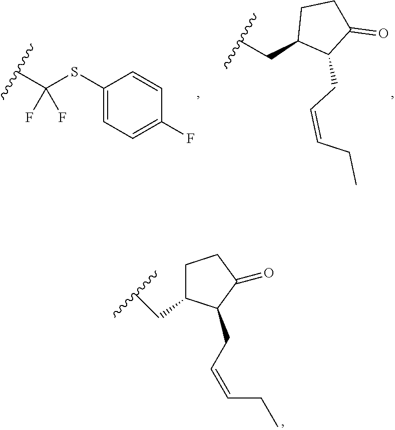 [SMILES: *C(F)(F)Sc1ccc(F)cc1 ; *C[C@H]1CCC(=O)[C@@H]1C/C=C\CC ; *C[C@@H]1CCC(=O)[C@H]1C/C=C\CC]
—CF₂CN, —(CH₂)₃CF₃, —C(CF₃)₂F, —CCl₃, 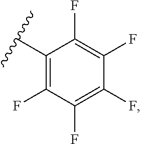 [SMILES: *c1c(F)c(F)c(F)c(F)c1F]
—(CH₂)₄CF₃, 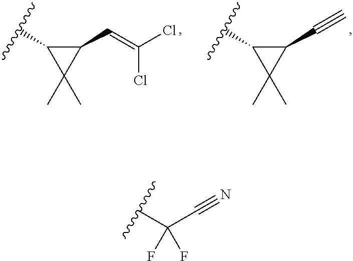
—CHF₂, —CF₃, and —CF₂CF₃.

23. The compound of claim 22, wherein X₂ is C(O) and R₁₀ is hydrogen or methyl.

24-26. (canceled)

27. The compound of claim 23, wherein R₁₀ in hydrogen and
(a) R₁ is hydrogen, X₁ is CR₃ wherein R₃ is fluorine, R₂ and R₄ are each fluorine, and J is selected from the group consisting of —(CF₂)₂CF₃, 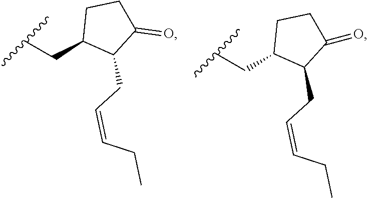 [SMILES: *C[C@H]1CCC(=O)[C@@H]1C/C=C\CC ; *C[C@@H]1CCC(=O)[C@H]1C/C=C\CC]
—CF₂CF₂OCF₃, —CH₂SCF₃, —CF₂CN, and 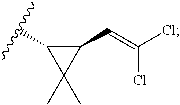 [SMILES: *[C@@H]1[C@@H](C=C(Cl)Cl)C1(C)C]
(b) X₁ is CR₃, R₃ is fluorine, R₁, R₂, and R₄ are each fluorine, and J is selected from the group consisting of —CHF₂, —CF₃, —CF₂CHF₂, —CF₂CF₃, and —(CF₂)₂CF₃;
(c) R₁ is hydrogen, X₁ is N, R₄ and R₂ are each chlorine, and J is selected from the group consisting of —CHF₂, —CF₃, —CF₂CF₃, —(CF₂)₂CF₃, a 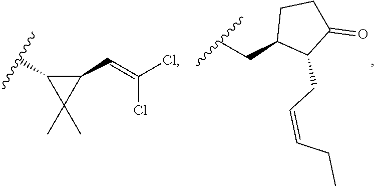
—(CF₂)₄CF₃, —(CF₂)₃CF₃, —(CH₂)₃CF₃, —C(CF₃)₂F, —(CH₂)₄CH₃, —(CF₂)₂OCF₃, 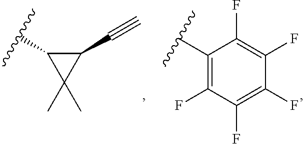
—CCl₃, 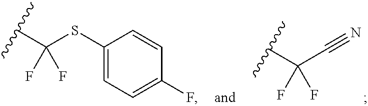 [SMILES: *C(F)(F)Sc1ccc(F)cc1 ; *C(F)(F)C#N]
(d) R₁ is hydrogen, X₁ is N, R₄ and R₂ are each fluorine, and J is selected from the group consisting of —CHF₂, —CF₃, —CF₂CF₃, and —(CF₂)CF₃;
(e) R₁ is hydrogen, X₁ is CR₃ wherein R₃ is hydrogen, R₄ is chlorine, R₂ is chlorine, and J is 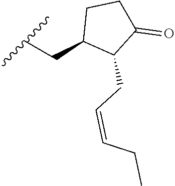 [SMILES: *C[C@H]1CCC(=O)[C@@H]1C/C=C\CC]
or —(CF₂)₂OCF₃;
(f) R₁ is hydrogen, X₁ is N, R₄ and R₂ are each bromine, and J is selected from the group consisting of —CF₂H, —CF₃, —CF₂CF₃, —(CF₂)₂CF₃, and —(CF₂)₄CF₃;
(g) R₁ is hydrogen, X₁ is CR₃ wherein R₃ is hydrogen, one of R₄ and R₂ is chlorine and the other is —CF₃, and J is selected from the group consisting of —(CF₂)₂OCF₃, —CHF₂, —CF₃, —(CF₂)CF₃, —(CF₂)₂CF₃, —(CF₂)₄CF₃, —(CH₂)₄CH₃, and —CF(CF₃)₂;
(h) R₁ is hydrogen, X₁ is N, R₄ and R₂ are each methyl, and J is selected from the group consisting of —CF₃, —CF₂CF₃, —CHF₂, and —(CF₂)₄CF₃; or
(i) R₁ is hydrogen, X₁ is CR₃ wherein R₃ is hydrogen, R₄ is —CF₃, R₂ is —CF₃, and J is —(CF₂)₂CF₃.

28-29. (canceled)

30. The compound of claim 24, wherein R₁₀ is methyl and
(a) R₁ is hydrogen, X₁ is CR₃ wherein R₃ is fluorine, R₂ and R₄ are each fluorine, and J is selected from the group consisting of —(CF₂)₃CF₃, —(CF₂)₂CF₃, and —CF₂CF₂OCF₃;
(b) R₁ is hydrogen, X₁ is N, R₄ and R₂ are each chlorine, and J is —(CF₂)₂CF₃ or —(CF₂)₂OCF₃; or
(c) R₁ is hydrogen, X₁ is CR₃ wherein R₃ is hydrogen, one of R₄ and R₂ is chlorine and the other is —CF₃, and J is —(CF₂)₂CF₃.

31-40. (canceled)

41. The compound of claim 22, wherein said compound has a structure selected from the group consisting of:

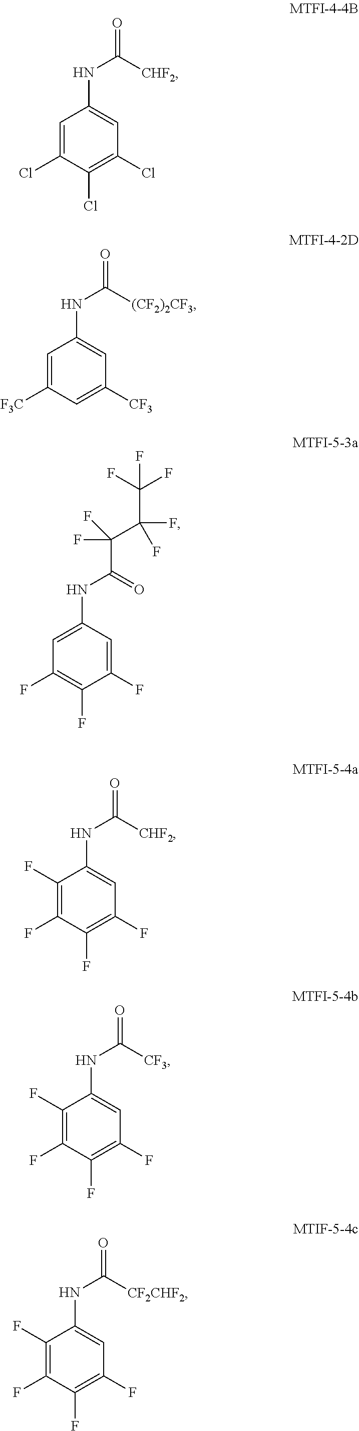

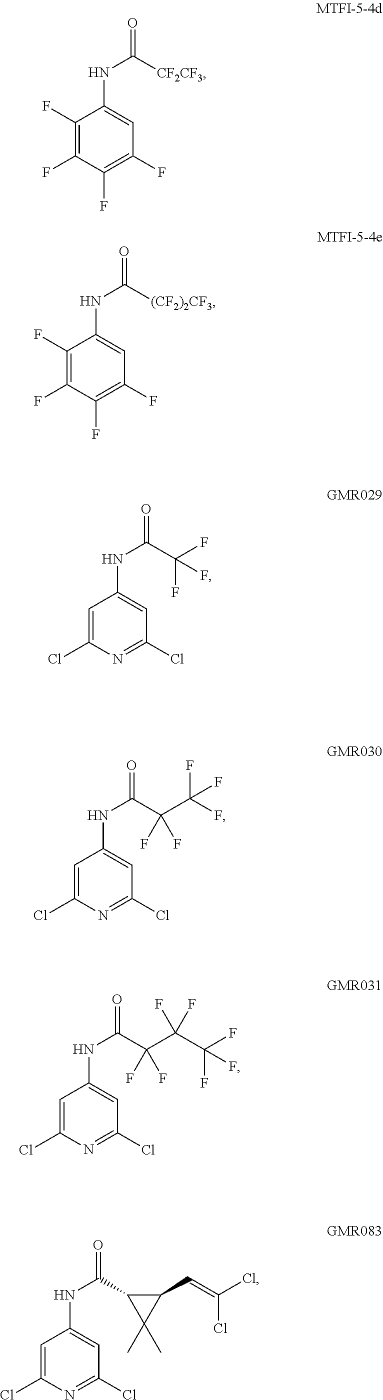

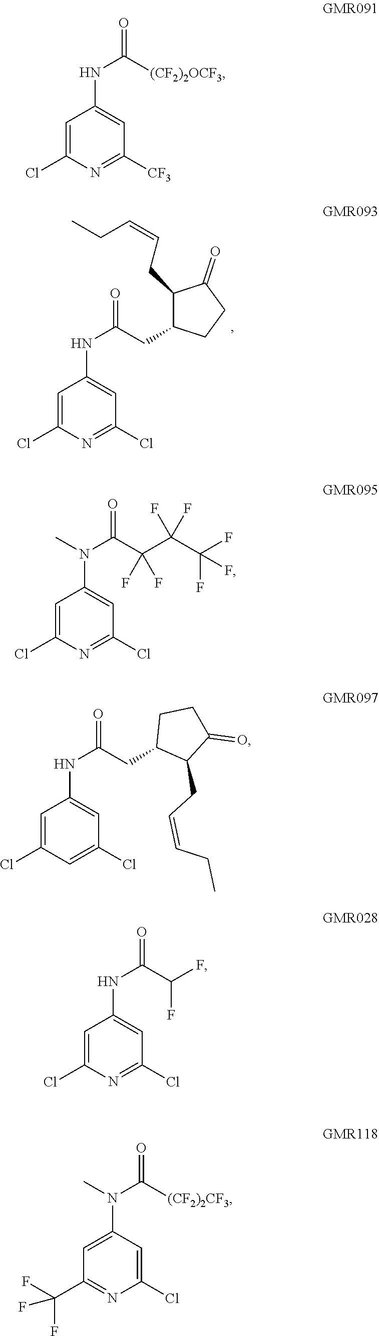

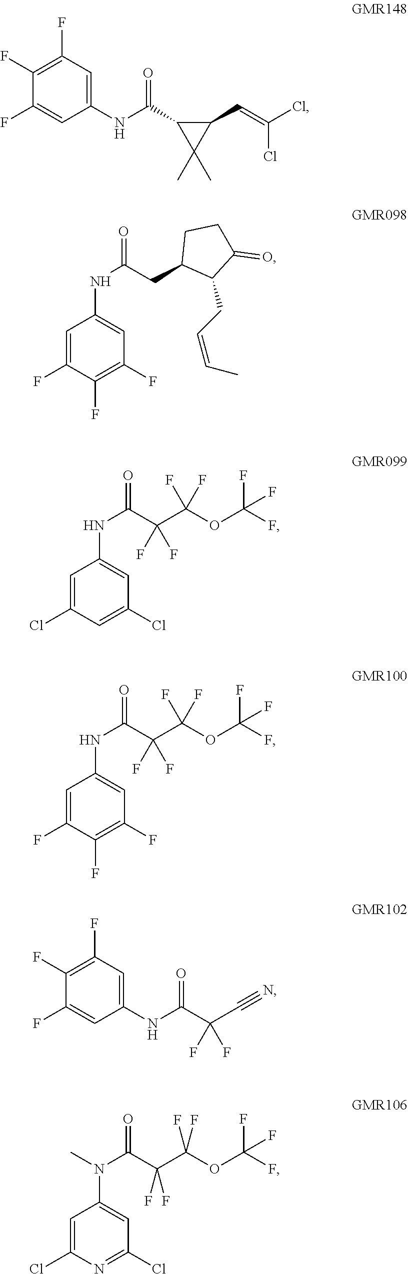

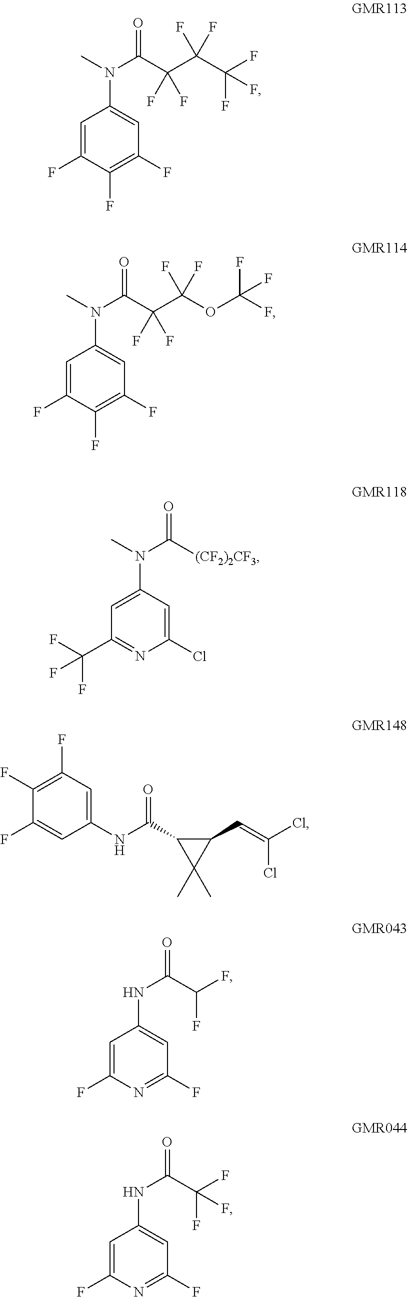

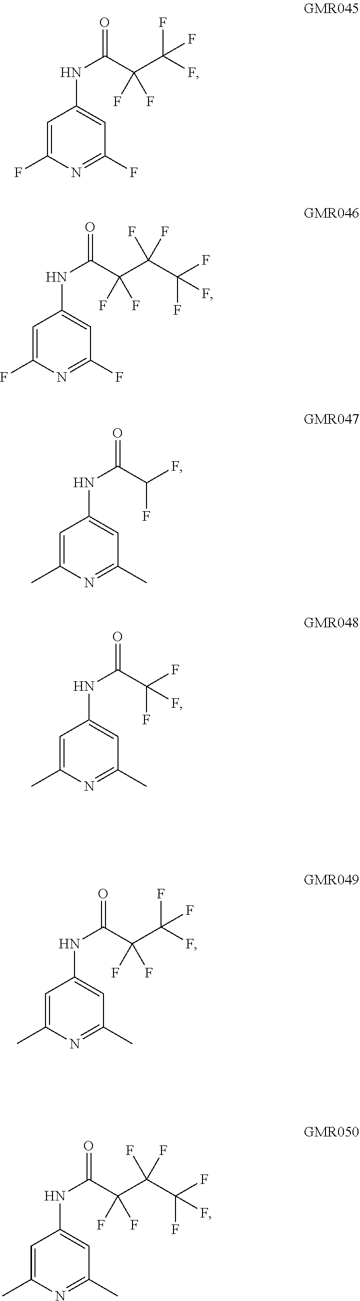 [SMILES: O=C(Nc1cc(F)nc(F)c1)C(F)(F)C(F)(F)F ; O=C(Nc1cc(F)nc(F)c1)C(F)(F)C(F)(F)C(F)(F)F ; Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1]

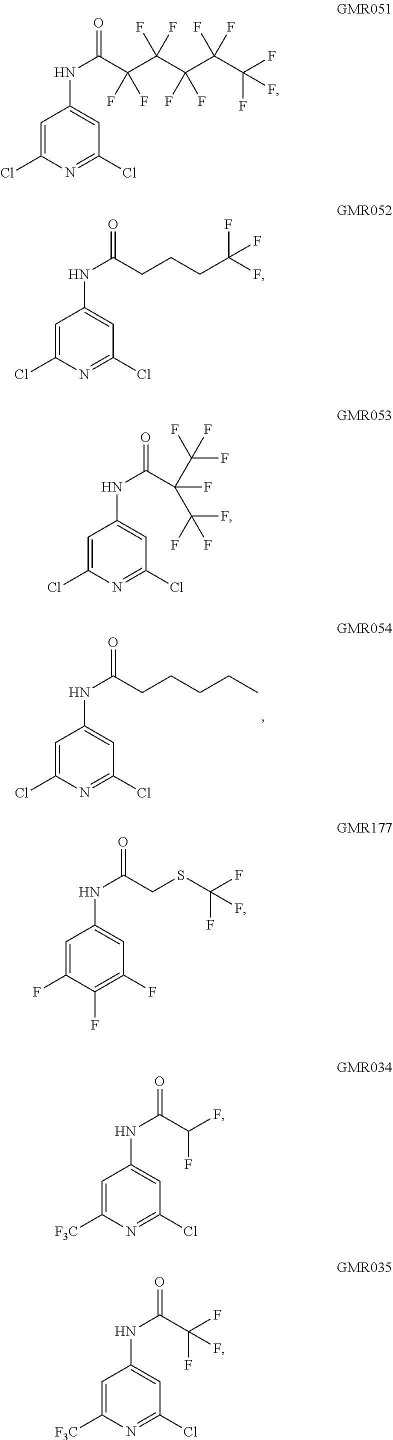

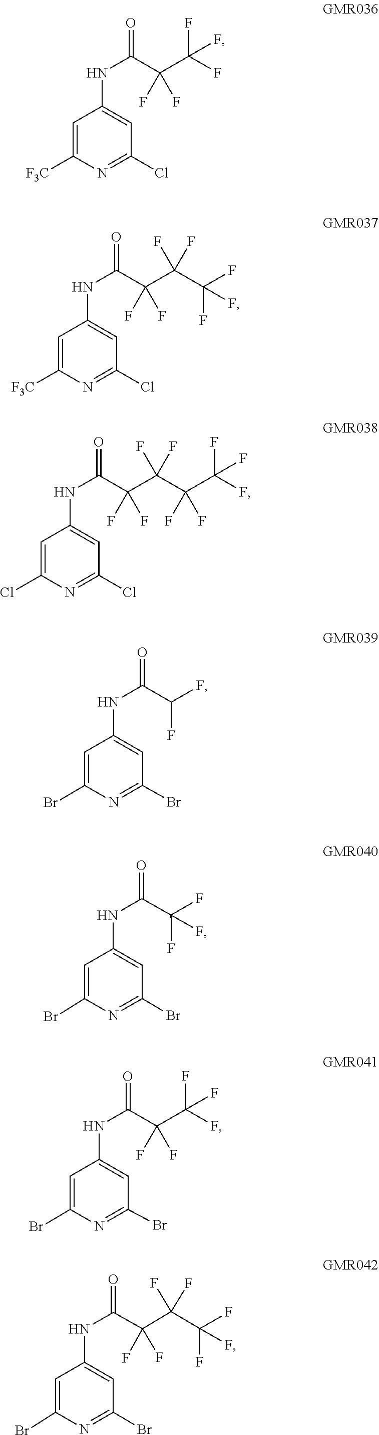

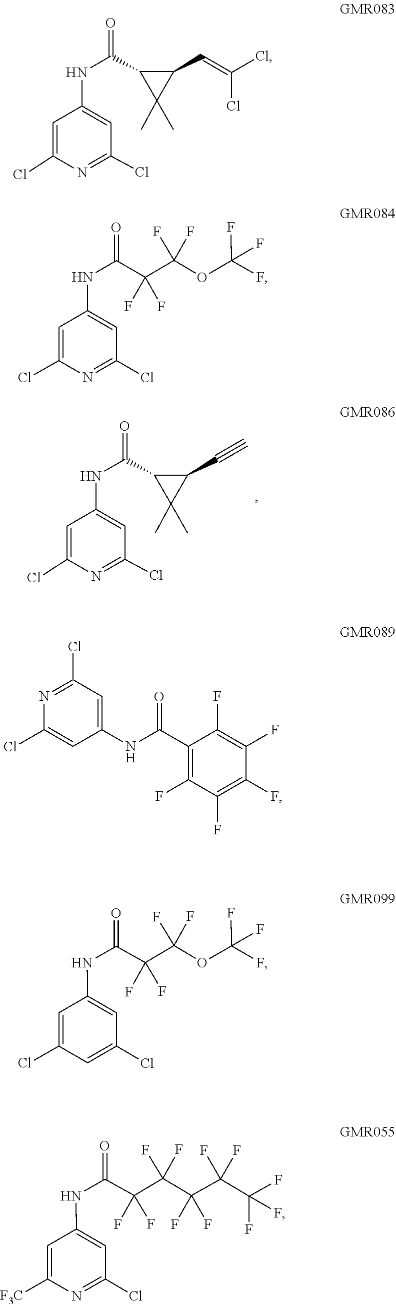 [SMILES: CC1(C)[C@H](C=C(Cl)Cl)[C@H]1C(=O)Nc1cc(Cl)nc(Cl)c1 ; O=C(Nc1cc(Cl)nc(Cl)c1)C(F)(F)C(F)(F)OC(F)(F)F ; C#C[C@@H]1[C@@H](C(=O)Nc2cc(Cl)nc(Cl)c2)C1(C)C ; O=C(Nc1cc(Cl)nc(Cl)c1)c1c(F)c(F)c(F)c(F)c1F ; O=C(Nc1cc(Cl)cc(Cl)c1)C(F)(F)C(F)(F)OC(F)(F)F ; O=C(Nc1cc(Cl)nc(C(F)(F)F)c1)C(F)(F)C(F)(F)C(F)(F)C(F)(F)C(F)(F)F]

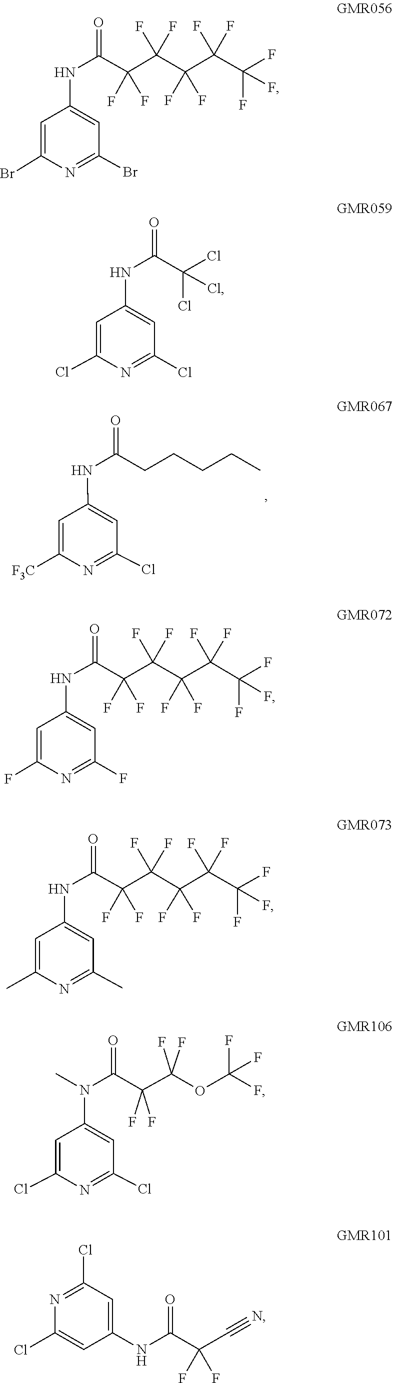

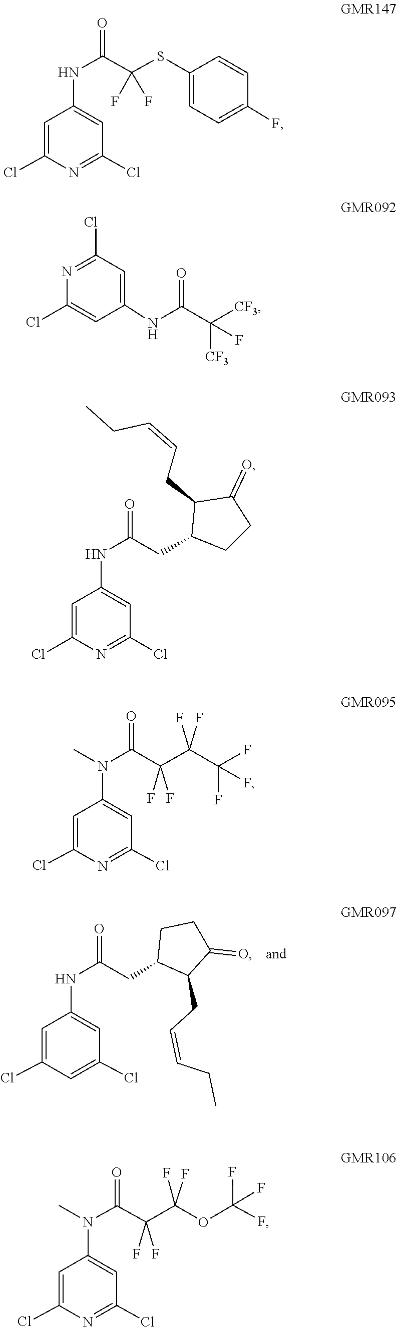

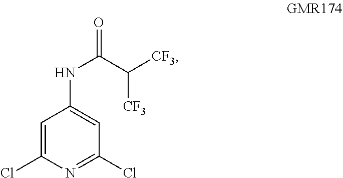 [SMILES: O=C(Nc1cc(Cl)nc(Cl)c1)C(C(F)(F)F)C(F)(F)F]

42-54. (canceled)

55. A compound, selected from the group consisting of:

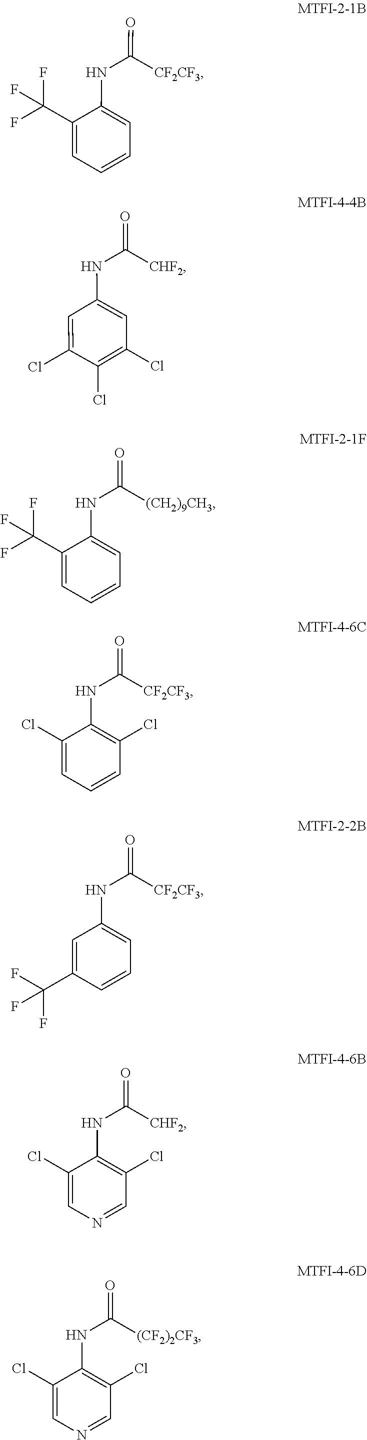

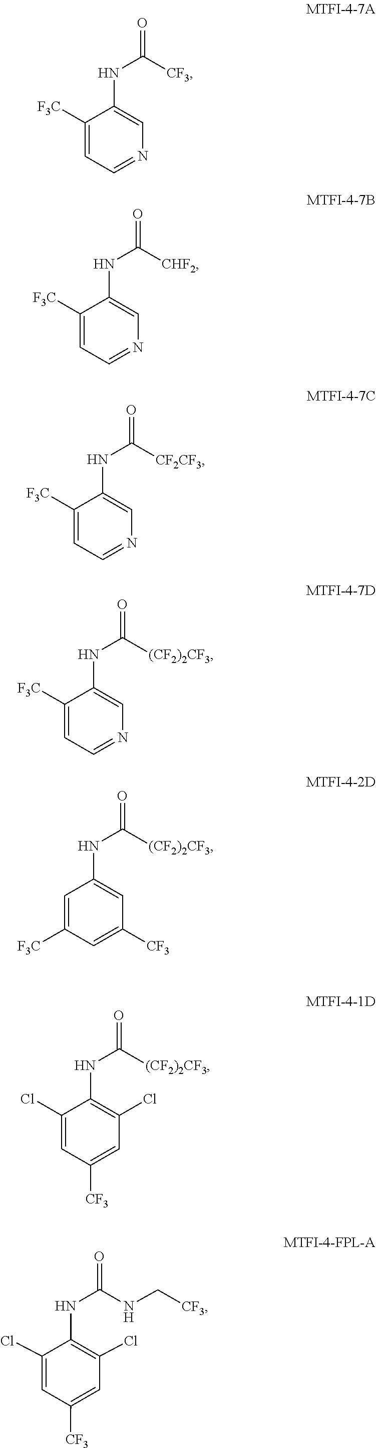

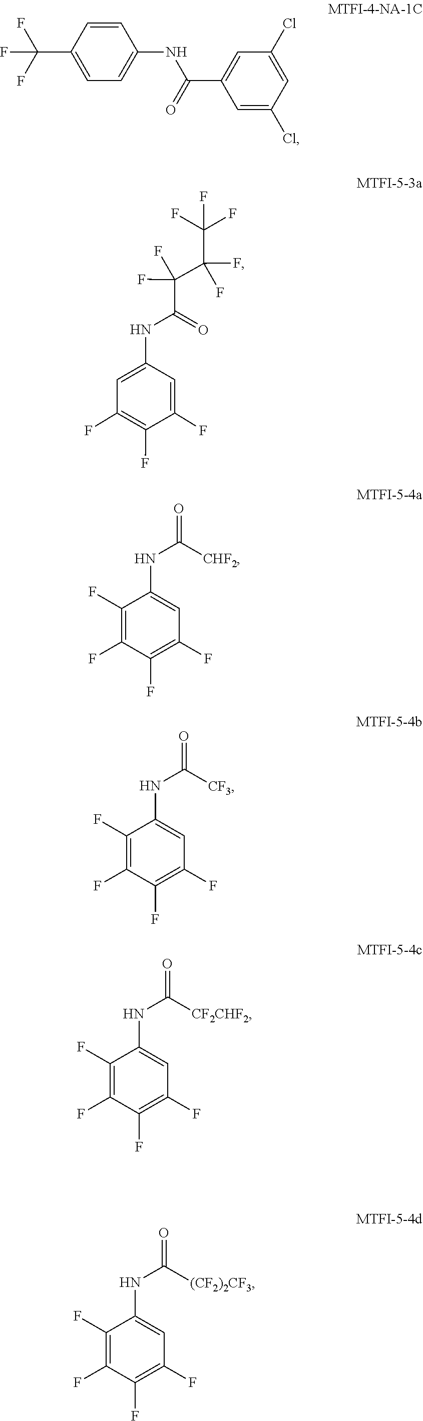

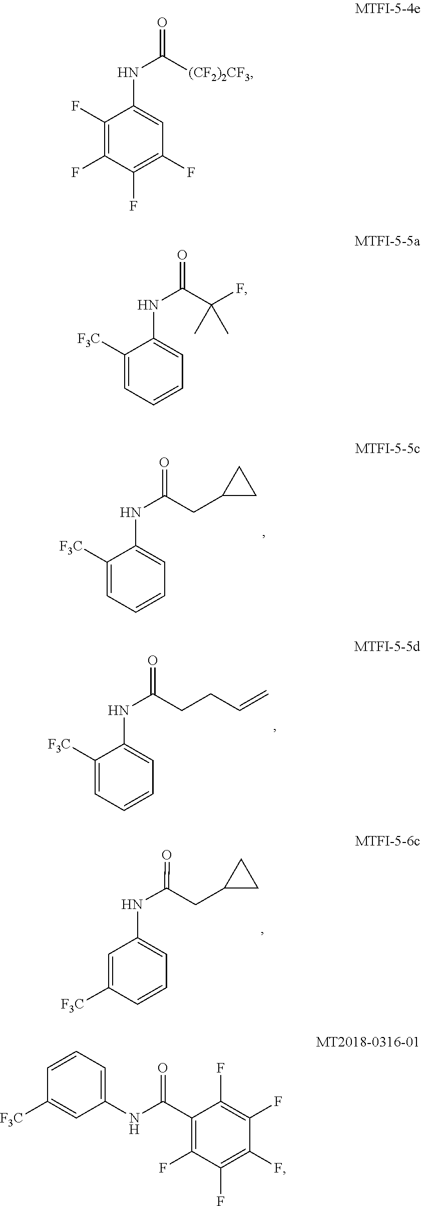

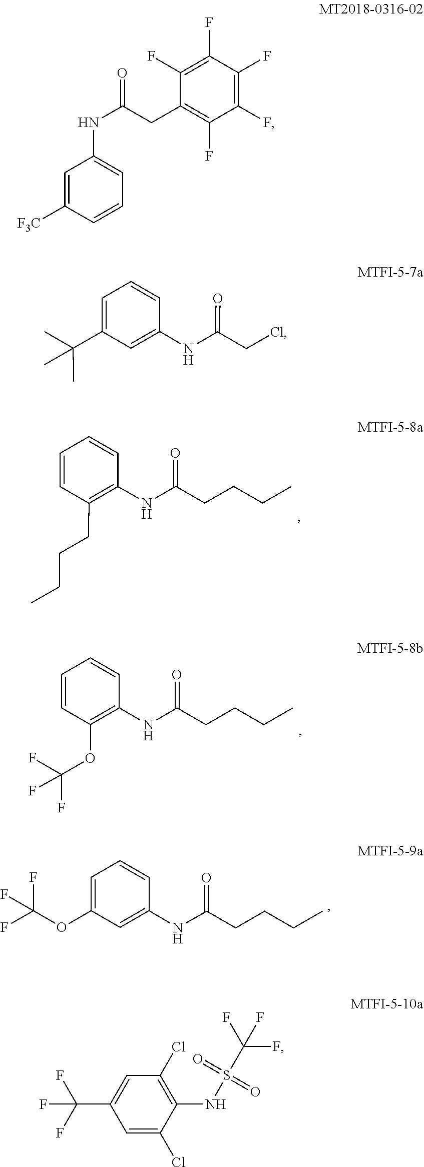 [SMILES: O=C(Cc1c(F)c(F)c(F)c(F)c1F)Nc1cccc(C(F)(F)F)c1 ; CC(C)(C)c1cccc(NC(=O)CCl)c1 ; CCCCC(=O)Nc1ccccc1CCCC ; CCCCC(=O)Nc1ccccc1OC(F)(F)F ; CCCCC(=O)Nc1cccc(OC(F)(F)F)c1 ; O=S(=O)(Nc1c(Cl)cc(C(F)(F)F)cc1Cl)C(F)(F)F]

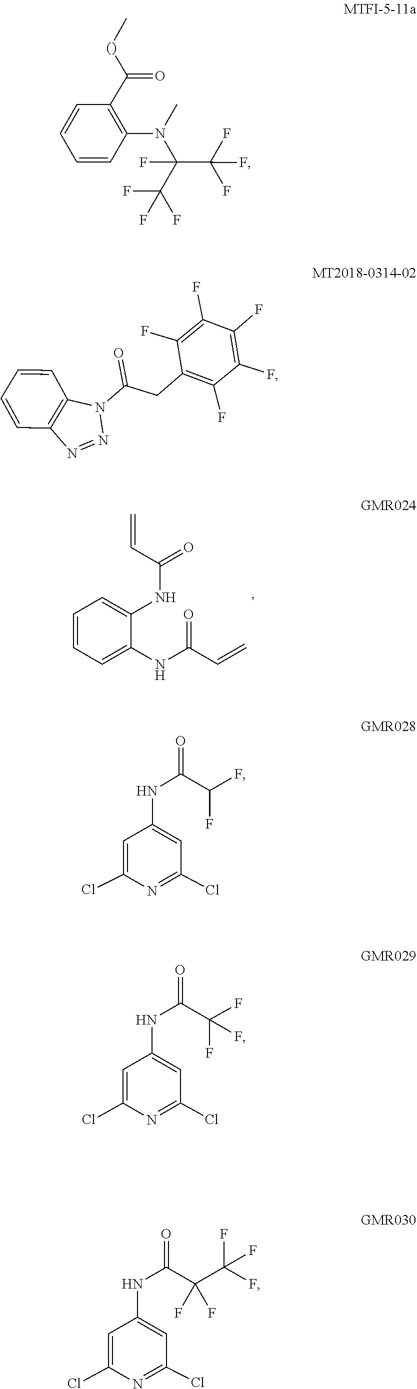

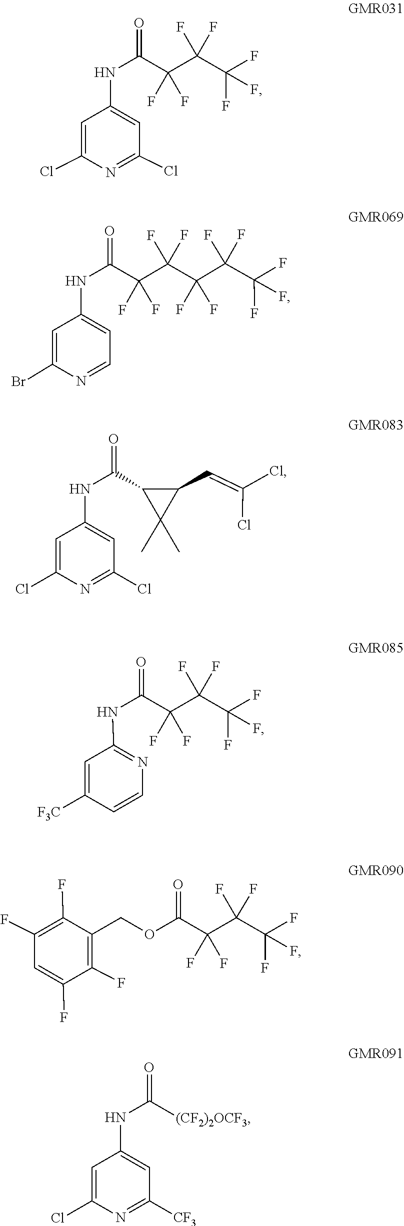

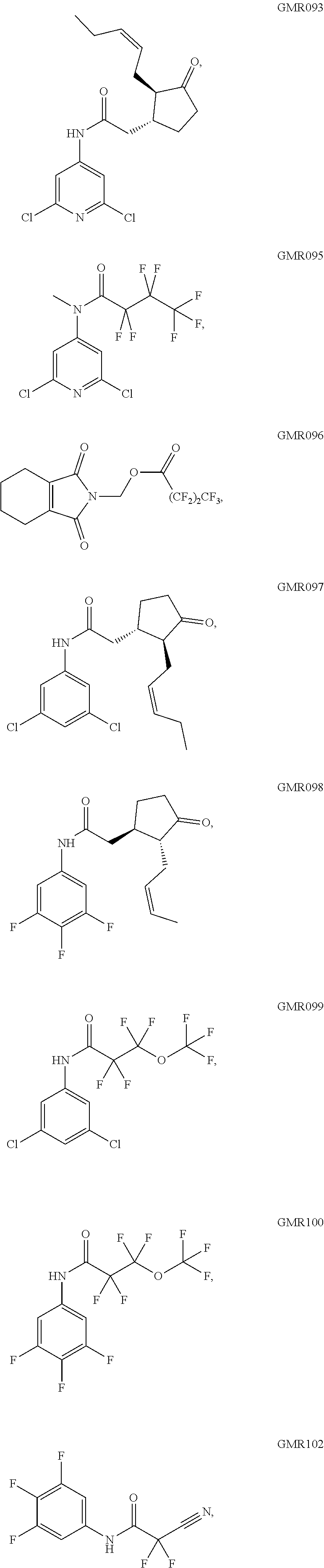

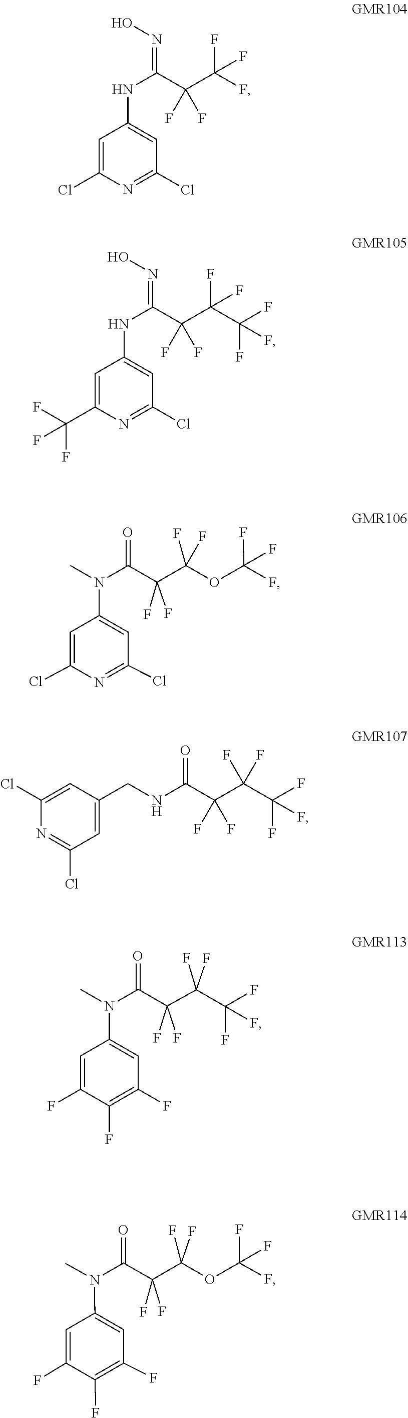 [SMILES: O/N=C(\Nc1cc(Cl)nc(Cl)c1)C(F)(F)C(F)(F)F ; O/N=C(\Nc1cc(Cl)nc(C(F)(F)F)c1)C(F)(F)C(F)(F)C(F)(F)F ; CN(C(=O)C(F)(F)C(F)(F)OC(F)(F)F)c1cc(Cl)nc(Cl)c1 ; O=C(NCc1cc(Cl)nc(Cl)c1)C(F)(F)C(F)(F)C(F)(F)F ; CN(C(=O)C(F)(F)C(F)(F)C(F)(F)F)c1cc(F)c(F)c(F)c1 ; CN(C(=O)C(F)(F)C(F)(F)OC(F)(F)F)c1cc(F)c(F)c(F)c1]

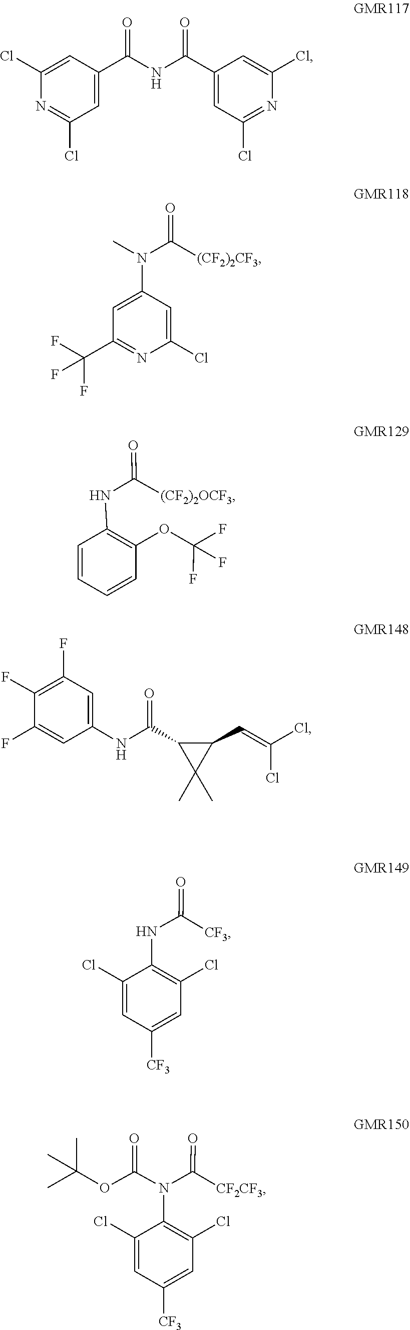

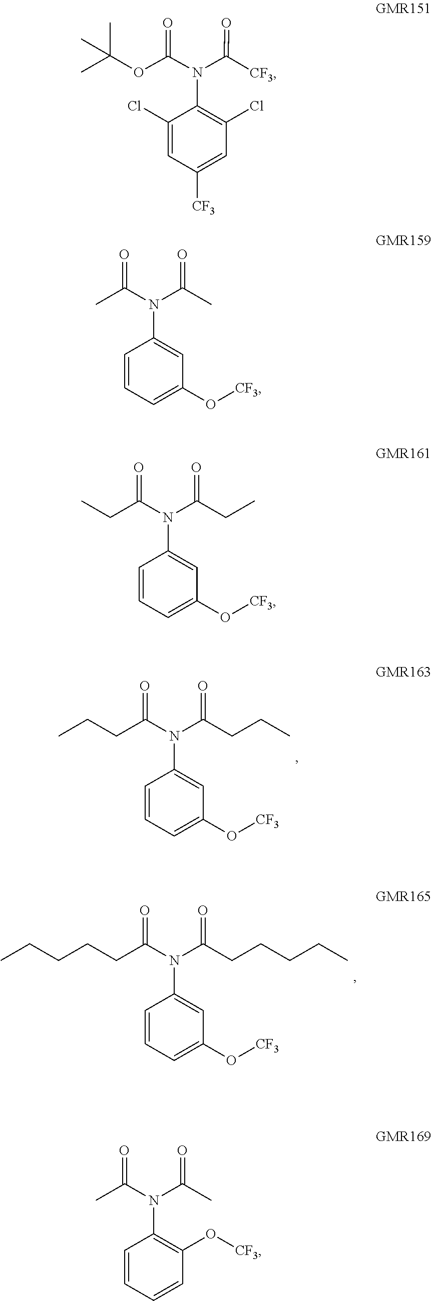

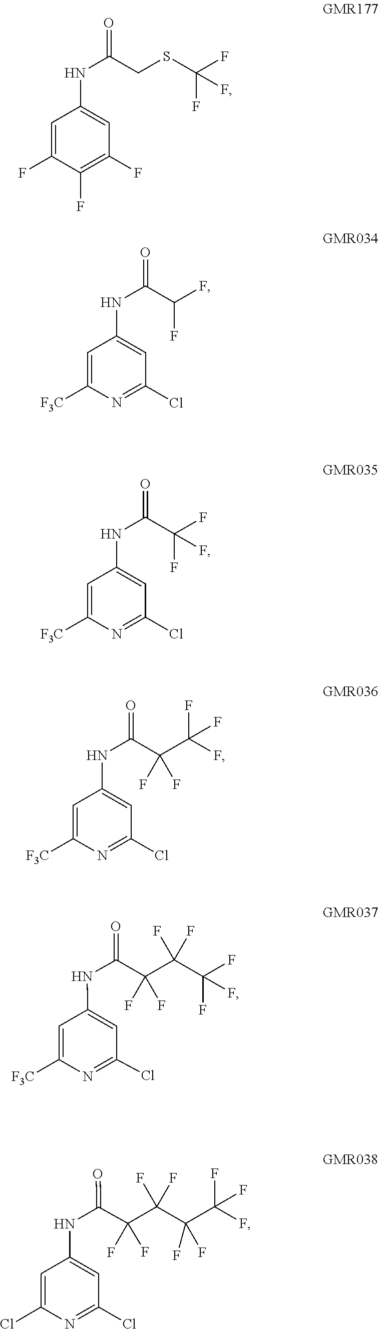 [SMILES: O=C(CSC(F)(F)F)Nc1cc(F)c(F)c(F)c1 ; O=C(Nc1cc(Cl)nc(C(F)(F)F)c1)C(F)F ; O=C(Nc1cc(Cl)nc(C(F)(F)F)c1)C(F)(F)F ; O=C(Nc1cc(Cl)nc(C(F)(F)F)c1)C(F)(F)C(F)(F)F ; O=C(Nc1cc(Cl)nc(C(F)(F)F)c1)C(F)(F)C(F)(F)C(F)(F)F ; O=C(Nc1cc(Cl)nc(Cl)c1)C(F)(F)C(F)(F)C(F)(F)C(F)(F)F]

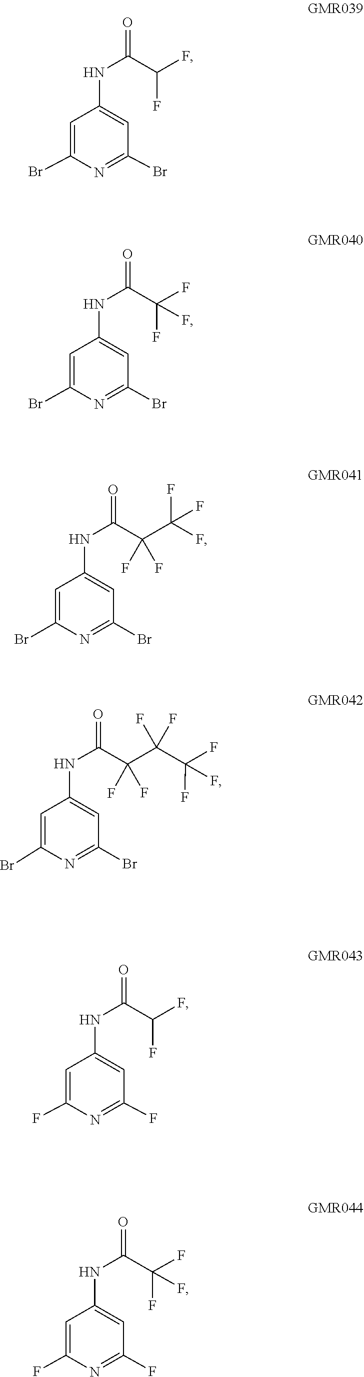 [SMILES: O=C(Nc1cc(Br)nc(Br)c1)C(F)F ; O=C(Nc1cc(Br)nc(Br)c1)C(F)(F)F ; O=C(Nc1cc(Br)nc(Br)c1)C(F)(F)C(F)(F)F ; O=C(Nc1cc(Br)nc(Br)c1)C(F)(F)C(F)(F)C(F)(F)F ; O=C(Nc1cc(F)nc(F)c1)C(F)F ; O=C(Nc1cc(F)nc(F)c1)C(F)(F)F]

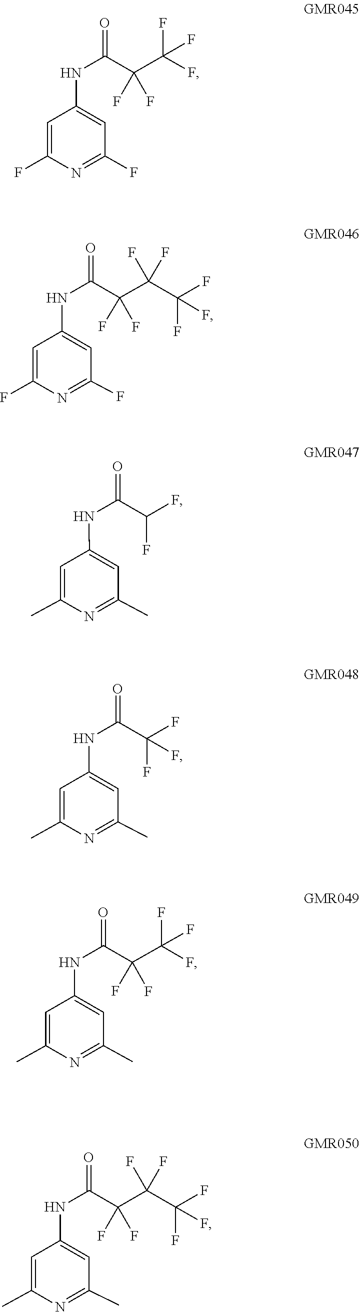 [SMILES: O=C(Nc1cc(F)nc(F)c1)C(F)(F)C(F)(F)F ; O=C(Nc1cc(F)nc(F)c1)C(F)(F)C(F)(F)C(F)(F)F ; Cc1cc(NC(=O)C(F)F)cc(C)n1 ; Cc1cc(NC(=O)C(F)(F)F)cc(C)n1 ; Cc1cc(NC(=O)C(F)(F)C(F)(F)F)cc(C)n1 ; Cc1cc(NC(=O)C(F)(F)C(F)(F)C(F)(F)F)cc(C)n1]

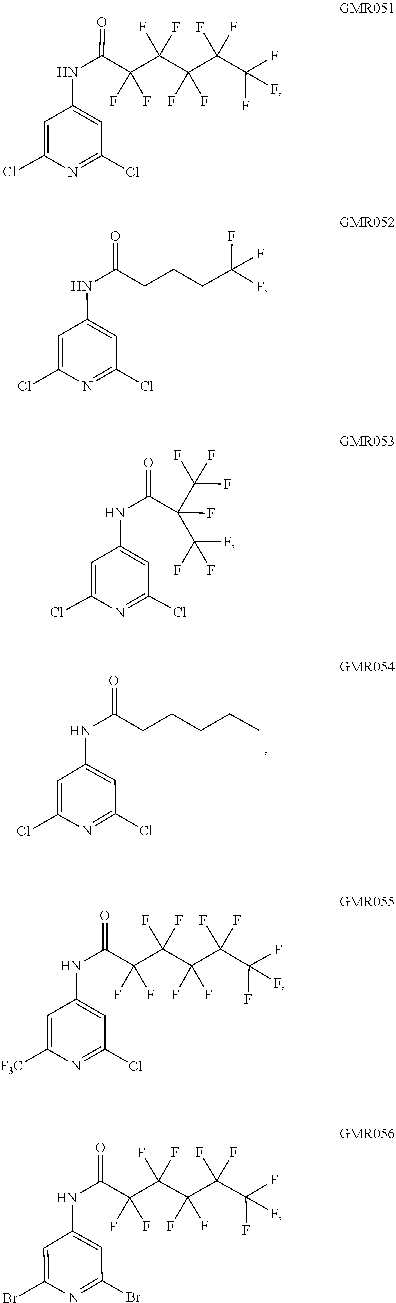 [SMILES: O=C(Nc1cc(Cl)nc(Cl)c1)C(F)(F)C(F)(F)C(F)(F)C(F)(F)C(F)(F)F ; O=C(CCCC(F)(F)F)Nc1cc(Cl)nc(Cl)c1 ; O=C(Nc1cc(Cl)nc(Cl)c1)C(F)(C(F)(F)F)C(F)(F)F ; CCCCCC(=O)Nc1cc(Cl)nc(Cl)c1 ; O=C(Nc1cc(Cl)nc(C(F)(F)F)c1)C(F)(F)C(F)(F)C(F)(F)C(F)(F)C(F)(F)F ; O=C(Nc1cc(Br)nc(Br)c1)C(F)(F)C(F)(F)C(F)(F)C(F)(F)C(F)(F)F]

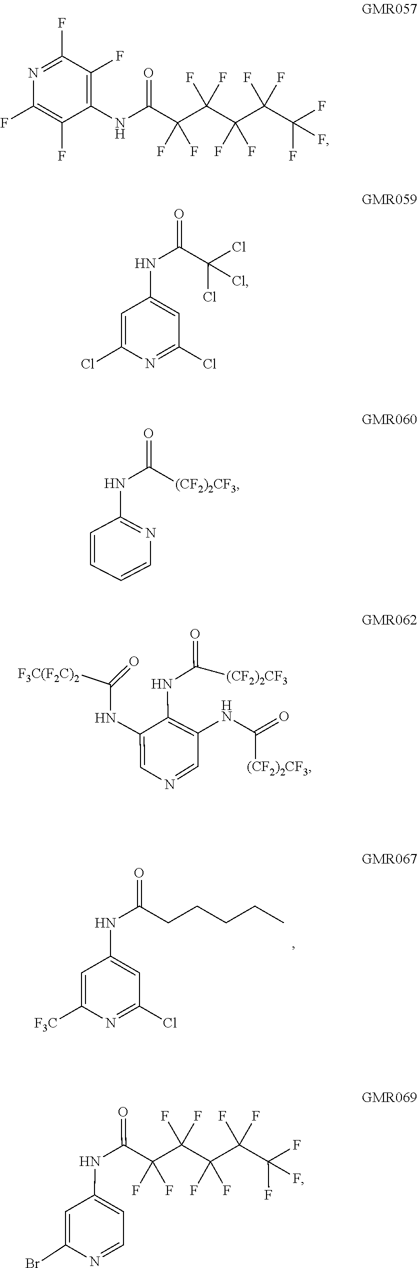

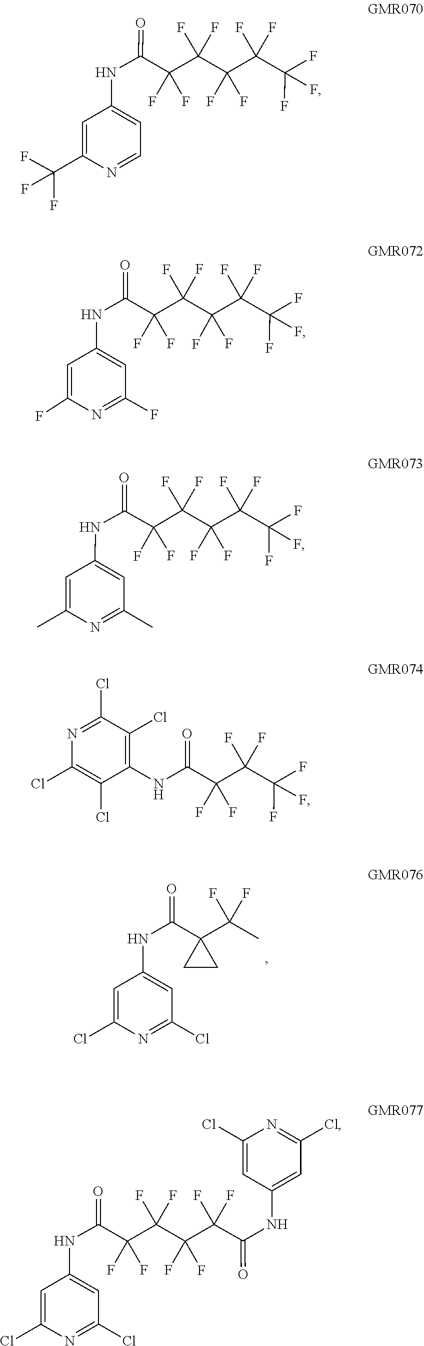 [SMILES: O=C(Nc1ccnc(C(F)(F)F)c1)C(F)(F)C(F)(F)C(F)(F)C(F)(F)C(F)(F)F ; O=C(Nc1cc(F)nc(F)c1)C(F)(F)C(F)(F)C(F)(F)C(F)(F)C(F)(F)F ; Cc1cc(NC(=O)C(F)(F)C(F)(F)C(F)(F)C(F)(F)C(F)(F)F)cc(C)n1 ; O=C(Nc1c(Cl)c(Cl)nc(Cl)c1Cl)C(F)(F)C(F)(F)C(F)(F)F ; CC(F)(F)C1(C(=O)Nc2cc(Cl)nc(Cl)c2)CC1 ; O=C(Nc1cc(Cl)nc(Cl)c1)C(F)(F)C(F)(F)C(F)(F)C(F)(F)C(=O)Nc1cc(Cl)nc(Cl)c1]

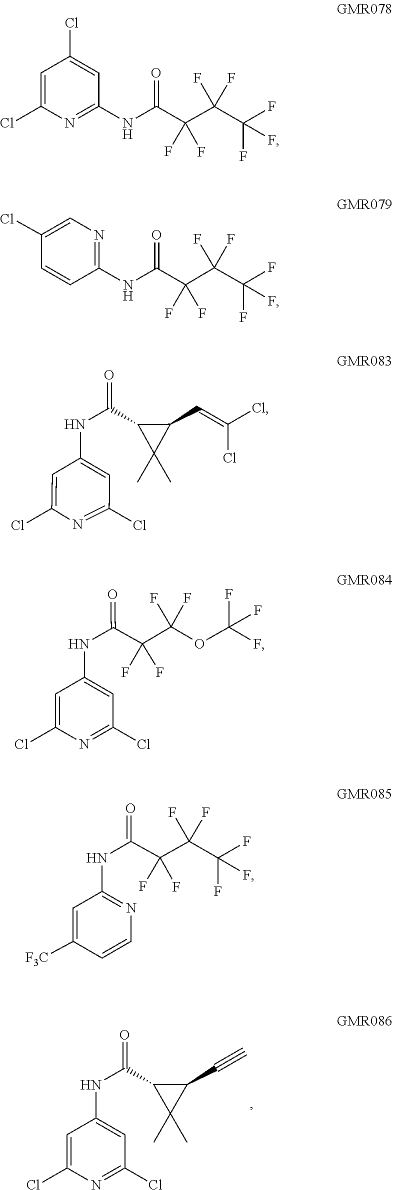

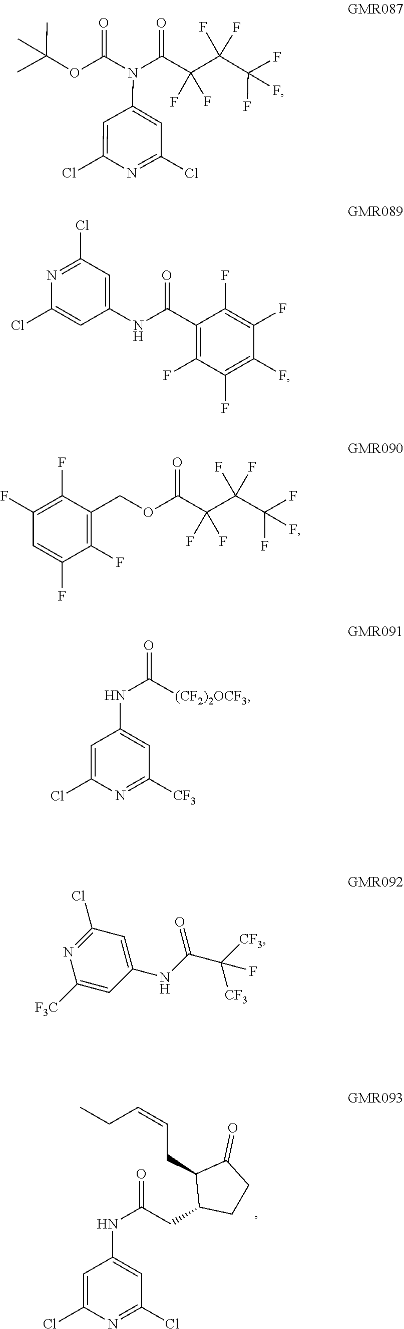

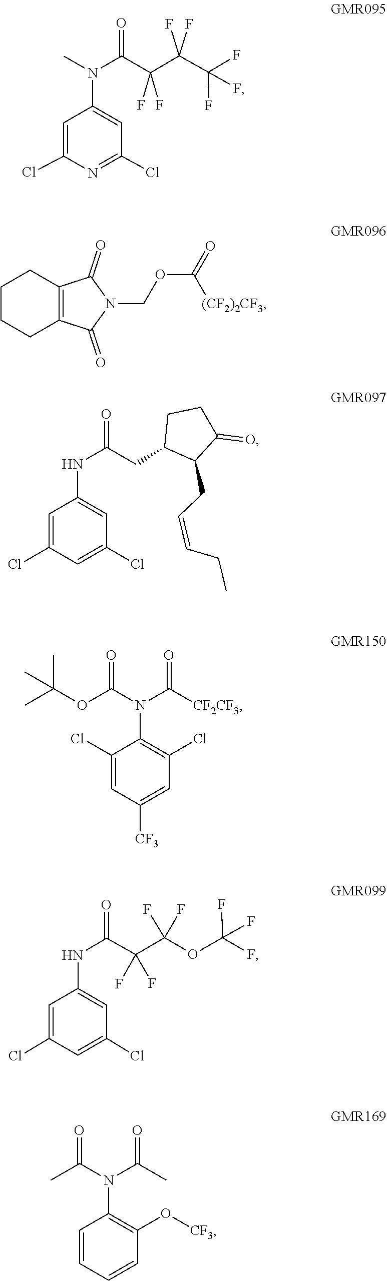

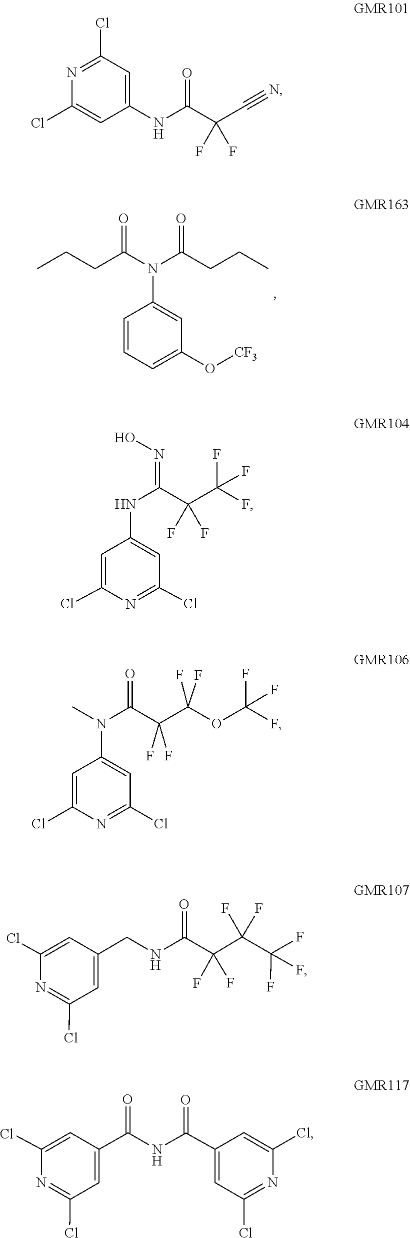 [SMILES: N#CC(F)(F)C(=O)Nc1cc(Cl)nc(Cl)c1 ; CCCC(=O)N(C(=O)CCC)c1cccc(OC(F)(F)F)c1 ; O/N=C(\Nc1cc(Cl)nc(Cl)c1)C(F)(F)C(F)(F)F ; CN(C(=O)C(F)(F)C(F)(F)OC(F)(F)F)c1cc(Cl)nc(Cl)c1 ; O=C(NCc1cc(Cl)nc(Cl)c1)C(F)(F)C(F)(F)C(F)(F)F ; O=C(NC(=O)c1cc(Cl)nc(Cl)c1)c1cc(Cl)nc(Cl)c1]

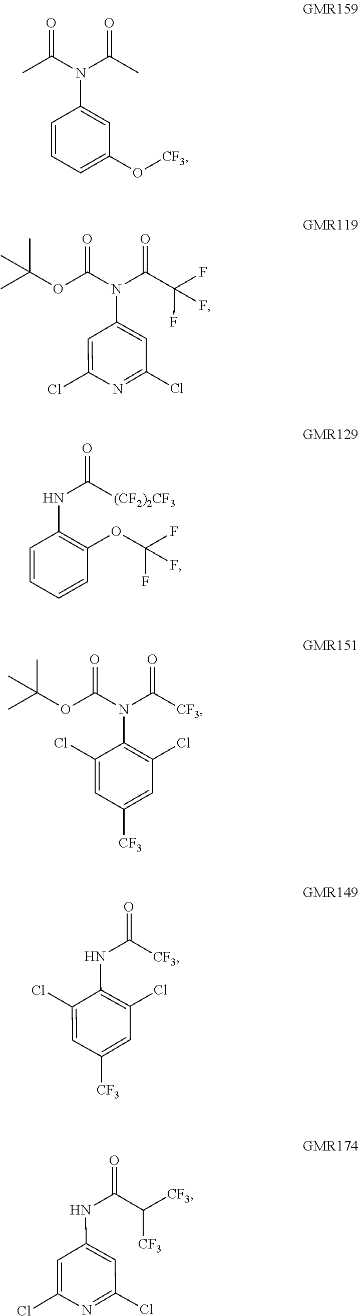

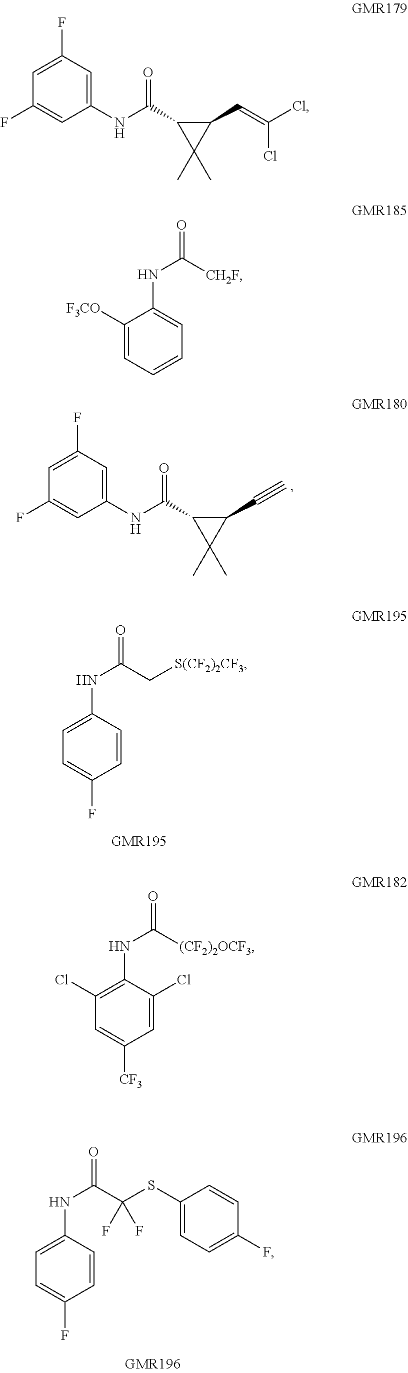

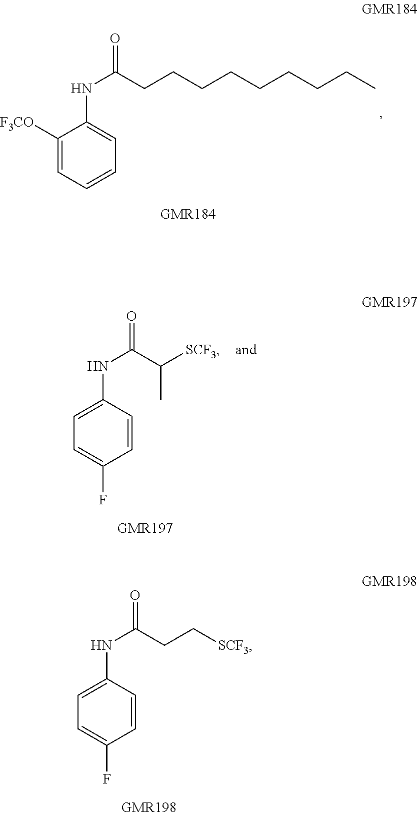

56. A composition comprising a compound of claim 55 and a carrier.

57. The composition of claim 56 for use in a method of repelling an arthropod from a locus.

58. (canceled)

59. The composition of claim 57, wherein the composition is a solution, dust, granular formulation, liquid solution, a vaporized mist or aerosol, or an emulsion.

60. (canceled)

61. The composition of claim 57, wherein said arthropod is an insect, fly, spider, butterfly, crab, mosquito, centipede, tick, millipede, or scorpion.

62-64. (canceled)

65. The composition of claim 57, where the locus is an area, an environment, or the skin of an animal.

66-69. (canceled)

70. The method of claim 1, wherein the composition further comprises:
a compound of Formula IA, IB, IC, ID, IE, IF, or IG;
an essential oil selected from the group consisting of citronella oil, Amyris oil, dill seed oil, galbanum oil, cade oil, ginger root oil, fir needle oil, guaiacwood oil, cypress oil, cinnamon bark oil, patchouli oil, cedarleaf oil, peppermint oil, lemongrass oil, orange oil, lavender oil, rosemary oil, cedarwood oil, lemon eucalyptus oil, catnip oil, geranium oil, castor oil, clove oil, soybean oil, basil oil, neem oil, vetiver oil, and tea tree oil; and/or
a second compound selected from the group consisting of a pyrethroid, a compound of Formula II′, and a compound of Formula III′: 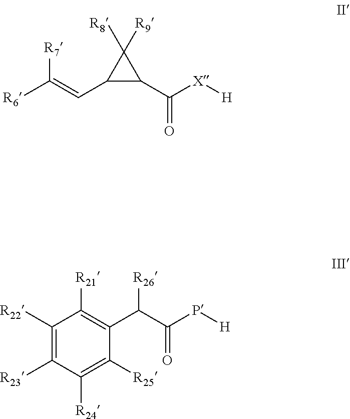
wherein,
X″ is O, NH, or S;
R_{6′} and R_{7′} are each independently selected from the group consisting of hydrogen, halo, haloalkyl, C₆-C₁₂ heteroaryl, haloaryl, C₆-C₁₂ aryl, alkenyl, alkynyl, —CO(O)CH₃, and C₁-C₃ alkyl; or
R_{6′} and R_{7′} are taken together with the carbon to which they are attached to form a C₆-C₁₂ heteroaryl, C₆-C₁₂ aryl, C₃-C₁₂ cycloalkyl, or C₃-C₁₂ heterocycloalkyl;
R_{8′} and R_{9′} are each independently selected from hydrogen, haloalkyl, alkenyl, alkynyl, and C₁-C₆ alkyl;
R_{21′}, R_{22′}, R_{23′}, R_{24′}, and R_{25′} are each independently selected from the group consisting of hydrogen, alkoxy, halo, C₁-C₆ alkyl, haloalkyl, alkenyl, alkynyl, and C₁-C₃ alkyl-alkoxy;
P′ is O, NH, or S;
R_{26′} is selected from the group consisting of hydrogen, haloalkyl, alkenyl, alkynyl, and linear or branched C₁-C₆ alkyl; and,
wherein the composition is synergistic, in admixture with a carrier.

71-112. (canceled)
